SEQUENCE LISTING (581 sequences)

SEQ ID NO: 1 - protein, 265 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 2 - protein, 257 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVSS

SEQ ID NO: 3 - protein, 257 aa, synthetic construct
EVQLLESGGGLVQAGGSLRLSCAASGYSNCAYDMTWYRQAPGKEREFVSAIHSDGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVSS

SEQ ID NO: 4 - protein, 257 aa, synthetic construct
EVQLLESGGGLVQAGGSLRLSCAASGYSNCAYDMTWYRQAPGKEREFVSAIHSSGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVSS

SEQ ID NO: 5 - protein, 265 aa, synthetic construct
EVQLQESGGGSVQPGGSLRLSCAASGYSNCAYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 6 - protein, 265 aa, synthetic construct
EVQLQESGGGLVQAGGSLRLSCAASGYSNCAYDMTWYRQAPGKEREFVSAIHSDGSTRYADSVKGRFFISQDNSKNTVYLQMNSLKPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 7 - protein, 265 aa, synthetic construct
EVQLQESGGGLVQAGGSLRLSCAASGYSNCAYDMTWYRQAPGKEREFVSAIHSDGSTRYADSVKGRFFISRDNSKNTVYLQMNSLKPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 8 - protein, 265 aa, synthetic construct
EVQLLESGGGLVQAGGSLRLSCAASGYSNCAYDMTWYRQAPGKEREFVSAIHSDGSTRYADSVKGRFFISQDNSKNTVYLQMNSLKPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 9 - protein, 265 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLKPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 10 - protein, 265 aa, synthetic construct
EVQLQESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 11 - protein, 265 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 12 - protein, 265 aa, synthetic construct
EVQLLESGGGLVQAGGSLRLSCAASGYSNCAYDMTWYRQAPGKEREFVSAIHSDGSTRYADSVKGRFFISRDNSKNTLYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 13 - protein, 265 aa, synthetic construct
EVQLLESGGGLVQAGGSLRLSCAASGYSNCAYDMTWYRQAPGKEREFVSAIHSDGSTRYADSVKGRFFISRDNSKNTLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 14 - protein, 265 aa, synthetic construct
EVQLLESGGGLVQAGGSLRLSCAASGYSNCAYAMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 15 - protein, 265 aa, synthetic construct
EVQLLESGGGLVQAGGSLRLSCAASGYSNCAYDMSWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 16 - protein, 265 aa, synthetic construct
EVQLLESGGGLVQAGGSLRLSCAASGYSNCAYDMTWYRQAPGKEREFVSAISSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 17 - protein, 265 aa, synthetic construct
EVQLLESGGGLVQAGGSLRLSCAASGYSNCAYDMTWYRQAPGKEREFVSAIHGDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 18 - protein, 265 aa, synthetic construct
EVQLLESGGGLVQAGGSLRLSCAASGYSNCAYDMTWYRQAPGKEREFVSAIHSSGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 19 - protein, 265 aa, synthetic construct
EVQLLESGGGLVQAGGSLRLSCAASGYSNCAYDMTWYRQAPGKEREFVSAIHSDGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 20 - protein, 265 aa, synthetic construct
EVQLLESGGGLVQAGGSLRLSCAASGYSNCAYAMSWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 21 - protein, 265 aa, synthetic construct
EVQLLESGGGLVQAGGSLRLSCAASGYSNCAYDMTWYRQAPGKEREFVSAISGSGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 22 - protein, 265 aa, synthetic construct
EVQLLESGGGLVQAGGSLRLSCAASGYSNCAYAMSWYRQAPGKEREFVSAISGSGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 23 - protein, 265 aa, synthetic construct
EVQLLESGGGLVQPGGSLRLSCAASGYSNCAYDMTWYRQAPGKEREFVSAIHSDGSTRYADSVKGRFFISRDNSKNTLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 24 - protein, 259 aa, synthetic construct
EVQLLESGGGSVQPGGSLRLSCAASGYSNCAYDMTWYRQAPGKEREFVSAIHSDGSTRYADSVKGRFFISRDNSKNTLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSAAS

SEQ ID NO: 25 - protein, 127 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSS

SEQ ID NO: 26 - protein, 127 aa, synthetic construct; identical to SEQ ID NO: 25

SEQ ID NO: 27 - protein, 127 aa, synthetic construct
EVQLLESGGGLVQAGGSLRLSCAASGYSNCAYDMTWYRQAPGKEREFVSAIHSDGSTYYADSVKGRFFISRDNSKNTVYLQMNSLRPEDTAVYYCKTDPLHCRAHGGSWYSVRANYWGQGTLVTVSS

SEQ ID NO: 28 - protein, 127 aa, synthetic construct
EVQLLESGGGLVQAGGSLRLSCAASGYSNCAYDMTWYRQAPGKEREFVSAIHSSGSTYYADSVKGRFFISRDNSKNTVYLQMNSLRPEDTAVYYCKTDPLHCRAHGGSWYSVRANYWGQGTLVTVSS

SEQ ID NO: 29 - protein, 127 aa, synthetic construct
EVQLQESGGGSVQPGGSLRLSCAASGYSNCAYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSS

SEQ ID NO: 30 - protein, 127 aa, synthetic construct
EVQLQESGGGLVQAGGSLRLSCAASGYSNCAYDMTWYRQAPGKEREFVSAIHSDGSTRYADSVKGRFFISQDNSKNTVYLQMNSLKPEDTAVYYCKTDPLHCRAHGGSWYSVRANYWGQGTLVTVSS

SEQ ID NO: 31 - protein, 127 aa, synthetic construct
EVQLQESGGGLVQAGGSLRLSCAASGYSNCAYDMTWYRQAPGKEREFVSAIHSDGSTRYADSVKGRFFISRDNSKNTVYLQMNSLKPEDTAVYYCKTDPLHCRAHGGSWYSVRANYWGQGTLVTVSS

SEQ ID NO: 32 - protein, 127 aa, synthetic construct
EVQLLESGGGLVQAGGSLRLSCAASGYSNCAYDMTWYRQAPGKEREFVSAIHSDGSTRYADSVKGRFFISQDNSKNTVYLQMNSLKPEDTAVYYCKTDPLHCRAHGGSWYSVRANYWGQGTLVTVSS

SEQ ID NO: 33 - protein, 127 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLKPEDTAVYYCKTDPLHCRAHGGSWYSVRANYWGQGTLVTVSS

SEQ ID NO: 34 - protein, 127 aa, synthetic construct
EVQLQESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSS

SEQ ID NO: 35 - protein, 127 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRAEDTAVYYCKTDPLHCRAHGGSWYSVRANYWGQGTLVTVSS

SEQ ID NO: 36 - protein, 126 aa, synthetic construct
VQLLESGGGLVQAGGSLRLSCAASGYSNCAYDMTWYRQAPGKEREFVSAIHSDGSTRYADSVKGRFFISRDNSKNTLYLQMNSLRPEDTAVYYCKTDPLHCRAHGGSWYSVRANYWGQGTLVTVSS

SEQ ID NO: 37 - protein, 127 aa, synthetic construct
EVQLLESGGGLVQAGGSLRLSCAASGYSNCAYDMTWYRQAPGKEREFVSAIHSDGSTRYADSVKGRFFISRDNSKNTLYLQMNSLRAEDTAVYYCKTDPLHCRAHGGSWYSVRANYWGQGTLVTVSS

SEQ ID NO: 38 - protein, 127 aa, synthetic construct
EVQLLESGGGLVQAGGSLRLSCAASGYSNCAYAMTWYRQAPGKEREFVSAIHSDGSTRYADSVKGRFFISRDNSKNTVYLQMNSLRPEDTAVYYCKTDPLHCRAHGGSWYSVRANYWGQGTLVTVSS

SEQ ID NO: 39 - protein, 127 aa, synthetic construct
EVQLLESGGGLVQAGGSLRLSCAASGYSNCAYDMSWYRQAPGKEREFVSAIHSDGSTRYADSVKGRFFISRDNSKNTVYLQMNSLRPEDTAVYYCKTDPLHCRAHGGSWYSVRANYWGQGTLVTVSS

SEQ ID NO: 40 - protein, 127 aa, synthetic construct
EVQLLESGGGLVQAGGSLRLSCAASGYSNCAYDMTWYRQAPGKEREFVSAISSDGSTRYADSVKGRFFISRDNSKNTVYLQMNSLRPEDTAVYYCKTDPLHCRAHGGSWYSVRANYWGQGTLVTVSS

SEQ ID NO: 41 - protein, 127 aa, synthetic construct
EVQLLESGGGLVQAGGSLRLSCAASGYSNCAYDMTWYRQAPGKEREFVSAIHGDGSTRYADSVKGRFFISRDNSKNTVYLQMNSLRPEDTAVYYCKTDPLHCRAHGGSWYSVRANYWGQGTLVTVSS

SEQ ID NO: 42 - protein, 127 aa, synthetic construct
EVQLLESGGGLVQAGGSLRLSCAASGYSNCAYDMTWYRQAPGKEREFVSAIHSSGSTRYADSVKGRFFISRDNSKNTVYLQMNSLRPEDTAVYYCKTDPLHCRAHGGSWYSVRANYWGQGTLVTVSS

SEQ ID NO: 43 - protein, 127 aa, synthetic construct; identical to SEQ ID NO: 27

SEQ ID NO: 44 - protein, 127 aa, synthetic construct
EVQLLESGGGLVQAGGSLRLSCAASGYSNCAYAMSWYRQAPGKEREFVSAIHSDGSTRYADSVKGRFFISRDNSKNTVYLQMNSLRPEDTAVYYCKTDPLHCRAHGGSWYSVRANYWGQGTLVTVSS

SEQ ID NO: 45 - protein, 127 aa, synthetic construct
EVQLLESGGGLVQAGGSLRLSCAASGYSNCAYDMTWYRQAPGKEREFVSAISGSGSTYYADSVKGRFFISRDNSKNTVYLQMNSLRPEDTAVYYCKTDPLHCRAHGGSWYSVRANYWGQGTLVTVSS

SEQ ID NO: 46 - protein, 127 aa, synthetic construct
EVQLLESGGGLVQAGGSLRLSCAASGYSNCAYAMSWYRQAPGKEREFVSAISGSGSTYYADSVKGRFFISRDNSKNTVYLQMNSLRPEDTAVYYCKTDPLHCRAHGGSWYSVRANYWGQGTLVTVSS

SEQ ID NO: 47 - protein, 127 aa, synthetic construct
EVQLLESGGGLVQPGGSLRLSCAASGYSNCAYDMTWYRQAPGKEREFVSAIHSDGSTRYADSVKGRFFISRDNSKNTLYLQMNSLRAEDTAVYYCKTDPLHCRAHGGSWYSVRANYWGQGTLVTVSS

SEQ ID NO: 48 - protein, 127 aa, synthetic construct
EVQLLESGGGSVQPGGSLRLSCAASGYSNCAYDMTWYRQAPGKEREFVSAIHSDGSTRYADSVKGRFFISRDNSKNTLYLQMNSLRAEDTAVYYCKTDPLHCRAHGGSWYSVRANYWGQGTLVTVSS

SEQ ID NO: 49 - protein, 126 aa, synthetic construct
EVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 50 - protein, 126 aa, synthetic construct
EVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVSS

SEQ ID NO: 51 - protein, 126 aa, synthetic construct
EVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSA

SEQ ID NO: 52-120 - skipped (69 entries)

SEQ ID NO: 121 - protein, 257 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLGTVSS

SEQ ID NO: 122 - protein, 257 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLLTVSS

SEQ ID NO: 123 - protein, 259 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLLTVSSAA

SEQ ID NO: 124 - protein, 257 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSA

SEQ ID NO: 125 - protein, 259 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSAAA

SEQ ID NO: 126 - protein, 257 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVCS

SEQ ID NO: 127 - protein, 258 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVCSA

SEQ ID NO: 128 - protein, 259 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVCSAA

SEQ ID NO: 129 - protein, 257 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSC

SEQ ID NO: 130 - protein, 259 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSCAA

SEQ ID NO: 131 - protein, 257 aa, synthetic construct
EVQLLESGGGSVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGSVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLLTVSS

SEQ ID NO: 132 - protein, 258 aa, synthetic construct
EVQLLESGGGSVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGSVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLLTVSSA

SEQ ID NO: 133 - protein, 259 aa, synthetic construct
EVQLLESGGGSVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGSVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLLTVSSAA

SEQ ID NO: 134 - protein, 257 aa, synthetic construct
EVQLLESGGGSVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGSVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVTT

SEQ ID NO: 135 - protein, 259 aa, synthetic construct
EVQLLESGGGSVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGSVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVTTAA

SEQ ID NO: 136-137 - skipped (2 entries)

SEQ ID NO: 138 - protein, 257 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTLTVSS

SEQ ID NO: 139 - protein, 258 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTLTVSSA

SEQ ID NO: 140 - protein, 259 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTLTVSSAA

SEQ ID NO: 141 - protein, 260 aa, synthetic construct
EVQLLESGGGLVQAGGSLRLSCAASGYSNCAYDMTWYRQAPGKEREFVSAIHSDHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTGTVSS

SEQ ID NO: 142-143 - skipped (2 entries)

SEQ ID NO: 144 - protein, 257 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVSA

SEQ ID NO: 145 - protein, 259 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVSAAA

SEQ ID NO: 146 - protein, 257 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVCS

SEQ ID NO: 147 - protein, 258 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVCSA

SEQ ID NO: 148 - protein, 259 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVCSAA

SEQ ID NO: 149 - protein, 257 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVSC

SEQ ID NO: 150 - protein, 259 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVSCAA

SEQ ID NO: 151 - protein, 257 aa, synthetic construct; identical to SEQ ID NO: 131

SEQ ID NO: 152 - protein, 258 aa, synthetic construct; identical to SEQ ID NO: 132

SEQ ID NO: 153 - protein, 259 aa, synthetic construct; identical to SEQ ID NO: 133

SEQ ID NO: 154 - protein, 257 aa, synthetic construct; identical to SEQ ID NO: 134

SEQ ID NO: 155 - protein, 259 aa, synthetic construct; identical to SEQ ID NO: 135

SEQ ID NO: 156-157 - skipped (2 entries)

SEQ ID NO: 158 - protein, 257 aa, synthetic construct; identical to SEQ ID NO: 138

SEQ ID NO: 159 - protein, 258 aa, synthetic construct; identical to SEQ ID NO: 139

SEQ ID NO: 160 - protein, 259 aa, synthetic construct; identical to SEQ ID NO: 140

SEQ ID NO: 161 - protein, 257 aa, synthetic construct
EVQLLESGGGLVQAGGSLRLSCAASGYSNCAYDMTWYRQAPGKEREFVSAIHSDGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTGTVSS

SEQ ID NO: 162 - protein, 257 aa, synthetic construct
EVQLLESGGGLVQAGGSLRLSCAASGYSNCAYDMTWYRQAPGKEREFVSAIHSDGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTLTVSS

SEQ ID NO: 163 - protein, 259 aa, synthetic construct
EVQLLESGGGLVQAGGSLRLSCAASGYSNCAYDMTWYRQAPGKEREFVSAIHSDGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTLTVSSAA

SEQ ID NO: 164 - protein, 257 aa, synthetic construct
EVQLLESGGGLVQAGGSLRLSCAASGYSNCAYDMTWYRQAPGKEREFVSAIHSDGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVSA

SEQ ID NO: 165 - protein, 259 aa, synthetic construct
EVQLLESGGGLVQAGGSLRLSCAASGYSNCAYDMTWYRQAPGKEREFVSAIHSDGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVSAAA

SEQ ID NO: 166 - protein, 257 aa, synthetic construct
EVQLLESGGGLVQAGGSLRLSCAASGYSNCAYDMTWYRQAPGKEREFVSAIHSDGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVCS

SEQ ID NO: 167 - protein, 258 aa, synthetic construct
EVQLLESGGGLVQAGGSLRLSCAASGYSNCAYDMTWYRQAPGKEREFVSAIHSDGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVCSA

SEQ ID NO: 168 - protein, 259 aa, synthetic construct
EVQLLESGGGLVQAGGSLRLSCAASGYSNCAYDMTWYRQAPGKEREFVSAIHSDGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVCSAA

SEQ ID NO: 169 - protein, 257 aa, synthetic construct
EVQLLESGGGLVQAGGSLRLSCAASGYSNCAYDMTWYRQAPGKEREFVSAIHSDGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVSC

SEQ ID NO: 170 - protein, 259 aa, synthetic construct
EVQLLESGGGLVQAGGSLRLSCAASGYSNCAYDMTWYRQAPGKEREFVSAIHSDGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVSCAA

SEQ ID NO: 171 - protein, 257 aa, synthetic construct
EVQLLESGGGSVQAGGSLRLSCAASGYSNCAYDMTWYRQAPGKEREFVSAIHSDGSTYYADSVKGRFFISRDNSKNTVYLQMNSLRPEDTAVYYCKTDPLHCRAHGGSWYSVRANYWGQGTLVTVSSGGGSEVQLVESGGGSVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLLTVSS

SEQ ID NO: 172 - protein, 258 aa, synthetic construct
EVQLLESGGGSVQAGGSLRLSCAASGYSNCAYDMTWYRQAPGKEREFVSAIHSDGSTYYADSVKGRFFISRDNSKNTVYLQMNSLRPEDTAVYYCKTDPLHCRAHGGSWYSVRANYWGQGTLVTVSSGGGSEVQLVESGGGSVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLLTVSSA

SEQ ID NO: 173 - protein, 259 aa, synthetic construct
EVQLLESGGGSVQAGGSLRLSCAASGYSNCAYDMTWYRQAPGKEREFVSAIHSDGSTYYADSVKGRFFISRDNSKNTVYLQMNSLRPEDTAVYYCKTDPLHCRAHGGSWYSVRANYWGQGTLVTVSSGGGSEVQLVESGGGSVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLLTVSSAA

SEQ ID NO: 174 - protein, 257 aa, synthetic construct
EVQLLESGGGSVQAGGSLRLSCAASGYSNCAYDMTWYRQAPGKEREFVSAIHSDGSTYYADSVKGRFFISRDNSKNTVYLQMNSLRPEDTAVYYCKTDPLHCRAHGGSWYSVRANYWGQGTLVTVSSGGGSEVQLVESGGGSVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVTT

SEQ ID NO: 175 - protein, 259 aa, synthetic construct
EVQLLESGGGSVQAGGSLRLSCAASGYSNCAYDMTWYRQAPGKEREFVSAIHSDGSTYYADSVKGRFFISRDNSKNTVYLQMNSLRPEDTAVYYCKTDPLHCRAHGGSWYSVRANYWGQGTLVTVSSGGGSEVQLVESGGGSVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVTTAA

SEQ ID NO: 176-178 - skipped (3 entries)

SEQ ID NO: 179 - protein, 258 aa, synthetic construct
EVQLLESGGGLVQAGGSLRLSCAASGYSNCAYDMTWYRQAPGKEREFVSAIHSDGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTLTVSSA

SEQ ID NO: 180 - skipped

SEQ ID NO: 181 - protein, 257 aa, synthetic construct
EVQLLESGGGLVQAGGSLRLSCAASGYSNCAYDMTWYRQAPGKEREFVSAIHSSGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTGTVSS

SEQ ID NO: 182 - protein, 257 aa, synthetic construct
EVQLLESGGGLVQAGGSLRLSCAASGYSNCAYDMTWYRQAPGKEREFVSAIHSSGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTLTVSS

SEQ ID NO: 183 - protein, 259 aa, synthetic construct
EVQLLESGGGLVQAGGSLRLSCAASGYSNCAYDMTWYRQAPGKEREFVSAIHSSGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTLTVSSAA

SEQ ID NO: 184 - protein, 257 aa, synthetic construct
EVQLLESGGGLVQAGGSLRLSCAASGYSNCAYDMTWYRQAPGKEREFVSAIHSSGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVSA

SEQ ID NO: 185 - protein, 259 aa, synthetic construct
EVQLLESGGGLVQAGGSLRLSCAASGYSNCAYDMTWYRQAPGKEREFVSAIHSSGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVSAAA

SEQ ID NO: 186 - protein, 257 aa, synthetic construct
EVQLLESGGGLVQAGGSLRLSCAASGYSNCAYDMTWYRQAPGKEREFVSAIHSSGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVCS

SEQ ID NO: 187 - protein, 258 aa, synthetic construct
EVQLLESGGGLVQAGGSLRLSCAASGYSNCAYDMTWYRQAPGKEREFVSAIHSSGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVCSA

SEQ ID NO: 188 - protein, 259 aa, synthetic construct
EVQLLESGGGLVQAGGSLRLSCAASGYSNCAYDMTWYRQAPGKEREFVSAIHSSGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVCSAA

SEQ ID NO: 189 - protein, 257 aa, synthetic construct
EVQLLESGGGLVQAGGSLRLSCAASGYSNCAYDMTWYRQAPGKEREFVSAIHSSGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVSC

SEQ ID NO: 190 - protein, 259 aa, synthetic construct
EVQLLESGGGLVQAGGSLRLSCAASGYSNCAYDMTWYRQAPGKEREFVSAIHSSGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVSCAA

SEQ ID NO: 191 - protein, 257 aa, synthetic construct
EVQLLESGGGSVQAGGSLRLSCAASGYSNCAYDMTWYRQAPGKEREFVSAIHSSGSTYYADSVKGRFFISRDNSKNTVYLQMNSLRPEDTAVYYCKTDPLHCRAHGGSWYSVRANYWGQGTLVTVSSGGGSEVQLVESGGGSVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLLTVSS

SEQ ID NO: 192 - protein, 258 aa, synthetic construct
EVQLLESGGGSVQAGGSLRLSCAASGYSNCAYDMTWYRQAPGKEREFVSAIHSSGSTYYADSVKGRFFISRDNSKNTVYLQMNSLRPEDTAVYYCKTDPLHCRAHGGSWYSVRANYWGQGTLVTVSSGGGSEVQLVESGGGSVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLLTVSSA

SEQ ID NO: 193 - protein, 259 aa, synthetic construct
EVQLLESGGGSVQAGGSLRLSCAASGYSNCAYDMTWYRQAPGKEREFVSAIHSSGSTYYADSVKGRFFISRDNSKNTVYLQMNSLRPEDTAVYYCKTDPLHCRAHGGSWYSVRANYWGQGTLVTVSSGGGSEVQLVESGGGSVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLLTVSSAA

SEQ ID NO: 194 - protein, 257 aa, synthetic construct
EVQLLESGGGSVQAGGSLRLSCAASGYSNCAYDMTWYRQAPGKEREFVSAIHSSGSTYYADSVKGRFFISRDNSKNTVYLQMNSLRPEDTAVYYCKTDPLHCRAHGGSWYSVRANYWGQGTLVTVSSGGGSEVQLVESGGGSVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVTT

SEQ ID NO: 195 - protein, 259 aa, synthetic construct
EVQLLESGGGSVQAGGSLRLSCAASGYSNCAYDMTWYRQAPGKEREFVSAIHSSGSTYYADSVKGRFFISRDNSKNTVYLQMNSLRPEDTAVYYCKTDPLHCRAHGGSWYSVRANYWGQGTLVTVSSGGGSEVQLVESGGGSVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVTTAA

SEQ ID NO: 196-198 - skipped (3 entries)

SEQ ID NO: 199 - protein, 258 aa, synthetic construct
EVQLLESGGGLVQAGGSLRLSCAASGYSNCAYDMTWYRQAPGKEREFVSAIHSSGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTLTVSSA

SEQ ID NO: 200 - DNA, 774 bp, synthetic construct
  source 1..774: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccattccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgct

SEQ ID NO: 201 - DNA, 771 bp, synthetic construct
  source 1..771: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccattccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccacggtaactgtctcgtct

SEQ ID NO: 202 - DNA, 771 bp, synthetic construct
  source 1..771: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccattccgatgggagcacatattacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccacggtaactgtctcgtct

SEQ ID NO: 203 - DNA, 771 bp, synthetic construct
  source 1..771: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccattcctctgggagcacatattacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccacggtaactgtctcgtct

SEQ ID NO: 204 - DNA, 774 bp, synthetic construct
  source 1..774: mol_type = other DNA
gaagtccagctccaggaatcaggaggtggctctgtgcaacccggtggctccctgcgcctcagctgtgccgcttctggatattctaactgcgcttacgacatgacttggtataggcaggcaccgggcaaggagcgcgagttcgtttctgccattcactctgatggatctacccgctatgccgatagcgtgaagggtcgctttttcatctcccaggataacgctaaaaatactgtgtaccttcagatgaacagcctgaaacctgaagacaccgccatgtactattgtaagacagacccgttgcattgcagagcccacggaggctcatggtattccgtccgggcgaactactggggccagggcactcaggtgaccgtatcctctggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgct

SEQ ID NO: 205 - DNA, 774 bp, synthetic construct
  source 1..774: mol_type = other DNA
gaggttcaactccaggagagcggcgggggcctggtgcaagctgggggcagtctgcgcttgagctgcgctgcatccggctattccaactgtgcctatgacatgacgtggtataggcaggcccctgggaaggagcgtgagttcgtgagtgctatccacagcgacggctccactagatacgcggactccgttaaaggaagatttttcatcagtcaggacaacagcaagaataccgtgtatttgcagatgaatagtctgaagcccgaggacacagcagtctactattgtaagactgacccactgcactgtcgtgcgcatggaggctcttggtactccgtgagagccaattattgggggcagggcacccttgttaccgtatcctccggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgct

SEQ ID NO: 206 - DNA, 774 bp, synthetic construct
  source 1..774: mol_type = other DNA
gaggtgcagctccaggagagcggcggtggcctcgtccaagccgggggctccctgcgtttgtcttgcgcggccagcggatattccaactgtgcgtatgacatgacatggtatcgccaggctccaggcaaggagcgtgagtttgtgtctgcaatccactctgatggctccacacgctacgccgacagcgtgaagggccgcttctttatctccagagataacagcaagaacactgtctatctccagatgaacagcctgaagcccgaagacaccgccgtttattactgtaagaccgaccctctgcattgcagagcccacggcggttcctggtattccgtccgcgctaactattggggacaggggacccttgtgacagtcagctccggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgct

SEQ ID NO: 207 - DNA, 774 bp, synthetic construct
  source 1..774: mol_type = other DNA
gaggtgcagttgctggagtctggtgggggcctcgtgcaggccggaggctccctgcgcctgtcctgcgctgcctctggatactctaattgcgcatacgacatgacatggtatcggcaggcacccggaaaggagcgcgagttcgtatcagcaatccatagtgacggttctactcgctatgcagactctgtgaaaggacgcttctttattagccaggacaactctaaaaacactgtctatctccaaatgaactccctcaagccagaagatactgccgtgtattactgcaagaccgacccactgcattgtcgtgcgcatggagggtcttggtattcagtacgtgcaaactactggggacagggcaccctggtcactgtgtcctctggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgct

SEQ ID NO: 208 - DNA, 774 bp, synthetic construct
  source 1..774: mol_type = other DNA
gaggtacagctcttggagtccggcggtggcttggtgcaggctggaggtagcttgcgcctgtcctgcgccgcttccggctacagcaactgcgcctacgacatgacgtggtatcggcaggctccaggtaaggagcgcgagttcgtcagtgccattcactccgacgggagcactagatatgccgactctgtcaagggacgttttttcattagtcgcgacaactctaagaacaccgtgtaccttcagatgaacagcctgaagcccgaggacactgccgtgtactattgcaagaccgaccccctccattgcagggctcatgggggctcttggtattcagtgagagccaactattggggacagggtacgctggtgaccgtgtcatctggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgct

SEQ ID NO: 209 - DNA, 774 bp, synthetic construct; identical to SEQ ID NO: 208
  source 1..774: mol_type = other DNA

SEQ ID NO: 210 - DNA, 774 bp, synthetic construct
  source 1..774: mol_type = other DNA
gaggttcagttgctggaatctgggggcggtctggtccaggctgggggctctctgaggctgagctgcgccgcatctggctactccaactgcgcctatgatatgacttggtacaggcaggctcccggcaaggagcgcgagttcgtgagcgccattcactctgatggttccacaaggtatgctgatagtgttaaaggacgctttttcattagccgggacaatagcaaaaacacagtctatctccagatgaactccctccgggcggaggataccgctgtctattactgcaagactgaccctttgcactgtagagcacacggaggcagctggtattctgtgagagccaactattggggacaaggaaccctcgtcacggtgtctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgct

SEQ ID NO: 211 - DNA, 774 bp, synthetic construct
  source 1..774: mol_type = other DNA
gaggtgcagctcctggagtctggcggtggcctggtgcaagctggcggttcccttcgtctgtcctgcgcagccagtggctattctaactgtgcttacgacatgacatggtatcgtcaggcaccggggaaggagcgcgagttcgtatcagctatccactctgacggcagtaccagatatgccgacagcgtcaaagggcgttttttcatctcccgcgacaactccaaaaataccctgtatcttcagatgaactccctcaggcctgaggacactgccgtctattactgcaagactgaccccctccactgtcgcgctcatgggggtagttggtactctgtgcgtgctaattactgggggcagggcaccctggtgaccgttagctcaggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgct

SEQ ID NO: 212 - DNA, 774 bp, synthetic construct
  source 1..774: mol_type = other DNA
gaggtgcagttgctggagagcgggggcgggctcgtgcaggcgggaggcagcttgcgcttgtcatgtgctgccagcggttatagcaactgcgcctacgacatgacatggtatcgccaggctcccggtaaagagagggagtttgtatccgcgatccacagtgacggtagcacccgctacgcggattccgtgaaaggacgtttctttatttctagggataatagcaagaatacactgtacttgcaaatgaactccctccgcgccgaggacactgctgtgtattactgcaaaactgatccactgcactgtcgcgctcacgggggttcttggtattcagtacgcgccaactactggggccagggtactctcgtgacggtgagcagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgct

SEQ ID NO: 213 - DNA, 774 bp, synthetic construct
  source 1..774: mol_type = other DNA
gaggtgcagctcctggaatctggcgggggcctggtccaggctggcggtagcctccgccttagttgtgctgccagcggttacagcaactgcgcttacgccatgacgtggtatcgccaggccccagggaaggagagggagttcgtcagcgctatccattccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgct

SEQ ID NO: 214 - DNA, 774 bp, synthetic construct; identical to SEQ ID NO: 213
  source 1..774: mol_type = other DNA

SEQ ID NO: 215 - DNA, 774 bp, synthetic construct
  source 1..774: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatctcttccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgct

SEQ ID NO: 216 - DNA, 772 bp, synthetic construct
  source 1..772: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatctcttccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctg

SEQ ID NO: 217 - DNA, 772 bp, synthetic construct
  source 1..772: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccattccagtgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctg

SEQ ID NO: 218 - DNA, 774 bp, synthetic construct
  source 1..774: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccattccgatgggagcacatactacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgct

SEQ ID NO: 219 - DNA, 774 bp, synthetic construct
  source 1..774: mol_type = other DNA
gaggtgcagctcctggaatctggcgggggcctggtccaggctggcggtagcctccgccttagttgtgctgccagcggttacagcaactgcgcttacgccatgtcgtggtatcgccaggccccagggaaggagagggagttcgtcagcgctatccattccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgct

SEQ ID NO: 220 - DNA, 774 bp, synthetic construct
  source 1..774: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatctctggcagtgggagcacatactacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgct

SEQ ID NO: 221 - DNA, 774 bp, synthetic construct
  source 1..774: mol_type = other DNA
gaggtgcagctcctggaatctggcgggggcctggtccaggctggcggtagcctccgccttagttgtgctgccagcggttacagcaactgcgcttacgccatgtcgtggtatcgccaggccccagggaaggagagggagttcgtcagcgctatctctggcagtgggagcacatactacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgct

SEQ ID NO: 222 - DNA, 774 bp, synthetic construct
  source 1..774: mol_type = other DNA
gaggtccagttgcaggagtccggtggcgggttggtccaagcaggcggaagcctccgccttagttgtgcggcctctggctactctaactgcgcctatgatatgacgtggtatcgccaagctccaggcaaagaaagagagttcgtcagcgccattcactctgatggctccactaggtacgcagacagcgtcaagggtcgttttttcatctcccgcgataactccaagaacaccgtgtacctccaaatgaactccctgagaccggaggacactgctgtgtactattgcaagaccgatcctttgcactgtagggcgcatggaggcagttggtactccgttagagccaactactgggggcaaggaaccctggtcactgtcagctccggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgct

SEQ ID NO: 223 - DNA, 774 bp, synthetic construct
  source 1..774: mol_type = other DNA
gaagtgcagctcctggagagcggcggtggctcggtgcagccaggcggttctctgcgcttgtcctgtgctgcctccggctactcaaactgtgcttacgatatgacttggtatcgccaggcccctggtaaggagcgcgagttcgtctctgccattcattctgacggctcaacccgttatgcagactccgttaaggggcgttttttcatttcccgcgacaactccaagaacaccctgtaccttcagatgaactccctccgcgccgaggataccgccgtatactattgcaagaccgaccccctgcattgcagggctcacggcgggtcctggtactccgtgcgtgccaactattggggacaggggactctggtcacagtctcgagtggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcggcagct

SEQ ID NO: 224 - protein, 9 aa, synthetic construct
YSNCAYDMT

SEQ ID NO: 225 - protein, 9 aa, synthetic construct
YSNCAYAMT

SEQ ID NO: 226 - protein, 9 aa, synthetic construct
YSNCAYDMS

SEQ ID NO: 227 - protein, 9 aa, synthetic construct
YSNCAYAMS

SEQ ID NO: 228 - protein, 8 aa, synthetic construct
YSNCAYDM

SEQ ID NO: 229 - protein, 16 aa, synthetic construct
AIHSDGSTRYADSVKG

SEQ ID NO: 230 - protein, 16 aa, synthetic construct
AIHSDGSTYYADSVKG

SEQ ID NO: 231 - protein, 16 aa, synthetic construct
AIHSSGSTYYADSVKG

SEQ ID NO: 232 - protein, 16 aa, synthetic construct
AISSDGSTRYADSVKG

SEQ ID NO: 233 - protein, 16 aa, synthetic construct
AIHGDGSTRYADSVKG

SEQ ID NO: 234 - protein, 16 aa, synthetic construct
AIHSSGSTRYADSVKG

SEQ ID NO: 235 - protein, 16 aa, synthetic construct
AISGSGSTYYADSVKG

SEQ ID NO: 236 - protein, 21 aa, synthetic construct
KTDPLHCRAHGGSWYSVRANY

SEQ ID NO: 237-295 - skipped (59 entries)

SEQ ID NO: 296 - protein, 9 aa, synthetic construct
YTHSSYCMG

SEQ ID NO: 297 - protein, 16 aa, synthetic construct
AIDVDGSTTYADSVKG

SEQ ID NO: 298 - protein, 20 aa, synthetic construct
AAEFADCSSNYFLPPGAVRY

SEQ ID NO: 299-367 - skipped (69 entries)

SEQ ID NO: 368 - DNA, 381 bp, synthetic construct
  source 1..381: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccattccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagc

SEQ ID NO: 369 - DNA, 382 bp, synthetic construct
  source 1..382: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccattccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcg

SEQ ID NO: 370 - DNA, 381 bp, synthetic construct
  source 1..381: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccattccgatgggagcacatactacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagc

SEQ ID NO: 371 - DNA, 381 bp, synthetic construct
  source 1..381: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccattcctctgggagcacatattacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagc

SEQ ID NO: 372 - DNA, 381 bp, synthetic construct
  source 1..381: mol_type = other DNA
gaagtccagctccaggaatcaggaggtggctctgtgcaacccggtggctccctgcgcctcagctgtgccgcttctggatattctaactgcgcttacgacatgacttggtataggcaggcaccgggcaaggagcgcgagttcgtttctgccattcactctgatggatctacccgctatgccgatagcgtgaagggtcgctttttcatctcccaggataacgctaaaaatactgtgtaccttcagatgaacagcctgaaacctgaagacaccgccatgtactattgtaagacagacccgttgcattgcagagcccacggaggctcatggtattccgtccgggcgaactactggggccagggcactcaggtgaccgtatcctct

SEQ ID NO: 373 - DNA, 381 bp, synthetic construct
  source 1..381: mol_type = other DNA
gaggttcaactccaggagagcggcgggggcctggtgcaagctgggggcagtctgcgcttgagctgcgctgcatccggctattccaactgtgcctatgacatgacgtggtataggcaggcccctgggaaggagcgtgagttcgtgagtgctatccacagcgacggctccactagatacgcggactccgttaaaggaagatttttcatcagtcaggacaacagcaagaataccgtgtatttgcagatgaatagtctgaagcccgaggacacagcagtctactattgtaagactgacccactgcactgtcgtgcgcatggaggctcttggtactccgtgagagccaattattgggggcagggcacccttgttaccgtatcctcc

SEQ ID NO: 374 - DNA, 381 bp, synthetic construct
  source 1..381: mol_type = other DNA
gaggtgcagctccaggagagcggcggtggcctcgtccaagccgggggctccctgcgtttgtcttgcgcggccagcggatattccaactgtgcgtatgacatgacatggtatcgccaggctccaggcaaggagcgtgagtttgtgtctgcaatccactctgatggctccacacgctacgccgacagcgtgaagggccgcttctttatctccagagataacagcaagaacactgtctatctccagatgaacagcctgaagcccgaagacaccgccgtttattactgtaagaccgaccctctgcattgcagagcccacggcggttcctggtattccgtccgcgctaactattggggacaggggacccttgtgacagtcagctcc

SEQ ID NO: 375 - DNA, 381 bp, synthetic construct
  source 1..381: mol_type = other DNA
gaggtgcagttgctggagtctggtgggggcctcgtgcaggccggaggctccctgcgcctgtcctgcgctgcctctggatactctaattgcgcatacgacatgacatggtatcggcaggcacccggaaaggagcgcgagttcgtatcagcaatccatagtgacggttctactcgctatgcagactctgtgaaaggacgcttctttattagccaggacaactctaaaaacactgtctatctccaaatgaactccctcaagccagaagatactgccgtgtattactgcaagaccgacccactgcattgtcgtgcgcatggagggtcttggtattcagtacgtgcaaactactggggacagggcaccctggtcactgtgtcctct

SEQ ID NO: 376 - DNA, 381 bp, synthetic construct
  source 1..381: mol_type = other DNA
gaggtacagctcttggagtccggcggtggcttggtgcaggctggaggtagcttgcgcctgtcctgcgccgcttccggctacagcaactgcgcctacgacatgacgtggtatcggcaggctccaggtaaggagcgcgagttcgtcagtgccattcactccgacgggagcactagatatgccgactctgtcaagggacgttttttcattagtcgcgacaactctaagaacaccgtgtaccttcagatgaacagcctgaagcccgaggacactgccgtgtactattgcaagaccgaccccctccattgcagggctcatgggggctcttggtattcagtgagagccaactattggggacagggtacgctggtgaccgtgtcatct

SEQ ID NO: 377 - DNA, 381 bp, synthetic construct
  source 1..381: mol_type = other DNA
gaggtccagttgcaggagtccggtggcgggttggtccaagcaggcggaagcctccgccttagttgtgcggcctctggctactctaactgcgcctatgatatgacgtggtatcgccaagctccaggcaaagaaagagagttcgtcagcgccattcactctgatggctccactaggtacgcagacagcgtcaagggtcgttttttcatctcccgcgataactccaagaacaccgtgtacctccaaatgaactccctgagaccggaggacactgctgtgtactattgcaagaccgatcctttgcactgtagggcgcatggaggcagttggtactccgttagagccaactactgggggcaaggaaccctggtcactgtcagctcc

SEQ ID NO: 378 - DNA, 381 bp, synthetic construct
  source 1..381: mol_type = other DNA
gaggttcagttgctggaatctgggggcggtctggtccaggctgggggctctctgaggctgagctgcgccgcatctggctactccaactgcgcctatgatatgacttggtacaggcaggctcccggcaaggagcgcgagttcgtgagcgccattcactctgatggttccacaaggtatgctgatagtgttaaaggacgctttttcattagccgggacaatagcaaaaacacagtctatctccagatgaactccctccgggcggaggataccgctgtctattactgcaagactgaccctttgcactgtagagcacacggaggcagctggtattctgtgagagccaactattggggacaaggaaccctcgtcacggtgtctagc

SEQ ID NO: 379 - DNA, 381 bp, synthetic construct
  source 1..381: mol_type = other DNA
gaggtgcagctcctggagtctggcggtggcctggtgcaagctggcggttcccttcgtctgtcctgcgcagccagtggctattctaactgtgcttacgacatgacatggtatcgtcaggcaccggggaaggagcgcgagttcgtatcagctatccactctgacggcagtaccagatatgccgacagcgtcaaagggcgttttttcatctcccgcgacaactccaaaaataccctgtatcttcagatgaactccctcaggcctgaggacactgccgtctattactgcaagactgaccccctccactgtcgcgctcatgggggtagttggtactctgtgcgtgctaattactgggggcagggcaccctggtgaccgttagctca

SEQ ID NO: 380 - DNA, 381 bp, synthetic construct
  source 1..381: mol_type = other DNA
gaggtgcagttgctggagagcgggggcgggctcgtgcaggcgggaggcagcttgcgcttgtcatgtgctgccagcggttatagcaactgcgcctacgacatgacatggtatcgccaggctcccggtaaagagagggagtttgtatccgcgatccacagtgacggtagcacccgctacgcggattccgtgaaaggacgtttctttatttctagggataatagcaagaatacactgtacttgcaaatgaactccctccgcgccgaggacactgctgtgtattactgcaaaactgatccactgcactgtcgcgctcacgggggttcttggtattcagtacgcgccaactactggggccagggtactctcgtgacggtgagcagc

SEQ ID NO: 381 - DNA, 381 bp, synthetic construct
  source 1..381: mol_type = other DNA
gaggtgcagctcctggaatctggcgggggcctggtccaggctggcggtagcctccgccttagttgtgctgccagcggttacagcaactgcgcttacgccatgacgtggtatcgccaggccccagggaaggagagggagttcgtcagcgctatccattccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagc

SEQ ID NO: 382 - skipped

SEQ ID NO: 383 - DNA, 381 bp, synthetic construct
  source 1..381: mol_type = other DNA
gaggtgcagctcctggaatctggcgggggcctggtccaggctggcggtagcctccgccttagttgtgctgccagcggttacagcaactgcgcttacgacatgtcgtggtatcgccaggccccagggaaggagagggagttcgtcagcgctatccattccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagc

SEQ ID NO: 384 - DNA, 381 bp, synthetic construct
  source 1..381: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatctcttccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagc

SEQ ID NO: 385 - DNA, 381 bp, synthetic construct
  source 1..381: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccatggcgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagc

SEQ ID NO: 386 - DNA, 381 bp, synthetic construct
  source 1..381: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccattccagtgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagc

SEQ ID NO: 387 - skipped

SEQ ID NO: 388 - DNA, 381 bp, synthetic construct
  source 1..381: mol_type = other DNA
gaggtgcagctcctggaatctggcgggggcctggtccaggctggcggtagcctccgccttagttgtgctgccagcggttacagcaactgcgcttacgccatgtcgtggtatcgccaggccccagggaaggagagggagttcgtcagcgctatccattccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagc

SEQ ID NO: 389 - DNA, 381 bp, synthetic construct
  source 1..381: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatctctggcagtgggagcacatactacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagc

SEQ ID NO: 390 - skipped

SEQ ID NO: 391 - DNA, 381 bp, synthetic construct
  source 1..381: mol_type = other DNA
gaagtgcagctcctggagagcggcggtggctcggtgcagccaggcggttctctgcgcttgtcctgtgctgcctccggctactcaaactgtgcttacgatatgacttggtatcgccaggcccctggtaaggagcgcgagttcgtctctgccattcattctgacggctcaacccgttatgcagactccgttaaggggcgttttttcatttcccgcgacaactccaagaacaccctgtaccttcagatgaactccctccgcgccgaggataccgccgtatactattgcaagaccgaccccctgcattgcagggctcacggcgggtcctggtactccgtgcgtgccaactattggggacaggggactctggtcacagtctcgagt

SEQ ID NO: 392 - DNA, 416 bp, synthetic construct
  source 1..416: mol_type = other DNA
gaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgctagccaccatcaccatcaccactgataagcggccgc

SEQ ID NO: 393 - DNA, 392 bp, synthetic construct
  source 1..392: mol_type = other DNA
gaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccacggtaactgtctcgtcttagtgagcggccgc

SEQ ID NO: 394-414 - skipped (21 entries)

SEQ ID NO: 415 - DNA, 384 bp, synthetic construct
  source 1..384: mol_type = other DNA
gaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcggcagctagc

SEQ ID NO: 416 - protein, 5 aa, synthetic construct
GGGGS

SEQ ID NO: 417 - protein, 6 aa, synthetic construct
GGGGGS

SEQ ID NO: 418 - protein, 4 aa, synthetic construct
GGSG

SEQ ID NO: 419 - protein, 4 aa, synthetic construct
SGGG

SEQ ID NO: 420 - protein, 4 aa, synthetic construct
GSGS

SEQ ID NO: 421 - protein, 6 aa, synthetic construct
GSGSGS

SEQ ID NO: 422 - protein, 8 aa, synthetic construct
GSGSGSGS

SEQ ID NO: 423 - protein, 10 aa, synthetic construct
GSGSGSGSGS

SEQ ID NO: 424 - protein, 6 aa, synthetic construct
GGSGGS

SEQ ID NO: 425 - protein, 9 aa, synthetic construct
GGSGGSGGS

SEQ ID NO: 426 - protein, 10 aa, synthetic construct
GGSGGSGGSG

SEQ ID NO: 427 - protein, 8 aa, synthetic construct
GGSGGGSG

SEQ ID NO: 428 - protein, 12 aa, synthetic construct
GGSGGGSGGGSG

SEQ ID NO: 429 - protein, 16 aa, synthetic construct
GGSGGGSGGGSGGGSG

SEQ ID NO: 430 - protein, 20 aa, synthetic construct
GGSGGGSGGGSGGGSGGGSG

SEQ ID NO: 431 - protein, 10 aa, synthetic construct
GGGGSGGGGS

SEQ ID NO: 432 - protein, 8 aa, synthetic construct
GGGSGGGS

SEQ ID NO: 433 - protein, 5 aa, synthetic construct
GGSGG

SEQ ID NO: 434 - protein, 5 aa, synthetic construct
GSGGS

SEQ ID NO: 435 - protein, 5 aa, synthetic construct
GSGSG

SEQ ID NO: 436 - protein, 4 aa, synthetic construct
GSGG

SEQ ID NO: 437 - protein, 5 aa, synthetic construct
GGGSG

SEQ ID NO: 438 - protein, 5 aa, synthetic construct
GSSSG

SEQ ID NO: 439 - protein, 4 aa, synthetic construct
GGGS

SEQ ID NO: 440 - protein, 232 aa, synthetic construct
ASRVESKYGPPCPPCPAPEFLGGPSVFLFPPKPKDTLMISRTPEVTCVVVDVSQEDPEVQFNWYVDGVEVHNAKTKPREEQFGSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 441-443 - skipped (3 entries)

SEQ ID NO: 444 - protein, 2000 aa, synthetic construct
  VARIANT 1..20: note = This region may encompass 1-20 residues
  VARIANT 21..40: note = This region may encompass 1-20 residues
  VARIANT 41..60: note = This region may encompass 1-20 residues
  VARIANT 61..80: note = This region may encompass 1-20 residues
  VARIANT 81..100: note = This region may encompass 1-20 residues
  VARIANT 101..120: note = This region may encompass 1-20 residues
  VARIANT 121..140: note = This region may encompass 1-20 residues
  VARIANT 141..160: note = This region may encompass 1-20 residues
  VARIANT 161..180: note = This region may encompass 1-20 residues
  VARIANT 181..200: note = This region may encompass 1-20 residues
  VARIANT 201..220: note = This region may encompass 1-20 residues
  VARIANT 221..240: note = This region may encompass 1-20 residues
  VARIANT 241..260: note = This region may encompass 1-20 residues
  VARIANT 261..280: note = This region may encompass 1-20 residues
  VARIANT 281..300: note = This region may encompass 1-20 residues
  VARIANT 301..320: note = This region may encompass 1-20 residues
  VARIANT 321..340: note = This region may encompass 1-20 residues
  VARIANT 341..360: note = This region may encompass 1-20 residues
  VARIANT 361..380: note = This region may encompass 1-20 residues
  VARIANT 381..400: note = This region may encompass 1-20 residues
  VARIANT 401..420: note = This region may encompass 1-20 residues
  VARIANT 421..440: note = This region may encompass 1-20 residues
  VARIANT 441..460: note = This region may encompass 1-20 residues
  VARIANT 461..480: note = This region may encompass 1-20 residues
  VARIANT 481..500: note = This region may encompass 1-20 residues
  VARIANT 501..520: note = This region may encompass 1-20 residues
  VARIANT 521..540: note = This region may encompass 1-20 residues
  VARIANT 541..560: note = This region may encompass 1-20 residues
  VARIANT 561..580: note = This region may encompass 1-20 residues
  VARIANT 581..600: note = This region may encompass 1-20 residues
  VARIANT 601..620: note = This region may encompass 1-20 residues
  VARIANT 621..640: note = This region may encompass 1-20 residues
  VARIANT 641..660: note = This region may encompass 1-20 residues
  VARIANT 661..680: note = This region may encompass 1-20 residues
  VARIANT 681..700: note = This region may encompass 1-20 residues
  VARIANT 701..720: note = This region may encompass 1-20 residues
  VARIANT 721..740: note = This region may encompass 1-20 residues
  VARIANT 741..760: note = This region may encompass 1-20 residues
  VARIANT 761..780: note = This region may encompass 1-20 residues
  VARIANT 781..800: note = This region may encompass 1-20 residues
  VARIANT 801..820: note = This region may encompass 1-20 residues
  VARIANT 821..840: note = This region may encompass 1-20 residues
  VARIANT 841..860: note = This region may encompass 1-20 residues
  VARIANT 861..880: note = This region may encompass 1-20 residues
  VARIANT 881..900: note = This region may encompass 1-20 residues
  VARIANT 901..920: note = This region may encompass 1-20 residues
  VARIANT 921..940: note = This region may encompass 1-20 residues
  VARIANT 941..960: note = This region may encompass 1-20 residues
  VARIANT 961..980: note = This region may encompass 1-20 residues
  VARIANT 981..1000: note = This region may encompass 1-20 residues
  VARIANT 1001..1020: note = This region may encompass 1-20 residues
  VARIANT 1021..1040: note = This region may encompass 1-20 residues
  VARIANT 1041..1060: note = This region may encompass 1-20 residues
  VARIANT 1061..1080: note = This region may encompass 1-20 residues
  VARIANT 1081..1100: note = This region may encompass 1-20 residues
  VARIANT 1101..1120: note = This region may encompass 1-20 residues
  VARIANT 1121..1140: note = This region may encompass 1-20 residues
  VARIANT 1141..1160: note = This region may encompass 1-20 residues
  VARIANT 1161..1180: note = This region may encompass 1-20 residues
  VARIANT 1181..1200: note = This region may encompass 1-20 residues
  VARIANT 1201..1220: note = This region may encompass 1-20 residues
  VARIANT 1221..1240: note = This region may encompass 1-20 residues
  VARIANT 1241..1260: note = This region may encompass 1-20 residues
  VARIANT 1261..1280: note = This region may encompass 1-20 residues
  VARIANT 1281..1300: note = This region may encompass 1-20 residues
  VARIANT 1301..1320: note = This region may encompass 1-20 residues
  VARIANT 1321..1340: note = This region may encompass 1-20 residues
  VARIANT 1341..1360: note = This region may encompass 1-20 residues
  VARIANT 1361..1380: note = This region may encompass 1-20 residues
  VARIANT 1381..1400: note = This region may encompass 1-20 residues
  VARIANT 1401..1420: note = This region may encompass 1-20 residues
  VARIANT 1421..1440: note = This region may encompass 1-20 residues
  VARIANT 1441..1460: note = This region may encompass 1-20 residues
  VARIANT 1461..1480: note = This region may encompass 1-20 residues
  VARIANT 1481..1500: note = This region may encompass 1-20 residues
  VARIANT 1501..1520: note = This region may encompass 1-20 residues
  VARIANT 1521..1540: note = This region may encompass 1-20 residues
  VARIANT 1541..1560: note = This region may encompass 1-20 residues
  VARIANT 1561..1580: note = This region may encompass 1-20 residues
  VARIANT 1581..1600: note = This region may encompass 1-20 residues
  VARIANT 1601..1620: note = This region may encompass 1-20 residues
  VARIANT 1621..1640: note = This region may encompass 1-20 residues
  VARIANT 1641..1660: note = This region may encompass 1-20 residues
  VARIANT 1661..1680: note = This region may encompass 1-20 residues
  VARIANT 1681..1700: note = This region may encompass 1-20 residues
  VARIANT 1701..1720: note = This region may encompass 1-20 residues
  VARIANT 1721..1740: note = This region may encompass 1-20 residues
  VARIANT 1741..1760: note = This region may encompass 1-20 residues
  VARIANT 1761..1780: note = This region may encompass 1-20 residues
  VARIANT 1781..1800: note = This region may encompass 1-20 residues
  VARIANT 1801..1820: note = This region may encompass 1-20 residues
  VARIANT 1821..1840: note = This region may encompass 1-20 residues
  VARIANT 1841..1860: note = This region may encompass 1-20 residues
  VARIANT 1861..1880: note = This region may encompass 1-20 residues
  VARIANT 1881..1900: note = This region may encompass 1-20 residues
  VARIANT 1901..1920: note = This region may encompass 1-20 residues
  VARIANT 1921..1940: note = This region may encompass 1-20 residues
  VARIANT 1941..1960: note = This region may encompass 1-20 residues
  VARIANT 1961..1980: note = This region may encompass 1-20 residues
  VARIANT 1981..2000: note = This region may encompass 1-20 residues
  VARIANT 1..2000: note = This sequence may encompass 1-20 GmSoGmSoGm repeating units, wherein m = 1 to 20, o = 1 to 20
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGG

SEQ ID NO: 445-447 - skipped (3 entries)

SEQ ID NO: 448 - protein, 24 aa, synthetic construct
  VARIANT 11: note = Any amino acid other than proline
  VARIANT 12: note = Any amino acid other than proline
  VARIANT 13: note = Any amino acid other than proline
  VARIANT 14: note = Any amino acid other than proline
  VARIANT 1..10: note = This region may encompass 0-10 residues
  VARIANT 11..14: note = This region may encompass 0-4 residues
  VARIANT 15..24: note = This region may encompass 0-10 residues
HHHHHHHHHHXXXXHHHHHHHHHH

SEQ ID NO: 449 - protein, 8 aa, synthetic construct
ASHHHHHH

SEQ ID NO: 450 - protein, 5 aa, synthetic construct
HHHHH

SEQ ID NO: 451 - protein, 6 aa, synthetic construct
HHHHHH

SEQ ID NO: 452 - protein, 7 aa, synthetic construct
HHHHHHH

SEQ ID NO: 453 - protein, 8 aa, synthetic construct
HHHHHHHH

SEQ ID NO: 454 - protein, 9 aa, synthetic construct
HHHHHHHHH

SEQ ID NO: 455 - protein, 5 aa, synthetic construct
HWHMY

SEQ ID NO: 456 - protein, 5 aa, synthetic construct
HHHMY

SEQ ID NO: 457 - protein, 5 aa, synthetic construct
HHHHY

SEQ ID NO: 458 - protein, 5 aa, synthetic construct
HWHWH

SEQ ID NO: 459 - protein, 5 aa, synthetic construct
HWHHH

SEQ ID NO: 460 - protein, 9 aa, synthetic construct
HHHHYMHHH

SEQ ID NO: 461 - protein, 4 aa, synthetic construct
HHHH

SEQ ID NO: 462 - protein, 5 aa, synthetic construct
HWHHH

SEQ ID NO: 463 - protein, 9 aa, synthetic construct
HGHGGGHGH

SEQ ID NO: 464 - protein, 12 aa, synthetic construct
HGHGGGGGGHGH

SEQ ID NO: 465 - protein, 266 aa, synthetic construct
DEVQLQESGGGSVQAGGSLRLSCAASGYSNCAYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGGSQVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSSASHHHHHH

SEQ ID NO: 466 - protein, 128 aa, synthetic construct
DEVQLQESGGGSVQAGGSLRLSCAASGYSNCAYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSS

SEQ ID NO: 467 - protein, 9 aa, synthetic construct
YSNCAYDMT

SEQ ID NO: 468-469 - skipped (2 entries)

SEQ ID NO: 470 - protein, 126 aa, synthetic construct
QVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSS

SEQ ID NO: 471 - protein, 9 aa, synthetic construct
YTHSSYCMG

SEQ ID NO: 472 - protein, 16 aa, synthetic construct
AIDVDGSTTYADSVKG

SEQ ID NO: 473 - protein, 20 aa, synthetic construct
AAEFADCSSNYFLPPGAVRY

SEQ ID NO: 474 - protein, 5 aa, synthetic construct
VTVSS

SEQ ID NO: 475 - protein, 7 aa, synthetic construct
VTVSSAA

SEQ ID NO: 476 - protein, 5 aa, synthetic construct
GTVSS

SEQ ID NO: 477 - protein, 5 aa, synthetic construct
LTVSS

SEQ ID NO: 478 - protein, 7 aa, synthetic construct
LTVSSAA

SEQ ID NO: 479 - protein, 5 aa, synthetic construct
VTVSA

SEQ ID NO: 480 - protein, 7 aa, synthetic construct
VTVSAAA

SEQ ID NO: 481 - protein, 5 aa, synthetic construct
VTVCS

SEQ ID NO: 482 - protein, 6 aa, synthetic construct
VTVCSA

SEQ ID NO: 483 - protein, 5 aa, synthetic construct
VTVSC

SEQ ID NO: 484 - protein, 7 aa, synthetic construct
VTVSCAA

SEQ ID NO: 485 - protein, 6 aa, synthetic construct
LLTVSS

SEQ ID NO: 486 - protein, 7 aa, synthetic construct
LLTVSSA

SEQ ID NO: 487 - protein, 8 aa, synthetic construct
LLTVSSAA

SEQ ID NO: 488 - protein, 6 aa, synthetic construct
LLTVSS

SEQ ID NO: 489 - protein, 7 aa, synthetic construct
LLTVSSA

SEQ ID NO: 490 - protein, 8 aa, synthetic construct
LLTVSSAA

SEQ ID NO: 491 - protein, 6 aa, synthetic construct
LVTVTT

SEQ ID NO: 492 - protein, 8 aa, synthetic construct
LVTVTTAA

SEQ ID NO: 493 - protein, 6 aa, synthetic construct
LVTVTT

SEQ ID NO: 494 - protein, 8 aa, synthetic construct
LVTVTTAA

SEQ ID NO: 495 - protein, 6 aa, synthetic construct
TLTVSS

SEQ ID NO: 496 - protein, 7 aa, synthetic construct
TLTVSSA

SEQ ID NO: 497 - protein, 8 aa, synthetic construct
TLTVSSAA

SEQ ID NO: 498 - protein, 7 aa, synthetic construct
VTVSAAS

SEQ ID NO: 499 - protein, 6 aa, synthetic construct
LVTVSS

SEQ ID NO: 500 - protein, 257 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 501 - protein, 257 aa, synthetic construct; identical to SEQ ID NO: 2

SEQ ID NO: 502 - protein, 257 aa, synthetic construct; identical to SEQ ID NO: 3

SEQ ID NO: 503 - protein, 257 aa, synthetic construct; identical to SEQ ID NO: 4

SEQ ID NO: 504 - protein, 257 aa, synthetic construct
EVQLQESGGGSVQPGGSLRLSCAASGYSNCAYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 505 - protein, 257 aa, synthetic construct
EVQLQESGGGLVQAGGSLRLSCAASGYSNCAYDMTWYRQAPGKEREFVSAIHSDGSTRYADSVKGRFFISQDNSKNTVYLQMNSLKPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 506 - protein, 257 aa, synthetic construct
EVQLQESGGGLVQAGGSLRLSCAASGYSNCAYDMTWYRQAPGKEREFVSAIHSDGSTRYADSVKGRFFISRDNSKNTVYLQMNSLKPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 507 - protein, 257 aa, synthetic construct
EVQLLESGGGLVQAGGSLRLSCAASGYSNCAYDMTWYRQAPGKEREFVSAIHSDGSTRYADSVKGRFFISQDNSKNTVYLQMNSLKPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 508 - protein, 256 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLKPEDTAVYYCKTDPLHCRAHGGSWYSVRANYWGQGTLVTVSSGGGSEVQLVESGGGLVQPGGSLLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 509 - protein, 257 aa, synthetic construct
EVQLQESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 510 - protein, 257 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 511 - protein, 257 aa, synthetic construct
EVQLLESGGGLVQAGGSLRLSCAASGYSNCAYDMTWYRQAPGKEREFVSAIHSDGSTRYADSVKGRFFISRDNSKNTLYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 512 - protein, 257 aa, synthetic construct
EVQLLESGGGLVQAGGSLRLSCAASGYSNCAYDMTWYRQAPGKEREFVSAIHSDGSTRYADSVKGRFFISRDNSKNTLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 513 - protein, 257 aa, synthetic construct
EVQLLESGGGLVQAGGSLRLSCAASGYSNCAYAMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 514 - protein, 257 aa, synthetic construct
EVQLLESGGGLVQAGGSLRLSCAASGYSNCAYDMSWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 515 - protein, 257 aa, synthetic construct
EVQLLESGGGLVQAGGSLRLSCAASGYSNCAYDMTWYRQAPGKEREFVSAISSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 516 - protein, 257 aa, synthetic construct
EVQLLESGGGLVQAGGSLRLSCAASGYSNCAYDMTWYRQAPGKEREFVSAIHGDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 517 - protein, 257 aa, synthetic construct
EVQLLESGGGLVQAGGSLRLSCAASGYSNCAYDMTWYRQAPGKEREFVSAIHSSGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 518 - protein, 257 aa, synthetic construct
EVQLLESGGGLVQAGGSLRLSCAASGYSNCAYDMTWYRQAPGKEREFVSAIHSDGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 519 - protein, 257 aa, synthetic construct
EVQLLESGGGLVQAGGSLRLSCAASGYSNCAYAMSWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 520 - protein, 257 aa, synthetic construct
EVQLLESGGGLVQAGGSLRLSCAASGYSNCAYDMTWYRQAPGKEREFVSAISGSGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 521 - protein, 257 aa, synthetic construct
EVQLLESGGGLVQAGGSLRLSCAASGYSNCAYAMSWYRQAPGKEREFVSAISGSGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 522 - protein, 257 aa, synthetic construct
EVQLLESGGGLVQPGGSLRLSCAASGYSNCAYDMTWYRQAPGKEREFVSAIHSDGSTRYADSVKGRFFISRDNSKNTLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 523 - protein, 257 aa, synthetic construct
EVQLLESGGGSVQPGGSLRLSCAASGYSNCAYDMTWYRQAPGKEREFVSAIHSDGSTRYADSVKGRFFISRDNSKNTLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 524 - DNA, 795 bp, synthetic construct
  source 1..795: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccattccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgctagccaccatcaccatcaccac

SEQ ID NO: 525 - DNA, 795 bp, synthetic construct
  source 1..795: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccattccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccacggtaactgtctcgtctgctagccaccatcaccatcaccac

SEQ ID NO: 526 - DNA, 795 bp, synthetic construct
  source 1..795: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccattccgatgggagcacatattacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccacggtaactgtctcgtctgctagccaccatcaccatcaccac

SEQ ID NO: 527 - DNA, 795 bp, synthetic construct
  source 1..795: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccattcctctgggagcacatattacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccacggtaactgtctcgtctgctagccaccatcaccatcaccac

SEQ ID NO: 528 - DNA, 795 bp, synthetic construct
  source 1..795: mol_type = other DNA
gaagtccagctccaggaatcaggaggtggctctgtgcaacccggtggctccctgcgcctcagctgtgccgcttctggatattctaactgcgcttacgacatgacttggtataggcaggcaccgggcaaggagcgcgagttcgtttctgccattcactctgatggatctacccgctatgccgatagcgtgaagggtcgctttttcatctcccaggataacgctaaaaatactgtgtaccttcagatgaacagcctgaaacctgaagacaccgccatgtactattgtaagacagacccgttgcattgcagagcccacggaggctcatggtattccgtccgggcgaactactggggccagggcactcaggtgaccgtatcctctggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgctagccaccatcaccatcaccac

SEQ ID NO: 529 - DNA, 795 bp, synthetic construct
  source 1..795: mol_type = other DNA
gaggttcaactccaggagagcggcgggggcctggtgcaagctgggggcagtctgcgcttgagctgcgctgcatccggctattccaactgtgcctatgacatgacgtggtataggcaggcccctgggaaggagcgtgagttcgtgagtgctatccacagcgacggctccactagatacgcggactccgttaaaggaagatttttcatcagtcaggacaacagcaagaataccgtgtatttgcagatgaatagtctgaagcccgaggacacagcagtctactattgtaagactgacccactgcactgtcgtgcgcatggaggctcttggtactccgtgagagccaattattgggggcagggcacccttgttaccgtatcctccggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgctagccaccatcaccatcaccac

SEQ ID NO: 530 - DNA, 795 bp, synthetic construct
  source 1..795: mol_type = other DNA
gaggtgcagctccaggagagcggcggtggcctcgtccaagccgggggctccctgcgtttgtcttgcgcggccagcggatattccaactgtgcgtatgacatgacatggtatcgccaggctccaggcaaggagcgtgagtttgtgtctgcaatccactctgatggctccacacgctacgccgacagcgtgaagggccgcttctttatctccagagataacagcaagaacactgtctatctccagatgaacagcctgaagcccgaagacaccgccgtttattactgtaagaccgaccctctgcattgcagagcccacggcggttcctggtattccgtccgcgctaactattggggacaggggacccttgtgacagtcagctccggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgctagccaccatcaccatcaccac

SEQ ID NO: 531 - DNA, 795 bp, synthetic construct
  source 1..795: mol_type = other DNA
gaggtgcagttgctggagtctggtgggggcctcgtgcaggccggaggctccctgcgcctgtcctgcgctgcctctggatactctaattgcgcatacgacatgacatggtatcggcaggcacccggaaaggagcgcgagttcgtatcagcaatccatagtgacggttctactcgctatgcagactctgtgaaaggacgcttctttattagccaggacaactctaaaaacactgtctatctccaaatgaactccctcaagccagaagatactgccgtgtattactgcaagaccgacccactgcattgtcgtgcgcatggagggtcttggtattcagtacgtgcaaactactggggacagggcaccctggtcactgtgtcctctggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgctagccaccatcaccatcaccac

SEQ ID NO: 532 - DNA, 795 bp, synthetic construct
  source 1..795: mol_type = other DNA
gaggtacagctcttggagtccggcggtggcttggtgcaggctggaggtagcttgcgcctgtcctgcgccgcttccggctacagcaactgcgcctacgacatgacgtggtatcggcaggctccaggtaaggagcgcgagttcgtcagtgccattcactccgacgggagcactagatatgccgactctgtcaagggacgttttttcattagtcgcgacaactctaagaacaccgtgtaccttcagatgaacagcctgaagcccgaggacactgccgtgtactattgcaagaccgaccccctccattgcagggctcatgggggctcttggtattcagtgagagccaactattggggacagggtacgctggtgaccgtgtcatctggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgctagccaccatcaccatcaccac

SEQ ID NO: 533 - DNA, 795 bp, synthetic construct
  source 1..795: mol_type = other DNA
gaggtccagttgcaggagtccggtggcgggttggtccaagcaggcggaagcctccgccttagttgtgcggcctctggctactctaactgcgcctatgatatgacgtggtatcgccaagctccaggcaaagaaagagagttcgtcagcgccattcactctgatggctccactaggtacgcagacagcgtcaagggtcgttttttcatctcccgcgataactccaagaacaccgtgtacctccaaatgaactccctgagaccggaggacactgctgtgtactattgcaagaccgatcctttgcactgtagggcgcatggaggcagttggtactccgttagagccaactactgggggcaaggaaccctggtcactgtcagctccggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgctagccaccatcaccatcaccac

SEQ ID NO: 534 - DNA, 795 bp, synthetic construct
  source 1..795: mol_type = other DNA
gaggttcagttgctggaatctgggggcggtctggtccaggctgggggctctctgaggctgagctgcgccgcatctggctactccaactgcgcctatgatatgacttggtacaggcaggctcccggcaaggagcgcgagttcgtgagcgccattcactctgatggttccacaaggtatgctgatagtgttaaaggacgctttttcattagccgggacaatagcaaaaacacagtctatctccagatgaactccctccgggcggaggataccgctgtctattactgcaagactgaccctttgcactgtagagcacacggaggcagctggtattctgtgagagccaactattggggacaaggaaccctcgtcacggtgtctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgctagccaccatcaccatcaccac

SEQ ID NO: 535 - DNA, 795 bp, synthetic construct
  source 1..795: mol_type = other DNA
gaggtgcagctcctggagtctggcggtggcctggtgcaagctggcggttcccttcgtctgtcctgcgcagccagtggctattctaactgtgcttacgacatgacatggtatcgtcaggcaccggggaaggagcgcgagttcgtatcagctatccactctgacggcagtaccagatatgccgacagcgtcaaagggcgttttttcatctcccgcgacaactccaaaaataccctgtatcttcagatgaactccctcaggcctgaggacactgccgtctattactgcaagactgaccccctccactgtcgcgctcatgggggtagttggtactctgtgcgtgctaattactgggggcagggcaccctggtgaccgttagctcaggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgctagccaccatcaccatcaccac

SEQ ID NO: 536 - DNA, 795 bp, synthetic construct
  source 1..795: mol_type = other DNA
gaggtgcagttgctggagagcgggggcgggctcgtgcaggcgggaggcagcttgcgcttgtcatgtgctgccagcggttatagcaactgcgcctacgacatgacatggtatcgccaggctcccggtaaagagagggagtttgtatccgcgatccacagtgacggtagcacccgctacgcggattccgtgaaaggacgtttctttatttctagggataatagcaagaatacactgtacttgcaaatgaactccctccgcgccgaggacactgctgtgtattactgcaaaactgatccactgcactgtcgcgctcacgggggttcttggtattcagtacgcgccaactactggggccagggtactctcgtgacggtgagcagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgctagccaccatcaccatcaccac

SEQ ID NO: 537 - DNA, 795 bp, synthetic construct
  source 1..795: mol_type = other DNA
gaggtgcagctcctggaatctggcgggggcctggtccaggctggcggtagcctccgccttagttgtgctgccagcggttacagcaactgcgcttacgccatgacgtggtatcgccaggccccagggaaggagagggagttcgtcagcgctatccattccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgctagccaccatcaccatcaccac

SEQ ID NO: 538 - DNA, 795 bp, synthetic construct
  source 1..795: mol_type = other DNA
gaggtgcagctcctggaatctggcgggggcctggtccaggctggcggtagcctccgccttagttgtgctgccagcggttacagcaactgcgcttacgacatgtcgtggtatcgccaggccccagggaaggagagggagttcgtcagcgctatccattccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgctagccaccatcaccatcaccac

SEQ ID NO: 539 - DNA, 795 bp, synthetic construct
  source 1..795: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatctcttccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgctagccaccatcaccatcaccac

SEQ ID NO: 540 - DNA, 795 bp, synthetic construct
  source 1..795: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccatggcgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgctagccaccatcaccatcaccac

SEQ ID NO: 541 - DNA, 795 bp, synthetic construct
  source 1..795: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccattccagtgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgctagccaccatcaccatcaccac

SEQ ID NO: 542 - DNA, 795 bp, synthetic construct
  source 1..795: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccattccgatgggagcacatactacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgctagccaccatcaccatcaccac

SEQ ID NO: 543 - DNA, 795 bp, synthetic construct
  source 1..795: mol_type = other DNA
gaggtgcagctcctggaatctggcgggggcctggtccaggctggcggtagcctccgccttagttgtgctgccagcggttacagcaactgcgcttacgccatgtcgtggtatcgccaggccccagggaaggagagggagttcgtcagcgctatccattccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgctagccaccatcaccatcaccac

SEQ ID NO: 544 - DNA, 795 bp, synthetic construct
  source 1..795: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatctctggcagtgggagcacatactacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgctagccaccatcaccatcaccac

SEQ ID NO: 545 - DNA, 795 bp, synthetic construct
  source 1..795: mol_type = other DNA
gaggtgcagctcctggaatctggcgggggcctggtccaggctggcggtagcctccgccttagttgtgctgccagcggttacagcaactgcgcttacgccatgtcgtggtatcgccaggccccagggaaggagagggagttcgtcagcgctatctctggcagtgggagcacatactacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtctgctagccaccatcaccatcaccac

SEQ ID NO: 546 - DNA, 795 bp, synthetic construct; identical to SEQ ID NO: 533
  source 1..795: mol_type = other DNA

SEQ ID NO: 547 - DNA, 795 bp, synthetic construct
  source 1..795: mol_type = other DNA
gaagtgcagctcctggagagcggcggtggctcggtgcagccaggcggttctctgcgcttgtcctgtgctgcctccggctactcaaactgtgcttacgatatgacttggtatcgccaggcccctggtaaggagcgcgagttcgtctctgccattcattctgacggctcaacccgttatgcagactccgttaaggggcgttttttcatttcccgcgacaactccaagaacaccctgtaccttcagatgaactccctccgcgccgaggataccgccgtatactattgcaagaccgaccccctgcattgcagggctcacggcgggtcctggtactccgtgcgtgccaactattggggacaggggactctggtcacagtctcgagtggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcggcagctagccaccatcaccatcaccac

SEQ ID NO: 548 - DNA, 771 bp, synthetic construct
  source 1..771: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccattccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtct

SEQ ID NO: 549 - DNA, 771 bp, synthetic construct; identical to SEQ ID NO: 201
  source 1..771: mol_type = other DNA

SEQ ID NO: 550 - DNA, 771 bp, synthetic construct; identical to SEQ ID NO: 202
  source 1..771: mol_type = other DNA

SEQ ID NO: 551 - DNA, 771 bp, synthetic construct; identical to SEQ ID NO: 203
  source 1..771: mol_type = other DNA

SEQ ID NO: 552 - DNA, 771 bp, synthetic construct
  source 1..771: mol_type = other DNA
gaagtccagctccaggaatcaggaggtggctctgtgcaacccggtggctccctgcgcctcagctgtgccgcttctggatattctaactgcgcttacgacatgacttggtataggcaggcaccgggcaaggagcgcgagttcgtttctgccattcactctgatggatctacccgctatgccgatagcgtgaagggtcgctttttcatctcccaggataacgctaaaaatactgtgtaccttcagatgaacagcctgaaacctgaagacaccgccatgtactattgtaagacagacccgttgcattgcagagcccacggaggctcatggtattccgtccgggcgaactactggggccagggcactcaggtgaccgtatcctctggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtct

SEQ ID NO: 553 - DNA, 771 bp, synthetic construct
  source 1..771: mol_type = other DNA
gaggttcaactccaggagagcggcgggggcctggtgcaagctgggggcagtctgcgcttgagctgcgctgcatccggctattccaactgtgcctatgacatgacgtggtataggcaggcccctgggaaggagcgtgagttcgtgagtgctatccacagcgacggctccactagatacgcggactccgttaaaggaagatttttcatcagtcaggacaacagcaagaataccgtgtatttgcagatgaatagtctgaagcccgaggacacagcagtctactattgtaagactgacccactgcactgtcgtgcgcatggaggctcttggtactccgtgagagccaattattgggggcagggcacccttgttaccgtatcctccggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtct

SEQ ID NO: 554 - DNA, 771 bp, synthetic construct
  source 1..771: mol_type = other DNA
gaggtgcagctccaggagagcggcggtggcctcgtccaagccgggggctccctgcgtttgtcttgcgcggccagcggatattccaactgtgcgtatgacatgacatggtatcgccaggctccaggcaaggagcgtgagtttgtgtctgcaatccactctgatggctccacacgctacgccgacagcgtgaagggccgcttctttatctccagagataacagcaagaacactgtctatctccagatgaacagcctgaagcccgaagacaccgccgtttattactgtaagaccgaccctctgcattgcagagcccacggcggttcctggtattccgtccgcgctaactattggggacaggggacccttgtgacagtcagctccggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtct

SEQ ID NO: 555 - DNA, 771 bp, synthetic construct
  source 1..771: mol_type = other DNA
gaggtgcagttgctggagtctggtgggggcctcgtgcaggccggaggctccctgcgcctgtcctgcgctgcctctggatactctaattgcgcatacgacatgacatggtatcggcaggcacccggaaaggagcgcgagttcgtatcagcaatccatagtgacggttctactcgctatgcagactctgtgaaaggacgcttctttattagccaggacaactctaaaaacactgtctatctccaaatgaactccctcaagccagaagatactgccgtgtattactgcaagaccgacccactgcattgtcgtgcgcatggagggtcttggtattcagtacgtgcaaactactggggacagggcaccctggtcactgtgtcctctggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtct

SEQ ID NO: 556 - DNA, 771 bp, synthetic construct
  source 1..771: mol_type = other DNA
gaggtacagctcttggagtccggcggtggcttggtgcaggctggaggtagcttgcgcctgtcctgcgccgcttccggctacagcaactgcgcctacgacatgacgtggtatcggcaggctccaggtaaggagcgcgagttcgtcagtgccattcactccgacgggagcactagatatgccgactctgtcaagggacgttttttcattagtcgcgacaactctaagaacaccgtgtaccttcagatgaacagcctgaagcccgaggacactgccgtgtactattgcaagaccgaccccctccattgcagggctcatgggggctcttggtattcagtgagagccaactattggggacagggtacgctggtgaccgtgtcatctggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtct

SEQ ID NO: 557 - DNA, 771 bp, synthetic construct
  source 1..771: mol_type = other DNA
gaggtccagttgcaggagtccggtggcgggttggtccaagcaggcggaagcctccgccttagttgtgcggcctctggctactctaactgcgcctatgatatgacgtggtatcgccaagctccaggcaaagaaagagagttcgtcagcgccattcactctgatggctccactaggtacgcagacagcgtcaagggtcgttttttcatctcccgcgataactccaagaacaccgtgtacctccaaatgaactccctgagaccggaggacactgctgtgtactattgcaagaccgatcctttgcactgtagggcgcatggaggcagttggtactccgttagagccaactactgggggcaaggaaccctggtcactgtcagctccggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtct

SEQ ID NO: 558 - DNA, 771 bp, synthetic construct
  source 1..771: mol_type = other DNA
gaggttcagttgctggaatctgggggcggtctggtccaggctgggggctctctgaggctgagctgcgccgcatctggctactccaactgcgcctatgatatgacttggtacaggcaggctcccggcaaggagcgcgagttcgtgagcgccattcactctgatggttccacaaggtatgctgatagtgttaaaggacgctttttcattagccgggacaatagcaaaaacacagtctatctccagatgaactccctccgggcggaggataccgctgtctattactgcaagactgaccctttgcactgtagagcacacggaggcagctggtattctgtgagagccaactattggggacaaggaaccctcgtcacggtgtctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtct

SEQ ID NO: 559 - DNA, 771 bp, synthetic construct
  source 1..771: mol_type = other DNA
gaggtgcagctcctggagtctggcggtggcctggtgcaagctggcggttcccttcgtctgtcctgcgcagccagtggctattctaactgtgcttacgacatgacatggtatcgtcaggcaccggggaaggagcgcgagttcgtatcagctatccactctgacggcagtaccagatatgccgacagcgtcaaagggcgttttttcatctcccgcgacaactccaaaaataccctgtatcttcagatgaactccctcaggcctgaggacactgccgtctattactgcaagactgaccccctccactgtcgcgctcatgggggtagttggtactctgtgcgtgctaattactgggggcagggcaccctggtgaccgttagctcaggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtct

SEQ ID NO: 560 - DNA, 771 bp, synthetic construct
  source 1..771: mol_type = other DNA
gaggtgcagttgctggagagcgggggcgggctcgtgcaggcgggaggcagcttgcgcttgtcatgtgctgccagcggttatagcaactgcgcctacgacatgacatggtatcgccaggctcccggtaaagagagggagtttgtatccgcgatccacagtgacggtagcacccgctacgcggattccgtgaaaggacgtttctttatttctagggataatagcaagaatacactgtacttgcaaatgaactccctccgcgccgaggacactgctgtgtattactgcaaaactgatccactgcactgtcgcgctcacgggggttcttggtattcagtacgcgccaactactggggccagggtactctcgtgacggtgagcagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtct

SEQ ID NO: 561 - DNA, 771 bp, synthetic construct
  source 1..771: mol_type = other DNA
gaggtgcagctcctggaatctggcgggggcctggtccaggctggcggtagcctccgccttagttgtgctgccagcggttacagcaactgcgcttacgccatgacgtggtatcgccaggccccagggaaggagagggagttcgtcagcgctatccattccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtct

SEQ ID NO: 562 - DNA, 771 bp, synthetic construct
  source 1..771: mol_type = other DNA
gaggtgcagctcctggaatctggcgggggcctggtccaggctggcggtagcctccgccttagttgtgctgccagcggttacagcaactgcgcttacgacatgtcgtggtatcgccaggccccagggaaggagagggagttcgtcagcgctatccattccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtct

SEQ ID NO: 563 - DNA, 771 bp, synthetic construct
  source 1..771: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatctcttccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtct

SEQ ID NO: 564 - DNA, 771 bp, synthetic construct
  source 1..771: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccatggcgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtct

SEQ ID NO: 565 - DNA, 771 bp, synthetic construct
  source 1..771: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccattccagtgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtct

SEQ ID NO: 566 - DNA, 771 bp, synthetic construct
  source 1..771: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatccattccgatgggagcacatactacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtct

SEQ ID NO: 567 - DNA, 771 bp, synthetic construct
  source 1..771: mol_type = other DNA
gaggtgcagctcctggaatctggcgggggcctggtccaggctggcggtagcctccgccttagttgtgctgccagcggttacagcaactgcgcttacgccatgtcgtggtatcgccaggccccagggaaggagagggagttcgtcagcgctatccattccgatgggagcacacgctacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtct

SEQ ID NO: 568 - DNA, 771 bp, synthetic construct
  source 1..771: mol_type = other DNA
gaggtgcagctcctggaatctggcgggggcctggtccaggctggcggtagcctccgccttagttgtgctgccagcggttacagcaactgcgcttacgacatgacgtggtatcgccaggccccagggaaggagagggagttcgtcagcgctatctctggcagtgggagcacatactacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtct

SEQ ID NO: 569 - DNA, 771 bp, synthetic construct
  source 1..771: mol_type = other DNA
gaggtgcagctcctggaatctggcgggggcctggtccaggctggcggtagcctccgccttagttgtgctgccagcggttacagcaactgcgcttacgccatgtcgtggtatcgccaggccccagggaaggagagggagttcgtcagcgctatctctggcagtgggagcacatactacgctgactccgtgaaagggaggttctttatctctcgtgacaacagcaaaaacacagtgtacctccagatgaactcattgagaccagaggatacagctgtgtattactgtaagacagaccctctgcactgtcgcgcccacgggggctcctggtattctgtgcgtgccaactattggggccagggaacactggttaccgtctctagcggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtct

SEQ ID NO: 570 - DNA, 771 bp, synthetic construct; identical to SEQ ID NO: 557
  source 1..771: mol_type = other DNA

SEQ ID NO: 571 - DNA, 771 bp, synthetic construct
  source 1..771: mol_type = other DNA
gaagtgcagctcctggagagcggcggtggctcggtgcagccaggcggttctctgcgcttgtcctgtgctgcctccggctactcaaactgtgcttacgatatgacttggtatcgccaggcccctggtaaggagcgcgagttcgtctctgccattcattctgacggctcaacccgttatgcagactccgttaaggggcgttttttcatttcccgcgacaactccaagaacaccctgtaccttcagatgaactccctccgcgccgaggataccgccgtatactattgcaagaccgaccccctgcattgcagggctcacggcgggtcctggtactccgtgcgtgccaactattggggacaggggactctggtcacagtctcgagtggaggaggatccgaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcggca

SEQ ID NO: 572 - protein, 6 aa, synthetic construct
  VARIANT 1..6: note = This sequence may encompass 3-6 residues
HHHHHH

SEQ ID NO: 573 - protein, 800 aa, synthetic construct
  VARIANT 1..20: note = This region may encompass 1-20 residues
  VARIANT 21..40: note = This region may encompass 1-20 residues
  VARIANT 41..60: note = This region may encompass 1-20 residues
  VARIANT 61..80: note = This region may encompass 1-20 residues
  VARIANT 81..100: note = This region may encompass 1-20 residues
  VARIANT 101..120: note = This region may encompass 1-20 residues
  VARIANT 121..140: note = This region may encompass 1-20 residues
  VARIANT 141..160: note = This region may encompass 1-20 residues
  VARIANT 161..180: note = This region may encompass 1-20 residues
  VARIANT 181..200: note = This region may encompass 1-20 residues
  VARIANT 201..220: note = This region may encompass 1-20 residues
  VARIANT 221..240: note = This region may encompass 1-20 residues
  VARIANT 241..260: note = This region may encompass 1-20 residues
  VARIANT 261..280: note = This region may encompass 1-20 residues
  VARIANT 281..300: note = This region may encompass 1-20 residues
  VARIANT 301..320: note = This region may encompass 1-20 residues
  VARIANT 321..340: note = This region may encompass 1-20 residues
  VARIANT 341..360: note = This region may encompass 1-20 residues
  VARIANT 361..380: note = This region may encompass 1-20 residues
  VARIANT 381..400: note = This region may encompass 1-20 residues
  VARIANT 401..420: note = This region may encompass 1-20 residues
  VARIANT 421..440: note = This region may encompass 1-20 residues
  VARIANT 441..460: note = This region may encompass 1-20 residues
  VARIANT 461..480: note = This region may encompass 1-20 residues
  VARIANT 481..500: note = This region may encompass 1-20 residues
  VARIANT 501..520: note = This region may encompass 1-20 residues
  VARIANT 521..540: note = This region may encompass 1-20 residues
  VARIANT 541..560: note = This region may encompass 1-20 residues
  VARIANT 561..580: note = This region may encompass 1-20 residues
  VARIANT 581..600: note = This region may encompass 1-20 residues
  VARIANT 601..620: note = This region may encompass 1-20 residues
  VARIANT 621..640: note = This region may encompass 1-20 residues
  VARIANT 641..660: note = This region may encompass 1-20 residues
  VARIANT 661..680: note = This region may encompass 1-20 residues
  VARIANT 681..700: note = This region may encompass 1-20 residues
  VARIANT 701..720: note = This region may encompass 1-20 residues
  VARIANT 721..740: note = This region may encompass 1-20 residues
  VARIANT 741..760: note = This region may encompass 1-20 residues
  VARIANT 761..780: note = This region may encompass 1-20 residues
  VARIANT 781..800: note = This region may encompass 1-20 residues
  VARIANT 1..800: note = This sequence may encompass 1-20 GmSo repeating units, wherein m = 1 to 20, o = 1 to 20
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSS

SEQ ID NO: 574 - protein, 1200 aa, synthetic construct
  VARIANT 1..20: note = This region may encompass 1-20 residues
  VARIANT 21..40: note = This region may encompass 1-20 residues
  VARIANT 41..60: note = This region may encompass 1-20 residues
  VARIANT 61..80: note = This region may encompass 1-20 residues
  VARIANT 81..100: note = This region may encompass 1-20 residues
  VARIANT 101..120: note = This region may encompass 1-20 residues
  VARIANT 121..140: note = This region may encompass 1-20 residues
  VARIANT 141..160: note = This region may encompass 1-20 residues
  VARIANT 161..180: note = This region may encompass 1-20 residues
  VARIANT 181..200: note = This region may encompass 1-20 residues
  VARIANT 201..220: note = This region may encompass 1-20 residues
  VARIANT 221..240: note = This region may encompass 1-20 residues
  VARIANT 241..260: note = This region may encompass 1-20 residues
  VARIANT 261..280: note = This region may encompass 1-20 residues
  VARIANT 281..300: note = This region may encompass 1-20 residues
  VARIANT 301..320: note = This region may encompass 1-20 residues
  VARIANT 321..340: note = This region may encompass 1-20 residues
  VARIANT 341..360: note = This region may encompass 1-20 residues
  VARIANT 361..380: note = This region may encompass 1-20 residues
  VARIANT 381..400: note = This region may encompass 1-20 residues
  VARIANT 401..420: note = This region may encompass 1-20 residues
  VARIANT 421..440: note = This region may encompass 1-20 residues
  VARIANT 441..460: note = This region may encompass 1-20 residues
  VARIANT 461..480: note = This region may encompass 1-20 residues
  VARIANT 481..500: note = This region may encompass 1-20 residues
  VARIANT 501..520: note = This region may encompass 1-20 residues
  VARIANT 521..540: note = This region may encompass 1-20 residues
  VARIANT 541..560: note = This region may encompass 1-20 residues
  VARIANT 561..580: note = This region may encompass 1-20 residues
  VARIANT 581..600: note = This region may encompass 1-20 residues
  VARIANT 601..620: note = This region may encompass 1-20 residues
  VARIANT 621..640: note = This region may encompass 1-20 residues
  VARIANT 641..660: note = This region may encompass 1-20 residues
  VARIANT 661..680: note = This region may encompass 1-20 residues
  VARIANT 681..700: note = This region may encompass 1-20 residues
  VARIANT 701..720: note = This region may encompass 1-20 residues
  VARIANT 721..740: note = This region may encompass 1-20 residues
  VARIANT 741..760: note = This region may encompass 1-20 residues
  VARIANT 761..780: note = This region may encompass 1-20 residues
  VARIANT 781..800: note = This region may encompass 1-20 residues
  VARIANT 801..820: note = This region may encompass 1-20 residues
  VARIANT 821..840: note = This region may encompass 1-20 residues
  VARIANT 841..860: note = This region may encompass 1-20 residues
  VARIANT 861..880: note = This region may encompass 1-20 residues
  VARIANT 881..900: note = This region may encompass 1-20 residues
  VARIANT 901..920: note = This region may encompass 1-20 residues
  VARIANT 921..940: note = This region may encompass 1-20 residues
  VARIANT 941..960: note = This region may encompass 1-20 residues
  VARIANT 961..980: note = This region may encompass 1-20 residues
  VARIANT 981..1000: note = This region may encompass 1-20 residues
  VARIANT 1001..1020: note = This region may encompass 1-20 residues
  VARIANT 1021..1040: note = This region may encompass 1-20 residues
  VARIANT 1041..1060: note = This region may encompass 1-20 residues
  VARIANT 1061..1080: note = This region may encompass 1-20 residues
  VARIANT 1081..1100: note = This region may encompass 1-20 residues
  VARIANT 1101..1120: note = This region may encompass 1-20 residues
  VARIANT 1121..1140: note = This region may encompass 1-20 residues
  VARIANT 1141..1160: note = This region may encompass 1-20 residues
  VARIANT 1161..1180: note = This region may encompass 1-20 residues
  VARIANT 1181..1200: note = This region may encompass 1-20 residues
  VARIANT 1..1200: note = This sequence may encompass 1-20 GmSoGm repeating units, wherein m = 1 to 20, o = 1 to 20
GGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGG

SEQ ID NO: 575 - protein, 460 aa, synthetic construct
  VARIANT 4..23: note = This region may encompass 1-20 residues
  VARIANT 27..46: note = This region may encompass 1-20 residues
  VARIANT 50..69: note = This region may encompass 1-20 residues
  VARIANT 73..92: note = This region may encompass 1-20 residues
  VARIANT 96..115: note = This region may encompass 1-20 residues
  VARIANT 119..138: note = This region may encompass 1-20 residues
  VARIANT 142..161: note = This region may encompass 1-20 residues
  VARIANT 165..184: note = This region may encompass 1-20 residues
  VARIANT 188..207: note = This region may encompass 1-20 residues
  VARIANT 211..230: note = This region may encompass 1-20 residues
  VARIANT 234..253: note = This region may encompass 1-20 residues
  VARIANT 257..276: note = This region may encompass 1-20 residues
  VARIANT 280..299: note = This region may encompass 1-20 residues
  VARIANT 303..322: note = This region may encompass 1-20 residues
  VARIANT 326..345: note = This region may encompass 1-20 residues
  VARIANT 349..368: note = This region may encompass 1-20 residues
  VARIANT 372..391: note = This region may encompass 1-20 residues
  VARIANT 395..414: note = This region may encompass 1-20 residues
  VARIANT 418..437: note = This region may encompass 1-20 residues
  VARIANT 441..460: note = This region may encompass 1-20 residues
  VARIANT 1..460: note = This sequence may encompass 1-20 GGGSm repeating units, wherein m = 1 to 20
GGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSS

SEQ ID NO: 576 - DNA, 378 bp, synthetic construct
  source 1..378: mol_type = other DNA
gaggtgcagctggtggagagcggaggagggttagtgcagccgggcggttctctcagacttagctgcgccgcctccgggtacacgcactcttcctattgcatgggctgggtcaggcaggcgcctgggaagggcctggagggagttagtgccatcgatgtcgatggctcaacaacctatgcagacagcgttaagggtcggttcaccatttcacgcgacaatagcaaaaacactctgtatttgcagatgaacagtctccgtgccgaagacacagctgtctactactgtgcagctgaatttgctgattgttcctctaattatttcctacccccaggggcagtgcgatactggggacaaggcaccctggtaactgtctcgtct

SEQ ID NO: 577 - protein, 100 aa, synthetic construct
  VARIANT 1..100: note = This sequence may encompass 1-20 GSGGS repeating units
GSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGS

SEQ ID NO: 578 - protein, 480 aa, synthetic construct
  VARIANT 5..24: note = This region may encompass 1-20 residues
  VARIANT 29..48: note = This region may encompass 1-20 residues
  VARIANT 53..72: note = This region may encompass 1-20 residues
  VARIANT 77..96: note = This region may encompass 1-20 residues
  VARIANT 101..120: note = This region may encompass 1-20 residues
  VARIANT 125..144: note = This region may encompass 1-20 residues
  VARIANT 149..168: note = This region may encompass 1-20 residues
  VARIANT 173..192: note = This region may encompass 1-20 residues
  VARIANT 197..216: note = This region may encompass 1-20 residues
  VARIANT 221..240: note = This region may encompass 1-20 residues
  VARIANT 245..264: note = This region may encompass 1-20 residues
  VARIANT 269..288: note = This region may encompass 1-20 residues
  VARIANT 293..312: note = This region may encompass 1-20 residues
  VARIANT 317..336: note = This region may encompass 1-20 residues
  VARIANT 341..360: note = This region may encompass 1-20 residues
  VARIANT 365..384: note = This region may encompass 1-20 residues
  VARIANT 389..408: note = This region may encompass 1-20 residues
  VARIANT 413..432: note = This region may encompass 1-20 residues
  VARIANT 437..456: note = This region may encompass 1-20 residues
  VARIANT 461..480: note = This region may encompass 1-20 residues
  VARIANT 1..480: note = This sequence may encompass 1-20 GSGGSm repeating units, wherein m = 1 to 20
GSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSS

SEQ ID NO: 579 - protein, 480 aa, synthetic construct
  VARIANT 4..23: note = This region may encompass 1-20 residues
  VARIANT 28..47: note = This region may encompass 1-20 residues
  VARIANT 52..71: note = This region may encompass 1-20 residues
  VARIANT 76..95: note = This region may encompass 1-20 residues
  VARIANT 100..119: note = This region may encompass 1-20 residues
  VARIANT 124..143: note = This region may encompass 1-20 residues
  VARIANT 148..167: note = This region may encompass 1-20 residues
  VARIANT 172..191: note = This region may encompass 1-20 residues
  VARIANT 196..215: note = This region may encompass 1-20 residues
  VARIANT 220..239: note = This region may encompass 1-20 residues
  VARIANT 244..263: note = This region may encompass 1-20 residues
  VARIANT 268..287: note = This region may encompass 1-20 residues
  VARIANT 292..311: note = This region may encompass 1-20 residues
  VARIANT 316..335: note = This region may encompass 1-20 residues
  VARIANT 340..359: note = This region may encompass 1-20 residues
  VARIANT 364..383: note = This region may encompass 1-20 residues
  VARIANT 388..407: note = This region may encompass 1-20 residues
  VARIANT 412..431: note = This region may encompass 1-20 residues
  VARIANT 436..455: note = This region may encompass 1-20 residues
  VARIANT 460..479: note = This region may encompass 1-20 residues
  VARIANT 1..480: note = This sequence may encompass 1-20 GSGSmG repeating units, wherein m = 1 to 20
GSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSG

SEQ ID NO: 580 - DNA, 824 bp, synthetic construct
  source 1..824: mol_type = other DNA
accggtgtccactctgaggtgcagctgcaagagagcggaggaggaagcgtccaagccggaggctctctgagactgagctgtgccgccagcggctactccaactgcgcctacgacatgacttggtataggcaagcccccggcaaggagagggagttcgtgtccgccatccacagcgacggcagcactagatacgccgacagcgtgaagggaaggttcttcatcagccaagataacgccaagaacacagtgtatctgcagatgaactccctcaagccagaggacactgccatgtactactgcaagacagacccactgcactgcagagcccatggcggcagctggtatagcgtgagggccaactactggggccaaggcacacaagtgacagtctcgagcggcggaggatcccaagtgcagctgcaagagagcggaggaggcagcgtcgaagctggaggatctctgaggctgagctgtgctgccagcggctacactcacagcagctactgtatgggctggttcagacaagcccccggcaaggagagggaaggcgtggctgccatcgacgtggatggcagcactacttacgccgacagcgtgaagggaaggttcactatcagcaaggacaacgccaagaacacactctatctgcagatgaacagcctcaagccagaggacactggcatgtactactgcgccgccgagttcgccgattgcagcagcaactactttctgcctcccggcgccgtcagatattggggccaaggcactcaagtgacagtctcgtctgctagccaccatcaccatcaccactgataagcggccgc

SEQ ID NO: 581 - protein, 6 aa, synthetic construct
  VARIANT 1: note = T or L
XVTVSS

(19) United States
(12) Patent Application Publication
(10) Pub. No.: US 2026/0317435 A1
(43) Pub. Date: Oct. 15, 2026

(54) HUMANIZED IL-10 RECEPTOR BINDING MOLECULES AND METHODS OF USE

(71) Applicant: Synthekine, Inc., Menlo Park, CA (US)

(72) Inventors: Patrick J. Lupardus, Menlo Park, CA (US); Andrew Morin, San Mateo, CA (US); Mahalakshmi Ramadass, Fremont, CA (US); Deepti Rokkam, Sunnyvale, CA (US); Michael Totagrande, San Leandro, CA (US); Sandro Vivona, Emerald Hills, CA (US)

(21) Appl. No.: 19/490,228

(22) PCT Filed: Jun. 7, 2024
(86) PCT No.: PCT/US2024/033162

§ 371 Date: Dec. 4, 2025

Related U.S. Application Data
(60) Provisional application No. 63/506,798, filed on Jun. 7, 2023.
(60) Provisional application No. 63/511,122, filed on Jun. 29, 2023.

(51) Int. Cl. C07K 16/28

(52) U.S. Cl. CPC C07K 16/2866; C07K 2317/24; C07K 2317/31; C07K 2317/569

(57) ABSTRACT

Provided herein are humanized IL-10 agonists that bind to IL-10Rα and IL-10Rβ and comprise an IL-10Rα single-domain antibody and an IL-10Rβ single-domain antibody.

### CROSS-REFERENCE TO RELATED APPLICATIONS

The present application is a U.S. National Phase Application of PCT/US2024/033162, filed on Jun. 7, 2024, which claims priority to U.S. Provisional Patent Application No. 63/506,798, filed on Jun. 7, 2023, and U.S. Provisional Patent Application No. 63/511,122, filed on Jun. 29, 2023, the disclosures of which are incorporated herein by reference in their entirety for all purposes.

### SEQUENCE LISTING

The contents of the electronic sequence listing (106249-009220PC-1446129_SL.xml; size 558,534 bytes; and date of creation Aug. 27, 2024) is incorporated by reference.

### BACKGROUND OF THE DISCLOSURE

Cytokine and growth-factor ligands typically signal through the multimerization of cell surface receptor subunits. In some instances, cytokines act as multispecific (e.g., bispecific or trispecific) ligands that facilitate the association of such receptor subunits, bringing intracellular domains into proximity such that intracellular signaling may occur. The cytokine determines which receptor subunits are associated to form the cytokine receptor complex. Cytokines thus act to bridge the individual receptor subunits into a receptor complex that results in intracellular signaling.

One useful approach to cytokine engineering has been to use single-domain antibodies (sdAbs), such as single variable domain (sFv) antibodies and variable heavy domain (VHH) antibodies. VHHs are sdAbs comprising only the variable domains derived from heavy chain antibodies derived from camelid species.

Consequently, there is a need in the art to engineer and generate single-domain molecules that have activity similar to their surrogate or cognate cytokine or that engage with the cytokine receptor subunits by preferentially binding a particular subunit (i.e., “biased” signaling) and that do not have independent off-target activity, such as single-domain antibodies (sdAbs) and dimers of sdAbs having two independent binding domains (i.e., two sdAbs having similar or distinct binding specificity) that independently bind different receptors or receptor subunits.

### SUMMARY OF THE DISCLOSURE

The present disclosure provides IL-10 agonist compounds and compositions comprising a humanized single-domain antibody (sdAb) that binds to IL-10Rα (such as an anti-IL-10Rα sdAb or VHH joined to a humanized single-domain antibody (sdAb) that binds to IL-10Rβ (such as an anti-IL-10Rα sdAb or VHH). Such compositions are useful in the pairing of cellular receptors to generate desirable effects useful in the treatment of disease in mammalian subjects.

The present disclosure provides IL-10 agonist compounds comprising at least a first domain (e.g., a first single-domain antibody polypeptide) that specifically binds to a first receptor subunit (e.g., IL-10Rα) and a second domain (e.g., second single-domain antibody polypeptide) that specifically binds to a second receptor subunit (e.g., IL-10Rβ). In some embodiments, contacting the IL-10 agonist compound with a cell expressing the first and second receptor subunits results in the functional association of the first and second receptor subunits, thereby triggering their interaction and resulting in downstream signaling. In some embodiments, the downstream signaling is different from the downstream signaling resulting from the native ligand binding to the native receptor subunits. In some embodiments, the first and second receptor subunits occur in proximity in response to the cognate ligand binding and are referred to herein as “natural” cytokine receptor pairs.

In some embodiments, the present disclosure provides IL-10 agonist compounds that comprise a first domain (e.g., a first single-domain antibody polypeptide) that binds to IL-10Rα of the IL-10 receptor and a second domain (e.g., a second single-domain antibody polypeptide) that binds to IL-10Rβ of the IL-10 receptor. In some embodiments, contacting the IL-10 agonist compound with a cell expressing IL-10Rα of the IL-10 receptor and IL-10Rβ of the IL-10 receptor results in the functional association of IL-10Rα and IL-10Rβ, thereby resulting in functional dimerization of the receptors and downstream signaling.

In some embodiments, the present disclosure provides IL-10 agonist compounds comprising a first single-domain antibody polypeptide joined to (e.g., covalently linked, such as via a polypeptide linker or a chemical linker, or stably associated via a non-covalent linkage via an Fc construct, as described in more detail herein) a second single-domain antibody polypeptide, wherein the first single-domain antibody polypeptide that specifically binds to the a subunit of the IL-10 receptor (IL10Rα) comprises: a CDR1 comprising an amino acid sequence selected from the group consisting of SEQ ID NOS:224-228; a CDR2 comprising an amino acid sequence selected from the group consisting of SEQ ID NO:229-235; and a CDR3 comprising the amino acid sequence of of SEQ ID NO:236; and wherein the second single-domain antibody polypeptide that specifically binds to the R subunit of the IL-10 receptor (IL10Rβ) comprises: a CDR1 comprising an amino acid sequence selected from the group consisting of SEQ ID NO:296; a CDR2 comprising an amino acid sequence selected from the group consisting of SEQ ID NO:297; and a CDR3 comprising an amino acid sequence selected from the group consisting of SEQ ID NO:298.

In some embodiments, the present disclosure provides IL-10 agonist compounds, wherein the first single-domain antibody and the second single-domain antibody are joined by a linker. In some embodiments, the present disclosure provides IL-10 agonist compounds comprising a single polypeptide chain, wherein the IL-10 agonist compounds comprise a first single-domain antibody that binds to IL10Rα and a second single-domain antibody that binds that binds to IL10Rβ, optionally wherein the first single-domain antibody and the second single-domain antibody are joined by a polypeptide linker, wherein the polypeptide linker comprises 1-50, alternatively 1-30, alternatively 1-20, alternatively 1-15, alternatively 1-12 alternatively 1-10, alternatively 1-8, alternatively 1-6, alternatively 1-5, alternatively 1-4, alternatively 1-3, alternatively 1-2, alternatively 1 amino acid(s).

In some embodiments, the present disclosure provides IL-10 agonist compounds comprising a first single-domain antibody comprising an amino acid sequence selected from the group consisting of SEQ ID NOS:25-48; and a second single-domain antibody comprising an amino acid sequence selected from the group consisting of SEQ ID NOS:49 and 50. In some embodiments, the present disclosure provides IL-10 agonist compounds comprising a first single-domain antibody comprising an amino acid sequence selected from the group consisting of SEQ ID NOS:25-48; and a second single-domain antibody comprising an amino acid sequence selected from the group consisting of SEQ ID NOS:49 and 50, optionally wherein the first single-domain antibody and the second single-domain antibody are joined by a polypeptide linker comprising 1-50, alternatively 1-30, alternatively 1-20, alternatively 1-15, alternatively 1-12 alternatively 1-10, alternatively 1-8, alternatively 1-6, alternatively 1-5, alternatively 1-4, alternatively 1-3, alternatively 1-2, alternatively 1 amino acid(s). In some embodiments, the present disclosure provides IL-10 agonist compounds comprising a first single-domain antibody comprising an amino acid sequence selected from the group consisting of SEQ ID NOS:25-48; and a second single-domain antibody comprising an amino acid sequence selected from the group consisting of SEQ ID NOS:49 and 50 wherein the first single-domain antibody and the second single-domain antibody are joined by a polypeptide linker selected from the group consisting of SEQ ID NOS:416-439.

In some embodiments, the present disclosure provides IL-10 agonist compounds, wherein the C-terminus of the first single-domain antibody is joined to the N-terminus of the second single-domain antibody, optionally wherein the C-terminus of the first single-domain antibody is joined to the N-terminus of the second single-domain antibody via a linker.

In some embodiments, the present disclosure provides IL-10 agonist compounds, wherein the N-terminus of the first single-domain antibody is joined to the C-terminus of the second single-domain antibody, optionally wherein the N-terminus of the first single-domain antibody is joined to the C-terminus of the second single-domain antibody via a linker. In some embodiments, the present disclosure provides IL-10 agonist compounds comprising a first single-domain antibody that binds to the extracellular domain of IL10Rα and second single-domain antibody that binds to the extracellular domain of IL10Rβ, wherein the N-terminus of the first single-domain antibody is joined to the C-terminus of the second single-domain antibody that binds to IL10Rβ, optionally wherein the N-terminus of the first single-domain antibody is joined to the C-terminus of the second single-domain antibody via a polypeptide linker comprising 1-50, alternatively 1-30, alternatively 1-20, alternatively 1-15, alternatively 1-12 alternatively 1-10, alternatively 1-8, alternatively 1-6, alternatively 1-5, alternatively 1-4, alternatively 1-3, alternatively 1-2, alternatively 1 amino acid(s). In some embodiments, the present disclosure provides IL-10 agonist compounds comprising a first single-domain antibody that binds to the extracellular domain of IL10Rβ and second single-domain antibody that binds to the extracellular domain of IL10Rα, wherein the N-terminus of the first single-domain antibody is joined to the C-terminus of the second single-domain antibody that binds to IL10Rβ, optionally wherein the N-terminus of the first single-domain antibody is joined to the C-terminus of the second single-domain antibody via a polypeptide linker comprising 1-50, alternatively 1-30, alternatively 1-20, alternatively 1-15, alternatively 1-12 alternatively 1-10, alternatively 1-8, alternatively 1-6, alternatively 1-5, alternatively 1-4, alternatively 1-3, alternatively 1-2, alternatively 1 amino acid(s).

In some embodiments, the present disclosure provides IL-10 agonist compounds comprising a polypeptide selected from the group consisting of: SEQ ID NOS:1-24 and 500-523.

In some embodiments, the present disclosure provides IL-10 agonist compounds comprising a polypeptide comprising at least 95% amino acid sequence identity to an amino acid sequence selected from the group consisting of: SEQ ID NOS:1-24 and 500-523. In some embodiments, the present disclosure provides IL-10 agonist compounds comprising a polypeptide comprising at least 95%, alternatively at least 96%, alternatively at least 97%, alternatively at least 98%, alternatively at least 99%, or alternatively 100% amino acid sequence identity to an amino acid sequence selected from the group consisting of: SEQ ID NOS:1-24 and 500-523. In some embodiments, the present disclosure provides IL-10 agonist compounds comprising a polypeptide comprising at least 95%, alternatively at least 96%, alternatively at least 97%, alternatively at least 98%, alternatively at least 99%, or alternatively 100% amino acid sequence identity to an amino acid sequence selected from the group consisting of: SEQ ID NOS:1-24 and 500-523. In some embodiments, the present disclosure provides IL-10 agonist compounds comprising a polypeptide comprising at least 95%, alternatively at least 96%, alternatively at least 97%, alternatively at least 98%, alternatively at least 99%, or alternatively 100% amino acid sequence identity to an amino acid of SEQ ID NO:1 (DR2463). In some embodiments, the present disclosure provides IL-10 agonist compounds comprising a polypeptide comprising at least 95%, alternatively at least 96%, alternatively at least 97%, alternatively at least 98%, alternatively at least 99%, or alternatively 100% amino acid sequence identity to an amino acid of SEQ ID NO:2 (DR2485). In some embodiments, the present disclosure provides IL-10 agonist compounds comprising a polypeptide comprising at least 95%, alternatively at least 96%, alternatively at least 97%, alternatively at least 98%, alternatively at least 99%, or alternatively 100% amino acid sequence identity to an amino acid of SEQ ID NO:3 (DR2519). In some embodiments, the present disclosure provides IL-10 agonist compounds comprising a polypeptide comprising at least 95%, alternatively at least 96%, alternatively at least 97%, alternatively at least 98%, alternatively at least 99%, or alternatively 100% amino acid sequence identity to an amino acid of SEQ ID NO:4 (DR2520).

In some embodiments, the present disclosure provides IL-10 agonist compounds comprising a polypeptide comprising at least 95%, alternatively at least 96%, alternatively at least 97%, alternatively at least 98%, alternatively at least 99%, or alternatively 100% amino acid sequence identity to an amino acid of SEQ ID NO:500 (DR2463, non-his tagged). In some embodiments, the present disclosure provides IL-1 agonist compounds comprising a polypeptide comprising at least 9500 alternatively at least 960%, alternatively at least 9700, alternatively at least 9800, alternatively at least 9900, or alternatively 10000 amino acid sequence identity to an amino acid of SEQ ID NO:501 (DR2485, non-his tagged). In some embodiments, the present disclosure provides IL-10 agonist compounds comprising a polypeptide comprising at least 9500 alternatively at least 960%, alternatively at least 9700, alternatively at least 980%, alternatively at least 9900 or alternatively 1000% amino acid sequence identity to an amino acid of SEQ ID NO:502 (DR2519, non-his tagged). In some embodiments, the present disclosure provides IL-10 agonist compounds comprising a polypeptide comprising at least 9500 alternatively at least 960%, alternatively at least 9700 alternatively at least 980%, alternatively at least 9900 or alternatively 1000% amino acid sequence identity to an amino acid of SEQ ID NO:503 (DR2520, non his-tagged).

In some embodiments, the present disclosure provides IL-10 agonist compounds, wherein the first single-domain antibody polypeptide comprises a combination of CDR1, CDR2, and CDR3 amino acid sequences selected from one of the rows of the following table:

IL-10Rα VHH CDRs

| CDR1 SEQ ID NO: | CDR2 SEQ ID NO: | CDR3 SEQ ID NO: |
|---|---|---|
| 224 | 229 | 236 |
| 224 | 230 | 236 |
| 224 | 231 | 236 |
| 225 | 229 | 236 |
| 226 | 229 | 236 |
| 224 | 232 | 236 |
| 224 | 233 | 236 |
| 224 | 234 | 236 |
| 227 | 229 | 236 |
| 224 | 235 | 236 |
| 227 | 235 | 236 |
| 228 | 229 | 236 |

and wherein the second single-domain antibody polypeptide comprises a combination of CDR1, CDR2, and CDR3 amino acid sequences selected from one of the rows of the following table:

IL-10Rβ VHH CDRs

| CDR1 SEQ ID NO: | CDR2 SEQ ID NO: | CDR3 SEQ ID NO: |
|---|---|---|
| 296 | 297 | 298 |

In some embodiments, the present disclosure provides IL-10 agonist compounds comprising a first single-domain antibody polypeptide and a second single-domain antibody polypeptide, wherein the first single-domain antibody polypeptide binds to the extracellular domain of IL10Rα and comprises a CDR1, CDR2 and CDR3 wherein:
(a) CDR1 is a polypeptide comprising the amino acid sequence of SEQ ID NO:224, CDR2 is a polypeptide comprising the amino acid sequence of SEQ ID NO:229 and CDR3 is a polypeptide comprising the amino acid sequence of SEQ ID NO:236; or
CDR1 is a polypeptide comprising the amino acid sequence of SEQ ID NO:224, CDR2 is a polypeptide comprising the amino acid sequence of SEQ ID NO:230, and CDR3 is a polypeptide comprising the amino acid sequence of SEQ ID NO:236; or
CDR1 is a polypeptide comprising the amino acid sequence of SEQ ID NO:224, CDR2 is a polypeptide comprising the amino acid sequence of SEQ ID NO:231 and CDR3 is a polypeptide comprising the amino acid sequence of SEQ ID NO:236;
CDR1 is a polypeptide comprising the amino acid sequence of SEQ ID NO:224, CDR2 is a polypeptide comprising the amino acid sequence of SEQ ID NO:231, and CDR3 is a polypeptide comprising the amino acid sequence of SEQ ID NO:236; or
CDR1 is a polypeptide comprising the amino acid sequence of SEQ ID NO:224, CDR2 is a polypeptide comprising the amino acid sequence of SEQ ID NO:232, and CDR3 is a polypeptide comprising the amino acid sequence of SEQ ID NO:236; or
CDR1 is a polypeptide comprising the amino acid sequence of SEQ ID NO:224, CDR2 is a polypeptide comprising the amino acid sequence of SEQ ID NO:233, and CDR3 is a polypeptide comprising the amino acid sequence of SEQ ID NO:236; or
CDR1 is a polypeptide comprising the amino acid sequence of SEQ ID NO:224, CDR2 is a polypeptide comprising the amino acid sequence of SEQ ID NO:234, and CDR3 is a polypeptide comprising the amino acid sequence of SEQ ID NO:236; or
CDR1 is a polypeptide comprising the amino acid sequence of SEQ ID NO:225, CDR2 is a polypeptide comprising the amino acid sequence of SEQ ID NO:229, and CDR3 is a polypeptide comprising the amino acid sequence of SEQ ID NO:236; or
CDR1 is a polypeptide comprising the amino acid sequence of SEQ ID NO:225, CDR2 is a polypeptide comprising the amino acid sequence of SEQ ID NO:235, and CDR3 is a polypeptide comprising the amino acid sequence of SEQ ID NO:236; or
CDR1 is a polypeptide comprising the amino acid sequence of SEQ ID NO:226, CDR2 is a polypeptide comprising the amino acid sequence of SEQ ID NO:230, and CDR3 is a polypeptide comprising the amino acid sequence of SEQ ID NO:236;
CDR1 is a polypeptide comprising the amino acid sequence of SEQ ID NO:228, CDR2 is a polypeptide comprising the amino acid sequence of SEQ ID NO:229, and CDR3 is a polypeptide comprising the amino acid sequence of SEQ ID NO:236; and/or
(b) the second single-domain antibody polypeptide binds to the extracellular domain of IL10Rβ and comprises a CDR1, CDR2 and CDR3 wherein:
CDR1 is a polypeptide comprising the amino acid sequence of SEQ ID NO:296, CDR2 is a polypeptide comprising the amino acid sequence of SEQ ID NO:297 and CDR3 is a polypeptide comprising the amino acid sequence of SEQ ID NO:298,
optionally wherein the first single-domain antibody polypeptide that binds to the extracellular domain of IL-10Rα (IL-10Rα sdAb) is at least 80% humanized relative to UniProt V3-23 (UniProt No. P01764), and/or
optionally wherein the second single-domain antibody polypeptide that binds to IL-10Rβ (IL-10Rβ sdAb) is at least 89% humanized relative to Uniprot VH3-66 (UniProt A0A0C4DH42), and/or
further optionally wherein the N-terminus of the first single-domain antibody polypeptide is joined to the C-terminus of the second single-domain antibody polypeptide, or alternatively wherein the C-terminus of the first single-domain antibody polypeptide, is joined to the N-terminus of the second single-domain antibody polypeptide, via a polypeptide linker comprising 1-50, alternatively 1-30, alternatively 1-20, alternatively 1-15, alternatively 1-12 alternatively 1-10, alternatively 1-8, alternatively 1-6, alternatively 1-5, alternatively 1-4, alternatively 1-3, alternatively 1-2, alternatively 1 amino acid(s).

In some embodiments, the present disclosure provides and IL-10 agonist compound comprising a first single-domain antibody polypeptide and a second single-domain antibody polypeptide, wherein the first single-domain antibody polypeptide binds to the extracellular domain of IL10Rα and comprises a CDR1, CDR2 and CDR3 wherein CDR1 is a polypeptide comprising the amino acid sequence of SEQ ID NO:224, CDR2 is a polypeptide comprising the amino acid sequence of SEQ ID NO:229 and CDR3 is a polypeptide comprising the amino acid sequence SEQ ID NO:236 and optionally wherein the first single domain antibody polypeptide that binds to the extracellular domain of IL-10Rα (IL-10Rα sdAb) is at least 80% humanized relative to UniProt V3-23 (UniProt No. P01764), and (b) the second single-domain antibody polypeptide binds to the extracellular domain of IL10Rβ and comprises a CDR1, CDR2 and CDR3 wherein: CDR1 is a polypeptide comprising the amino acid sequence of SEQ ID NO:296, CDR2 is a polypeptide comprising the amino acid sequence of SEQ ID NO:297 and CDR3 is a polypeptide comprising the amino acid sequence of SEQ ID NO:298, and optionally wherein the second single domain antibody polypeptide that binds to IL-10Rβ (IL-10Rβ sdAb) is at least 89% humanized relative to Uniprot VH3-66 (UniProt A0A0C4DH42) and further optionally wherein the C-terminus of the first single-domain antibody polypeptide is joined to the N-terminus of second single-domain antibody polypeptide via a polypeptide linker comprising 1-50, alternatively 1-30, alternatively 1-20, alternatively 1-15, alternatively 1-12 alternatively 1-10, alternatively 1-8, alternatively 1-6, alternatively 1-5, alternatively 1-4, alternatively 1-3, alternatively 1-2, alternatively 1 amino acid(s).

In some embodiments, the present disclosure provides and IL-10 agonist compound comprising a first single-domain antibody polypeptide and a second single-domain antibody polypeptide, wherein the first single-domain antibody polypeptide binds to the extracellular domain of IL10Rα and comprises a CDR1, CDR2 and CDR3 wherein CDR1 is a polypeptide comprising the amino acid sequence of SEQ ID NO:224, CDR2 is a polypeptide comprising the amino acid sequence of SEQ ID NO:230 and CDR3 is a polypeptide comprising the amino acid sequence SEQ ID NO:236 and optionally wherein the first single domain antibody polypeptide that binds to the extracellular domain of IL-10Rα (IL-10Rα sdAb) is at least 80% humanized relative to UniProt V3-23 (UniProt No. P01764), and (b) the second single-domain antibody polypeptide binds to the extracellular domain of IL10Rβ and comprises a CDR1, CDR2 and CDR3 wherein: CDR1 is a polypeptide comprising the amino acid sequence of SEQ ID NO:296, CDR2 is a polypeptide comprising the amino acid sequence of SEQ ID NO:297 and CDR3 is a polypeptide comprising the amino acid sequence of SEQ ID NO:298, and optionally wherein the second single domain antibody polypeptide that binds to IL-10Rβ (IL-10Rβ sdAb) is at least 89% humanized relative to Uniprot VH3-66 (UniProt A0A0C4DH42) and further optionally wherein the C-terminus of the first single-domain antibody polypeptide is joined to the N-terminus of second single-domain antibody polypeptide via a polypeptide linker comprising 1-50, alternatively 1-30, alternatively 1-20, alternatively 1-15, alternatively 1-12 alternatively 1-10, alternatively 1-8, alternatively 1-6, alternatively 1-5, alternatively 1-4, alternatively 1-3, alternatively 1-2, alternatively 1 amino acid(s).

In some embodiments, the present disclosure provides and IL-10 agonist compound comprising a first single-domain antibody polypeptide and a second single-domain antibody polypeptide, wherein the first single-domain antibody polypeptide binds to the extracellular domain of IL10Rα and comprises a CDR1, CDR2 and CDR3 wherein CDR1 is a polypeptide comprising the amino acid sequence of SEQ ID NO:224, CDR2 is a polypeptide comprising the amino acid sequence of SEQ ID NO:231 and CDR3 is a polypeptide comprising the amino acid sequence SEQ ID NO:236 and optionally wherein the first single domain antibody polypeptide that binds to the extracellular domain of IL-10Rα (IL-10Rα sdAb) is at least 80% humanized relative to UniProt V3-23 (UniProt No. P01764), and (b) the second single-domain antibody polypeptide binds to the extracellular domain of IL10Rβ and comprises a CDR1, CDR2 and CDR3 wherein: CDR1 is a polypeptide comprising the amino acid sequence of SEQ ID NO:296, CDR2 is a polypeptide comprising the amino acid sequence of SEQ ID NO:297 and CDR3 is a polypeptide comprising the amino acid sequence SEQ ID NO:298, optionally wherein the second single domain antibody polypeptide that binds to IL-10Rβ (IL-10Rβ sdAb) is at least 89% humanized relative to Uniprot VH3-66 (UniProt A0A0C4DH42) and further optionally wherein the C-terminus of the first single-domain antibody polypeptide is joined to the N-terminus of second single-domain antibody polypeptide via a polypeptide linker comprising 1-50, alternatively 1-30, alternatively 1-20, alternatively 1-15, alternatively 1-12 alternatively 1-10, alternatively 1-8, alternatively 1-6, alternatively 1-5, alternatively 1-4, alternatively 1-3, alternatively 1-2, alternatively 1 amino acid(s).

In some embodiments, the present disclosure provides IL-10 agonist compounds, wherein the first single-domain antibody polypeptide comprises: a CDR1 comprising the amino acid sequence of SEQ ID NO:224; a CDR2 comprising the amino acid sequence of SEQ ID NO:229; and a CDR3 comprising the amino acid sequence of SEQ ID NO:236; and wherein the second single-domain antibody polypeptide comprises: a CDR1 comprising the amino acid sequence of SEQ ID NO:296; a CDR2 comprising the amino acid sequence of SEQ ID NO:297; and a CDR3 comprising the amino acid sequence of SEQ ID NO:298.

In some embodiments, the present disclosure provides IL-10 agonist compounds, wherein the first single-domain antibody polypeptide comprises: a CDR1 comprising the amino acid sequence of SEQ ID NO:224; a CDR2 comprising the amino acid sequence of SEQ ID NO:230; and a CDR3 comprising the amino acid sequence of SEQ ID NO:236; and wherein the second single-domain antibody polypeptide comprises: a CDR1 comprising the amino acid sequence of SEQ ID NO:296; a CDR2 comprising the amino acid sequence of SEQ ID NO:297; and a CDR3 comprising the amino acid sequence of SEQ ID NO:298.

In some embodiments, the present disclosure provides IL-10 agonist compounds, wherein the first single-domain antibody polypeptide comprises: a CDR1 comprising the amino acid sequence of SEQ ID NO:224; a CDR2 comprising the amino acid sequence of SEQ ID NO:231; and a CDR3 comprising the amino acid sequence of SEQ ID NO:236; and wherein the second single-domain antibody polypeptide comprises: a CDR1 comprising the amino acid sequence of SEQ ID NO:296; a CDR2 comprising the amino acid sequence of SEQ ID NO:297; and a CDR3 comprising the amino acid sequence of SEQ ID NO:298.

In some embodiments, the present disclosure provides IL-10 agonist compounds comprising a polypeptide comprising an amino acid sequence selected from the group consisting of SEQ ID NOS:1-24 and 500-523.

In some embodiments, the present disclosure provides IL-10 agonist compounds comprising a polypeptide comprising the amino acid sequence of SEQ ID NO:1 (DR2463). In some embodiments, the present disclosure provides IL-10 agonist compounds comprising a polypeptide comprising the amino acid sequence of SEQ ID NO:2 (DR2485). In some embodiments, the present disclosure provides IL-10 agonist compounds comprising a polypeptide comprising the amino acid sequence of SEQ ID NO:3 (DR2519). In some embodiments, the present disclosure provides IL-10 agonist compounds comprising a polypeptide comprising the amino acid sequence of SEQ ID NO:4 (DR2520).

In some embodiments, the present disclosure provides IL-10 agonist compounds comprising a polypeptide comprising the amino acid sequence of SEQ ID NO:500 (DR2463, non-his tagged). In some embodiments, the present disclosure provides IL-10 agonist compounds comprising a polypeptide comprising the amino acid sequence of SEQ ID NO:501 (DR2485, non-his tagged). In some embodiments, the present disclosure provides IL-10 agonist compounds comprising a polypeptide comprising the amino acid sequence of SEQ ID NO:502 (DR2519, non-his tagged). In some embodiments, the present disclosure provides IL-10 agonist compounds comprising a polypeptide comprising the amino acid sequence of SEQ ID NO:4 (DR2520, non-his tagged).

In some embodiments, the present disclosure provides pharmaceutically acceptable formulations of an IL-10 agonist compound disclosed herein.

In some embodiments, the present disclosure provides nucleic acid sequences encoding an IL-10 agonist compound disclosed herein. In some embodiments, the present disclosure provides recombinant vectors comprising such nucleic acids.

The IL-10 agonist compounds (e.g., single-domain antibody polypeptides of the IL-10 agonist compounds) of the present disclosure are useful in the treatment or prevention of disease in mammalian subjects. In some embodiments, the present disclosure provides methods of treating a mammalian subject suffering from an autoimmune disease, infectious disease, or inflammatory disease by the administration of a therapeutically effective amount of an IL-10 agonist compound disclosed herein. In some embodiments, the present disclosure provides for the treatment or prevention of infectious disease, including viral and chronic viral infections, in a mammalian subject by the administration of a therapeutically effective amount of an IL-10 agonist compound of the present disclosure. In some embodiments, the present disclosure provides methods of treating a mammalian subject suffering from a neoplastic disease by the administration of a therapeutically effective amount of an IL-10 agonist compound disclosed herein.

In another aspect, the disclosure provides a method for treating neoplastic diseases, such as cancer in a subject in need thereof, comprising administering to the subject the IL-10 agonist protein described herein, wherein the IL-10 agonist protein binds to and activates CD8⁺ T cells, CD4⁺ T cells, macrophages, and/or Treg cells. In some embodiments, the IL-10 agonist protein provides longer therapeutic efficacy than a pegylated IL-10. In some embodiments, the cancer is a solid tumor cancer.

In some embodiments, the present disclosure provides means for inducing intracellular signaling in a cell expressing IL-10Rα and IL-10Rβ, wherein the means comprises (i) a single domain antibody polypeptide that binds to IL-10Rα (IL-10Rα sdAb) and is at least 80% humanized relative to UniProt V3-23 (UniProt No. P01764), joined to (ii) a single domain antibody polypeptide that binds to IL-10Rβ (IL-10Rβ sdAb) and that is at least 89% humanized relative to Uniprot VH3-66 (UniProt A0A0C4DH42); and a pharmaceutically acceptable carrier.

In some embodiments, the present disclosure provides compositions comprising IL-10 agonist compounds described herein, wherein the cell expressing IL-10Rα and IL-10Rβ is a monocyte.

In some embodiments, the present disclosure provides compositions, wherein dimerizing the extracellular domains of an IL-10Rα subunit and an IL-10Rβ subunit of the IL-10 receptor (IL-10R) on a cell induces pSTAT3 signaling.

In some embodiments, the present disclosure provides compositions wherein the intracellular pSTAT-3 signaling in the monocyte cell expressing IL10Rα and IL10R is greater than the pSTAT-3 signaling in a T-cell expressing IL10Rα and IL10Rβ.

In some embodiments, the present disclosure provides compositions wherein the IL-10 agonist compound (e.g., a single-domain antibody polypeptide of an IL-10 agonist compound) comprises a C-terminal amino acid modification that reduces immunogenicity resulting from pre-existing antibodies. In some embodiments, the first single-domain antibody polypeptide of the IL-10 agonist compound comprises the C-terminal modification. In some embodiments, the second single-domain antibody polypeptide of the IL-10 agonist compound comprises the C-terminal modification. In some embodiments, the C-terminal polypeptide selected from the first single-domain antibody polypeptide and the second single-domain antibody polypeptide comprises the C-terminal modification.

In some embodiments, the present disclosure provides compositions, wherein the IL-10 agonist compound (e.g., a single-domain antibody polypeptide of an IL-10 agonist compound) comprises a C-terminal amino acid modification comprising an amino acid sequence selected from the group consisting of SEQ ID NOS:474-499.

In some embodiments, the present disclosure provides compositions wherein the compound is selected from the group consisting of SEQ ID NOS:121-135, 138-141, 144-155, 158-175, 179, and 182-195, and 199.

In another aspect, the disclosure provides a method of treating a mammalian subject suffering from an autoimmune disease, infectious disease, or inflammatory disease by the administration of a therapeutically effective amount of an IL-10 agonist compound. In some embodiments, one sdAb of the binding molecule is an scFv and the other sdAb is a VHH.

In some embodiments, the first and second sdAbs are covalently bound via a chemical linkage.

In some embodiments, the first and second sdAbs are provided as single continuous polypeptide.

In some embodiments, the the IL-10 agonist compound (e.g., a single-domain antibody polypeptide of an IL-10 agonist compound) is provided as single continuous polypeptide.

The present invention provides IL-10 agonist compounds that are synthetic ligands of the IL-10 receptor.

In some embodiments, the invention provides and IL-10 agonist compound comprising an IL-10Rα sdAb having least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative to any one of SEQ ID NOS:25-48. In some embodiments, the IL-10Rα sdAb comprises a C-terminal amino acid modification that reduces immunogenicity resulting from pre-existing antibodies. In some embodiments, the IL-10Rα sdAb comprises a C-terminal amino acid modification comprising an amino acid sequence selected from the group consisting of SEQ ID NOS:475-497.

In some embodiments, the invention provides and IL-10 agonist compound comprising an IL-10Rβ sdAb having least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative to any one of SEQ ID NOS:49 and 50. In some embodiments, the IL-10Rβ sdAb comprises a C-terminal amino acid modification that reduces immunogenicity resulting from pre-existing antibodies. In some embodiments, the IL-10Rβ sdAb comprises a C-terminal amino acid modification comprising an amino acid sequence selected from the group consisting of SEQ ID NOS:475-497.

In some embodiments, the present invention provides IL-10 agonist compounds having at least 90% (e.g., 91%, 92%, 93%, 94%, 95%, 96%, 97%, 98%, 99%, or 100%) sequence identity to the sequence of any one of the IL-10 agonist compounds (e.g., single-domain antibody polypeptides of the IL-10 agonist compounds) of SEQ ID NOS:1-24 and 500-523. In some embodiments, the present invention provides IL-10 agonist compounds substantially identical to any one of the IL-10 agonist compounds (e.g., single-domain antibody polypeptides of the IL-10 agonist compounds) of SEQ ID NOS:1-24 and 500-523. In some embodiments, the present invention provides IL-10 agonist compounds identical to a sequence of any one of the IL-10 agonist compounds (e.g., single-domain antibody polypeptides of the IL-10 agonist compounds) of SEQ ID NOS:1-24 and 500-523. In some embodiments, the IL-10 agonist compound comprises a C-terminal amino acid modification that reduces immunogenicity resulting from pre-existing antibodies. In some embodiments, the IL-10 agonist compound comprises a C-terminal amino acid modification comprising an amino acid sequence selected from the group consisting of SEQ ID NOS:475-497.

In one embodiment, the present disclosure provides an IL-10Rα binding molecule that preferentially activates T cells, such as CD8+ T cells, relative to monocytes. In one embodiment, the present disclosure provides an IL-10Rα binding molecule of the formula (#1) wherein the affinity of the IL-10Rα sdAb has a higher affinity for the extracellular domain of IL-10Rα than the affinity of the IL-10Rβ sdAb for the extracellular domain of IL-10Rβ.

In some embodiments, the present disclosure provides an IL-10 agonist compound modified to provide prolonged duration of action in vivo in a mammalian subject and pharmaceutically acceptable formulations thereof. In some embodiments, the present invention provides a IL-10 agonist compounds that are PEGylated, wherein the PEG is conjugated to the IL-10 agonist compound (e.g., a single-domain antibody polypeptide of an IL-10 agonist compound). In some embodiments, the pegylation is at the C-terminal end of the IL-10R VHH2, and the PEG is a linear or branched PEG molecule having an average molecular weight from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, or alternatively about 30,000 to about 40,000 daltons. In one embodiment of the disclosure, the PEG is a 40 kD branched PEG comprising two 20 kD arms.

The present disclosure further provides a pharmaceutically acceptable formulation of an IL-10 agonist compound for the administration to a mammalian subject. The present disclosure further provides a pharmaceutically acceptable composition for administration to a mammalian subject the composition comprising a nucleic acid sequence encoding a polypeptide IL-10 agonist compound, a recombinant viral or non-viral vector encoding or polypeptide IL-10 agonist compound, or a recombinantly modified mammalian cell comprising a nucleic acid sequence encoding a polypeptide IL-10 agonist compound, in each case the nucleic acid sequence operably linked to one or more expression control elements functional in a mammalian cell.

The present disclosure provides nucleic acid sequences encoding polypeptide IL-10 agonist compounds. The present disclosure further provides a recombinant vector comprising a nucleic acid sequence encoding polypeptide IL-10 agonist compounds. The present disclosure further provides a recombinantly modified mammalian cell comprising a nucleic acid encoding a polypeptide IL-10 agonist compound. The present disclosure further provides methods for the recombinant production, isolation, purification and characterization of a polypeptide IL-10 agonist compound of recombinant vectors comprising a provides nucleic acid sequences encoding polypeptide IL-10 agonist compounds.

The disclosure also provides expression vectors comprising a nucleic acid encoding the bispecific IL-10 agonist compound operably linked to one or more expression control sequence. The disclosure also provides isolated host cells comprising the expression vector comprising a nucleic acid encoding the IL-10 agonist compound (e.g., a single-domain antibody polypeptide of an IL-10 agonist compound) operably linked to one or more expression control sequences functional in the host cell.

In another aspect, the disclosure provides a pharmaceutical composition comprising the IL-10 agonist compound (e.g., a single-domain antibody polypeptide of an IL-10 agonist compound) described herein, and a pharmaceutically acceptable carrier.

In another aspect, the disclosure provides a method of treating an autoimmune or inflammatory disease, disorder, or condition or a viral infection in a subject in need thereof, comprising administering to the subject a therapeutically effective amount of an IL-10 agonist compound described herein or a pharmaceutical composition described herein.

Several advantages flow from the binding molecules described herein. The natural ligand of the IL-10 receptor, IL-10, causes IL-10Rα and IL-10Rβ to come into proximity by their simultaneous binding of IL-10. However, when IL-10 is used as a therapeutic in mammalian, particularly human, subjects, it may also trigger a number of adverse and undesirable effects by a variety of mechanisms including the presence of IL-10Rα and IL-10Rβ on other cell types and the binding to IL-10Rα and IL-10Rβ on the other cell types may result in undesirable effects and/or undesired signaling on cells expressing IL-10Rα and IL-10Rβ. The present disclosure is directed to methods and compositions that modulate the multiple undesirable adverse effects of IL-10 binding to IL-10Rα and IL-10Rβ so that desired therapeutic signaling occurs, particularly in a desired cellular or tissue subtype, while minimizing undesired activity and/or intracellular signaling.

In some embodiments, the IL-10 agonist compounds (e.g., single-domain antibody polypeptides of the IL-10 agonist compounds) described herein are partial agonists of the IL-10 receptor. In some embodiments, the binding molecules described herein are designed such that the binding molecules are full agonists. In some embodiments, the binding molecules described herein are designed such that the binding molecules are super agonists.

In some embodiments, the binding molecules provide the maximal desired IL-10 intracellular signaling from binding to IL-10Rα and IL-10Rβ on the desired cell types, while providing significantly less IL-10 signaling on other undesired cell types. This can be achieved, for example, by selection of binding molecules having differing affinities or causing different Emax for IL-10Rα and IL-10Rβ as compared to the affinity of IL-10 for IL-10Rα and IL-10Rβ.

Because different cell types respond to the binding of ligands to its cognate receptor with different sensitivity, by modulating the affinity of the dimeric ligand (or its individual binding moieties) for the IL-10 receptor relative to wild-type IL-10 binding facilitates the stimulation of desired activities while reducing undesired activities on non-target cells.

### BRIEF DESCRIPTION OF THE DRAWINGS

FIG. 1 of the attached drawings is a graph showing that the humanized IL-10Rα/IL-10Rβ VHH dimers DR1525 and DR2096 were less potent than the parental llama molecule DR841 in an LPS-induced monocyte secretion assay, as measured by inhibiting the production of IL1.

FIG. 2 of the attached drawings is a graph showing that the humanized IL-10Rα/IL-10Rβ VHH dimers DR1525 and DR2096 were less potent than the parental llama molecule DR841 in an LPS-induced monocyte secretion assay, as measured by inhibiting the production of TNFα.

FIG. 3 of the attached drawings is a graph showing the humanized IL-10Rα/IL-10Rβ VHH dimer DR2503 has a comparable potency to the parental llama molecule DR841 in an LPS-induced monocyte secretion assay, as measured by inhibiting the production of IL1β.

FIG. 4 of the attached drawings is a graph showing the humanized IL-10Rα/IL-10Rβ VHH dimer DR2503 has a comparable potency to the parental llama molecule DR841 in an LPS-induced monocyte secretion assay, as measured by inhibiting the production of TNFα. The graph also shows functionality of IL10Rα/IL10Rβ VHH dimers DR2463 and DR2485.

FIG. 5 of the attached drawings shows that humanized IL10Rα/IL10Rβ VHH dimers DR2485 (SEQ ID NO:2), DR2519 (SEQ ID NO:3), and DR2520 (SEQ ID NO:4) of Table 1 retain the ability to suppress the secretion of proinflammatory cytokines IL-10 similar to the non-humanized parental VHH dimer DR841 (Table 14; SEQ ID NO:465).

FIG. 6 of the attached drawings show that humanized IL10Rα/IL10Rβ VHH dimers DR2485 (SEQ ID NO:2), DR2519 (SEQ ID NO:3), and DR2520 (SEQ ID NO:4) of Table 1 retain the ability to suppress the secretion of proinflammatory cytokines TNFα similar to the non-humanized parental VHH dimer DR841 (Table 14; SEQ ID NO:465).

FIG. 7 of the attached drawings shows that humanized IL10Rα/IL10Rβ VHH dimers DR2485 (SEQ ID NO:2), DR2519 (SEQ ID NO:3), and DR2520 (SEQ ID NO:4) of Table 1 suppress induction of IFN-γ in T-cells.

FIG. 8 of the attached drawings shows that humanized IL10Rα/IL10Rβ VHH dimers DR2485 (SEQ ID NO:2), DR2519 (SEQ ID NO:3), and DR2520 (SEQ ID NO:4) of Table 1 suppress the production of granzyme B in T-cells.

### DETAILED DESCRIPTION OF THE INVENTION

To facilitate the understanding of present disclosure, certain terms and phrases are defined below, as well as throughout the specification. The definitions provided herein are non-limiting and should be read in view of the knowledge of one of skill in the art would know.

The present invention is not limited to a particular method or composition described, as such may, of course, vary. Furthermore, the terminology used herein is for the purpose of describing specific embodiments only and such specific embodiments are not intended to be limiting.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, for example, polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication date which may need to be independently confirmed.

Unless indicated otherwise, parts are parts by weight, molecular weight is the average molecular weight, temperature is in degrees Celsius (° C.), and pressure is at or near atmospheric. Standard abbreviations are used, including the following: bp=base pair(s); kb=kilobase(s); pl=picoliter(s); s or sec=second(s); min=minute(s); h or hr=hour(s); AA or aa=amino acid(s); kb=kilobase(s); nt=nucleotide(s); pg=picogram; ng=nanogram; μg=microgram; mg=milligram; g=gram; kg=kilogram; dl or dL=deciliter; μl or μL=microliter; ml or mL=milliliter; l or L=liter; μM=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=once weekly; QM=once monthly; HPLC=high performance liquid chromatography; BW=body weight; U=unit; ns=not statistically significant; PBS=phosphate-buffered saline; PCR=polymerase chain reaction; HSA=human serum albumin; MSA=mouse serum albumin; DMEM=Dulbeco's Modification of Eagle's Medium; EDTA=ethylenediaminetetraacetic acid.

It will be appreciated that throughout this disclosure reference is made to amino acids according to the single letter or three letter codes. For the reader's convenience, the single and three letter amino acid codes are provided in Table 13.

Standard methods in molecular biology are described in the scientific literature (see, for example, Sambrook and Russell. 2001. Molecular Cloning, 3rd ed., Cold Spring Harbor Laboratory Press, Cold Spring Harbor, N.Y.; and Ausubel et al. 2001. Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)). The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, for example, Coligan et al. 2000. Current Protocols in Protein Science. Vols. 1-2, John Wiley and Sons, Inc., NY).

### Definitions

Unless otherwise indicated, the following terms are intended to have the meaning set forth below. Other terms are defined elsewhere throughout the specification.

Activate: As used herein the term “activate” is used in reference to a receptor or receptor complex to reflect a biological effect, directly and/or by participation in a multicomponent signaling cascade, arising from the binding of an agonist ligand to a receptor responsive to the binding of the ligand.

Activity: As used herein, the term “activity” is used with respect to a molecule to describe a property of the molecule with respect to a test system (e.g., an assay) or biological or chemical property (e.g., the degree of binding of the molecule to another molecule) or of a physical property of a material or cell (e.g., modification of cell membrane potential). Examples of such biological functions include but are not limited to catalytic activity of a biological agent, the ability to stimulate intracellular signaling, gene expression, cell proliferation, the ability to modulate immunological activity such as inflammatory response. “Activity” is typically expressed as a level of a biological activity per unit of agent tested such as [catalytic activity]/[mg protein], [immunological activity]/[mg protein], international units (IU) of activity, [STAT5 phosphorylation]/[mg protein], [T-cell proliferation]/[mg protein], plaque forming units (pfu), etc. As used herein, the term “proliferative activity” refers to an activity that promotes cell proliferation and replication.

Administer/Administration: The terms “administration” and “administer” are used interchangeably herein to refer the act of contacting a subject, including contacting a cell, tissue, organ, or biological fluid of the subject in vitro, in vivo or ex vivo with an agent (e.g., an ortholog, an IL-10 ortholog, an engineered cell expressing an orthogonal receptor, an engineered cell expressing an orthogonal IL-10 receptor, a CAR-T cell expressing an orthogonal IL-10 receptor, a chemotherapeutic agent, an antibody, or a pharmaceutical formulation comprising one or more of the foregoing). Administration of an agent may be achieved through any of a variety of art recognized methods including but not limited to the topical administration, intravascular injection (including intravenous or intraarterial infusion), intradermal injection, subcutaneous injection, intramuscular injection, intraperitoneal injection, inhalation and the like. The term “administration” includes contact of an agent to the cell, tissue or organ as well as the contact of an agent to a fluid, where the fluid is in contact with the cell, tissue or organ.

Affinity: As used herein the term “affinity” refers to the degree of specific binding of a first molecule (e.g., a ligand) to a second molecule (e.g., a receptor) and is measured by the equilibrium dissociation constant K_{D}, a ratio of the dissociation rate constant between the molecule and its target (k_{off}) and the association rate constant between the molecule and its target (kₒₙ).

Agonist: As used herein, the term “agonist” refers to a first agent that specifically binds a second agent (“target”) and interacts with the target to cause or promote an increase in the activation of the target. In some instances, agonists are activators of receptor proteins that modulate cell activation, enhance activation, sensitize cells to activation by a second agent, or up-regulate the expression of one or more genes, proteins, ligands, receptors, biological pathways that may result in cell proliferation or pathways that result in cell cycle arrest or cell death such as by apoptosis. In some embodiments, an agonist is an agent that binds to a receptor and alters the receptor state, resulting in a biological response. The response mimics the effect of the endogenous activator of the receptor. The term “agonist” includes partial agonists, full agonists and superagonists. An agonist may be described as a “full agonist” when such agonist which leads to a substantially full biological response (i.e., the response associated with the naturally occurring ligand/receptor binding interaction) induced by receptor under study, or a partial agonist. In contrast to agonists, antagonists may specifically bind to a receptor but do not result the signal cascade typically initiated by the receptor and may to modify the actions of an agonist at that receptor. Inverse agonists are agents that produce a pharmacological response that is opposite in direction to that of an agonist. A “superagonist” is a type of agonist that is capable of producing a maximal response greater than the endogenous agonist for the target receptor, and thus has an activity of more than 100% of the native ligand. A super agonist is typically a synthetic molecule that exhibits greater than 110%, alternatively greater than 120%, alternatively greater than 130%, alternatively greater than 140%, alternatively greater than 150%, alternatively greater than 160%, or alternatively greater than 170% of the response in an evaluable quantitative or qualitative parameter of the naturally occurring form of the molecule when evaluated at similar concentrations in a comparable assay.

Antagonist: As used herein, the term “antagonist” or “inhibitor” refers a molecule that opposes the action(s) of an agonist. An antagonist prevents, reduces, inhibits, or neutralizes the activity of an agonist, and an antagonist can also prevent, inhibit, or reduce constitutive activity of a target, for example, a target receptor, even where there is no identified agonist. Inhibitors are molecules that decrease, block, prevent, delay activation, inactivate, desensitize, or down-regulate, for example, a gene, protein, ligand, receptor, biological pathway, or cell.

Antibody (Ab): As used herein, the term “antibody” means any form of antibody (also known as an immunoglobulin (Ig)) that exhibits the desired biological activity of binding to an antigen epitope, as described herein. The term “antibody” specifically covers, but is not limited to, polyclonal antibodies, monoclonal antibodies (including full length monoclonal antibodies comprising two light chains and two heavy chains), multispecific antibodies (e.g., bispecific antibodies that bind to two or more antigens or antigen epitopes on a single antigen), fully human antibodies (huAb), humanized antibodies (hzAb), chimeric antibodies, single chain variable fragment antibodies (scFv), single-domain antibodies (sdAb), variable heavy (VH) domain antibodies, diabodies (dAb), and antigen-binding fragments of heavy chain only antibodies (VHH), comprising the amino acid sequences of the variable region, as described herein. As used herein, the term “antibody” refers collectively to: (a) glycosylated and non-glycosylated immunoglobulins (including but not limited to mammalian immunoglobulin classes IgG1, IgG2, IgG3, and IgG4) that specifically bind to a target molecule, such as an antigen, and (b) immunoglobulin derivatives including but not limited to IgG(1-4)deltaC_{H}2, F(ab′)₂, Fab, ScFv, V_{H}, V_{L}, tetrabodies, triabodies, diabodies, dsFv, F(ab′)₃, scFv-Fc and (scFv)₂ that compete with the immunoglobulin from which it was derived for binding to the target molecule. The term antibody is not restricted to immunoglobulins derived from any particular mammalian species and includes murine, human, equine, camelids, and uman antibodies. The term antibody includes “heavy chain antibodies” and “VHHs” as typically obtained from immunization of camelids (including camels, llamas, and alpacas, such as described by e.g., Hamers-Casterman et al. 1993. Nature. 363:446-448, as described in greater detail below in the definition of “VHH.” Antibodies having a given specificity may also be derived from non-mammalian sources, such as VHHs obtained from immunization of cartilaginous fishes, including, but not limited to, sharks. The term “antibody” encompasses antibodies isolatable from natural sources or from animals following immunization with an antigen, as well as engineered antibodies including monoclonal antibodies, bispecific antibodies, tri-specific, chimeric antibodies, humanized antibodies, human antibodies, CDR-grafted, veneered, or deimmunized (e.g., to remove B and/or T-cell epitopes) antibodies.

Human Antibody (hAb): The term “human antibody” includes antibodies obtained from human beings as well as antibodies obtained from transgenic mammals comprising human immunoglobulin genes such that, upon stimulation with an antigen the transgenic animal produces antibodies comprising amino acid sequences characteristic of antibodies produced by human beings. The term antibody includes both the parent antibody and its derivatives such as affinity matured, veneered, CDR grafted (including CDR grafted VHHs), humanized, camelized (in the case of non-camel derived VHHs), or binding molecules comprising binding domains of antibodies (e.g., CDRs) in non-immunoglobulin scaffolds. The term “antibody” is not limited to any particular means of synthesis and includes naturally occurring antibodies isolatable from natural sources and as well as engineered antibodies molecules that are prepared by “recombinant” means including antibodies isolated from transgenic animals that are transgenic for human immunoglobulin genes or a hybridoma prepared therefrom, antibodies isolated from a host cell transformed with a nucleic acid construct that results in expression of an antibody, antibodies isolated from a combinatorial antibody library including phage display libraries or chemically synthesized (e.g., solid phase protein synthesis). In one embodiment, an “antibody” is a mammalian immunoglobulin. In some embodiments, the antibody is a “full-length antibody” comprising variable and constant domains providing binding and effector functions. In some embodiments, a full-length antibody comprises two light chains and two heavy chains, each light chain comprising a variable region and a constant region. In some embodiments, the term “full length antibody” is used to refer to conventional IgG immunoglobulin structures comprising two light chains and two heavy chains, each light chain comprising a variable region and a constant region providing binding and effector functions. The term antibody includes antibody conjugates comprising modifications to prolong duration of action such as fusion proteins or conjugation to polymers (e.g., PEGylated) as described in more detail below.

Heavy Chain (H): The term “heavy chain” when used in reference to an antibody means a polypeptide chain comprising a variable region and a constant region that can be combined with a light chain. A heavy chain can be a human heavy chain sequence derived from a human heavy chain, or a heavy chain that has been humanized by introducing amino acid residue substitutions at specific locations in the amino acid sequence to generate a polypeptide having an amino acid sequence that is similar to a human polypeptide.

Constant Region (C): The term “constant region” or “constant domain” refers to a carboxy terminal portion of the light and heavy chain that is not directly involved in binding of the antibody to an antigen target, but exhibits various effector functions, such as interaction with an Fc receptor. The amino acid sequence residues of a “constant region” or “constant domain” are generally more conserved (i.e., less variable) relative to the other more variable region of the immunoglobulin, which contain the antigen binding site. The constant region contains the CH1, CH2, and CH3 regions of the heavy chain and the CL region of the light chain.

Variable Region (V): The term “variable region,” “variable domain,” “V region,” or “V domain” refers to a part of either the variable light (VL) or variable heavy (VH) chain of an antibody that is located at the amino-terminal end of the light or heavy chain, and which determine the binding specificity of each particular antibody for its particular antigen. The variable regions of the light chain (VL) and heavy chain (VH) can together or individually form a binding site (referred to as the paratope) that binds to a target (referred to as the antigen epitope). The binding region can be comprised of one of or both the VH and VL chains, for example, forming a bifunctional or bispecific antibodies, having two identical binding sites that bind to the same epitope or two different binding sites that bind to different epitopes.

Framework Region (FR): As used herein, the term “framework region,” “framework,” or “FR”, as used in the context of antibodies, means those amino acid sequence regions and amino acid residues having amino acid residues that are less variable in comparison to other amino acid residues in the antibody variable region and constant region. The framework regions flank hypervariable regions (HVRs), which together constitute the variable region of the antibody or antibody fragment. A hypervariable region (HVR) is also referred to as a complementarity determining region (CDR), and such terms may be used interchangeable herein. Framework regions may also be defined as those amino acid sequence residues that are not hypervariable amino acid residues or are not part of the amino acid sequence of a hypervariable region.

Complementarity Determining Region (CDR): The term “complementarity determining region,” and “CDR” (also referred to, as described above, a “hypervariable region,” or “HVR”) mean segments of the variable regions having amino acid residues that are more variable in comparison to other amino acid residues in the antibody variable region and constant region. As noted above, the term “complementarity-determining region” “CDR” is synonymous with the term hypervariable region (HVR) and may be used interchangeably. Although the term “complementarity-determining region” and “CDR” is generally used in the art to describe those amino acid residues of an antibody variable region (VL or VH) that are believed to mediate or participate in the interactions and binding between an antibody and an antigen epitope, or that have some structural and/or functional properties (i.e., are “complementary” or have amino acid residues that are physically or chemically “complementary” to amino acid residues on an epitope), the use of the term CDR herein shall not be construed to mean that every amino acid residue of such CDR is necessarily physically or chemically complementarity, as some CDR amino acid residues within a CDR region may not necessarily mediate or participate in binding of the CDR region to an antigen epitope, nor shall the term CDR be construed to mean that the CDR amino acid residues of such CDR constitute all of the amino acid residues that mediate or participate in binding of the CDR region to an antigen epitope, as it is known that some amino acid residues outside the CDR region (i.e., in a framework region (FR)) may participate in or influence binding of the antibody to an antigen epitope. Those skilled in the art understand that the amino acid residues of a CDR sequence, however defined, may not all necessarily engage in antigen epitope contact, and that additional amino acid residues not assigned to be within a stated CDR region (i.e., amino acid residues within FR1, FR2, FR3, or FR4 framework regions) may also engage in antigen epitope contact or otherwise be involved in target antigen epitope binding. Accordingly, as used herein, the term CDR shall be construed to mean a region of an antibody variable heavy (VH) region or variable light (VL) region that generally is more highly variable (i.e., hypervariable) than adjoining framework regions (which are also relatively “variable” in comparison to antibody constant regions) and as a consequence of such CDR variability a “CDR” region, regardless of any actual or predicted structural, physical, chemical, or binding properties of the CDR region, is suitable for purposes of characterizing the chemical, physical, structural, and/or binding properties of an antibody (e.g., the structure, function, and/or binding properties of the antibody), and is therefore useful in differentiating and establishing the novelty of the antibody from other antibodies.

Single-domain antibodies, such as an scFv or a VHH have distinct physical and chemical properties compared to a standard antibody, and, accordingly, the CDR regions of a sdAb may be defined differently than standard antibodies. A sdAb VL or VH chain typically comprises three hypervariable regions (CDR1, CDR2, and CDR3) interspersed between four framework regions (FR1, FR2, FR3, and FR4), forming a polypeptide sequence comprising the linear polypeptide structure FR1-CDR1-FR2-CDR2-FR3-CDR3-FR4.

The amino acid residues that comprise the CDR regions of an antibody are commonly defined in accordance with the IMGT system, using the Kabat numbering system. The Kabat numbering system for conventional IgG molecules is described, for example, in Kabat et al. 1991. Sequences of Proteins of Immunological Interest. National Institutes of Health, Bethesda, Md. 5^{th} ed. NIH Publ. No. 91-3242; Kabat. 1978. Adv Prot Chem. 32:1-75; and Kabat et al. 1977. J Biol Chem. 252:6609-6616; Wu, TT and Kabat, E A. 1970. Analysis of the sequences of the variable regions of Bence Jones proteins and myeloma light chains and their implications for antibody complementarity. J Exp Med. 132:211-250; and Kabat, EA and Wu, TT. 1991. Identical V region amino acid sequences and segments of sequences in antibodies of different specificities. Relative contributions of VH and VL genes, minigenes, and complementarity determining regions to binding of antibody combining sites. J Immunol. 147:1709-1719. More recently, a Kabat numbering scheme specific for single-domain antibodies (sdAbs) has been proposed by Sulea, T. 2022. Chapter 14, Humanization of Camelid Single-Domain Antibodies. In: Hussack, G and Henry, KA, editors. Single-Domain Antibodies: Methods and Protocols, Methods in Molecular Biology. 1st ed. New York (NY): Humana. Vol. 2446. p. 299-312 (Sulea (2022)).

The general numbering used in the Kabat numbering system for standard antibodies is described in Kabat et al., Sequence of proteins of immunological interest. US Public Health Services, NIH Bethesda, Md., Publication No. 91. The standard Kabat numbering scheme, has been applied to VHH domains from Camelids, as described by Riechmann and Muyldermans. 2000. J Immunol Methods 240(1-2):185-195. According to this numbering scheme, amino acid residues are numbered as follows (amino acid residue numbers in parentheses): FR1 (1-30), CDR1 (31-35B), FR2 (36-49), CDR2 (50-65), FR3 (66-92), CDR3 (93-102), and FR4 (103-113). It is known in the art that for VH and VHH polypeptides derived from single-domain antibodies the total number of amino acid residues in each of the CDR's may vary and may not correspond exactly to the total number of amino acid residues indicated by the standard Kabat numbering scheme (that is, one or more positions according to the Kabat numbering may not be occupied in the actual sequence, or the actual sequence may contain more amino acid residues than the number allowed for by the Kabat numbering).

In accordance with the Kabat antibody numbering scheme for sdAbs, the amino acid sequence of each of the three CDR regions of the sdAb variable regions (including VHHs and scFvs) is defined in the following paragraphs, below.

CDR1: The term “CDR1” means the amino acid sequence beginning with amino acid residue 31 and ending with the Kabat amino acid residue immediately preceding amino acid residue 36. Because CDR1 may include amino acid insertions resulting from an immunoglobulin VDJ gene segment recombination, CDR1 may alternatively be defined as the amino acid sequence beginning with the amino acid immediately following amino acid residue 30 and ending with the amino acid immediately preceding amino acid residue 36 (i.e., the amino acid sequence between amino acid residue 30, the last amino acid residue of FR1, and amino acid residue 36, the first amino acid residue of FR2).

CDR2: The term “CDR2” means the amino acid sequence beginning with amino acid residue 50 and ending with the amino acid residue immediately preceding amino acid residue 66. CDR2 may alternatively be defined as the amino acid sequence beginning with the amino acid residue immediately following amino acid residue 49 and end with the amino acid residue immediately preceding amino acid residue 66 (i.e., the amino acid sequence between amino acid residue 49, the last amino acid residue of FR2, and amino acid residue 66, the first amino acid residue of FR3.

CDR3: The term “CDR3” means the amino acid sequence beginning with amino acid residue 93 and ending with the amino acid residue 102. Because CDR3 may include amino acid insertions resulting from an immunoglobulin VDJ gene segment recombination, CDR3 may alternatively be defined as the amino acid sequence beginning with the amino acid residue immediately following amino acid residue 92 and ending with the amino acid residue immediately preceding amino acid residue 103 (i.e., the amino acid sequence between amino acid residue 92, the last amino acid residue of FR3, and amino acid residue 103, the first amino acid residue of FR4).

Single-Domain Antibody: The term “single-domain antibody” or “sdAb” means an antibody fragment consisting of a single monomeric variable antibody domain, which may comprise one variable heavy domain (VH) of a heavy-chain antibody or of a common IgG molecule. A sdAb is able to bind selectively to a specific antigen. One specific type of sdAb is a VHH molecule. Single-domain antibodies can be obtained by immunization of dromedaries, camels, llamas, alpacas, or sharks with the desired antigen and subsequent isolation of the mRNA coding for the variable region (VNAR and VHH) of heavy-chain antibodies. Alternatively, sdAbs can be made from common murine, rabbit, or human IgG with four chains. Humans can also produce sdAbs by the random creation of a stop codon in the light chain. The term “single-domain antibody” or “sdAb” refers to an antibody having a single (only one) monomeric variable antibody domain.

VHH: The term “VHH” as used herein means a heavy chain-only variable domain fragment that is obtained from or originated or derived from a heavy chain antibody. Heavy chain antibodies are functional antibodies that have two heavy chains and no light chains. Heavy chain antibodies exist in and are obtainable from camelids (e.g., camels and alpacas), members of the biological family Camelidae. VHH antibodies have originally been described as the antigen-binding immunoglobulin (variable) domain of “heavy chain antibodies” (i.e., of “antibodies devoid of light chains”; Hamers-Casterman et al., Nature 363: 446-448 (1993). The term “VHH domain” is used to distinguish these variable domains from the heavy chain variable domains that are present in conventional four-chain antibodies (which are referred to herein as “VH domains” or “VH”) and from the light chain variable domains that are present in conventional four-chain antibodies (which are referred to herein as “VL domains” or “VL”). For a further description of VHHs, reference is made to the review article by Muyldermans (Reviews in Molec. Biotechnol. 74: 277-302, (2001), as well as to the following patent applications, which are mentioned as general background art: international patent publications numbers WO9404678, WO9504079 and WO9634103 of the Vrije Universiteit Brussel; WO9425591, WO9937681, WO0040968, WO0043507, WO0065057, WO0140310, WO0144301, EP1134231 and WO0248193 of Unilever; WO9749805, WO0121817, WO03035694, WO03054016 and WO03055527 of the Vlaams Instituut voor Biotechnologie (VI B); WO03050531 of Algonomics N.V. and Ablynx N.V.; WO0190190 by the National Research Council of Canada; WO03025020 (EP 1433793) by the Institute of Antibodies; as well as international patent publications numbers WO2004041867, WO2004041862, WO2004041865, WO2004041863, WO2004062551, WO2005044858, WO200640153, WO2006079372, WO2006122786, WO 06122787, WO2006122825, WO2008101985, WO2008142164, and WO2015173325 by Ablynx N.V. and the further published patent applications by Ablynx N.V. Reference is also made to the further prior art mentioned in these applications, and in particular to the list of references mentioned on pages 41-43 of the International application WO2006040153, which list and references are incorporated herein by reference. Methods of obtaining VHH domains binding to a specific antigen or epitope have been described earlier, for example, in WO2006/040153 and WO2006/122786. As also described therein in detail, VHH domains derived from camelids can be “humanized” or made “human-like” by being engineered, for example, by replacing one or more amino acid residues in the amino acid sequence of the original VHH sequence by one or more of the amino acid residues that occur at the corresponding position(s) in a VH domain from a conventional 4-chain antibody from a human being. A humanized VHH domain can contain one or more fully human framework region sequences, and, in an even more specific embodiment, can contain human framework region sequences derived from DP-29, DP-47, DP-51, or parts thereof, optionally combined with JH sequences, such as JH5. VHH CDRs can be grafted into multiple types of binding proteins (e.g., antibodies) and the CDRs retain binding. When VHH CDRs are grafted to a framework, it is engineered so as to have potentially more advantageous binding behavior. For example, the VHH can be linked genetically to Fc-domains, other VHHs, peptide tags, or toxins, and can be conjugated chemically at a specific site to drugs, radionuclides, photosensitizers, and nanoparticles. See Bannas et al. 2017. Front Immunol. 8: 1603. In certain embodiments of the method, the binding protein is selected from: a single-chain antibody (scFv); a recombinant camelid heavy-chain-only antibody (VHH); a shark heavy-chain-only antibody (VNAR); a microprotein; a darpin; an anticalin; an adnectin; an aptamer; a Sac7d derivative (affitins, for example, NANOFITINS, see 2008. Journal of Molecular Biology 383(5):1058-68, the contents of which are hereby incorporated by reference), a Fv; a Fab; a Fab′; and a F(ab′)2. In an embodiment, the binding protein is heterodimeric, for example the binding protein has greater potency than each individual monomer. In alternative embodiments, the heteromultimeric neutralizing binding protein is multimeric and the multimeric components are associated non-covalently or covalently. VHHs are antibody-derived therapeutic proteins that contain the unique structural and functional properties of naturally occurring heavy-chain antibodies. VHH technology is based on fully functional antibodies from camelids that lack light chains. These heavy-chain antibodies contain a single variable domain (VHH) and two constant domains (CH2 and CH3). The cloned and isolated VHH domain is a stable polypeptide harboring the antigen-binding capacity of the original heavy-chain antibody. See Castorman et al., U.S. Pat. No. 5,840,526 issued Nov. 24, 1998; and Castorman et al., U.S. Pat. No. 6,015,695 issued Jan. 18, 2000, each of which is incorporated by reference herein in its entirety. VHHs are commercially available from Ablynx Inc. (Ghent, Belgium) under the trademark of NANOBODIES™. Suitable methods of producing or isolating antibody fragments having the requisite binding specificity and affinity are described herein and include for example, methods which select recombinant antibody from a library, by PCR (See Ladner, U.S. Pat. No. 5,455,030 issued Oct. 3, 1995, and Devy et al., U.S. Pat. No. 7,745,587 issued Jun. 29, 2010, each of which is incorporated by reference herein in its entirety).

Functional fragments of antibodies, including fragments of chimeric; humanized, primatized, veneered, or single-chain antibodies, can also be produced. Functional fragments or portions of the foregoing antibodies include those which are reactive with the disease agent. For example, antibody fragments capable of binding to the disease agent or portion thereof, include, but are not limited to scFvs, Fabs, VHHs, Fv, Fab, Fab′ and F(ab′)2 are encompassed by the invention. Such fragments can be produced by enzymatic cleavage or by recombinant techniques. For instance, papain or pepsin cleavage may be used to generate Fab or F(ab′)2 fragments, respectively. Antibody fragments are produced in a variety of truncated forms using antibody genes in which one or more stop codons have been introduced upstream of the natural stop site. For example, a chimeric gene encoding an F(ab′)2 heavy chain peptide portion can be designed to include DNA sequences encoding the CHI peptide domain and hinge region of the heavy domain. Accordingly, the present invention encompasses polynucleic acids that encode the IL-10 agonist compound (e.g., a single-domain antibody polypeptide of an IL-10 agonist compound) binding protein described herein. IL-10 agonist compound binding proteins in certain embodiments are made as part of a multimeric protein, the monomer or single binding region (for example, antibody fragments, microproteins, darpins, anticalins, adnectins, peptide mimetic molecules, aptamers, synthetic molecules, etc.) can be linked. Any combination of binding protein or binding region types can be linked. In an embodiment, the monomer or binding region of a multimeric binding protein can be linked covalently. In another embodiment, a monomer binding protein can be modified, for example, by attachment to another monomer binding protein, directly (i.e., the C-terminus of one monomer covalently bound to the N-terminus of the other monomer) or indirectly (e.g., via a linker or spacer). A monomer in various embodiments is attached or genetically fused to another monomer (e.g., by a recombinant protein that is engineered to contain extra amino add sequences that constitute the monomers). Thus, the DNA encoding one monomer is joined, in reading frame, with the DNA encoding the second monomer, and so on. The DNA may therefore include additional nucleotides that encode additional amino acids between the monomers to produce an unstructured region separating the different monomers to better promote the independent folding of each monomer into its active conformation or shape. Commercially available techniques for fusing proteins are used in various embodiments to join the monomer into a multimeric binding protein of the present invention.

F: An “Fc” region means an antibody fragment that contains two heavy chain fragments comprising the CH1 and CH2 domains of an antibody. The two heavy chain fragments are held together by two or more disulfide bonds and by hydrophobic interactions of the CH3 domains. An Fc region is the C-terminal region of an immunoglobulin heavy chain, including, for example, native sequence Fc regions, recombinant Fc regions, and variant Fc regions. Although the boundaries of the Fc region of an immunoglobulin heavy chain may vary, the human IgG heavy chain Fc region is often defined to extend from an amino acid residue at position Cys226, or from Pro230, to the carboxyl-terminus thereof. The C-terminal lysine (residue 447 according to the EU numbering system for IgG molecules) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

Fragments: The present disclosure also includes functional antigen-binding fragments and methods of use thereof. As used herein, unless otherwise indicated, antibody “fragment” or antigen-binding “fragment” refers to antigen-binding fragments of antibodies or bispecific antibodies, for example, antibody fragments that retain the ability to bind specifically to the antigen bound by the full-length antibody, for example, fragments that retain one or more CDR regions. Examples of antigen-binding fragments include but are not limited to Fab, Fab′, F(ab′)2, and Fv fragments; diabodies; linear antibodies; single-chain antibody molecules, for example, scFv; half bispecific molecules comprising the heavy and light chain of one antigen-binding arm. A “Fab fragment” means an antibody fragment that is comprised of one light chain and the CH1 and variable regions of one heavy chain. The heavy chain of a Fab molecule cannot form a disulfide bond with another heavy chain molecule. An “Fab fragment” can be the product of papain cleavage of an antibody. A “Fab′ fragment” means an antibody fragment that contains one light chain and a portion or fragment of one heavy chain that contains the VH domain and the CH1 domain and also the region between the CH1 and CH2 domains, such that an interchain disulfide bond can be formed between the two heavy chains of two Fab′ fragments to form a F(ab′)2 molecule. A “F(ab′)₂ fragment” means an antibody fragment that contains two light chains and two heavy chains containing a portion of the constant region between the CH1 and CH2 domains, such that an interchain disulfide bond is formed between the two heavy chains. A F(ab′)2 fragment thus is composed of two Fab′ fragments that are held together by a disulfide bond between the two heavy chains. An “F(ab′)2 fragment” can be the product of pepsin cleavage of an antibody. An “Fv fragment” or “Fv region” means an antibody fragment that comprises the variable regions from both the heavy and light chains, but lacks the constant regions.

Single Chain Fv: The term “single-chain Fv” or “scFv” antibody refers to antibody fragments comprising the VH and VL domains of an antibody, wherein these domains are present in a single polypeptide chain. Generally, the Fv polypeptide further comprises a polypeptide linker between the VH and VL domains which enables the scFv to form the desired structure for antigen-binding. For a review of scFv, see Pluckthun. 1994. The Pharmacology of Monoclonal Antibodies, vol. 113, Rosenburg and Moore eds. Springer-Verlag, New York, pp. 269-315. See also, International Publ. No. WO 88/01649 and U.S. Pat. Nos. 4,946,778 and 5,260,203. In one embodiment, the scFv comprises from N to C terminal the VHregion, the peptide linker and the VL region. In another embodiment, the scFv comprises from N to C terminal the VL region, the peptide linker and the VH region.

Fab: A “Fab” is comprised of the VH and CH1 regions of a heavy chain and the VL and CL regions of a light chain, which are typically joined together by disulfide bonds and have a single antigen binding site. The VH, CH1, VL and CL regions in a Fab can be arranged in various ways to confer an antigen binding capability according to the present disclosure. For example, the VH and CH1 regions can be on one polypeptide, and the VL and CL regions can be on a separate polypeptide. Alternatively, VH, CH1, VL and CL regions can all be on the same polypeptide, optionally arranged in different orders.

Diabody: The present disclosure includes diabodies and methods of use thereof. As used herein, the term “diabody” or “diabodies” refers to small antibody fragments with two antigen-binding sites, which fragments comprise a heavy chain variable domain (VH) connected to a light chain variable domain (VL) in the same polypeptide chain (VH-VL or VL-VH). By using a linker that is too short to allow pairing between the two domains on the same chain, the domains are forced to pair with the complementary domains of another chain and create two antigen-binding sites. Diabodies are described more fully in, for example, EP 404097; WO 93/11161; and Holliger et al. 1993. Proc Natl Acad Sci. USA 90: 6444-6448. For a review of engineered antibody variants generally see Holliger and Hudson. 2005. Nat Biotechnol. 23:1126-1136. “Domain antibodies,” also known as “dAbs” (the terms “Domain Antibodies” and “dAbs” being used as trademarks by the GlaxoSmithKline group of companies) have been described in more detail in Ward, E. S. et al. 1989. Binding activities of a repertoire of single immunoglobulin variable domains secreted from Escherichia coli. Nature 341: 544-546; Holt, L. J. et al. 2003. Domain antibodies: proteins for therapy. Trends in Biotechnology 21(11): 484-490; and WO2003/002609. Domain antibodies essentially correspond to the VH or VL domains of non-camelid mammalians, in particular human 4-chain antibodies. In order to bind an epitope as a single antigen binding domain, i.e., without being paired with a VH or VL domain, respectively, specific selection for such antigen binding properties is required, for example, by using libraries of human single VH or VL domain sequences. Domain antibodies have, like VHHs, a molecular weight of approximately 13 to approximately 16 kDa and, if derived from fully human sequences, do not require humanization for e.g., therapeutic use in humans. As in the case of VHH domains, they are well expressed also in prokaryotic expression systems, providing a significant reduction in overall manufacturing cost. Domain antibodies, as well as VHH domains, can be subjected to affinity maturation by introducing one or more alterations in the amino acid sequence of one or more CDRs, which alterations result in an improved affinity of the resulting immunoglobulin single variable domain for its respective antigen, as compared to the respective parent molecule. Affinity-matured immunoglobulin single variable domain molecules of the invention may be prepared by methods known in the art, for example, as described by Marks et al. 1992. Biotechnology 10:779-783, or Barbas et al. 1994. Proc Nat Acad Sci. USA 91: 3809-3813; Shier et al. 1995. Gene 169:147-155; Yelton et al. 1995. Immunol. 155:1994-2004; Jackson et al. 1995. J Immunol. 154(7):3310-9; and Hawkins et al. 1992. J Mol Biol. 226(3):889896; and KS Johnson and RE Hawkins. 1996. Affinity maturation of antibodies using phage display. Oxford University Press.

Bispecific Antibody: A “bispecific antibody” of the disclosure comprises an antigen-binding arm comprising the heavy and light chain variable regions of any of the claimed antibodies or antigen-binding fragments thereof, and another antigen-binding arm that recognizes a different antigen. In one embodiment, the bispecific antibody is a heterodimer with an antigen-binding arm comprising a heavy and light chain, and another antigen-binding arm binding to a different antigen comprising a heavy and light chain. The two antigen-binding arms associate to form a heterodimer via the two heavy chain constant regions that have mutations in the CH3 region (see for example FIG. X). A “multispecific antibody” comprises a bispecific antibody, and further comprises additional antigen-binding arms comprising heavy and light chain variable regions targeting at least one other antigen.

Binding Molecule: As used herein, the term “binding molecule” refers to a bivalent molecule that can bind to the extracellular domain of two cell surface receptors. In some embodiments, a binding molecule specifically binds to two different receptors (or domains or subunits thereof) such that the receptors (or domains or subunits) are maintained in proximity to each other such that the receptors (or domains or subunits), including domains thereof (e.g., intracellular domains) interact with each other and result in downstream signaling.

Comparable: As used herein, the term “comparable” is used to describe the degree of difference in two measurements of an evaluable quantitative or qualitative parameter. For example, where a first measurement of an evaluable quantitative parameter and a second measurement of the evaluable parameter do not deviate beyond a range that the skilled artisan would recognize as not producing a statistically significant difference in effect between the two results in the circumstances, the two measurements would be considered “comparable.” In some instances, measurements may be considered “comparable” if one measurement deviates from another by less than 30%, alternatively by less than 25%, alternatively by less than 20%, alternatively by less than 15%, alternatively by less than 10%, alternatively by less than 7%, alternatively by less than 5%, alternatively by less than 4%, alternatively by less than 3%, alternatively by less than 2%, or by less than 1%. In particular embodiments, one measurement is comparable to a reference standard if it deviates by less than 15%, alternatively by less than 10%, or alternatively by less than 5% from the reference standard. The term “comparable” is also used to describe the properties of chemical or biological entities that have similar or equivalent biological activities, functions, or results.

Conservative Substitutions: As used herein, the term “conservative amino acid substitution,” “conservatively modified variants,” “conservative variants,” “functionally conserved variants,” or “conservative amino acid substitutions” refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see, for example, Watson et al. 1987. Molecular Biology of the Gene, 4^{th} ed. The Benjamin/Cummings Pub. Co. p. 224). For example, the amino acids in each of the following groups can be considered as conservative amino acids of each other: (1) hydrophobic amino acids: alanine, isoleucine, leucine, tryptophan, phenylalanine, valine, proline, and glycine; (2) polar amino acids: glutamine, asparagine, histidine, serine, threonine, tyrosine, methionine, and cysteine; (3) basic amino acids: lysine and arginine; and (4) acidic amino acids: aspartic acid and glutamic acid. In addition, substitutions of structurally or functionally similar amino acids are less likely to disrupt biological activity. Exemplary conservative amino acid substitutions are set forth in Table 1, herein. Typically, an antibody, bispecific antibody or antigen-binding fragment of the invention which is modified in some way retains at least 10% of its binding activity (when compared to the parental antibody) when that activity is expressed on a molar basis. Preferably, an antibody or bispecific antibody or antigen-binding fragment of the invention retains at least 20%, 50%, 70%, 80%, 90%, 95% or 100% or more of the antigen target binding affinity as the parental antibody. It is also intended that an antibody, bispecific antibody or antigen-binding fragment of the invention can include conservative or non-conservative amino acid substitutions that do not substantially alter its biologic activity.

Isolated: The present disclosure provides isolated antibodies, antigen-binding fragments, and nucleotides. As used herein, the term “isolated,” as used in the context of an “isolated nucleic acid molecule” or an “isolated polynucleotide” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. is used in reference to a polypeptide of interest that, if naturally occurring, is in an environment different from that in which it can naturally occur. “Isolated” is meant to include polypeptides that are within samples that are substantially enriched for the polypeptide of interest and/or in which the polypeptide of interest is partially or substantially purified. Where the polypeptide is not naturally occurring, “isolated” indicates that the polypeptide has been separated from an environment in which it was synthesized, for example isolated from a recombinant cell culture comprising cells engineered to express the polypeptide or by a solution resulting from solid phase synthetic means. “Isolated” antibodies or bispecific antibodies or antigen-binding fragments thereof are at least partially free of other biological molecules from the cells or cell cultures in which they are produced. Such biological molecules include nucleic acids, proteins, lipids, carbohydrates, or other material such as cellular debris and growth medium. An isolated antibody or antigen-binding fragment may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antibodies or fragments. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

Control Sequences: The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

Operably Linked: The term “operably linked” is used herein to refer to the relationship between molecules, typically polypeptides or nucleic acids, which are arranged in a construct such that each of the functions of the component molecules is retained although the operable linkage may result in the modulation of the activity, either positively or negatively, of the individual components of the construct. For example, the operable linkage of a polyethylene glycol (PEG) molecule to a wild-type protein may result in a construct where the biological activity of the protein is diminished relative to the to the wild-type molecule, however the two are nevertheless considered operably linked. When the term “operably linked” is applied to the relationship of multiple nucleic acid sequences encoding differing functions, the multiple nucleic acid sequences when combined into a single nucleic acid molecule that, for example, when introduced into a cell using recombinant technology, provides a nucleic acid which is capable of effecting the transcription and/or translation of a particular nucleic acid sequence in a cell. For example, the nucleic acid sequence encoding a signal sequence may be considered operably linked to DNA encoding a polypeptide if it results in the expression of a preprotein whereby the signal sequence facilitates the secretion of the polypeptide; a promoter or enhancer is considered operably linked to a coding sequence if it affects the transcription of the sequence; or a ribosome binding site is considered operably linked to a coding sequence if it is positioned so as to facilitate translation. Generally in the context of nucleic acid molecules, the term “operably linked” means that the nucleic acid sequences being linked are contiguous, and, in the case of a secretory leader or associated subdomains of a molecule, contiguous and in reading phase. However, certain genetic elements such as enhancers may function at a distance and need not be contiguous with respect to the sequence to which they provide their effect but nevertheless may be considered operably linked.

Cell: As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

Derived From: As used herein in the term “derived from,” in the context of an amino acid sequence or polynucleotide sequence (e.g., an amino acid sequence “derived from”), is meant to indicate that the polypeptide or nucleic acid has a sequence that is based on that of a reference polypeptide or nucleic acid (e.g., a naturally occurring polypeptide or an encoding nucleic acid), and is not meant to be limiting as to the source or method in which the protein or nucleic acid is made. By way of example, the term “derived from” includes homologs or variants of reference amino acids or DNA sequences.

Effective Concentration: As used herein, the terms “effective concentration” or its abbreviation “EC” are used interchangeably to refer to the concentration of an agent (e.g., an anti-IL-10Rα-IL-10Rβ VHH dimer) in an amount sufficient to effect a change in a given parameter in a test system. The abbreviation “E” refers to the magnitude of a given biological effect observed in a test system when that test system is exposed to a test agent. When the magnitude of the response is expressed as a factor of the concentration (“C”) of the test agent, the abbreviation “EC” is used. In the context of biological systems, the term Emax refers to the maximal magnitude of a given biological effect observed in response to a saturating concentration of an activating test agent. When the abbreviation EC is provided with a subscript (e.g., EC₄₀, EC₅₀, etc.) the subscript refers to the percentage of the Emax of the biological observed at that concentration. For example, the concentration of a test agent sufficient to result in the induction of a measurable biological parameter in a test system that is 30% of the maximal level of such measurable biological parameter in response to such test agent, this is referred to as the “EC₃₀” of the test agent with respect to such biological parameter. Similarly, the term “EC₁₀₀” is used to denote the effective concentration of an agent that results the maximal (100%) response of a measurable parameter in response to such agent. Similarly, the term EC₅₀ (which is commonly used in the field of pharmacodynamics) refers to the concentration of an agent sufficient to results in the half-maximal (50%) change in the measurable parameter. The term “saturating concentration” refers to the maximum possible quantity of a test agent that can dissolve in a standard volume of a specific solvent (e.g., water) under standard conditions of temperature and pressure. In pharmacodynamics, a saturating concentration of a drug is typically used to denote the concentration sufficient of the drug such that all available receptors are occupied by the drug, and EC₅₀ is the drug concentration to give the half-maximal effect. The EC of a particular effective concentration of a test agent may be abbreviated with respect to the with respect to particular parameter and test system.

Enriched: As used herein in the term “enriched” refers to a sample that is non-naturally manipulated so that a species (e.g., a molecule or cell) of interest is present in: (a) a greater concentration (e.g., at least 3-fold greater, alternatively at least 5-fold greater, alternatively at least 10-fold greater, alternatively at least 50-fold greater, alternatively at least 100-fold greater, or alternatively at least 1000-fold greater) than the concentration of the species in the starting sample, such as a biological sample (e.g., a sample in which the molecule naturally occurs or in which it is present after administration); or (b) a concentration greater than the environment in which the molecule was made (e.g., a recombinantly modified bacterial or mammalian cell).

Extracellular Domain: As used herein the term “extracellular domain” or its abbreviation “ECD” refers to the portion of a cell surface protein (e.g., a cell surface receptor) which is outside of the plasma membrane of a cell. The term “ECD” may include the extra-cytoplasmic portion of a transmembrane protein or the extra-cytoplasmic portion of a cell surface (or membrane associated protein).

Percent Identity: As used herein, the term “percent (%) sequence identity” or “substantially identical” used in the context of nucleic acids or polypeptides, refers to a sequence that has at least 50% sequence identity with a reference sequence. Alternatively, percent sequence identity can be any integer from 50% to 100%. In some embodiments, a sequence has at least 50%, 55%, 60%, 65%, 70%, 75%, 80%, 85%, 90%, 91%, 92%, 93%, 94%, 95%, 96%, 97%, 98%, or 99% sequence identity to the reference sequence as determined with BLAST using standard parameters, as described below. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. A comparison window includes reference to a segment of any one of the number of contiguous positions, for example, a segment of at least 10 residues. In some embodiments, the comparison window has from 10 to 600 residues, for example, about 10 to about 30 residues, about 10 to about 20 residues, about 50 to about 200 residues, or about 100 to about 150 residues, in which a sequence may be compared to a reference sequence of the same number of contiguous positions after the two sequences are optimally aligned. Algorithms that are suitable for determining percent sequence identity and sequence similarity are the BLAST and BLAST 2.0 algorithms, which are described in Altschul et al. 1990. J Mol Biol. 215: 403-410 and Altschul et al. 1977. Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). This initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff. 1989. Proc Natl Acad Sci. USA 89:10915). The BLAST algorithm also performs a statistical analysis of the similarity between two sequences (see, for example, Karlin & Altschul. 1993. Proc Nat'l Acad Sci. USA 90:5873-5787). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference sequence if the smallest sum probability in a comparison of the test amino acid sequence to the reference amino acid sequence is less than about 0.01, more preferably less than about 10⁻⁵, and most preferably less than about 10⁻²⁰.

Percent Human: The terms “percent human,” “percent humanization,” or “humanized” in the context of the degree to which an IL-10 agonist compound amino acid sequence is modified to be more human-like means the percentage of amino acid residues of a subject polypeptide sequence that are identical to a closely matched naturally occurring human polypeptide reference sequence, such as a similar human germline sequence. For example, as used herein in reference to a polypeptide sequence such as a sdAb, scFv, or VHH sequence, “percent human” means the percentage of amino acid residues of the sdAb, scFv, or VHH amino acid sequence that are identical to a closely matched human germline reference sequence. There are numerous human germline sequences that are commonly used and may be selected as a reference sequence for determining “percent humanization.” “Percent humanization” of the anti-IL-10Rα VHH antibody sequences disclosed herein is the percentage of anti-IL-10Rα. VHH amino acid residues that are identical to the amino acid sequence of the closely matched germline sequence V3-23 (UniProt immunoglobulin heavy variable sequence 3-23, Entry No. P01764) (i.e., the ratio of the number of identical amino acid residues to the total number of amino acid residues). “Percent humanization” of the anti-IL-10Rβ VHH sequences disclosed herein is the percentage of anti-IL-10Rα VHH amino acid residues that are identical to the amino acid sequences of the closely matched germline sequence VH3-66 (UniProt Immunoglobulin heavy variable sequence 3-66, Entry No. A0A0C4DH42) (i.e., the ratio of the number of identical amino acid residues to the total number of amino acid residues). For purposes of calculating percent humanization, the V segment of the selected human germline sequence (V3-23 or VH3-66, as described above) is used as the reference germline sequence, by comparing to the reference germline sequence of the IL-10Rα and IL-10Rβ humanized framework region, including CDR1 and CDR2.

Intracellular Signaling: As used herein, the terms “intracellular signaling” and “downstream signaling” are used interchangeably to refer to the to the cellular signaling process that is caused by the interaction of the intracellular domains (ICDs) of two or more cell surface receptors that are in proximity of each other. In receptor complexes via the JAK/STAT pathway, the association of the ICDS of the receptor subunits brings the JAK domains of the ICDs into proximity which initiates a phosphorylation cascade in which STAT molecules are phosphorylated and translocate to the nucleus associating with particular nucleic acid sequences resulting in the activation and expression of particular genes in the cell. The IL-10 agonist compounds (e.g., single-domain antibody polypeptides of the IL-10 agonist compounds) of the present disclosure provide intracelluar signaling characteristic of the IL-10 receptor receptor when activated by its natural cognate IL-10. To measure downstream signaling activity, a number of methods are available. For example, in some embodiments, one can measure JAK/STAT signaling by the presence of phosphorylated receptors and/or phosphorylated STATs. In other embodiments, the expression of one or more downstream genes, whose expression levels can be affected by the level of downstream signaling caused by the binding molecule, can also be measured.

Amount Sufficient to Cause a Response: As used herein the phrase “in an amount sufficient to cause a response” is used in reference to the amount of a test agent sufficient to provide a detectable change in the level of an indicator measured before (e.g., a baseline level) and after the application of a test agent to a test system. In some embodiments, the test system is a cell, tissue or organism. In some embodiments, the test system is an in vitro test system such as a fluorescent assay. In some embodiments, the test system is an in vivo system which involves the measurement of a change in the level of a parameter of a cell, tissue, or organism reflective of a biological function before and after the application of the test agent to the cell, tissue, or organism. In some embodiments, the indicator is reflective of biological function or state of development of a cell evaluated in an assay in response to the administration of a quantity of the test agent. In some embodiments, the test system involves the measurement of a change in the level an indicator of a cell, tissue, or organism reflective of a biological condition before and after the application of one or more test agents to the cell, tissue, or organism. The term “in an amount sufficient to effect a response” may be sufficient to be a therapeutically effective amount but may also be more or less than a therapeutically effective amount.

In Need of Treatment: The term “in need of treatment” as used herein refers to a judgment made by a physician or other caregiver with respect to a subject that the subject requires or will potentially benefit from treatment. This judgment is made based on a variety of factors that are in the realm of the physician's or caregiver's expertise.

Ligand: As used herein, the term “ligand” refers to a molecule that exhibits specific binding to a receptor and results in a change in the biological activity of the receptor so as to effect a change in the activity of the receptor to which it binds. In one embodiment, the term “ligand” refers to a molecule, or complex thereof, that can act as an agonist or antagonist of a receptor. As used herein, the term “ligand” encompasses natural and synthetic ligands. “Ligand” also encompasses small molecules, for example, peptide mimetics of cytokines and peptide mimetics of antibodies. The complex of a ligand and receptor is termed a “ligand-receptor complex.”

Joined: As used herein, the term “joined” means that two elements, such as two protein domains, are joined together or are otherwise in stable association with each other. Two proteins may be “joined” directly, with the C-terminus of one protein domain covalently joined to the N-terminus of a second protein domain. Alternatively, two proteins may be “joined” by means of a linker polypeptide or other chemical linker compound.

Linker: As used herein, the term “linker” refers to a linkage between two elements, for example, protein domains. A linker can be a covalent bond or a peptide linker. The term “bond” refers to a chemical bond, for example, an amide bond or a disulfide bond, or any kind of bond created from a chemical reaction, for example, chemical conjugation. The term “peptide linker” refers to an amino acid or polypeptide that may be employed to link two protein domains to provide space and/or flexibility between the two protein domains.

Modulate: As used herein, the terms “modulate,” and “modulation” and the like refer to the ability of a test agent to cause a response, either positive or negative or directly or indirectly, in a system, including a biological system, or biochemical pathway. The term modulator includes both agonists (including partial agonists, full agonists and superagonists) and antagonists.

Multimerization: As used herein, the term “multimerization” refers to two or more cell surface receptors, or domains or subunits thereof, being brought in close proximity to each other such that the receptors, or domains or subunits thereof, can interact with each other and cause intracellular signaling.

N-Terminus and C-Terminus: As used herein in the context of the structure of a polypeptide, “N-terminus” (or “amino terminus”) and “C-terminus” (or “carboxyl terminus”) refer to the extreme (final) amino and carboxyl ends of the polypeptide, respectively, while the terms “N-terminal” and “C-terminal” refer to relative positions in the amino acid sequence of the polypeptide near or toward the N-terminus and the C-terminus, respectively, and can include the residues at or near the N-terminus and C-terminus, respectively. The terms “immediately N-terminal” refers to a position of a first amino acid residue relative to a second amino acid residue in a contiguous polypeptide sequence, where the first and second amino acid residues are covalently bound to provide a contiguous amino acid sequence and where the first amino acid is closer to the N-terminus of the polypeptide. “Immediately C-terminal” refers to the position of a first amino acid residue relative to a second amino acid residue in a contiguous polypeptide sequence, the first amino acid being closer to the C-terminus of the polypeptide.

Neoplastic Disease: As used herein and as discussed in more detail below, the term “neoplastic disease” refers to disorders or conditions in a subject arising from cellular hyper-proliferation or unregulated (or dysregulated) cell replication. The term neoplastic disease refers to disorders arising from the presence of neoplasms in the subject. Neoplasms may be classified as: (1) benign (2) pre-malignant (or “pre-cancerous”); and (3) malignant (or “cancerous”). The term “neoplastic disease” includes neoplastic-related diseases, disorders and conditions referring to conditions that are associated, directly or indirectly, with neoplastic disease, and includes, for example, angiogenesis and precancerous conditions such as dysplasia or smoldering multiple myeloma. Examples of benign disorders arising from dysregulated cell replication include hypertrophic scars such as keloid scars.

Nucleic Acid: The terms “nucleic acid,” “nucleic acid molecule,” “polynucleotide,” “nucleotide,” and the abbreviation “nt” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the

Partial Agonist: As used herein, the term “partial agonist” refers to a molecule that specifically binds that bind to and activate a given receptor but possess only partial activation the receptor relative to a full agonist. Partial agonists may display both agonistic and antagonistic effects. For example, when both a full agonist and partial agonist are present, the partial agonist acts as a competitive antagonist by competing with the full agonist for the receptor binding resulting in net decrease in receptor activation relative to the contact of the receptor with the full agonist in the absence of the partial agonist. Clinically, partial agonists can be used to activate receptors to give a desired submaximal response when inadequate amounts of the endogenous ligand are present, or they can reduce the overstimulation of receptors when excess amounts of the endogenous ligand are present. The maximum response (Emax) produced by a partial agonist is called its intrinsic activity and may be expressed on a percentage scale where a full agonist produced a 100% response. In some embodiments, the IL-10 agonist compound (e.g., a single-domain antibody polypeptide of an IL-10 agonist compound) has a reduced Emax compared to the Emax caused by IL-10. Emax reflects the maximum response level in a cell type that can be obtained by a ligand (e.g., a binding molecule described herein or the native cytokine (e.g., IL-10)). In some embodiments, the IL-10 agonist compound (e.g., a single-domain antibody polypeptide of an IL-10 agonist compound) described herein has at least 1% (e.g., between 1% and 100%, between 10% and 100%, between 20% and 100%, between 30% and 100%, between 40% and 100%, between 50% and 100%, between 60% and 100%, between 70% and 100%, between 80% and 100%, between 90% and 100%, between 1% and 90%, between 1% and 80%, between 1% and 70%, between 1% and 60%, between 1% and 50%, between 1% and 40%, between 1% and 30%, between 1% and 20%, or between 1% and 10%) of the Emax caused by IL-10. In other embodiments, the Eₘₐₓ of the IL-10 agonist compound (e.g., a single-domain antibody polypeptide of an IL-10 agonist compound) described herein is greater (e.g., at least 1%, 5%, 1, 15%, 20%, 25%, 30%, 35%, 40%, 45%, or 50% greater) than the Emax of the natural ligand, IL-10. In some embodiments, by varying the linker length of the IL-10 agonist compounds (e.g., single-domain antibody polypeptides of the IL-10 agonist compounds), the Emax of the IL-10 agonist compound (e.g., a single-domain antibody polypeptide of an IL-10 agonist compound) can be changed. The IL-10 agonist compound (e.g., a single-domain antibody polypeptide of an IL-10 agonist compound) can cause Eₘₐₓ in the most desired cell types, and a reduced Eₘₐₓ in other cell types.

Polypeptide: As used herein the terms “polypeptide,” “peptide,” and “protein,” used interchangeably herein, refer to a polymeric form of amino acids or amino acid sequence of any length, which can include genetically coded and non-genetically coded amino acids, chemically or biochemically modified or derivatized amino acids, and polypeptides having modified polypeptide backbones. The term polypeptide includes fusion proteins, including, but not limited to, fusion proteins with a heterologous amino acid sequence; fusion proteins with heterologous and homologous leader sequences; fusion proteins with or without N-terminal methionine residues; fusion proteins with amino acid sequences that facilitate purification such as chelating peptides; fusion proteins with immunologically tagged proteins; fusion proteins comprising a peptide with immunologically active polypeptide fragment (e.g., antigenic diphtheria or tetanus toxin or toxoid fragments) and the like.

Prevent: As used herein the terms “prevent,” “preventing,” “prevention” and the like refer to a course of action initiated with respect to a subject prior to the onset of a disease, disorder, condition or symptom thereof so as to prevent, suppress, inhibit, or reduce, either temporarily or permanently, a subject's risk of developing a disease, disorder, condition or the like (as determined by, for example, the absence of clinical symptoms) or delaying the onset thereof, generally in the context of a subject predisposed due to genetic, experiential or environmental factors to having a particular disease, disorder or condition. In certain instances, the terms “prevent,” “preventing,” and “prevention” are also used to refer to the slowing of the progression of a disease, disorder, or condition from a present state to a more deleterious state.

Proximity: As used herein, the term “proximity” refers to the spatial proximity or physical distance between two cell surface receptors, or domains or subunits thereof, after a binding molecule described herein binds to the two cell surface receptors, or domains or subunits thereof. In some embodiments, after the binding molecule binds to the cell surface receptors, or domains or subunits thereof, the spatial proximity between the cell surface receptors, or domains or subunits thereof, can be, for example, less than about 500 angstroms, such as e.g., a distance of about 5 angstroms to about 500 angstroms. In some embodiments, the spatial proximity amounts to less than about 5 angstroms, less than about 20 angstroms, less than about 50 angstroms, less than about 75 angstroms, less than about 100 angstroms, less than about 150 angstroms, less than about 250 angstroms, less than about 300 angstroms, less than about 350 angstroms, less than about 400 angstroms, less than about 450 angstroms, or less than about 500 angstroms. In some embodiments, the spatial proximity amounts to less than about 100 angstroms. In some embodiments, the spatial proximity amounts to less than about 50 angstroms. In some embodiments, the spatial proximity amounts to less than about 20 angstroms. In some embodiments, the spatial proximity amounts to less than about 10 angstroms. In some embodiments, the spatial proximity ranges from about 10 to 100 angstroms, from about 50 to 150 angstroms, from about 100 to 200 angstroms, from about 150 to 250 angstroms, from about 200 to 300 angstroms, from about 250 to 350 angstroms, from about 300 to 400 angstroms, from about 350 to 450 angstroms, or about 400 to 500 angstroms. In some embodiments, the spatial proximity amounts to less than about 250 angstroms, alternatively less than about 200 angstroms, alternatively less than about 150 angstroms, alternatively less than about 120 angstroms, alternatively less than about 100 angstroms, alternatively less than about 80 angstroms, alternatively less than about 70 angstroms, or alternatively less than about 50 angstroms.

Receptor: As used herein, the term “receptor” refers to a polypeptide having a domain that specifically binds a ligand that binding of the ligand results in a change to at least one biological property of the polypeptide. In some embodiments, the receptor is a “soluble” receptor that is not associated with a cell surface. In some embodiments, the receptor is a cell surface receptor that comprises an extracellular domain (ECD) and a membrane-associated domain which anchors the ECD to the cell surface. In some embodiments of cell surface receptors, the receptor is a membrane-spanning polypeptide comprising an intracellular domain (ICD) and extracellular domain (ECD) linked by a membrane-spanning domain typically referred to as a transmembrane domain. The binding of the ligand to the receptor results in a conformational change in the receptor resulting in a measurable biological effect. In some instances, where the receptor is a membrane-spanning polypeptide comprising an ECD, TM, and ICD, the binding of the ligand to the ECD results in a measurable intracellular biological effect mediated by one or more domains of the ICD in response to the binding of the ligand to the ECD. In some embodiments, a receptor is a component of a multi-component complex to facilitate intracellular signaling. For example, the ligand may bind a cell surface molecule not associated with any intracellular signaling alone but upon ligand binding facilitates the formation of a multimeric complex that results in intracellular signaling.

Recombinant: As used herein, the term “recombinant” is used as an adjective to refer to the method by a polypeptide, nucleic acid, or cell that was modified using recombinant DNA technology. A recombinant protein is a protein produced using recombinant DNA technology and may be designated as such using the abbreviation of a lowercase “r” (e.g., rhIL-10) to denote the method by which the protein was produced. Similarly, a cell is referred to as a “recombinant cell” if the cell has been modified by the incorporation (e.g., transfection, transduction, infection) of exogenous nucleic acids (e.g., ssDNA, dsDNA, ssRNA, dsRNA, mRNA, viral or non-viral vectors, plasmids, cosmids and the like) using recombinant DNA technology. The techniques and protocols for recombinant DNA technology are well known in the art such as those that can be found in Sambrook et al. 1989. Molecular Cloning: A Laboratory Manual (2d ed., Cold Spring Harbor Laboratory Press, Plainview, N.Y.) and other standard molecular biology laboratory manuals.

Response: The term “response,” for example, of a cell, tissue, organ, or organism, encompasses a quantitative or qualitative change in an evaluable biochemical or physiological parameter, (e.g., concentration, density, adhesion, proliferation, activation, phosphorylation, migration, enzymatic activity, level of gene expression, rate of gene expression, rate of energy consumption, level of or state of differentiation) where the change is correlated with the activation, stimulation, or treatment, with or contact with exogenous agents or internal mechanisms such as genetic programming. In certain contexts, the terms “activation”, “stimulation”, and the like refer to cell activation as regulated by internal mechanisms, as well as by external or environmental factors; whereas the terms “inhibition”, “down-regulation” and the like refer to the opposite effects. A “response” may be evaluated in vitro such as through the use of assay systems, surface plasmon resonance, enzymatic activity, mass spectroscopy, and amino acid or protein sequencing technologies. A “response” may be evaluated in vivo quantitatively by evaluation of objective physiological parameters such as body temperature, body weight, tumor volume, blood pressure, results of X-ray or other imaging technology or qualitatively through changes in reported subjective feelings of well-being, depression, agitation, or pain. In some embodiments, the level of proliferation of CD3-activated primary human T-cells may be evaluated in a bioluminescent assay that generates a luminescent signal that is proportional to the amount of ATP present which is directly proportional to the number of viable cells present in culture as described in Crouch et al. 1993. J Immunol Methods 160:81-8 or using commercially available assays such as the CellTiter-Glo® 2.0 Cell Viability Assay or CellTiter-Glo® 3D Cell Viability kits commercially available from Promega Corporation, Madison WI 53711 as catalog numbers G9241 and G9681 in substantial accordance with the instructions provided by the manufacturer. In some embodiments, the level of activation of T cells in response to the administration of a test agent may be determined by flow cytometric methods as described as determined by the level of STAT (e.g., STAT1, STAT3, STAT5) phosphorylation in accordance with methods well known in the art.

Significantly Reduced Binding: As used herein, the term “significantly reduced binding” is used with respect to a variant of a first molecule (e.g., a ligand) that exhibits a significant reduction in the affinity for a second molecule (e.g., receptor) relative to the parent form of the first molecule. With respect to antibody variants, an antibody variant “exhibits significantly reduced binding” if the variant binds to the native form of the receptor with an affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the parent antibody from which the variant was derived. Similarly, with respect to variant ligands, a variant ligand “exhibits significantly reduced binding” if the variant ligand binds to a receptor with an affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the parent ligand from which the variant ligand was derived. Similarly, with respect to variant receptors, a variant ligand “exhibits significantly reduced binding” if the affinity of the variant receptors binds to a with an affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the parent receptor from which the variant receptor was derived.

Specifically Binds: As used herein the term “specifically binds” refers to the degree of affinity for which a first molecule exhibits with respect to a second molecule. In the context of binding pairs (e.g., ligand/receptor, antibody/antigen) a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the first molecule of the binding pair when the affinity of the first molecule for the second molecule is at least two-fold greater, alternatively at least five times greater, alternatively at least ten times greater, alternatively at least 20-times greater, or alternatively at least 100-times greater than the affinity of the first molecule for other components present in the sample. In a particular embodiment, where the first molecule of the binding pair is an antibody, the antibody specifically binds to the antigen (or antigenic determinant (epitope) of a protein, antigen, ligand, or receptor) if the equilibrium dissociation constant between an antibody and the antigen is greater than about 10⁻⁶ M, alternatively greater than about 10⁸ M, alternatively greater than about 10⁻¹⁰ M, alternatively greater than about 10⁻¹¹ M, greater than about 10⁻¹² M as determined by, for example, Scatchard analysis (Munsen. et al. 1980. Analyt Biochem. 107:220-239). In one embodiment, a ligand specifically binds to a receptor if the dissociation is greater than about 10⁻⁵ M, alternatively greater than about 10⁻⁶ M, alternatively greater than about 10⁻⁷M, alternatively greater than about 10⁻⁸ M, alternatively greater than about 10⁻⁹M, alternatively greater than about 10⁻¹⁰ M, or alternatively greater than about 10⁻¹¹ M. Specific binding may be assessed using techniques known in the art including but not limited to competition ELISA assays, radioactive ligand binding assays (e.g., saturation binding, Scatchard plot, nonlinear curve fitting programs and competition binding assays); non-radioactive ligand binding assays (e.g., fluorescence polarization (FP), fluorescence resonance energy transfer (FRET); liquid phase ligand binding assays (e.g., real-time polymerase chain reaction (RT-qPCR), and immunoprecipitation); and solid phase ligand binding assays (e.g., multiwell plate assays, on-bead ligand binding assays, on-column ligand binding assays, and filter assays)) and surface plasmon resonance assays (see, for example, Drescher et al. 2009. Methods Mol Biol. 493:323-343 with commercially available instrumentation such as the Biacore 8+, Biacore S200, and Biacore T200 (GE Healthcare Bio-Sciences, 100 Results Way, Marlborough MA 01752). In some embodiments, the present disclosure provides molecules (e.g., IL-10R binding sdAbs) that specifically bind to IL-10R. As used herein, the binding affinity of an IL-10 agonist compound for the IL-10R, the binding affinity may be determined and/or quantified by surface plasmon resonance (“SPR”). In evaluating the binding affinity of an IL-10 agonist compound for IL-10Rα or IL-10Rβ subunits, either member of the binding pair may be immobilized, and the other element of the binding pair be provided in the mobile phase. In some embodiments, the sensor chip on which the protein of interest is to be immobilized is conjugated with a substance to facilitate binding of the protein of interest such as nitrilotriacetic acid (NTA) derivatized surface plasmon resonance sensor chips (e.g., Sensor Chip NTA available from Cytiva Global Life Science Solutions USA LLC, Marlborough MA as catalog number BR100407), as anti-His tag antibodies (e.g., anti-histidine CM5 chips commercially available from Cytiva, Marlborough MA), protein A or biotin. Consequently, to evaluate binding, it is frequently necessary to modify the protein to provide for binding to the substance conjugated to the surface of the chip. For example, the one member of the binding pair to be evaluated by incorporation of a chelating peptide comprising poly-histidine sequence (e.g., 6×His (SEQ ID NO: 451) or 8×His (SEQ ID NO: 453)) for retention on a chip conjugated with NTA. In some embodiments, the binding molecule may be immobilized on the chip and the receptor subunit (or ECD fragment thereof) is in the mobile phase. Alternatively, the receptor subunit (or ECD fragment thereof) may be immobilized on the chip and the binding molecule be provided in the mobile phase. In either circumstance, it should be noted that modifications of some proteins for immobilization on a coated SPR chip may interfere with the binding properties of one or both components of the binding pair to be evaluated by SPR. In such cases, it may be necessary to switch the mobile and bound elements of the binding pair or use a chip with a binding agent that facilitates non-interfering conjugation of the protein to be evaluated. Alternatively, when evaluating the binding affinity of the binding molecule for receptor subunit using SPR, the binding molecule may be derivatized by the C-terminal addition of a poly-His sequence (e.g., 6×His (SEQ ID NO: 451) or 8×His (SEQ ID NO: 453)) and immobilized on the NTA derivatized sensor chip and the receptor subunit for which the binding affinity is being evaluated is provided in the mobile phase. The means for incorporation of a poly-His sequence into the C-terminus of the binding molecule produced by recombinant DNA technology is well known to those of skill in the relevant art of biotechnology.

Recipient, Subject, Individual, or Patient: The terms “recipient,” “individual,” “subject,” and “patient,” are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is a human being.

Substantially Pure: As used herein, the term “substantially pure” indicates that a component of a composition makes up greater than about 50%, alternatively greater than about 60%, alternatively greater than about 70%, alternatively greater than about 80%, alternatively greater than about 90%, alternatively greater than about 95%, of the total content of the composition. A protein that is “substantially pure” comprises greater than about 50%, alternatively greater than about 60%, alternatively greater than about 70%, alternatively greater than about 80%, alternatively greater than about 90%, alternatively greater than about 95%, of the total content of the composition.

Suffering From: As used herein, the term “suffering from” refers to a determination made by a physician with respect to a subject based on the available information accepted in the field for the identification of a disease, disorder, or condition, including but not limited to X-ray, CT-scans, conventional laboratory diagnostic tests (e.g., blood count), genomic data, protein expression data, immunohistochemistry, that the subject requires or will benefit from treatment. The term “suffering from” is typically used in conjunction with a particular disease state, such as “suffering from a neoplastic disease,” which refers to a subject that has been diagnosed with the presence of a neoplasm.

T-Cell: As used herein, the term “T-cell” or “T cell” is used in its conventional sense to refer to a lymphocyte that differentiates in the thymus, possesses specific cell-surface antigen receptors, and includes some that control the initiation or suppression of cell-mediated and humoral immunity and others that lyse antigen-bearing cells. In some embodiments, the T cell includes without limitation naïve CD8⁺ T cells, cytotoxic CD8⁺ T cells, naïve CD4⁺ T cells, helper T cells, e.g., T_{H}1, T_{H}2, T_{H}9, T_{H}11, T_{H}22, T_{FH}; regulatory T cells, e.g., T_{R}1, Tregs, inducible Tregs; memory T cells, e.g., central memory T cells, effector memory T cells, NKT cells, tumor-infiltrating lymphocytes (TILs) and engineered variants of such T-cells including but not limited to CAR-T cells, recombinantly modified TILs and TCR engineered cells.

Therapeutically Effective Amount: As used herein, the term the phrase “therapeutically effective amount” is used in reference to the administration of an agent to a subject, either alone or as part of a pharmaceutical composition or treatment regimen, in a single dose or as part of a series of doses in an amount capable of having any detectable, positive effect on any symptom, aspect, or characteristic of a disease, disorder or condition when administered to the subject. The therapeutically effective amount can be ascertained by measuring relevant physiological effects, and it may be adjusted in connection with a dosing regimen and in response to diagnostic analysis of the subject's condition, and the like. The parameters for evaluation to determine a therapeutically effective amount of an agent are determined by the physician using art-accepted diagnostic criteria including but not limited to indicia such as age, weight, sex, general health, ECOG score, observable physiological parameters, blood levels, blood pressure, electrocardiogram, computerized tomography, X-ray, and the like. Alternatively, or in addition, other parameters commonly assessed in the clinical setting may be monitored to determine if a therapeutically effective amount of an agent has been administered to the subject, such as body temperature, heart rate, normalization of blood chemistry, normalization of blood pressure, normalization of cholesterol levels, or any symptom, aspect, or characteristic of the disease, disorder or condition, modification of biomarker levels, increase in the duration of survival, extended duration of progression-free survival, extension of the time to progression, increased time to treatment failure, extended duration of event-free survival, extension of time to next treatment, improvement objective response rate, improvement in the duration of response, and the like that that are relied upon by clinicians in the field for the assessment of an improvement in the condition of the subject in response to administration of an agent.

Regulatory T-Cell: The terms “regulatory T-cell” or “Treg cell” as used herein refers to a type of CD4⁺ T cell that can suppress the responses of other T cells including but not limited to effector T cells (Teff). Treg cells are characterized by the expression of CD4, the α-subunit of the IL2 receptor (CD25), and the transcription factor forkhead box P3 (FOXP3) (Sakaguchi. 2004. Annu Rev Immunol. 22:531-62. By “conventional CD4⁺ T cells” is meant CD4⁺ T cells other than regulatory T cells.

Transmembrane Domain: The term “transmembrane domain” or “TM” refers to the domain of a membrane-spanning polypeptide which, when the membrane-spanning polypeptide is associated with a cell membrane, is which is embedded in the cell membrane and is in peptidyl linkage with the extracellular domain (ECD) and the intracellular domain (ICD) of a membrane-spanning polypeptide. A transmembrane domain may be homologous (naturally associated with) or heterologous (not naturally associated with) with either or both of the extracellular and/or intracellular domains. A transmembrane domain may be homologous (naturally associated with) or heterologous (not naturally associated with) with either or both of the extracellular and/or intracellular domains. In some embodiments, where the receptor is a chimeric receptor comprising the intracellular domain derived from a first parental receptor and a second extracellular domain is derived from a second different parental receptor, the transmembrane domain of the chimeric receptor is the transmembrane domain normally associated with either the ICD or the ECD of the parent receptor from which the chimeric receptor is derived. Alternatively, the transmembrane domain of the receptor may be an artificial amino acid sequence that spans the plasma membrane. In some embodiments, where the receptor is a chimeric receptor comprising the intracellular domain derived from a first parental receptor and a second extracellular domain derived from a second different parental receptor, the transmembrane domain of the chimeric receptor is the transmembrane domain normally associated with either the ICD or the ECD of the parent receptor from which the chimeric receptor is derived.

Treat: The terms “treat,” “treating,” treatment,” and the like refer to a course of action (such as administering a binding molecule described herein, or a pharmaceutical composition comprising the same) initiated with respect to a subject after a disease, disorder or condition or a symptom thereof, has been diagnosed, observed, or the like in the subject so as to eliminate, reduce, suppress, mitigate, or ameliorate, either temporarily or permanently, at least one of the underlying causes of such disease, disorder, or condition afflicting a subject, or at least one of the symptoms associated with such disease, disorder, or condition. The treatment includes a course of action taken with respect to a subject suffering from a disease where the course of action results in the inhibition (e.g., arrests the development of the disease, disorder, or condition or ameliorates one or more symptoms associated therewith) of the disease in the subject.

Wild-type: As used herein, the term “wild-type” or “WT” or “native” is used to refer to an amino acid sequence or a nucleotide sequence that is found in nature and that has not been altered by the hand of man.

### Interleukin 10 Receptor Agonist Compounds

The present disclosure provides IL-10 agonist compounds that are synthetic ligands of the IL-10 receptor. In some embodiments, the IL-10 agonist compounds (e.g., single-domain antibody polypeptides of the IL-10 agonist compounds) are humanized IL-10 agonist compounds that are ligands of the human IL-10 receptor.

The IL-10 agonist compounds (e.g., single-domain antibody polypeptides of the IL-10 agonist compounds) of the present disclosure comprise two or more single-domain antibodies (sdAbs) that selectively bind to the extracellular domain of the IL-10Rα and IL-10Rβ receptor subunits. In one embodiment, the present disclosure provides an IL-10 receptor (IL-10R) agonist compound that binds to the IL-10R receptor, the IL-10R receptor binding molecule comprising: a first single-domain antibody (sdAb) that specifically binds to the extracellular domain of the IL-10Rα subunit of the IL-10 receptor (an “IL-10Rα sdAb”), and a second single-domain antibody that specifically binds to the extracellular domain of the IL-10Rβ subunit of the IL-10 receptor (an “IL-10Rβ sdAb”), wherein the first sdAb and second sdAb are in stable association (e.g., joined, for example by a linker, such as a polypeptide linker, or non-covalently via association of an IL-10Rα VHH-Fc polypeptide to an IL-10Rβ VHH-Fc polypeptide, as described herein). In some embodiments, the IL-10Rα and IL-10Rβ subunits of the IL-10 receptor are dimerized in response to contact with the IL-10 agonist compound, and contacting a cell expressing the IL-10Rα and IL-10Rβ with an effective amount of the IL-10 agonist compound (e.g., a single-domain antibody polypeptide of an IL-10 agonist compound) results in the intracellular domains of IL-10Rα and IL-10Rβ being brought into proximity and intracellular signaling.

The IL-10 agonist compounds (e.g., single-domain antibody polypeptides of the IL-10 agonist compounds) of the present disclosure are useful in the treatment or prevention of disease in mammalian subjects, In some embodiments, the present disclosure provides for the treatment or prevention of autoimmune disease in a mammalian subject by the administration of a therapeutically effective amount of an IL-10 agonist compound of the present disclosure. In some embodiments, the present disclosure provides for the treatment or prevention of infectious disease, including viral and chronic viral infections, in a mammalian subject by the administration of a therapeutically effective amount of an IL-10 agonist compound of the present disclosure. In some embodiments, the present disclosure provides for the treatment or prevention of neoplastic disease in a mammalian subject by the administration of a therapeutically effective amount of an IL-10 agonist compound of the present disclosure. In some embodiments, the present disclosure provides for the treatment or prevention of neoplastic, infectious, or autoimmune in a mammalian subject by the administration of a therapeutically effective amount of an IL-10 agonist compound of the present disclosure in combination with one or more supplementary therapeutic agents.

In some embodiments, the present disclosure provides an IL-10 agonist compound that is modified to provide the prolonged duration of action in vivo in a mammalian subject and pharmaceutically acceptable formulations thereof.

The present disclosure further provides a pharmaceutically acceptable formulation of an IL-10 agonist compound for administration to a mammalian subject. The present disclosure further provides a pharmaceutically acceptable composition for administration to a mammalian subject the composition comprising a nucleic acid sequence encoding a polypeptide IL-10 agonist compound, a recombinant viral or non-viral vector encoding or polypeptide IL-10 agonist compound, or a recombinantly modified mammalian cell comprising a nucleic acid sequence encoding a polypeptide IL-10 agonist compound, in each case the nucleic acid sequence operably linked to one or more expression control elements functional in a mammalian cell.

In some embodiments, the IL-10 agonist compounds (e.g., single-domain antibody polypeptides of the IL-10 agonist compounds) of the present disclosure are polypeptides The present disclosure provides nucleic acid sequences encoding polypeptide IL-10 agonist compounds. The present disclosure further provides a recombinant vector comprising a nucleic acid sequence encoding polypeptide IL-10 agonist compounds. The present disclosure further provides a recombinantly modified mammalian cell comprising a nucleic acid encoding a polypeptide IL-10 agonist compound. The present disclosure further provides methods for the recombinant production, isolation, purification, and characterization of a polypeptide IL-10 agonist compound.

The cognate ligand of the IL-10 receptor (IL-10R) is the cytokine IL-10. The term IL-10 includes human and murine (or mouse) IL-10. Human IL-10 (hIL-10) is a non-covalently linked homodimeric protein comprising two identical subunits. Each human IL-10 monomer is expressed as a 178 amino acid pre-protein comprising 18 amino acid signal sequence that is post-translationally removed to render a 160 amino acid mature protein. The canonical amino acid sequence of the mature human IL-10 protein is disclosed as UniProt Reference No. P22301. Mouse (or murine) IL-10 (mIL-10) is a non-covalently linked homodimeric protein comprising two identical subunits. Each murine IL-10 monomer is expressed as a 178 amino acid pre-protein comprising an 18 amino acid signal sequence which is post-translationally removed to render a 160 amino acid mature protein. The canonical amino acid sequence of the mature murine IL-10 protein is disclosed as UniProt Reference No. P18893) without the signal sequence (corresponding to amino acids 19-178 of the pre-protein). IL-10 agonist compounds activate IL-10 signaling in a cell expressing the IL-10 receptor. The present disclosure provides IL-10 agonist compounds engineered to provide selective levels of intracellular signaling in cells expressing the IL-10 receptor. The present invention provides IL-10 agonist compounds engineered to generate intracellular signaling in particular cell types.

The IL-10 receptor is a heterodimeric protein complex comprising the IL-10Rα and IL-10Rβ subunits. The interaction of the IL-10 on the surface of a mammalian cell expressing the IL-10Rα and IL-10Rβ subunits results in the dimerization of IL-10Rα and IL-10Rβ and intracellular signaling. The intracellular signaling characteristic of IL-10 mediated dimerization of the IL-10Rα and IL-10Rβ is the activation of the JAK/STAT pathway, in particular the phosphorylation of STAT3 molecule, which is a component of the intracellular signaling pathway that, in combination with other components of the signaling pathway results in modulation of gene expression. In some embodiments, the IL-10 receptor is the human IL-10 receptor. As used herein, the terms “IL-10 receptor” and “IL-10R” are used interchangeably to refer to a heterodimeric complex comprising the IL-10Rα and IL-10Rβ subunits of the IL-10R.

The IL-10Rα component of the human IL-10 receptor is the human IL-10R (hIL-10Rα) protein. The canonical full-length hIL-10Rα protein is described in the UniProt Database ID No. Q13651. The IL-10Rα component of the mouse IL-10 receptor is the mouse IL-10Rα (mIL-10Rα) protein. The canonical full-length mIL-10Rα is described in the UniProt Database, ID No. Q61727.

The IL10Rβ component of the human IL-10 receptor is the human IL019R (hIL-10Rβ) protein. The canonical full-length hIL-10Rβ precursor is described in the UniProt Database, ID No. Q08334. The canonical murine IL-10Rβ (mIL-10Rβ) is described in the UniProt Database, ID No. Q61190.

### Single-Domain Antibody

The IL-10 agonist compounds (e.g., single-domain antibody polypeptides of the IL-10 agonist compounds) of the present disclosure comprise two or more single-domain antibodies. The term “single-domain antibody” (sdAb) as used herein refers an antibody fragment consisting of a monomeric variable antibody domain that is able to bind specifically to an antigen and compete for binding with the parent antibody from which it is derived. The term “single-domain antibody” includes scFv and VHH molecules. In some embodiments, one or both of the sdAbs of the cytokine receptor binding molecule is an scFv. In some embodiments, one or both of the sdAbs is a VHH. In some embodiments, one or both of the sdAbs is an scFv.

The term single-domain antibody includes engineered sdAbs including but not limited to chimeric sdAbs, CDR-grafted sdAbs, and humanized sdAbs. In some embodiments, one or more of the sdAbs for incorporation into the IL-10 agonist compounds (e.g., single-domain antibody polypeptides of the IL-10 agonist compounds) of the present disclosure are CDR grafted. CDRs obtained from antibodies, heavy chain antibodies, and sdAbs derived therefrom may be grafted onto alternative frameworks as described in Saerens et al. 2005. J Mol Biol. 352:597-607 to generate CDR-grafted sdAbs. Any framework region can be used with the CDRs as described herein.

In some embodiments, one or more of the sdAbs for incorporation into the IL-10 agonist compounds (e.g., single-domain antibody polypeptides of the IL-10 agonist compounds) is a chimeric sdAb, in which the CDRs are derived from one species (e.g., camel) and the framework and/or constant regions are derived from another species (e.g., human or mouse). In specific embodiments, the framework regions are human or humanized sequences. Thus, IL-10 agonist compounds comprising one or more humanized sdAbs are considered within the scope of the present disclosure.

In some embodiments, one or more of the sdAb of the cytokine receptor agonist compounds of the present disclosure is a VHH. As used herein, the term “VHH” refers to a single-domain antibody derived from camelid antibody typically obtained from immunization of camelids (including camels, llamas, and alpacas (see, for example, Hamers-Casterman et al. 1993. Nature 363:446-448). VHHs are also referred to as heavy chain antibodies as single-domain antibodies may also be derived from non-mammalian sources such as VHHs obtained from IgNAR antibodies immunization of cartilaginous fishes including, but not limited to, sharks. A VHH is a type of single-domain antibody (sdAb) containing a single monomeric variable antibody domain. Like a full-length antibody, it is able to bind selectively to a specific antigen.

The complementary determining regions (CDRs) of VHHs are within a single-domain polypeptide. VHHs can be engineered from heavy-chain antibodies found in camelids. An exemplary VHH has a molecular weight of approximately 12-15 kDa, which is much smaller than traditional mammalian antibodies (150-160 kDa) composed of two heavy chains and two light chains. VHHs can be found in or produced from Camelidae mammals (e.g., camels, llamas, dromedary, alpaca, and guanaco), which are naturally devoid of light chains. Descriptions of sdAbs and VHHS can be found in, for example, De Greve et al. 2019. Curr Opin Biotechnol. 61:96-101; Ciccarese et al. 2019. Front Genet. 10:997; Chanier and Chames. 2019. Antibodies (Basel) 8(1); and De Vlieger et al. 2018. Antibodies (Basel) 8(1). The CDRs derived from camelid VHHs may be used to prepare CDR-grafted VHHs, which may be incorporated in the IL-10 agonist compounds (e.g., single-domain antibody polypeptides of the IL-10 agonist compounds).

In some embodiments, the VHH for incorporation into the IL-10 agonist compounds (e.g., single-domain antibody polypeptides of the IL-10 agonist compounds) of the present disclosure is a humanized VHH containing human framework regions or framework regions that have incorporated additional amino acid residues in common with human framework regions. Human framework regions useful in the preparation of humanized VHHs include, but are not limited to, VH3-23 (e.g., UniProt ID: P01764), VH3-74 (e.g., UniProt ID: A0A0B4J1X5), VH3-66 (e.g., UniProt ID: A0A0C4DH42), VH3-30 (e.g., UniProt ID: P01768), VH3-11 (e.g., UniProt ID: P01762), and VH3-9 (e.g., UniProt ID: P01782).

The IL-10 agonist compounds (e.g., single-domain antibody polypeptides of the IL-10 agonist compounds) of the present disclosure comprise a single-domain antibody that selectively binds to the extracellular domain of IL-10Rα (an “IL-10Rα sdAb”) in stable association with a single-domain antibody that selectively binds to the extracellular domain of IL-10Rβ (an “IL-10Rβ sdAb”). As used herein, the terms “stably associated” or “in stable association with” are used to refer to the various means by which one molecule (e.g., a polypeptide) may be thermodynamically and/or kinetically associated with another molecule. The stable association of one molecule to another may be achieved by a variety of means, including covalent bonding and non-covalent interactions.

In some embodiments, the stable association of the IL-10Rα sdAb and IL-10Rβ sdAb may be achieved by a covalent bond such as a peptide bond. In some embodiments, the covalent linkage between the first and second binding domains is a covalent bond between the C-terminus of the first binding domain and the N-terminus of the second binding domain.

In some embodiments, the covalent linkage of the IL-10Rα sdAb and IL-10Rβ sdAb of the IL-10 agonist protein is affected by a coordinate covalent linkage. The present disclosure provides examples of single-domain antibodies comprising a chelating peptide. The chelating peptide results in a coordinate covalent linkage to a transition metal ion. In some embodiments, a transition metal ion is capable of forming a coordinate covalent linkage with two or more chelating peptides. Consequently, the first and second binding domains may each comprise a chelating peptide and a stable association of the binding domains by each subunit forming a coordinate covalent complex with a transition metal ion. In some embodiments, the transition metal ion is selected from vanadium, manganese, iron, iridium, osmium, rhenium platinum, palladium, cobalt, chromium, or ruthenium. The N-terminal domain of the single-domain antibody is presented to the environment, facilitating enhanced exposure of the CDRs of the sdAb to the target cytokine receptor ECD. The formation of the coordinate covalent linkage between them is favored when the transition metal ion is in a kinetically labile oxidation state, for example, Co(II), Cr(II), or Ru(III). Following complexation, the oxidation state of the transition metal may be changed (oxidized or reduced) to a kinetically inert oxidation state. For example, Co(III), Cr(III), or Ru(II), provide a kinetically inert coordinate covalent complex. The formation of kinetically inert and kinetically labile coordinate covalent complexes between proteins comprising chelating peptides via a transition metal are described in more detail in Anderson, et al. U.S. Pat. No. 5,439,928.

In some embodiments, the covalent linkage of the IL-10Rα sdAb and IL-10Rβ sdAb of the IL-10 agonist compound (e.g., a single-domain antibody polypeptide of an IL-10 agonist compound) may further comprise a linker. Linkers are molecules selected from the group including, but not limited to, peptide linkers and chemical linkers. In some embodiments, the linker a joins the C-terminus of the IL-10Rα sdAb to the N-terminus of the IL-10Rβ sdAb. In some embodiments, the linker joins the C-terminus of the IL-10Rβ sdAb to the N-terminus of the IL-10Rα sdAb.

In some embodiments, the linker is a peptide linker. A peptide linker can include between 1 and 50 amino acids (e.g., between 2 and 50, between 5 and 50, between 10 and 50, between 15 and 50, between 20 and 50, between 25 and 50, between 30 and 50, between 35 and 50, between 40 and 50, between 45 and 50, between 2 and 45, between 2 and 40, between 2 and 35, between 2 and 30, between 2 and 25, between 2 and 20, between 2 and 15, between 2 and 10, between 2 and 5 amino acids). Glycine and glycine-serine polymers are relatively unstructured, and therefore may serve as a neutral tether between components. Examples of glycine polymers include (G)ₙ, glycine-alanine polymers, alanine-serine polymers, glycine-serine polymers (for example, (GₘSₒ)ₙ(SEQ ID NO: 573) (e.g., SEQ ID NO:416, 417, 420, 421, 422, 423), (GSGGS)ₙ(SEQ ID NO: 577), (GₘSₒGₘ)ₙ (SEQ ID NO: 574) (e.g., SEQ ID NO:418, 427, 428, 429, 430, 433, 445, 446, 447), (GₘSₒGₘSₒGₘ)ₙ (SEQ ID NO:444), (GSGGSₘ)ₙ (SEQ ID NO: 578), (GSGSₘG)ₙ (SEQ ID NO: 579) and (GGGSₘ)ₙ (SEQ ID NO: 575) (e.g., SEQ ID NO:432, 439), and combinations thereof, where m, n, and o are each independently selected from an integer of at least 1 to 20, for example, 1-18, 216, 3-14, 4-12, 5-10, 1, 2, 3, 4, 5, 6, 7, 8, 9, or 10), and other flexible linkers. Exemplary flexible peptide linkers useful in the preparation of the IL-10 agonist compounds (e.g., single-domain antibody polypeptides of the IL-10 agonist compounds) of the present disclosure include, but are not limited to, the linkers provided in Table 11.

In some embodiments, the covalent linkage of the first and second domains may be achieved by a chemical linker. Examples of chemical linkers include aryl acetylene, and ethylene glycol oligomers containing 2-10 monomer units, diamines, diacids, amino acids, or combinations thereof.

In some embodiments, the stable association of the IL-10Rα sdAb and IL-10Rβ sdAb of the IL-10 agonist protein is affected by non-covalent interaction. Examples of non-covalent interactions that provide a stable association between two molecules include electrostatic interactions (e.g., hydrogen bonding, ionic bonding, halogen bonding, dipole-dipole interactions, Van der Waals forces and p-effects including cation-p interactions, anion-p interactions, and p-p interactions) and hydrophobic/hydrophilic interactions. In some embodiments, the stable association of sdAbs of the binding molecules of the present disclosure may be affected by non-covalent interactions.

In one embodiment, the non-covalent stable association of the IL-10Rα sdAb and IL-10Rβ sdAb of the IL-10 agonist compound (e.g., a single-domain antibody polypeptide of an IL-10 agonist compound) may be achieved by conjugation of a sdAb each monomer of a “knob-into-hole” engineered Fc dimer. The knob-into-hole modification refers to a modification at the interface between two immunoglobulin heavy chains in the CH3 domain, wherein: i) in a CH3 domain of a first heavy chain, an amino acid residue is replaced with an amino acid residue having a larger side chain (e.g., tyrosine or tryptophan) creating a projection from the surface (“knob”) and ii) in the CH3 domain of a second heavy chain, an amino acid residue is replaced with an amino acid residue having a smaller side chain (e.g., alanine or threonine), thereby generating a cavity (“hole”) within at interface in the second CH3 domain within which the protruding side chain of the first CH3 domain (“knob”) is received by the cavity in the second CH3 domain. The knob-into-hole modification is more fully described in Ridgway et al. 1996. Protein Engineering 9(7):617-621 and U.S. Pat. No. 5,731,168, issued Mar. 24, 1998, U.S. Pat. No. 7,642,228, issued Jan. 5, 2010, U.S. Pat. No. 7,695,936, issued Apr. 13, 2010, and U.S. Pat. No. 8,216,805, issued Jul. 10, 2012. In one embodiment, the “knob-into-hole modification” comprises the amino acid substitution T366W and optionally the amino acid substitution S354C in one of the antibody heavy chains, and the amino acid substitutions T366S, L368A, Y407V, and optionally Y349C in the other one of the antibody heavy chains.

Furthermore, the Fc domains may be modified by the introduction of cysteine residues at positions S354 and Y349, which results in a stabilizing disulfide bridge between the two antibody-heavy chains in the Fe region (Carter et al. 2001. Immunol Methods 248:7-15). The knob-into-hole format is used to facilitate the expression of a first polypeptide (e.g., an IL-10Rβ binding sdAb) on a first Fc monomer with a “knob” modification and a second polypeptide on the second Fc monomer possessing a “hole” modification to facilitate the expression of heterodimeric polypeptide conjugates. The knob-into-hole format is used to facilitate the expression of a first polypeptide on a first Fc monomer with a “knob” modification and a second polypeptide on the second Fc monomer possessing a “hole” modification to facilitate the expression of heterodimeric polypeptide conjugates. One embodiment of an IL-10 agonist compound wherein the IL-10Rα sdAb and IL-10Rβ sdAb are in stable association, the non-covalent association is wherein each sdAb of the IL-10 agonist compound (e.g., a single-domain antibody polypeptide of an IL-10 agonist compound) covalently bonded, optionally including a linker, to each subunit of the knob-into-hole Fc dimer, as described above.

### Anti-IL-10R Single-Domain Antibodies

In some embodiments, the IL-10 agonist compounds (e.g., single-domain antibody polypeptides of the IL-10 agonist compounds) of the present disclosure are single-domain antibodies comprising an amino acid sequence having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS:1-24 of Table 1 and SEQ ID NOS:500-523. In other embodiments, the IL-10 agonist compounds (e.g., single-domain antibody polypeptides of the IL-10 agonist compounds) of the present disclosure are single-domain antibodies comprising an amino acid sequence having at least 90% (e.g., 91%, 92%, 93%, 94%, 95%, 96%, 97%, 98%, 99%, or 100%) sequence identity to the sequence of any one of SEQ ID NOS:1-24 of Table 1 and SEQ ID NOS:500-523. In other embodiments, the IL-10 agonist compounds (e.g., single-domain antibody polypeptides of the IL-10 agonist compounds) of the present disclosure are single-domain antibodies comprising an amino acid sequence having, 0, 1, 2, or 3 amino acid changes relative to the sequence of any one of SEQ ID NOS:1-24 of Table 1 and SEQ ID NOS:500-523. In other embodiments, the IL-10 agonist compounds (e.g., single-domain antibody polypeptides of the IL-10 agonist compounds) of the present disclosure are single-domain antibodies comprising an amino acid sequence having one or more conservative amino acid changes relative to the sequence of any one of SEQ ID NOS:1-24 of Table 1 and SEQ ID NOS:500-523.

In some embodiments, the IL-10 agonist compounds (e.g., single-domain antibody polypeptides of the IL-10 agonist compounds) of the present disclosure comprise a CDR1, CDR2, and CDR3 of the amino acid sequences of SEQ ID NOS 25-48 of Table 2. In some embodiments, the IL-10 agonist compounds (e.g., single-domain antibody polypeptides of the IL-10 agonist compounds) of the present disclosure comprise a CDR1, CDR2, and CDR3 of the amino acid sequences of SEQ ID NOS 49-51 of Table 3.

In some embodiments, the present disclosure provides IL-10 agonist compounds comprising a polypeptide comprising at least 95% amino acid sequence identity to an amino acid sequence selected from the group consisting of SEQ ID NOS 1-24 and 500-523.

In some embodiments, the present disclosure provides IL-10 agonist compounds, wherein the first single-domain antibody polypeptide comprises a combination of CDR1, CDR2, and CDR3 amino acid sequences selected from one of the rows of the following Table A:

TABLE A
Anti-IL-10Rα VHH CDRs

| CDR1 SEQ ID NO: | CDR2 SEQ ID NO: | CDR3 SEQ ID NO: |
|---|---|---|
| 224 | 229 | 236 |
| 224 | 230 | 236 |
| 224 | 231 | 236 |
| 225 | 229 | 236 |
| 226 | 229 | 236 |
| 224 | 232 | 236 |
| 224 | 233 | 236 |
| 224 | 234 | 236 |
| 227 | 229 | 236 |
| 224 | 235 | 236 |
| 227 | 235 | 236 |
| 228 | 229 | 236 |

and wherein the second single-domain antibody polypeptide comprises a combination of CDR1, CDR2, and CDR3 amino acid sequences selected from one of the rows of the following Table B:

TABLE B
Anti-IL-10Rβ VHH CDRs

| CDR1 SEQ ID NO: | CDR2 SEQ ID NO: | CDR3 SEQ ID NO: |
|---|---|---|
| 296 | 297 | 298 |

In some embodiments, the present disclosure provides IL-10 agonist compounds, wherein the first single-domain antibody polypeptide comprises: a CDR1 comprising the amino acid sequence of SEQ ID NO:224; a CDR2 comprising the amino acid sequence of SEQ ID NO:229; and a CDR3 comprising the amino acid sequence of SEQ ID NO:236. In some embodiments, the disclosure provides IL10 agonists compounds, wherein the second single-domain antibody polypeptide comprises: a CDR1 comprising the amino acid sequence of SEQ ID NO:296; a CDR2 comprising the amino acid sequence of SEQ ID NO:297; and a CDR3 comprising the amino acid sequence of SEQ ID NO:298.

In some embodiments, the present disclosure provides IL-10 agonist compounds, wherein the first single-domain antibody polypeptide comprises: a CDR1 comprising the amino acid sequence of SEQ ID NO:224; a CDR2 comprising the amino acid sequence of SEQ ID NO:230; and a CDR3 comprising the amino acid sequence of SEQ ID NO:236. In some embodiments, the disclosure provides IL10 agonists compounds, wherein the second single-domain antibody polypeptide comprises: a CDR1 comprising the amino acid sequence of SEQ ID NO:296; a CDR2 comprising the amino acid sequence of SEQ ID NO:297; and a CDR3 comprising the amino acid sequence of SEQ ID NO:298.

In some embodiments, the present disclosure provides IL-10 agonist compounds, wherein the first single-domain antibody polypeptide comprises: a CDR1 comprising the amino acid sequence of SEQ ID NO:224; a CDR2 comprising the amino acid sequence of SEQ ID NO:231; and a CDR3 comprising the amino acid sequence of SEQ ID NO:236. In some embodiments, the disclosure provides IL10 agonists compounds, wherein the second single-domain antibody polypeptide comprises: a CDR1 comprising the amino acid sequence of SEQ ID NO:296; a CDR2 comprising the amino acid sequence of SEQ ID NO:297; and a CDR3 comprising the amino acid sequence of SEQ ID NO:298.

In some embodiments, the present disclosure provides IL-10 agonist compounds comprising a polypeptide comprising an amino acid sequence selected from the group consisting of SEQ ID NOS:1-24 or 500-523. In some embodiments, the IL-10R binding proteins of the disclosure comprise amino acid sequences of one of SEQ ID NOS:1-24 or 500-523, wherein the amino acid sequences do not have a polyHis tag.

In some embodiments, the present disclosure provides IL-10 agonist compounds comprising a polypeptide comprising the amino acid sequence of SEQ ID NO:1.

In some embodiments, the present disclosure provides IL-10 agonist compounds comprising a polypeptide comprising the amino acid sequence of SEQ ID NO:2.

In some embodiments, the present disclosure provides IL-10 agonist compounds comprising a polypeptide comprising the amino acid sequence of SEQ ID NO:3.

In some embodiments, the present disclosure provides IL-10 agonist compounds comprising a polypeptide comprising the amino acid sequence of SEQ ID NO:4.

In some embodiments, the present disclosure provides IL-10 agonist compounds comprising a polypeptide having an amino acid sequence selected from the group consisting of SEQ ID NOS:500-523.

In some embodiments, the present disclosure provides IL-10 agonist compounds comprising a polypeptide having the amino acid sequence of SEQ ID NO:500.

In some embodiments, the present disclosure provides IL-10 agonist compounds comprising a polypeptide having the amino acid sequence of SEQ ID NO:501.

In some embodiments, the present disclosure provides IL-10 agonist compounds comprising a polypeptide having the amino acid sequence of SEQ ID NO:502.

In some embodiments, the present disclosure provides IL-10 agonist compounds comprising a polypeptide having the amino acid sequence of SEQ ID NO:503.

In some embodiments, the amino acid sequences of the IL-10 agonist compounds of the disclosure do not comprise a His tag or an ASH6 His tag (SEQ ID NO: 449) (e.g., where a His tag or an ASH6 His tag (SEQ ID NO: 449) is present, it is optionally removed).

### Anti-IL-10Rα Single-Domain Antibodies

In some embodiments, the IL-10Rα sdAb used in the IL-10 agonist compounds (e.g., single-domain antibody polypeptides of the IL-10 agonist compounds) of the present disclosure comprises a sequence having at least 90% (e.g., 91%, 92%, 93%, 94%, 95%, 96%, 97%, 98%, 99%, or 100%) sequence identity to a sequence of any one the of amino acid sequences of hIL-10Rα sdAbs disclosed in Table 2 (SEQ ID NOS:25-48). In certain embodiments, the IL-10Rα sdAb comprises a sequence that is substantially identical to a sequence of any one of the amino acid sequences of hIL-10Rα sdAbs provided in Table 2 (SEQ ID NOS:25-48). In certain embodiments, the IL-10Rα sdAb comprises a sequence that is identical to a sequence of any one of the amino acid sequences of hIL-10Rα sdAbs provided in Table 2 (SEQ ID NOS:25-48).

In some embodiments, the present disclosure provides an IL-10Rα sdAb comprising a polypeptide having the amino acid sequence of SEQ ID NO:25. In some embodiments, the present disclosure provides an IL-10Rα sdAb comprising a polypeptide having the amino acid sequence of SEQ ID NO:26. In some embodiments, the present disclosure provides an IL-10Rα sdAb comprising a polypeptide having the amino acid sequence of SEQ ID NO:27. In some embodiments, the present disclosure provides an IL-10Rα sdAb comprising a polypeptide having the amino acid sequence of SEQ ID NO:28.

Table 7 provides CDRs useful in the preparation of IL-10Rα sdAbs for incorporation into the IL-10 agonist compounds (e.g., single-domain antibody polypeptides of the IL-10 agonist compounds) of the present disclosure. In some embodiments, the IL-10Rα sdAbs specifically bind to the ECD of hIL-10Rα. In some embodiments, the IL-10Rα sdAb is a single-domain antibody comprising: a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS:224-228; a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS:229-235; and a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of SEQ ID NO:236.

In some embodiments, the IL-10Rα sdAb is a single-domain antibody comprising a CDR1 having at least 95% (e.g., 95%, 96%, 97%, 98%, 99%, or 100%) sequence identity, or having 0, 1, 2, or 3 amino acid changes, optionally conservative amino acid changes relative, to the sequence of any one of SEQ ID NOS:224-228 of Table 7. In some embodiments, the IL-10Rα sdAb is a single-domain antibody comprising a CDR1 having the sequence of any one of SEQ ID NOS:224-228 of Table 7.

In some embodiments, the IL-10Rα sdAb is a single-domain antibody comprising a CDR2 having at least 95% (e.g., 95%, 96%, 97%, 98%, 99%, or 100%) sequence identity, or having 0, 1, 2, or 3 amino acid changes, optionally conservative amino acid changes relative, to the sequence of any one of SEQ ID NOS:229-235 of Table 7. In some embodiments, the IL-10Rα sdAb is a single-domain antibody comprising a CDR2 having the sequence of any one of SEQ ID NOS:229, 230, or 231 of Table 7.

In some embodiments, the IL-10Rα sdAb is a single-domain antibody comprising a CDR3 having at least 95% (e.g., 95%, 96%, 97%, 98%, 99%, or 100%) sequence identity, or having 0, 1, 2, or 3 amino acid changes, optionally conservative amino acid changes relative, to the sequence of SEQ ID NO:236 of Table 7. In some embodiments, the IL-10Rα sdAb is a single-domain antibody comprising a CDR3 having the sequence of SEQ ID NO:236 of Table 7.

In some embodiments, the amino acid sequences of the IL-10Rα sdAbs of the disclosure do not comprise a His tag or an ASH6 His tag (SEQ ID NO: 449) (e.g., where a His tag or an ASH6 His tag (SEQ ID NO: 449) is present, it is optionally removed).

### Anti-IL-10Rβ Single-Domain Antibodies

In some embodiments, the IL-10Rβ sdAb used in the IL-10 agonist compounds (e.g., single-domain antibody polypeptides of the IL-10 agonist compounds) of the present disclosure comprises a sequence having at least 90% (e.g., 91%, 92%, 93%, 94%, 95%, 96%, 97%, 98%, 99%, or 100%) sequence identity to a sequence of any one the of amino acid sequences of hIL-10Rβ sdAbs disclosed in Table 3 (SEQ ID NOS:49-51). In certain embodiments, the IL-10Rβ sdAb comprises a sequence that is substantially identical to a sequence of any one of the amino acid sequences of hIL-10Rα sdAbs provided in Table 3 (SEQ ID NOS:49-51). In certain embodiments, the IL-10Rβ sdAb comprises a sequence that is identical to a sequence of any one of the amino acid sequences of hIL-10Rβ sdAbs provided in Table 3 (SEQ ID NOS:49-51).

In some embodiments, the present disclosure provides an IL-10Rβ sdAb comprising a polypeptide having the amino acid sequence of SEQ ID NO:49. In some embodiments, the present disclosure provides an IL-10Rβ sdAb comprising a polypeptide having the amino acid sequence of SEQ ID NO:50. In some embodiments, the present disclosure provides an IL-10Rβ sdAb comprising a polypeptide having the amino acid sequence of SEQ ID NO:51. In some embodiments, the present disclosure provides an IL-10Rβ sdAb comprising a polypeptide having the amino acid sequence of SEQ ID NO:52.

Table 8 provides CDRs useful in the preparation of IL-10Rβ sdAbs for incorporation into the IL-10 agonist compounds (e.g., single-domain antibody polypeptides of the IL-10 agonist compounds) of the present disclosure. In some embodiments, the IL-10Rβ sdAbs specifically bind to the ECD of hIL-10Rβ. In some embodiments, the IL-10Rβ sdAb is a single-domain antibody comprising: a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of SEQ ID NO:296; a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of SEQ ID NO:297; and a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of SEQ ID NO:298.

In some embodiments, the IL-10Rβ sdAb is a single-domain antibody comprising a CDR1 having at least 95% (e.g., 95%, 96%, 97%, 98%, 99%, or 100%) sequence identity, or having 0, 1, 2, or 3 amino acid changes, optionally conservative amino acid changes relative, to the sequence of SEQ ID NO:296 of Table 8. In some embodiments, the IL-10Rβ sdAb is a single-domain antibody comprising a CDR1 having the sequence of SEQ ID NO:296 of Table 8.

In some embodiments, the IL-10Rβ sdAb is a single-domain antibody comprising a CDR2 having at least 95% (e.g., 95%, 96%, 97%, 98%, 99%, or 100%) sequence identity, or having 0, 1, 2, or 3 amino acid changes, optionally conservative amino acid changes relative, to the sequence of SEQ ID NO:297 of Table 8. In some embodiments, the IL-10Rβ sdAb is a single-domain antibody comprising a CDR2 having the sequence of SEQ ID NO:297 of Table 8.

In some embodiments, the IL-10Rβ sdAb is a single-domain antibody comprising a CDR3 having at least 95% (e.g., 95%, 96%, 97%, 98%, 99%, or 100%) sequence identity, or having 0, 1, 2, or 3 amino acid changes, optionally conservative amino acid changes relative, to the sequence of SEQ ID NO:298 of Table 8. In some embodiments, the IL-10Rβ sdAb is a single-domain antibody comprising a CDR3 having the sequence of SEQ ID NO:298 of Table 8.

In some embodiments, the IL-10R binding proteins of the disclosure comprise amino acid sequences of one of SEQ ID NOS:500-523, as shown in Table 17, wherein the amino acid sequences do not have a polyHis tag.

Nucleic Acids Encoding Anti-IL-10 agonist Compounds

In some embodiments, the disclosure provides isolated nucleic acids encoding IL-10 agonist compounds having the amino acid sequence selected from one of SEQ ID NOS:1-24, and 500-523, as shown in Tables 1 and 17.

In another aspect, the disclosure provides isolated nucleic acids encoding the IL-10 agonist compounds (e.g., single-domain antibody polypeptides of the IL-10 agonist compounds) described herein. Table 6 provides DNA sequences (SEQ ID NOS:200-223 encoding the IL-10 agonist compounds (e.g., single-domain antibody polypeptides of the IL-10 agonist compounds) of Table 1 (SEQ ID NOS:1-24). In certain embodiments, the present disclosure provides an isolated nucleic acid comprising a sequence having at least 90% (e.g., 91%, 92%, 93%, 94%, 95%, 96%, 97%, 98%, 99%, or 100%) sequence identity to a DNA sequence of Table 6 (SEQ ID NOS:200-223). In certain embodiments, the present disclosure provides an isolated nucleic acid comprising a DNA sequence that is substantially identical to a DNA sequence of Table 6 (SEQ ID NOS:200-223). In certain embodiments, the present disclosure provides an isolated nucleic acid comprising a DNA sequence that is identical to a DNA sequence of Table 6 (SEQ ID NOS:200-223).

In another aspect, the disclosure provides an isolated nucleic acid encoding an IL-10Rα, sdAb described herein. In certain embodiments, the present disclosure provides an isolated nucleic acid comprising a sequence having at least 90% (e.g., 91%, 92%, 93%, 94%, 95%, 96%, 97%, 98%, 99%, or 100%) sequence identity to a DNA sequence encoding an IL-10Rα sdAb of Table 9 (SEQ ID NOS:368-391). In certain embodiments, the present disclosure provides an isolated nucleic acid comprising a DNA sequence that is substantially identical to a DNA sequence of Table 9 (SEQ ID NOS:368-391). In certain embodiments, the present disclosure provides an isolated nucleic acid comprising a DNA sequence that is identical to a DNA sequence of Table 9 (SEQ ID NOS:368-391).

In another aspect, the disclosure provides an isolated nucleic acid encoding IL-10Rβ sdAb described herein. In certain embodiments, the present disclosure provides an isolated nucleic acid comprising a sequence having at least 90% (e.g., 91%, 92%, 93%, 94%, 95%, 96%, 97%, 98%, 99%, or 100%) sequence identity to a DNA sequence of Table 10 (SEQ ID NOS:392-415 and 576). In certain embodiments, the present disclosure provides an isolated nucleic acid comprising a DNA sequence that is substantially identical to a DNA sequence of Table 10 (SEQ ID NOS:392-415 and 576). In certain embodiments, the present disclosure provides an isolated nucleic acid comprising a DNA sequence that is identical to a DNA sequence of Table 10 (SEQ ID NOS:392-415 and 576).

In some embodiments, the nucleic acid sequences that encode the IL-10R binding proteins of the disclosure comprise a nucleic acid sequence of one of SEQ ID NOS:524-547, wherein the nucleotide sequence encodes an amino acid sequences having a C-terminal Ala-Ser (“AS”) linker and a hexameric histidine (“H6”) polyHis tag (SEQ ID NO: 451) chelating peptide which is also referred to herein as “ASH6” (SEQ ID NO: 449) which is also referred to herein as a “His tag”.

In some embodiments, the nucleic acid sequences that encode the IL-10R binding proteins of the disclosure comprise a nucleic acid sequence of one of SEQ ID NOS:500-547, wherein the nucleotide sequence encodes an amino acid sequences that comprises a ASH6 His tag (SEQ ID NO: 449).

In some embodiments, the nucleic acid sequences that encode the IL-10R binding proteins of the disclosure comprise a nucleic acid sequence of one of SEQ ID NOS:548-571, wherein the nucleotide sequence encodes an amino acid sequences that is untagged.

In some embodiments, the disclosure provides isolated nucleic acids encoding IL-10 agonist compounds having the amino acid sequence selected from one of SEQ ID NOS:1-24, and 500-523.

### Generation of IL10R Polypeptide Binding Molecules

The amino acid sequences of the IL-10 agonist compounds (e.g., single-domain antibody polypeptides of the IL-10 agonist compounds) disclosed herein are described in Table 1 (SEQ ID NOS:1-24). The DNA sequences encoding these amino acid sequences that were used for the expression of the IL-10 agonist compounds (e.g., single-domain antibody polypeptides of the IL-10 agonist compounds) are provided in Table 6 (SEQ ID NOS:200-223).

The IL-10 agonist proteins of Table 1 were prepared and evaluated for IL-10 activity. Details regarding the expression and purification of these IL-10 agonist proteins is provided in the Examples. Briefly, nucleic acid sequences encoding SEQ ID NOS:1-24 were synthesized using DNA sequences of Table 7 (SEQ ID NOS:200-223), respectively, and were inserted into a recombinant expression vector and expressed in HEK293 cells and purified in substantial accordance with Examples 1 and 2. The supernatants containing the IL-10 receptor agonist proteins of SEQ ID NOS:1-24 were evaluated for binding activity, substantially as described in Example 3. The results of these binding experiments are provided in Tables 17 and 18 of Example 3. IL-10 receptor agonist proteins of SEQ ID NOS:1-24 were also evaluated for biological activity, substantially as described in Examples 4, 5, and 6.

### Modulation of Activity of Receptor Binding Molecules

In some embodiments, such as to achieve partial agonism or selective activation of particular cell types, the design of the IL-10 agonist compounds (e.g., single-domain antibody polypeptides of the IL-10 agonist compounds) of the present disclosure may be modulated by structural variations in the design of the receptor binding molecule. This variation in activity may be employed to modulate the binding and activity of the IL-10R receptor binding molecule to optimize the activity of the IL-10 agonist compound (e.g., a single-domain antibody polypeptide of an IL-10 agonist compound) to achieve partial agonism, selective cell type activation, or to provide molecules having increased or decreased binding relative to the cognate ligand for each of the IL-10Rα sdAb and IL-10Rβ sdAb for their respective receptor subunits.

The ability to modulate the activity of the IL-10 agonist compounds (e.g., single-domain antibody polypeptides of the IL-10 agonist compounds) of the present disclosure provides substantial benefits in multiple therapeutic applications. IL-10 is a pleiotropic cytokine that regulates multiple immune responses through actions on T cells, B cells, macrophages, and antigen-presenting cells (APC) and seemingly paradoxical activity which has limited its clinical development. IL-10 suppresses immune responses and inhibits the expression of IL-1α, IL-1β, IL-6, IL-8, TNF-α, GM-CSF, and G-CSF in activated monocytes and macrophages. IL-10 is associated with the suppression of IFN-γ production by NK cells. IL-10 also exhibits immuno-stimulatory properties, including enhancing the stimulation of IL-2- and IL-4-treated thymocytes, enhancing the viability of B cells, and stimulating the presentation of MHC class II antigens. As a result, the use of IL-10 has been identified as useful in the treatment of a broad range of diseases, disorders, and conditions, including inflammatory conditions, immune-related disorders, fibrotic disorders, metabolic disorders, and cancer.

The IL-10 agonist proteins of the present disclosure enable modulation of activity and provide significant benefits in the treatment of human disease. IL-10 agonist proteins described herein are useful in the treatment of neoplastic diseases, such as cancer (e.g., a solid tumor cancer; e.g., non-small-cell lung carcinoma (NSCLC), renal cell carcinoma (RCC), or melanoma) in a subject in need thereof. In some embodiments, the IL-10 agonist protein described herein can provide a longer therapeutic efficacy (e.g., lower effective dose, reduced toxicity) than IL-10. The IL-10 agonist compounds (e.g., single-domain antibody polypeptides of the IL-10 agonist compounds) of the present disclosure can trigger different levels of downstream signaling in different cell types. For example, by varying the length of the linker between the IL-10Rα sdAb antibody and the IL-10Rβ sdAb antibody in the IL-10R binding molecules, the IL-10 agonist compounds (e.g., single-domain antibody polypeptides of the IL-10 agonist compounds) provide a higher level of downstream signaling in desired cell types compared to undesired cell types. In some embodiments, the IL-10 agonist compound (e.g., a single-domain antibody polypeptide of an IL-10 agonist compound) is a partial IL-10 agonist that selectively activates T cells (e.g., CD8⁺ T cells) over macrophages. In some embodiments, activated T cells have an up-regulation of IFNgamma. In some embodiments, an IL-10 agonist protein that is a partial agonist can suppress autoimmune inflammatory diseases such as ulcerative colitis and Crohn's disease.

In one embodiment, the present disclosure provides an IL-10Rα binding molecule that preferentially activates T cells, such as CD8+ T cells, relative to monocytes. In one embodiment, the present disclosure provides an IL-10Rα binding molecule wherein the affinity of the IL-10Rα sdAb has a higher affinity for the extracellular domain of IL-10Rα than the affinity of the IL-10Rβ sdAb for the extracellular domain of IL-10Rβ. In some embodiments, the present disclosure provides an IL-10Rα molecule, wherein the affinity of the IL-10Rα sdAb has an affinity for the extracellular domain of IL-10Rα of from about 10⁻⁸ to about 10⁻¹⁰ M, alternatively from about 10⁻⁹ to about 10⁻¹⁰M, or alternatively about 10¹⁻⁰ M and the IL-10Rβ sdAb an affinity for the extracellular domain of IL-10Rβ of from about 10⁻⁶ to about 10⁻⁹ M, alternatively from about 10⁻⁷ to about 10⁻⁹ M, alternatively from about 10⁻⁷ to about 10⁻⁸ M, alternatively about 10⁻⁹ M, alternatively about 10⁻⁸ M. In some embodiments, the present disclosure provides a IL-10Rα molecule of formula #1, wherein the affinity of the IL-10Rα sdAb has an affinity for the extracellular domain of IL-10Rα. of from about 10⁻⁸ to about 10⁻¹⁰ M, alternatively from about 10⁻⁹ to about 10⁻¹⁰ M, or alternatively about 10⁻¹⁰ M and the IL-10Rβ sdAb an affinity for the extracellular domain of IL-10Rβ of from about 106 to about 10⁻⁹ M, alternatively from about 10⁻⁷ to about 10⁻⁹ M, alternatively from about 10⁻⁷ to about 10⁻⁸ M, alternatively about 10⁻⁹M, alternatively about 10⁻⁸M, and the affinity of the IL-10Rα sdAb for ECD of IL-10Rα is more than 2 fold higher, alternatively more than 5 fold higher, alternatively more than 10 fold higher, alternatively more than 20 fold higher, alternatively more than 40 fold higher, alternatively more than 50 fold higher, alternatively more than 60 fold higher, alternatively more than 70 fold higher, alternatively more than 80 fold higher, alternatively more than 90 fold higher, alternatively more than 100 fold higher, alternatively more than 150 fold higher, alternatively more than 200 fold higher or alternatively more than 500 fold higher than the affinity of the IL-10Rβ sdAb for ECD of IL-10Rβ.

In some embodiments, for example, by varying the linker length, an IL-10 agonist compound can cause a higher level of downstream signaling in T cells (e.g., CD8⁺ T cells) compared to the level of downstream signaling in macrophages, a cell type that expresses both IL-10Rα and IL-10Rβ receptors but when activated too potently can cause anemia. When the downstream signaling in macrophages is activated to a high level, these activated macrophages can then eliminate aging red blood cells, causing anemia. The ability to modulate the activity of the IL-10 agonist compound (e.g., a single-domain antibody polypeptide of an IL-10 agonist compound) provides a molecule with a higher level of downstream signaling in T cells (e.g., CD8⁺ T cells) compared to the level of downstream signaling in macrophages, such that anemia is avoided. In some embodiments, the IL-10 agonist compounds (e.g., single-domain antibody polypeptides of the IL-10 agonist compounds) of the present disclosure result in a level of downstream signaling in T cells (e.g., CD8⁺ T cells) that is at least 1.1, 1.5, 2, 3, 5, or 10 times the level of downstream signaling in macrophages. In other embodiments, different IL-10Rα sdAb antibodies with different binding affinities and different IL-10Rβ sdAb antibodies with different binding affinities can be used to tune the activity of the IL-10 agonist compound. Further, when the IL-10 agonist compound (e.g., a single-domain antibody polypeptide of an IL-10 agonist compound) is provided as a single polypeptide, the orientation of the two antibodies in the polypeptide can also be changed to make change the properties of the molecule. In some embodiments, it is desired to provide the IL-10 agonist protein having reduced Eₘₐₓ compared to the Eₘₐₓ caused by wild-type IL-10, the cognate ligand for the IL-10 receptor (i.e., an IL-10 agonist compound that is an IL-10 partial agonist). Eₘₐₓ reflects the maximum response level in a cell type that can be obtained by a ligand (e.g., a binding protein described herein or the cognate ligand (e.g., IL-10)). In some embodiments, the IL-10 agonist protein described herein has at least 1% (e.g., between 1% and 100%, between 10% and 100%, between 20% and 100%, between 30% and 100%, between 40% and 100%, between 50% and 100%, between 60% and 100%, between 70% and 100%, between 80% and 100%, between 90% and 100%, between 1% and 90%, between 1% and 80%, between 1% and 70%, between 1% and 60%, between 1% and 50%, between 1% and 40%, between 1% and 30%, between 1% and 20%, or between 1% and 10%) of the Eₘₐₓ caused by hIL-10.

Examples of the means by which the modulation of the activity and/or specificity of the receptor binding molecule of the present disclosure include, but are not limited to, altering the sequential orientation of the IL-10Rα sdAb and IL-10Rβ sdAb in polypeptide IL-10 agonist compounds, independently varying the of the binding affinity of each IL-10Rα sdAb and IL-10Rβ sdAbs with respect to their respective IL-10Rα and/or each target, and modulating the distance between the IL-10Rα sdAb and IL-10Rβ sdAbs, such as by employing linkers or varying lengths, which will affect the proximity of the intracellular signaling domains of the IL-10 receptor subunits and thereby achieve modulation, of the intracellular signaling characteristic of the binding of the cognate ligand to the receptor, for example, the level of phosphor-STAT3 induced in the cell. As illustrated by the data provided below, each of these variations may be used to tune the properties of IL-10 agonist compounds.

### Fc Conjugates

In some embodiments, the IL10R binding molecule of the present invention is a polypeptide of the following formula [#1]:

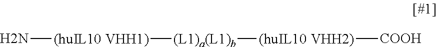

wherein: “-” represents a covalent bond; L1 is a linker; a and b are independently selected from the integers 0 or 1; “H2N” denotes the amino terminus; and “COOH” denotes the carboxy terminus of the polypeptide.

As previously discussed, the orientation of the sdAbs of the IL-10 agonist compounds (e.g., single-domain antibody polypeptides of the IL-10 agonist compounds) may be used to optimize desired characteristics of the molecules. The orientation of the sdAbs of the IL-10R may be provided in a variety of different structures, for example, as expressed formulaically below:

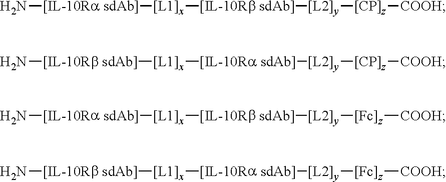

wherein “IL-10Rα sdAb” is a single-domain antibody against the IL-10Rα subunit, “IL-10Rβ sdAb” is a single-domain antibody against the IL-10Rβ subunit, L1 and L2 are independently selected polypeptide linkers of 1-50 amino acids and x=0 or 1, y=0 or 1; and CP is a chelating peptide; “Fc” is a monomeric Fc domain and y=0 or 1; non-covalent complexes of the structure:

[H₂N-[IL-10Rα sdAb]-[L1]ₓ-Fc1-COOH: H₂N-[IL-10Rβ sdAb]-[L2]_{y}-Fc2-COOH];

wherein L1 and L2 are independently selected polypeptide linkers of 1-50 amino acids and x=0 or 1, y=0 or 1; and CP is a chelating peptide; “Fc1” is a monomeric Fc domain “Fc2” is a monomeric Fc domain wherein Fc1 and Fc2 form a stable non-covalent association, and y=0 or 1, and coordinate covalent complexes of the structure:

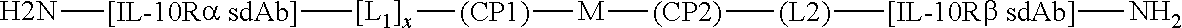

wherein L1 and L2 are independently selected polypeptide linkers of 1-50 amino acids and x and y are independently selected from 0 or 1; and CP1 is a first chelating peptide; CP2 is a second chelating peptide; and M is a transition metal ion.

As discussed in the present disclosure, in some embodiments, the IL-10 agonist compounds (e.g., single-domain antibody polypeptides of the IL-10 agonist compounds) may be conjugated to a subunit of an Fc domain and represented by the formula #2:

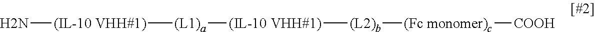

wherein: “IL-10 VHH #1” is a first IL10Rα or IL10Rβ VHH, “IL-10 VHH #2” is a second IL10Rα, or IL10Rβ VHH that is different from IL-10 VHH #1, “-” represents a covalent bond; L1 is a linker; a, b, and c are independently selected from the integers 0 or 1; “H2N” denotes the amino terminus; and “COOH” denotes the carboxy terminus of the polypeptide.

For example, representative IL-10 agonists of formula [#2] can be synthesized, in each case a=1, L1=G3S (SEQ ID NO:439), b=1 and L2=Ala-Ser; c=1; and Fc monomer can be prepared to contain variations in IL-10 VHH #2 while holding IL-10 VHH #1 sequence constant, such as DR2485 (SEQ ID NO:2).

In some embodiments, humanized IL-10 agonist compound variants can be prepared, represented by the formula [#1], wherein in each case a=1, L1=G3S (SEQ ID NO:439), b=1 and L2=Ala-Ser; CP=His×6 (SEQ ID NO:451), the IL-10 VHH #1 is the IL-10Rα VHH of DR2485 (SEQ ID NO:26) and the IL-10 VHH #2 is the IL-10Rβ VHH of DR2485 (SEQ ID NO:50).

In some embodiments, humanized IL-10 agonist compound variants can be prepared, represented by the formula [#1], wherein in each case a=1, L1=G3S (SEQ ID NO:439), b=1 and L2=Ala-Ser; CP=His×6 (SEQ ID NO:451), the IL-10 VHH #1 is the IL-10Rα VHH of DR2519) (SEQ ID NO:27) and the IL-10 VHH #2 is the IL-10Rβ of VHH DR2519 (SEQ ID NO:51).

In other embodiments, humanized IL-10 agonist compound variants can be prepared, represented by the formula [#1], wherein in each case a=1, L1=G3S (SEQ ID NO:439), b=1 and L2=Ala-Ser; C=His×6 (SEQ ID NO:451), the IL-10 VHH #1 is the IL-10Rα VHH of DR2520) (SEQ ID NO:28) and the IL-10 VHH #2 is the IL-10Rβ of VHH DR2520 (SEQ ID NO:52).

Humanization of sdAbs

The IL-10Rα and IL-10Rβ VHH sdAbs which are useful in the preparation of IL-10 agonist compounds of the present disclosure may be humanized. To demonstrate the utility of humanized versions of the VHH components of the IL-10 agonist compounds (e.g., single-domain antibody polypeptides of the IL-10 agonist compounds), humanized IL-10Rα-IL-10Rβ VHH dimers were prepared and evaluated for binding to the ECDs of IL-10Rα and IL-10Rβ.

When humanizing VHHs with respect to a target, a consideration for the design of the humanized VHH is the distribution of amino acids at each position of the non-human framework, which suggests amino acid residues, the modification of which may introduce substantial modifications in the secondary and tertiary structure of the protein. To identify amino acids of the camel VHH framework regions which are highly conserved, VHH sdAb sequences were obtained, and an “R Script” was used to evaluate distributions of amino acids at each position. An amino acid distribution chart was used to identify rare residues at each position. It was determined that certain residues of the camel VHH framework regions are highly conserved.

Table 1 provides examples of humanized IL-10 agonist compounds of the present disclosure (SEQ ID NOS:1-24).

Humanization of the IL-10 Rα-IL-10Rβ VHH dimer was conducted, first resulting in IL-10Rα-IL-10Rβ VHH dimer constructs DR2463, DR2485, DR2519, and DR2520. The amino acid sequences, nucleic acid sequences, and CDR domains (as defined by various CDR identification/numbering schemes) of the VHHs are set forth below in Tables 1-10 and 15-19.

In some embodiments, the present invention provides sdAbs having at least 90% (e.g., 91%, 92%, 93%, 94%, 95%, 96%, 97%, 98%, 99%, or 100%) sequence identity to the sequence of any one of the IL-10Rα and IL-10Rβ sdAbs of Tables 1, 2, 3, or 17. In some embodiments, the present invention provides sdAbs substantially identical to any one of the sdAbs of Tables 1, 2, 3, or 17. In some embodiments, the present invention provides sdAbs identical to any one of the sdAbs Table 1, 2, 3 or 17.

The above-referenced polypeptides are expressed recombinantly by transfection of a host cell with a vector comprising a synthetic nucleic acid sequence encoding the amino acid sequences of SEQ ID NOS:1-24 and 500-523, the synthetic nucleic acid sequence incorporating a 5′ nucleic acid sequence encoding IgH signal peptide of the amino acid sequence.

### Humanized IL-1H Agonist Compounds

The present disclosure provides IL-10 agonist compounds comprising a humanized anti-IL-10Rα sdAb and an anti-IL-10Rβ sdAb. As used herein, the terms IL-10Rα sdAb and IL-10Rβ sdAb are also used to refer to anti-IL-10Rα sdAbs and anti-IL-10Rβ sdAbs.

The following Tables C and D, below, indicate the percent of identical amino acids in a humanized IL-10R VHH dimer, relative to the V3-23 and VH3-66 germline immunoglobulin heavy chain sequences. Table 1 provides amino acid sequences of humanized IL-10 agonist compounds of this disclosure, comprising IL-10Rα and IL-10Rβ sdAbs, with CDRs underlined. Table 14 provides amino acid sequences of the non-humanized DR841 compound (SEQ ID NO:465), comprising a non-humanized IL-10Rα sdAb (SEQ ID NO:466) and a non-humanized IL-10Rβ sdAb (SEQ ID NO:470), with CDRs separately described in SEQ ID NOS:467-469, and 471-473, respectively, as well as the nucleic acid sequence encoding DR841 (SEQ NO: 580).

TABLE C
Human IL-10Rα VHH

| | Different # of Amino Acid | Percent | |
|---|---|---|---|
| | Residues | Human | Reference Sequence |
| DR841 | 14/70 | 80% | IGHV-3-23 |
| | 26/96 | 73% | IGHV3-23, |
| | | | including CDR1/2 |
| DR2519 | 8/70 | 89% | IGHV3-23 |
| | 19/96 | 80% | IGHV3-23, |
| | | | including CDR1/2 |

TABLE D
Human IL-10Rβ VHH

| | Different # of Amino Acid | Percent | |
|---|---|---|---|
| | Residues | Human | Reference Sequence |
| DR841 | 16/71 | 77% | IGHV3-66 |
| | 26/96 | 73% | IGHV3-66, |
| | | | including CDR1/2 |
| DR2519 | 1/71 | 99% | IGHV3-66 |
| | 11/96 | 89% | IGHV3-66, |
| | | | including CDR1/2 |

TABLE E
VHH DR241 (hIL10Rα)

| | | | |
|---|---|---|---|
| DR841 | 13/70 | 82% | Hz framework |
| | 26/96 | 73% | including CDR1/2 |
| DR2520 | 8/70 | 90% | Hz framework |
| | 18/96 | 81% | including CDR1/2 |

TABLE F
VHH DR246 (hIL10Rβ)

| | | | |
|---|---|---|---|
| DR841 | 16/70 | 77% | Hz framework |
| | 25/97 | 74% | including CDR1/2 |
| DR2463 | 1/70 | 99% | Hz framework |
| | 10/97 | 90% | including CDR1/2 |

In some embodiments, the present invention provides IL-10 agonist compounds having at least 90% (e.g., 91%, 92%, 93%, 94%, 95%, 96%, 97%, 98%, 99%, or 10%) sequence identity to the sequence of any one of the IL-10 agonist compounds (e.g., single-domain antibody polypeptides of the IL-10 agonist compounds) of Tables 1 or 17. In some embodiments, the present invention provides IL-10 agonist compounds substantially identical to any one of the IL-10 agonist compounds (e.g., single-domain antibody polypeptides of the IL-10 agonist compounds) of Tables 1 or 17. In some embodiments, the present invention provides IL-10 agonist compounds identical to a sequence of any one of the IL-10 agonist compounds (e.g., single-domain antibody polypeptides of the IL-10 agonist compounds) of Tables 1 or 17.

### Biased Activity

The inflammatory response is a series of biological events in a mammal initiated in response to an infectious and/or injurious stimulus that mitigates the potential for systemic infection. Typically, the mammalian inflammatory response is mediated by myeloid cells, particularly macrophages which are activated by foreign stimuli, for example, components of bacterial cell walls such as the lipopolysaccharide (LPS) of gram-negative bacteria. The activated myeloid cells act as the harbingers of infection and/or injury by secreting various pro-inflammatory signaling molecules, including but not limited to interleukin-6 (IL-6), interleukin-1 (IL-1, such as IL-1β) and tumor necrosis factor alpha (TNFα) that initiate and/or mediate the multiple biological processes associated with the inflammatory response.

Although the inflammatory response is essential for protecting the mammalian subject against infection, excessive and/or chronic activation of immune cells, such as myeloid cells, is associated with tissue damage, organ malfunction, and autoimmune disease. A wide variety of human diseases are associated with excessive and/or chronic inflammation, including but not limited to inflammatory bowel disease (IBD), rheumatoid arthritis (RA), Alzheimer's disease, asthma, type 1 and type 2 diabetes, and cancer). Among other immune modulatory molecules, Interleukin-10 (IL-10) suppresses inflammatory activity to prevent the deleterious effects associated with excess inflammation. For example, genetic loss of IL-10 in both mice and humans is associated with severe inflammatory bowel disease (IBD). Thus, expression and secretion of IL-10 are correlated with the suppression of the inflammatory response in immune cells, including but not limited to inhibiting the expression and/or secretion of proinflammatory cytokines and antigen presentation by activated myeloid cells.

In addition to the central role of IL-10 in suppressing the inflammatory response, IL-10 also promotes inflammatory activity in some cells, such as activated CD8+ T cells. Contacting IL-10 with activated CD8+ T cells results in enhanced secretion of pro-inflammatory cytokines, including interferon-gamma (IFNγ), granzyme A, and granzyme B. The concomitant pro-inflammatory and anti-inflammatory activity of IL-10 present significant challenges to the therapeutic use of IL-10 in the treatment of inflammatory disease in mammalian subjects.

In some embodiments of the present disclosure, the IL-10 agonist compounds (e.g., single-domain antibody polypeptides of the IL-10 agonist compounds) are “biased” IL-10 agonists or “partial” IL-10 agonists that alter the relative pro-inflammatory and anti-inflammatory properties of wild-type IL-10. As used herein, the term “biased” when used in the context of an IL-10 agonist compound (such as sdAb dimers or VHH dimers) means that the biased IL-10 agonist compound exhibits a greater fraction of the level of wild-type IL-10 activity in a first cell type than the level of wild-type IL-10 activity in second cell type, relative to wild-type IL-10. In one embodiment, the first cell type is a cell of myeloid origin, such as a myeloid cell. In some embodiments, the myeloid cell is a myelocyte, granulocyte (e.g., neutrophil, eosinophil, or basophil), mast cell, or monocyte. In some embodiments, the monocyte is a macrophage or dendritic cell. In some embodiments, the macrophage is a Kupffer cell. In one embodiment, the first cell type is an activated myeloid cell. In one embodiment, the first cell type is an LPS-activated human myeloid cell. In some embodiments, the second cell type is a T cell.

The IL-10 agonist compounds (e.g., single-domain antibody polypeptides of the IL-10 agonist compounds) of the present disclosure can inhibit pro-inflammatory responses and/or STAT3-mediated signaling in a cell-type dependent manner, such that inflammatory macrophage activation is inhibited, without substantially promoting the production of inflammatory cytokines such as interferon-γ by T cells. In some embodiments, an IL-10 agonist compound of the present disclosure retains the immunosuppressive functions of wild-type hIL-10, such as inhibiting the production of inflammatory cytokines, while decreasing the immunostimulatory functions of wild-type hIL-10, such as producing IFNγ by CD8⁺ T cells. For example, in some embodiments, the IL-10 agonist compounds (e.g., single-domain antibody polypeptides of the IL-10 agonist compounds) of the present disclosure retain activity comparable to wild-type hIL-10 and suppress myeloid cell activation (e.g., as demonstrated by increased STAT3-mediated signaling in myeloid cells), but substantially reduce activation in PBMCs, T cells, B cells, and NK cells (e.g., as demonstrated by decreased production of IFNγ). In some embodiments, the IL-10 agonist compounds (e.g., single-domain antibody polypeptides of the IL-10 agonist compounds) of the present disclosure are hIL-10 partial agonists.

### Pro-Inflammatory and Anti-Inflammatory Activity

In some embodiments, the IL-10 agonist compound (e.g., a single-domain antibody polypeptide of an IL-10 agonist compound) is a biased IL-10 partial agonist that (a) exhibits a significant level of at least one anti-inflammatory property of wild-type IL-10, and (b) exhibits a significantly reduced level of at least one pro-inflammatory property of wild-type IL-10. In some embodiments, “a significant level of at least one anti-inflammatory property” means that the Emax of the IL-10 agonist compounds (e.g., single-domain antibody polypeptides of the IL-10 agonist compounds) with respect to such anti-inflammatory property is greater than 10%, alternatively greater than 20%, alternatively greater than 30%, alternatively greater than 40%, alternatively greater than 50%, alternatively greater than 60%, alternatively greater than 70%, alternatively greater than 80%, alternatively greater than 90% of the Emax level of such anti-inflammatory property exhibited by wild-type IL-10 as determined in a test system. Examples of anti-inflammatory properties that may be measured in a test system include but are not limited to (a) the suppression of expression and/or secretion of hIL-1p by activated human myeloid cells, (b) the suppression of expression and/or secretion of hIL-6 by activated human myeloid cells, and (c) the suppression of expression and/or secretion of hTNFα by activated human myeloid cells. In some embodiments, activated human myeloid cells are obtained by isolating human monocytes from the buffy coat of a centrifuged anticoagulated human blood sample and activating the isolated monocytes by contacting the isolated monocytes with lipopolysaccharide (LPS) in accordance with procedures well-known in the art. The levels of hIL-1p, hIL-6, and hTNFα expressed and/or secreted by the activated monocytes may be determined by immunoassay or flow-cytometry methods in accordance with procedures well known in the art. One protocol for the evaluation of the suppression of expression and/or secretion of hIL-1β, hIL-6, and hTNFα by LPS-activated human monocytes is provided in the Examples described herein.

In some embodiments, “a significantly reduced level of at least one pro-inflammatory property” means that the Emax of the IL-10 agonist compound (e.g., a single-domain antibody polypeptide of an IL-10 agonist compound) with respect to such pro-inflammatory property that is less than 90%, alternatively less than 80%, alternatively less than 70%, alternatively less than 60%, alternatively less than 50%, alternatively less than 40%, alternatively less than 30%, alternatively less than 20%, alternatively less than 10% of the Emax of that pro-inflammatory property of wild-type IL-10 as determined in a test system. Examples of pro-inflammatory properties include but are not limited to (a) the suppression of expression and/or secretion of IFNγ by activated human CD8+ T cells, (b) the suppression of expression and/or secretion of granzyme A by activated human CD8+ T cells, and (c) the suppression of expression and/or secretion of granzyme A by activated human CD8+ T cells. In some embodiments, activated human T cells are obtained by isolating CD8+ T cells in human whole blood and activated by contacting the isolated CD8+ cells with anti-CD3 and anti-CD28 antibodies in accordance with procedures well known in the art. The levels of IFNγ, granzyme A and granzyme B expressed and/or secreted by the isolated CD8+ T cells may be determined by immunoassay or flow-cytometry methods in accordance with procedures well known in the art. One protocol for the evaluation of the expression and/or secretion of IFNγ, granzyme A and granzyme B expressed and/or secreted by CD3/CD28 activated CD8+ T cells is disclosed in the Examples herein.

In some embodiments, the IL-10 agonist compound (e.g., a single-domain antibody polypeptide of an IL-10 agonist compound) is a biased hIL-10 partial agonist exhibiting a significant level of at least one anti-inflammatory property of wild-type hIL-10, while concomitantly exhibiting a significantly reduced level of at least one pro-inflammatory property of wild-type IL-10, wherein the significant level of at least one anti-inflammatory property of wild-type hIL-10 is an Emax of at least one anti-inflammatory property greater than 30% of the Emax of such anti-inflammatory property exhibited by wild-type hIL-10, wherein the at least one anti-inflammatory property is selected from the group consisting of (i) suppression of expression and/or secretion of hIL-1β in LPS-activated human monocytes, (ii) suppression of expression and/or secretion of hIL-6 in LPS-activated human monocytes, or (iii) suppression of expression and/or secretion of hTNFα in LPS-activated human monocytes; and the significantly reduced level of at least one pro-inflammatory property of wild-type hIL-10 is an Emax of at least one anti-inflammatory property less than 30% of the Emax of such anti-inflammatory property exhibited by wild-type hIL-10, wherein the at least one pro-inflammatory property is selected from the group consisting of (i) suppression of expression and/or secretion of IFNγ by activated human CD8+ T cells, (ii) suppression of expression and/or secretion of granzyme A by activated human CD8+ T cells, and (iii) suppression of expression and/or secretion of granzyme A by activated human CD8+ T cells.

In some embodiments, the IL-10 agonist compound (e.g., a single-domain antibody polypeptide of an IL-10 agonist compound) of the present disclosure is a biased hIL-10 partial agonist exhibiting a significant level of at least one anti-inflammatory property of wild-type hIL-10, while concomitantly exhibiting a significantly reduced level of at least one pro-inflammatory property of wild-type IL-10, wherein the significant level of at least one anti-inflammatory property of wild-type hIL-10 is an Emax of at least one anti-inflammatory property greater than 30% of the Emax of such anti-inflammatory property exhibited by wild-type hIL-10, wherein the at least one anti-inflammatory property is selected from the group consisting of (i) suppression of expression and/or secretion of hIL-1β in LPS-activated human monocytes, (ii) suppression of expression and/or secretion of hIL-6 in LPS-activated human monocytes, or (iii) suppression of expression and/or secretion of hTNFα in LPS-activated human monocytes; and the significantly reduced level of at least one pro-inflammatory property of wild-type hIL-10 is an Emax of at least one anti-inflammatory property less than 30% of the Emax of such anti-inflammatory property exhibited by wild-type hIL-10 wherein the at least one pro-inflammatory property is selected from the group consisting of (i) suppression of expression and/or secretion of IFNγ by activated human CD8+ T cells, (ii) suppression of expression and/or secretion of granzyme A by activated human CD8+ T cells, and (iii) suppression of expression and/or secretion of granzyme A by activated human CD8+ T cells.

In some embodiments, the IL-10 agonist compound (e.g., a single-domain antibody polypeptide of an IL-10 agonist compound) of the present disclosure comprises an IL-10 agonist compound wherein the Emax of the IL-10 agonist compound (e.g., a single-domain antibody polypeptide of an IL-10 agonist compound) is greater than 30% of the Emax of wild-type hIL-10 in an assay of anti-inflammatory activity selected from the group consisting of (i) suppression of expression and/or secretion of hIL-10 in LPS-activated human monocytes, (ii) suppression of expression and/or secretion of hIL-6 in LPS-activated human monocytes, or (iii) suppression of expression and/or secretion of hTNFα in LPS-activated human monocytes; and the Emax of the IL-10 agonist compound (e.g., a single-domain antibody polypeptide of an IL-10 agonist compound) is less than 10% of the Emax of wild-type hIL-10 in an assay of pro-inflammatory activity selected from the group consisting of (i) suppression of expression and/or secretion of IFNγ by activated human CD8+ T cells, (ii) suppression of expression and/or secretion of granzyme A by activated human CD8+ T cells, and (iii) suppression of expression and/or secretion of granzyme A by activated human CD8+ T cells.

In some embodiments, the IL-10 agonist compound (e.g., a single-domain antibody polypeptide of an IL-10 agonist compound) of the present disclosure is a biased IL-10 agonist compound wherein the Emax of the IL-10 agonist compound (e.g., a single-domain antibody polypeptide of an IL-10 agonist compound) is greater than 50% of the Emax of wild-type hIL-10 in an assay of anti-inflammatory activity selected from the group consisting of (i) suppression of expression and/or secretion of hIL-10 in LPS-activated human monocytes, (ii) suppression of expression and/or secretion of hIL-6 in LPS-activated human monocytes, or (iii) suppression of expression and/or secretion of hTNFα in LPS-activated human monocytes; and the Emax of the IL-10 agonist compound (e.g., a single-domain antibody polypeptide of an IL-10 agonist compound) is less than 20% of the Emax of wild-type hIL-10 in an assay of pro-inflammatory activity selected from the group consisting of (i) suppression of expression and/or secretion of IFNγ by activated human CD8+ T cells, (ii) suppression of expression and/or secretion of granzyme A by activated human CD8+ T cells, and (iii) suppression of expression and/or secretion of granzyme A by activated human CD8+ T cells.

In some embodiments, the IL-10 agonist compound (e.g., a single-domain antibody polypeptide of an IL-10 agonist compound) of the present disclosure is a biased IL-10 agonist compound wherein: the Emax of the IL-10 agonist compound (e.g., a single-domain antibody polypeptide of an IL-10 agonist compound) is greater than 50% of the Emax of wild-type hIL-10 in an assay of anti-inflammatory activity selected from the group consisting of (i) suppression of expression and/or secretion of hIL-1β in LPS-activated human monocytes, (ii) suppression of expression and/or secretion of hIL-6 in LPS-activated human monocytes, or (iii) suppression of expression and/or secretion of hTNFα in LPS-activated human monocytes; and the Emax of the IL-10 agonist compound (e.g., a single-domain antibody polypeptide of an IL-10 agonist compound) is less than 10% of the Emax of wild-type hIL-10 in an assay of pro-inflammatory activity selected from the group consisting of (i) suppression of expression and/or secretion of IFNγ by activated human CD8+ T cells, (ii) suppression of expression and/or secretion of granzyme A by activated human CD8+ T cells, and (iii) suppression of expression and/or secretion of granzyme A by activated human CD8+ T cells.

### STAT3

As previously noted, the interaction of IL-10 with the IL-10 receptor results in intracellular signaling characterized by the enhanced intracellular production of phosphorylated STAT3 (phosphor-STAT3). Consequently, one measure of IL-10 activity may be evaluated using a cell expressing the IL-10 receptor (comprised of IL-10Rα and IL-10Rβ) is the intracellular production of phospho-STAT3.

In one embodiment, the IL-10 agonist compound (e.g., a single-domain antibody polypeptide of an IL-10 agonist compound) of the present invention is a biased hIL-10 partial agonist, the first cell type is an activated human myeloid cell, and the second cell type is an activated human T cell, wherein the level of IL-10 activity is measured by intracellular production of phospho-STAT3. In one embodiment, the IL-10 agonist compound (e.g., a single-domain antibody polypeptide of an IL-10 agonist compound) is a biased hIL-10 partial agonist that retains a greater fraction of hIL-10 activity on activated human monocytes than activated human CD8+ T cells, wherein the level of IL-10 activity is measured by intracellular production of phospho-STAT3. In some embodiments, the level of IL-10 activity in the first cell type and second cell type is measured by the production of phospho-STAT3 in a first cell type in response to contacting the first cell type and second cell type with an IL-10 agonist compound.

In some embodiments, the relative activation of STAT3 signaling of IL-10 agonist compounds described herein in a first cell type versus a second cell type is different from the relative activation of STAT3 signaling of a wild-type human or murine IL10 in the first cell type versus the second cell type. In some embodiments, the level of intracellular phospho-STAT3 induced in a human myeloid cell in response to contacting the myeloid cell with an effective amount of a human IL-10 agonist compound is at least 10 fold, alternatively at least 100 fold, alternatively at least 1000 fold, greater than the level of intracellular phospho-STAT3 induced in a human lymphocyte cell in response to contacting the human lymphocyte cell with the same amount of the human IL-10 agonist compound. In one embodiment, the ratio of the level of STAT3 signaling induced in a myeloid cell in response to contacting a myeloid cell with a human IL-10 agonist compound relative to the level of STAT3 signaling induced in a lymphocyte cell in response to contacting a lymphocyte with the IL-10 agonist compound (e.g., a single-domain antibody polypeptide of an IL-10 agonist compound) is different than (greater than or lesser than) the ratio of the level of STAT3 signaling induced in the myeloid cell in response to contacting the myeloid cell with wild-type hIL-10 relative to the level of STAT3 signaling induced in the lymphocyte in response to contacting the lymphocyte with wild-type hIL-10. In some embodiments, the ratio of the activity (as determined by the level of intracellular phospho-STAT3) of a human IL-10 agonist compound in human myeloid cells relative to human lymphocytes is greater than the ratio of the activity of wild-type human IL-10 in human myeloid cells relative to human lymphocytes. In some embodiments, the myeloid cell is a neutrophil, eosinophil, mast cell, basophil, or monocyte. In some embodiments, the monocyte is a macrophage or a dendritic cell. In some embodiments, the macrophage is a Kupffer cell. In some embodiments, the lymphocyte is a CD8+ T cell, a CD4+ T cell, a B cell, or an NK cell.

In some embodiments, the IL-10 agonist compounds (e.g., single-domain antibody polypeptides of the IL-10 agonist compounds) of the present disclosure have a pSTAT3 Eₘₐₓ of greater than 20%, greater than 30%, greater than 40%, greater than 50%, greater than 60%, or greater than 70% of the pSTAT3 Eₘₐₓ of wild-type hIL-10 in myeloid cells. In some embodiments, an IL-10 agonist compound of the present disclosure exhibits decreased STAT3-mediated signaling in lymphocytes such as T cells, B cells, or NK cells compared to wild-type hIL-10. In some embodiments, an IL-10 agonist compound of the present disclosure has a pSTAT3 Eₘₐₓ in a lymphocyte less than 70%, less than 60%, less than 50%, less than 40%, or less than 30%, of the pSTAT3 Eₘₐₓ of a wild-type hIL-10 in lymphocytes. In some embodiments, the IL-10 agonist compounds (e.g., single-domain antibody polypeptides of the IL-10 agonist compounds) result in a pSTAT3 Eₘₐₓ in a lymphocyte less than 70% (e.g., less than 70%, less than 60%, less than 50%, less than 40%, or less than 30%) but greater than 20% of the pSTAT3 Eₘₐₓ of wild-type or parental IL-10 polypeptide in the lymphocyte. In some embodiments, the lymphocyte is selected from a CD8+ T cell, a CD4+ T cell, a B cell, or an NK cell.

Modifications to the Binding Molecule and sdAb Components

### Chelating Peptides

In one embodiment, the present disclosure provides an IL-10 agonist compound comprising one or more transition metal chelating polypeptide sequences known as chelating peptides. A chelating peptide is a polypeptide of the formula:

(SEQ ID NO: 448)
(His)ₐ-(AA)_{b}-(His)_{c}

wherein “His” is the amino acid histidine; “AA” is an amino acid other than proline; is a histidine residue, a=an integer from 0 to 10; b=an integer from 0 to 4; c=an integer from 0-10; and random, block and alternating copolymers thereof. In some embodiments, the chelating peptide has an amino acid sequence selected from the group consisting of SEQ ID NOS:416-439. The incorporation of such a transition metal chelating domain facilitates purification immobilized metal affinity chromatography (IMAC) as described in Smith, et al. U.S. Pat. No. 4,569,794 issued Feb. 11, 1986. Examples of transition metal chelating polypeptides useful in the practice of the present IL-10Rβ binding molecule are described in Smith, et al. supra and Dobeli, et al. U.S. Pat. No. 5,320,663 issued May 10, 1995, the entire teachings of which are hereby incorporated by reference. Particular transition metal chelating polypeptides useful in the practice of the present IL-10 agonist compounds are polypeptides comprising 3-6 contiguous histidine residues (SEQ ID NO: 572) (e.g., SEQ ID NOS:451, 461) such as a six-histidine (His)₆ peptide (SEQ ID NO:451) and are frequently referred to in the art as “His-tags.” In addition to providing a purification “handle” for the recombinant proteins or facilitating immobilization on SPR sensor chips, the conjugation of the IL-10 agonist compound (e.g., a single-domain antibody polypeptide of an IL-10 agonist compound) to a chelating peptide facilitates the targeted delivery to IL-10R expressing cells of transition metal ions as kinetically inert or kinetically labile complexes in substantial accordance with the teaching of Anderson, et al., (U.S. Pat. No. 5,439,829 issued Aug. 8, 1995, and Hale, J. E. 1996. Analytical Biochemistry 231(1):46-49. Regarding the “kinetically inert complex”, the term “inert” refers to the degree of lability, which is the ability of a particular complexed ion to engage in reactions that result in replacing one or more ligands in its coordination sphere by others. In an aqueous environment, the unoccupied coordination positions of the transition metal are occupied by water molecules. These water molecules must be displaced by the chelating peptide or organic chelating agent in order to form the [transition metal:chelating peptide] complex. When such reactions occur rapidly, the reaction is termed “labile.” However, where such reactions occur slowly, the complex is said to be kinetically “inert.” Kinetic lability or inertness is related to the reaction rate and are not to be confused with thermodynamic stability or instability. A simple example of this (labile vs. stable) distinction is provided by the [Co(NH₃)₆]³⁺ ion, which will persist for days in an acid medium because of its kinetic inertness or lack of lability despite the fact that it is thermodynamically unstable, as the following equilibrium constant shows:

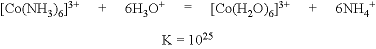

In contrast, the stability of [Ni(CN)₄]²⁻ is extremely high:

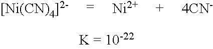

but the rate of exchange of CN⁻ ions with isotopically labeled cyanide ions added to the solution is immeasurably fast by ordinary techniques. Advanced Inorganic Chemistry, Cotton, F. A. and Wilkinson, G. (1972) 3rd ed. Interscience Publishers, p.652. In some embodiments, the transition metal ion is a reporter molecule such as a fluorescent compound or radioactive agent, including radiological imaging or therapeutic agents. In some embodiments, the chelating peptide is a chelating peptide.

### Elimination of N-Linked Glycosylation Sites

In some embodiments, it is possible that an amino acid sequence (particularly a CDR sequence) of the IL-10Rα or IL-10Rβ sdAb may contain a glycosylation motif, particularly an N-linked glycosylation motif of the sequence Asn-X-Ser (N-X-S) or Asn-X-Thr (N-X-T), wherein X is any amino acid except for proline. In such instances, it is desirable to eliminate such N-linked glycosylation motifs by modifying the sequence of the N-linked glycosylation motif to prevent glycosylation. In some embodiments, the elimination of the Asn-X-Ser (N-X-S) N-linked glycosylation motif may be achieved by the incorporation of conservative amino acid substitution of the Asn (N) residue and/or Ser (S) residue of the Asn-X-Ser (N-X-S) N-linked glycosylation motif. In some embodiments, the elimination of the Asn-X-Thr (N-X-T) N-linked glycosylation motif may be achieved by the incorporation of conservative amino acid substitution of the Asn (N) residue and/or Thr (T) residue of the Asn-X-Thr (N-X-T) N-linked glycosylation motif. In some embodiments, elimination of the glycosylation site is not required when the IL-10 agonist compound (e.g., a single-domain antibody polypeptide of an IL-10 agonist compound) comprising the IL-10Rα or IL-10Rβ sdAb is expressed in procaryotic host cells. Since procaryotic cells do not provide a mechanism for glycosylation of recombinant proteins, when employing a procaryotic expression system to produce a recombinant IL-10 agonist compound comprising the IL-10Rα or IL-10Rβ sdAb, the modification of the sequence to eliminate the N-linked glycosylation sites may be obviated.

Association with Carrier Molecules to Increase Duration of Action

The IL-10 agonist compound (e.g., a single-domain antibody polypeptide of an IL-10 agonist compound) described herein can be modified to provide for an extended lifetime in vivo and/or extended duration of action in a subject. In some embodiments, the binding molecule can be conjugated to carrier molecules to provide desired pharmacological properties such as an extended half-life. In some embodiments, the binding molecule can be covalently linked to the Fc domain of IgG, albumin, or other molecules to extend its half-life, for example, by pegylation, glycosylation, and the like as known in the art. In some embodiments, the IL-10 agonist compound (e.g., a single-domain antibody polypeptide of an IL-10 agonist compound) modified to provide an extended duration of action in a mammalian subject has a half-life in a mammalian of greater than 4 hours, alternatively greater than 5 hours, alternatively greater than 6 hours, alternatively greater than 7 hours, alternatively greater than 8 hours, alternatively greater than 9 hours, alternatively greater than 10 hours, alternatively greater than 12 hours, alternatively greater than 18 hours, alternatively greater than 24 hours, alternatively greater than 2 days, alternatively greater than 3 days, alternatively greater than 4 days, alternatively greater than 5 days, alternatively greater than 6 days, alternatively greater than 7 days, alternatively greater than 10 days, alternatively greater than 14 days, alternatively greater than 21 days, or alternatively greater than 30 days.

Modifications of the IL-10 agonist compound (e.g., a single-domain antibody polypeptide of an IL-10 agonist compound) to provide an extended duration of action in a mammalian subject include (but are not limited to): conjugation of the IL-10 agonist compound (e.g., a single-domain antibody polypeptide of an IL-10 agonist compound) to one or more carrier molecules; conjugation of IL-10 agonist compound to protein carriers molecules, optionally in the form of a fusion protein with additional polypeptide sequences (e.g., IL-10 agonist compound −Fc fusions) and conjugation to polymers, (e.g., water-soluble polymers to provide a PEGylated IL-10 agonist compound).

It should be noted that more than one type of modification that provides for an extended duration of action in a mammalian subject may be employed with respect to a given IL-10 agonist compound. For example, the IL-10 agonist compound (e.g., a single-domain antibody polypeptide of an IL-10 agonist compound) of the present disclosure may comprise both amino acid substitutions that provide for an extended duration of action as well as conjugation to a carrier molecule such as a polyethylene glycol (PEG) molecule.

### Protein Carrier Molecules

Examples of protein carrier molecules that may be covalently attached to the IL-10 agonist compound (e.g., a single-domain antibody polypeptide of an IL-10 agonist compound) to provide an extended duration of action in vivo include, but are not limited to albumins, antibodies, and antibody fragments such and Fc domains of IgG molecules

### Fc Fusions

In some embodiments, the IL-10 agonist compound (e.g., a single-domain antibody polypeptide of an IL-10 agonist compound) is conjugated to a functional domain of an Fc-fusion chimeric polypeptide molecule. Fc fusion conjugates have been shown to increase the systemic half-life of biopharmaceuticals, and thus the biopharmaceutical product can require less frequent administration. Fc binds to the neonatal Fc receptor (FcRn) in endothelial cells that line the blood vessels, and, upon binding, the Fc fusion molecule is protected from degradation and re-released into circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates. The “Fc region” useful in the preparation of Fc fusions can be a naturally occurring or synthetic polypeptide that is homologous to an IgG C-terminal domain produced by the digestion of IgG with papain. IgG Fc has a molecular weight of approximately 50 kDa. The binding molecule described herein can be conjugated to the entire Fc region, or a smaller portion that retains the ability to extend the circulating half-life of a chimeric polypeptide of which it is a part. In addition, full-length or fragmented Fc regions can be variants of the wild-type molecule. In a typical presentation, each monomer of the dimeric Fc can carry a heterologous polypeptide, the heterologous polypeptides being the same or different.

### Linkage of Binding Molecule to Fc Monomer

As indicated, the linkage of the IL-10 agonist compound (e.g., a single-domain antibody polypeptide of an IL-10 agonist compound) to the Fc subunit may incorporate a linker molecule as described below between the IL-10 agonist compound (e.g., a single-domain antibody polypeptide of an IL-10 agonist compound) and the Fc subunit. In some embodiments, the IL-10 agonist compound (e.g., a single-domain antibody polypeptide of an IL-10 agonist compound) is expressed as a fusion protein with the Fc domain incorporating an amino acid sequence of a hinge region of an IgG antibody. The Fc domains engineered in accordance with the foregoing may be derived from IgG1, IgG2, IgG3, and IgG4 mammalian IgG species. In some embodiments, the Fc domains may be derived from human IgG1, IgG2, IgG3, and IgG4 IgG species. In some embodiments, the hinge region is the hinge region of an IgG1. In one particular embodiment, the IL-10 agonist compound (e.g., a single-domain antibody polypeptide of an IL-10 agonist compound) is linked to an Fc domain using a human IgG1 hinge domain.

In some embodiments, the dimeric Fc molecule may be engineered to possess a “knob-in-hole modification,” as described in greater detail above.

### Albumin Carrier Molecules

In some embodiments, the IL-10 agonist compound (e.g., a single-domain antibody polypeptide of an IL-10 agonist compound) conjugated to an is albumin molecule (e.g., human serum albumin) which is known in the art to facilitate extended exposure in vivo. In one embodiment of the invention, the IL-10 agonist compound (e.g., a single-domain antibody polypeptide of an IL-10 agonist compound) is conjugated to albumin via chemical linkage or expressed as a fusion protein with an albumin molecule referred to herein as an IL-10 agonist compound albumin fusion.” The term “albumin” as used in the context hzIL-10Rα/IL-10Rβ mutein albumin fusions include albumins such as human serum albumin (HSA), cyano serum albumin, and bovine serum albumin (BSA). In some embodiments, the HSA the HSA comprises a C34S or K573P amino acid substitution relative to the wild-type HSA sequence According to the present disclosure, albumin can be conjugated to an IL-10 agonist compound at the carboxyl terminus, the amino terminus, both the carboxyl and amino termini and internally (see, for example, U.S. Pat. Nos. 5,876,969 and 7,056,701). In the HAS IL-10 agonist compound contemplated by the present disclosure, various forms of albumin can be used, such as albumin secretion pre-sequences and variants thereof, fragments and variants thereof, and HSA variants. Such forms generally possess one or more desired albumin activities. In additional embodiments, the present disclosure involves fusion proteins comprising an IL-10 agonist compound fused directly or indirectly to albumin, an albumin fragment, an albumin variant, etc., wherein the fusion protein has higher plasma stability than the unfused drug molecule and/or the fusion protein retains the therapeutic activity of the unfused drug molecule. As an alternative to the chemical linkage between the IL-10 agonist compound (e.g., a single-domain antibody polypeptide of an IL-10 agonist compound) and the albumin molecule, the IL-10 agonist compound-albumin complex may be provided as a fusion protein comprising an albumin polypeptide sequence and an IL-10 agonist compound recombinantly expressed in a host cell as a single polypeptide chain, optionally comprising a linker molecule between the albumin and IL-10 agonist compounds. Such fusion proteins may be readily prepared through recombinant technology to those of ordinary skill in the art. Nucleic acid sequences encoding such fusion proteins may be ordered from any of a variety of commercial sources. The nucleic acid sequence encoding the fusion protein is incorporated into an expression vector operably linked to one or more expression control elements, the vector is introduced into a suitable host cell, and the fusion protein is isolated from the host cell culture by techniques well-known in the art.

### Polymeric Carriers

In some embodiments, extended in vivo duration of action of the IL-10 agonist compound (e.g., a single-domain antibody polypeptide of an IL-10 agonist compound) may be achieved by conjugation to one or more polymeric carrier molecules such as XTEN polymers or water-soluble polymers.

### XTEN Conjugates

The IL-10 agonist compound (e.g., a single-domain antibody polypeptide of an IL-10 agonist compound) may further comprise an XTEN polymer. The XTEN polymer may be conjugated (either chemically or as a fusion protein) to the IL-10 agonist compound (e.g., a single-domain antibody polypeptide of an IL-10 agonist compound) to provide extended duration in vivo, akin to PEGylation, and may be produced as a recombinant fusion protein in E. coli. XTEN polymers are suitable for use in conjunction with the IL-10 agonist compound (e.g., a single-domain antibody polypeptide of an IL-10 agonist compound) of the present disclosure are disclosed in Podust et al. 2016. Extension of in vivo half-life of biologically active molecules by XTEN protein polymers., J Controlled Release 240:52-66 and Haeckel et al. 2016. XTEN as Biological Alternative to PEGylation Allows Complete Expression of a Protease-Activatable Killin-Based Cytostatic. PLOS ONE DOI:10.1371/journal.pone.0157193 Jun. 13, 2016. The XTEN polymer may fusion protein may incorporate a protease-sensitive cleavage site between the XTEN polypeptide and the IL-10 agonist compound, such as an MMP-2 cleavage site.

### Water Soluble Polymers

In some embodiments, the IL-10 agonist compound (e.g., a single-domain antibody polypeptide of an IL-10 agonist compound) can be conjugated to one or more water-soluble polymers. Examples of water-soluble polymers useful in the practice of the present disclosure include polyethylene glycol (PEG), polypropylene glycol (PPG), polysaccharides (polyvinylpyrrolidone, copolymers of ethylene glycol and propylene glycol, poly(oxyethylene polyol), polyolefinic alcohol,), polysaccharides), poly-alpha-hydroxy acid), polyvinyl alcohol (PVA), polyphosphazene, polyoxazolines (POZ), poly(N-acryloyl morpholine), or a combination thereof.

In some embodiments, the IL-10 agonist compound (e.g., a single-domain antibody polypeptide of an IL-10 agonist compound) can be conjugated to one or more polyethylene glycol molecules or “PEGylated.” Although the method or site of PEG attachment to the binding molecule may vary, in certain embodiments the PEGylation does not alter, or only minimally alters, the activity of the binding molecule.

PEGs suitable for conjugation to a polypeptide sequence are generally soluble in water at room temperature and have the general formula

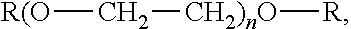

where R is hydrogen or a protective group such as an alkyl or an alkanol group, and where n is an integer from 1 to 1000. When R is a protective group, it generally has from 1 to 8 carbons.

The PEG can be linear or branched. Branched PEG derivatives, “star-PEGs,” and multi-armed PEGs are contemplated by the present disclosure.

In some instances, the sequences of IL-10 agonist compounds of the present disclosure provided in Tables 1, 2, and 3 possess an N-terminal glutamine (“1Q”) residue. N-terminal glutamine residues have been observed to spontaneously cyclize to form pyroglutamate (pE) at or near physiological conditions. (See, for example, Liu et al. 2011. J Biol Chem. 286(13):11211-11217). In some embodiments, the formation of pyroglutamate complicates N-terminal PEG conjugation, particularly when aldehyde chemistry is used for N-terminal PEGylation. Consequently, when PEGylating the IL-10 agonist compounds (e.g., single-domain antibody polypeptides of the IL-10 agonist compounds) of the present disclosure, particularly when aldehyde chemistry is to be employed, the IL-10 agonist compounds (e.g., single-domain antibody polypeptides of the IL-10 agonist compounds) possessing an amino acid at position 1 (e.g., 1Q) are substituted at position 1 with an alternative amino acid or are deleted at position 1 (e.g., des-1Q). In some embodiments, the IL-10 agonist compounds (e.g., single-domain antibody polypeptides of the IL-10 agonist compounds) of the present disclosure comprise an amino acid substitution selected from the group Q1E and Q1D.

In some embodiments, selective PEGylation of the IL-10 agonist compounds (e.g., single-domain antibody polypeptides of the IL-10 agonist compounds), for example, by the incorporation of non-natural amino acids having side chains to facilitate selective PEG conjugation, may be employed. Specific PEGylation sites can be chosen such that PEGylation of the binding molecule does not affect its binding to the target receptors.

In certain embodiments, the increase in half-life is greater than any decrease in biological activity. PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure.

The molecular weight of the PEG used in the present disclosure is not restricted to any particular range. The PEG component of the binding molecule can have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa, or from about 10 kDa to about 30 kDa. Linear or branched PEG molecules having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, or alternatively about 30,000 to about 40,000 daltons. In one embodiment of the disclosure, the PEG is a 40 kD branched PEG comprising two 20 kD arms.

The present disclosure also contemplates compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods known in the art. Chromatography may be used to resolve conjugate fractions, and a fraction is then identified which contains the conjugate having, for example, the desired number of PEGs attached, purified free from unmodified protein sequences and from conjugates having other numbers of PEGs attached.

PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons.

Two widely used first-generation activated monomethoxy PEGs (mPEGs) are succinimidyl carbonate PEG (SC-PEG; see, for example, Zalipsky et al. 1992. Biotechnol Appl Biochem. 15:100-114) and benzotriazole carbonate PEG (BTC-PEG; see, for example, Dolence et al. U.S. Pat. No. 5,650,234), which react preferentially with lysine residues to form a carbamate linkage but are also known to react with histidine and tyrosine residues. The use of a PEG-aldehyde linker targets a single site on the N-terminus of a polypeptide through reductive amination.

Pegylation most frequently occurs at the α-amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General PEGylation strategies known in the art can be applied herein.

The PEG can be bound to a binding molecule of the present disclosure via a terminal reactive group (a “spacer”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide.

In some embodiments, the PEGylation of the binding molecules is facilitated by the incorporation of non-natural amino acids bearing unique side chains to facilitate site-specific PEGylation. The incorporation of non-natural amino acids into polypeptides to provide functional moieties to achieve site-specific PEGylation of such polypeptides is known in the art. See e.g., Ptacin et al., PCT International Application No. PCT/US2018/045257 (WO 2019/028419A).

The PEG conjugated to the polypeptide sequence can be linear or branched. Branched PEG derivatives, “star-PEGs” and multi-armed PEGs are contemplated by the present disclosure. Specific embodiments PEGs useful in the practice of the present disclosure include a 10 kDa linear PEG-aldehyde (e.g., Sunbright® ME-100AL, NOF America Corporation, One North Broadway, White Plains, NY 10601 USA), 10 kDa linear PEG-NHS ester (e.g., Sunbright® ME-100CS, Sunbright® ME-100AS, Sunbright® ME-100GS, Sunbright® ME-100HS, NOF), a 20 kDa linear PEG-aldehyde (e.g., Sunbright® ME-200AL, NOF), a 20 kDa linear PEG-NHS ester (e.g., Sunbright® ME-200CS, Sunbright® ME-200AS, Sunbright® ME-200GS, Sunbright® ME-200HS, NOF), a 20 kDa 2-arm branched PEG-aldehyde the 20 kDA PEG-aldehyde comprising two 10 kDA linear PEG molecules (e.g., Sunbright® GL2-200AL3, NOF), a 20 kDa 2-arm branched PEG-NHS ester the 20 kDA PEG-NHS ester comprising two 10 kDA linear PEG molecules (e.g., Sunbright® GL2-200TS, Sunbright® GL200GS2, NOF), a 40 kDa 2-arm branched PEG-aldehyde the 40 kDA PEG-aldehyde comprising two 20 kDA linear PEG molecules (e.g., Sunbright® GL2-400AL3), a 40 kDa 2-arm branched PEG-NHS ester the 40 kDA PEG-NHS ester comprising two 20 kDA linear PEG molecules (e.g., Sunbright® GL2-400AL3, Sunbright® GL2-400GS2, NOF), a linear 30 kDa PEG-aldehyde (e.g., Sunbright® ME-300AL) and a linear 30 kDa PEG-NHS ester.

In some embodiments, a linker can be used to join the IL-10 agonist compound (e.g., a single-domain antibody polypeptide of an IL-10 agonist compound) and the PEG molecule. Suitable linkers include “flexible linkers” which are generally of sufficient length to permit some movement between the modified polypeptide sequences and the linked components and molecules. The linker molecules are generally about 6-50 atoms long. The linker molecules may also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids. Examples of flexible linkers are described in Section IV. Further, a multimer (e.g., 1, 2, 3, 4, 5, 6, 7, 8, 9, 10, 10-20, 20-30, or 30-50) of these linker sequences may be linked together to provide flexible linkers that may be used to conjugate two molecules. Alternative to a polypeptide linker, the linker can be a chemical linker, for example, a PEG-aldehyde linker. In some embodiments, the binding molecule is acetylated at the N-terminus by enzymatic reaction with N-terminal acetyltransferase and, for example, acetyl CoA. Alternatively, or in addition to N-terminal acetylation, the binding molecule can be acetylated at one or more lysine residues, for example, by enzymatic reaction with a lysine acetyltransferase. See, for example, Choudhary et al. 2009. Science 325 (5942):834840.

In some embodiments, the present invention provides an IL-10 agonist compound of formula #1 that is PEGylated, wherein the PEG is conjugated to the IL-10 agonist compound (e.g., a single-domain antibody polypeptide of an IL-10 agonist compound) and the PEG is a linear or branched PEG molecule having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, or alternatively about 30,000 to about 40,000 daltons. In one embodiment of the disclosure, the PEG is a 40 kD branched PEG comprising two 20 kD arms.

### Fatty Acid Carriers

In some embodiments, an IL-10 agonist compound having an extended duration of action in a mammalian subject and useful in the practice of the present disclosure is achieved by covalent attachment of the IL-10 agonist compound (e.g., a single-domain antibody polypeptide of an IL-10 agonist compound) to a fatty acid molecule as described in Resh. 2016. Progress in Lipid Research 63:120-131. Examples of fatty acids that may be conjugated include myristate, palmitate and palmitoleic acid. Myristoylate is typically linked to an N-terminal glycine but lysines may also be myristoylated. Palmitoylation is typically achieved by enzymatic modification of free cysteine —SH groups such as DHHC proteins catalyze S-palmitoylation. Palmitoleylation of serine and threonine residues is typically achieved enzymatically using PORCN enzymes. In some embodiments, the IL-10 agonist compound (e.g., a single-domain antibody polypeptide of an IL-10 agonist compound) is acetylated at the N-terminus by enzymatic reaction with N-terminal acetyltransferase and, for example, acetyl CoA. Alternatively, or in addition to N-terminal acetylation, the IL-10 agonist compound (e.g., a single-domain antibody polypeptide of an IL-10 agonist compound) is acetylated at one or more lysine residues, for example, by enzymatic reaction with a lysine acetyltransferase. See, for example, Choudhary et al. 2009. Science 325 5942:834.

### Modifications to Provide Additional Functions

In some embodiments, embodiment, the IL-10 agonist compound (e.g., a single-domain antibody polypeptide of an IL-10 agonist compound) of the present disclosure may comprise a functional domain of a chimeric polypeptide. IL-10 agonist compound fusion proteins of the present disclosure may be readily produced by recombinant DNA methodology by techniques known in the art by constructing a recombinant vector comprising a nucleic acid sequence encoding an IL-10 agonist compound in frame with a nucleic acid sequence encoding the fusion partner either at the N-terminus or C-terminus of the IL-10 agonist compounds (e.g., single-domain antibody polypeptides of the IL-10 agonist compounds), the sequence optionally further comprising a nucleic acid sequence in frame encoding a linker or spacer polypeptide.

### FLAG Tags

In other embodiments, the IL-10 agonist compound (e.g., a single-domain antibody polypeptide of an IL-10 agonist compound) can be modified to include an additional polypeptide sequence that functions as an antigenic tag, such as a FLAG sequence. FLAG sequences are recognized by biotinylated, highly specific, anti-FLAG antibodies, as described herein (see, e.g., Blanar et al. 1992. Science. 256:1014 and LeClair et al. 1992. PNAS-USA. 89:8145). In some embodiments, the binding molecule further comprises a C-terminal c-myc epitope tag.

### Targeting Moieties

In some embodiments, the IL-10 agonist compound (e.g., a single-domain antibody polypeptide of an IL-10 agonist compound) is conjugated to a molecule that provides a “targeting domain” to facilitate selective binding to a particular cell type or tissue expressing a cell surface molecule that specifically binds to the targeting domain, optionally incorporating a linker molecule of from 1-40 (alternatively 2-20, alternatively 5-20, alternatively 10-20) amino acids between IL-10 agonist compound sequence and the sequence of the targeting domain of the fusion protein.

In other embodiments, a chimeric polypeptide including an IL-10 agonist compound and an antibody or antigen-binding portion thereof can be generated. The antibody or antigen-binding component of the chimeric protein can serve as a targeting moiety. For example, it can be used to localize the chimeric protein to a particular subset of cells or target molecule. Methods of generating cytokine-antibody chimeric polypeptides are described, for example, in U.S. Pat. No. 6,617,135.

In some embodiments, the targeting moiety is an antibody that specifically binds to at least one cell surface molecule associated with a tumor cell (i.e. at least one tumor antigen), wherein the cell surface molecule associated with a tumor cell is selected from the group consisting of GD2, BCMA, CD19, CD33, CD38, CD70, GD2, IL3Rα2, CD19, mesothelin, Her2, EpCam, Muc1, ROR1, CD133, CEA, EGRFRVIII, PSCA, GPC3, Pan-ErbB and FAP

### C-Terminal Modifications to Reduce Immunogenicity

In some embodiments, the present disclosure provides modifications to the newly exposed C-terminal VTVSS amino acid sequence (SEQ ID NO:474) (e.g., SEQ ID NO:474-499) to eliminate or reduce recognition by pre-existing antibodies. The exposed C-terminal VTVSS amino acid sequence (SEQ ID NO:474) of camelid-derived single-domain antibody fragments, such as VHH and scFv fragments, is recognized by circulating pre-existing antibodies of the immune system and resulting in an immunogenic response, limiting the efficacy of therapeutic VHH and scFv drug therapeutics. This pre-existing antibody immune response may be reduced by modifying the C-terminal amino acid sequence of single-domain antibodies having an exposed C-terminal VTVSS amino acid sequence (SEQ ID NO:474). For example, Nieba et al. disclose mutating positions 11, 14, 41, 84, 87, and/or 89 of a VH region (amino acid position numbering according to Kabat). WO 11/07586 discloses mutating positions 99, 101, and/or 148 of a VL domain or positions 12, 97, 98, 99, 103, and/or 144 of a VH domain (corresponding to amino acid positions 11, 83, 84, 85, 89 and 103 according to Kabat).

In some embodiments, such amino acid sequence modifications to the C-terminal VTVSS amino acid sequence (SEQ ID NO:474) alter the neoepitope resulting from the newly exposed C-terminal VTVSS amino acid sequence (SEQ ID NO:474) on single-chain antibodies, such as a sdAb, VHH, a multi-domain antibody comprising fusions of IgGs or HSA with other single-chain antibodies, including bispecific antibodies comprising a single chain, an scFv, a sdAb, a Fab, a diabody, a scFab or any other antigen binding domain or Fc-fusion protein that will expose a normally unexposed N- or C-terminal sequence.

In one aspect, the disclosure relates to an isolated single-domain antibody comprising a C-terminal modification, wherein the C-terminal modification comprises the substitution, addition, or deletion of at least one amino acid residue such that the substitution, addition, or deletion of at least one amino acid residue to the single-domain antibody eliminates the interaction of at least one pre-existing antibody with the single-domain antibody without interfering with the binding of the single-domain antibody with its target.

In one embodiment, the C-terminal amino acid sequence of the single-domain antibody is exposed such that the exposed C-terminal is available for interaction with the pre-existing antibody, and wherein the C-terminal modification reduces the exposure of the C-terminal to the pre-existing antibody.

In one aspect, the present disclosure provides a polypeptide comprising a single-domain antibody (sdAb) comprising a modified C-terminal amino acid sequence aligning to the endogenous sdAb amino acid residues (T/L₁₀₈)V₁₀₉T₁₁₀V₁₁₁S₁₁₂S₁₁₃ (SEQ ID NO: 581) (numbered according to the Kabat numbering scheme for human VH carboxy-terminal amino acid residues). In accordance with the present disclosure, the modified amino acid sequence comprises the formula X₁₀₈X₁₀₉X₁₁₀V₁₁₁X₁₁₂ X₁₁₃Y, wherein:
X₁₀₈ is selected from the group consisting of L, T, and Q;
X₁₀₉ is selected from the group consisting of V, G, N, and L;
X₁₁₀ is selected from the group consisting of T and Q;
X₁₁₁ is V;
X₁₁₂ is selected from the group consisting of S, C, T, A, and G, or optionally absent; and
X₁₁₃ is selected from the group consisting of S, C, A, G, and T, or optionally absent; with the provisos that:
when X₁₀₉ is V, then X₁₁₂ and X₁₁₃ are not both S; and
X₁₁₂ and X₁₁₃ are not both C; and
Y is absent or comprises a polypeptide comprising from 1-5 amino acids and wherein such amino acids are independently selected from the group consisting of A, G, S, T, L, and V.

In some embodiments, the sdAb is further modified to comprise an amino acid substitution selected from the group consisting of L11S, L11Q, L11G, and P14A, numbered in accordance with the Kabat numbering scheme.

In some embodiments of the disclosure, X₁₀₈ is L; X₁₀₉ is selected from the group consisting of V, G, and L; X₁₁₀ is T; X₁₁₂ is selected from the group consisting of S, T, and C; X₁₁₃ is selected from the group consisting of S, T, C, and A; X₁₁₄ comprises a polypeptide comprising 1-5 amino acids independently selected from the group consisting of A, G, S, T, L, and V; and Y is absent or is a dipeptide of the amino acid sequence AA. In some embodiments, Y is AA. In other embodiments, the amino acid sequence X₁₀₉X₁₁₀V₁₁₁X₁₁₂ X₁₁₃ is V₁₀₉T₁₁₀V₁₁₁S₁₁₂ A₁₁₃ and Y is AA (“VTVSA” and “VTVSAAA” disclosed as SEQ ID NOS:479 and 480, respectively). In some embodiments, the sdAb comprises the amino acid sequence X₁₀₉X₁₁₀V₁₁₁X₁₁₂ X₁₁₃ is selected from the group consisting of SEQ ID NOS:121-135, 138-141, 144-155, 158-175, 179, 182-195, and 199 of Table 5, such as, for example, GTVSS (SEQ ID NO:476), LTVSS (SEQ ID NO:477), VTVCS (SEQ ID NO:481), and VTVSC (SEQ ID NO:483).

In some embodiments, the polypeptides of the disclosure exhibit reduced binding to pre-existing antibodies.

In another aspect, the disclosure relates to a polypeptide of the formula:

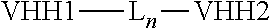

wherein VHH1 is a first VHH, L is a polypeptide linker comprising from 1-50 amino acids, n is 0 or 1, and VHH2 is a second VHH, which may be the same as or different from VHH1. In the formula VHH1-Ln-VHH2 described above, the linker molecule L may be a GS linker. Various suitable GS linkers are described and exemplified herein in Table 11.

In some embodiments, VHH1 and VHH2 each independently bind to the extracellular domain of a cytokine receptor. In some embodiments, the cytokine receptors to which VHH1 and VHH2 bind is selected from the group consisting of IL-2R, IL-2Rβ, IL-2Rγ, IL-10Rα, IL-10Rβ, IL-12Rβ1, IL-12Rβ2, IL-18Rα, IL-18Rβ, IL-22R1, IL-27Rα, gp130, IL-23R, IL-28Rα, IFNRγ1, and IFNRγ2. In some embodiments, VHH1 and VHH2 selectively bind to a pair of cytokine receptors selected from the following pairs: IL-10Rα/IL-10Rβ, IL-27Rα/gp130, IFNγR1/IFNγR2, IL-10Rβ/IL-28Rα, IL-2Rβ/IL2Rγ, IL-18Rα/IL-18Rβ, IL-22R1/IL-10Rβ, IL-10Rα/IL-2Rγ, IL-2Rβ/IL-2Rγ, IL-10R1/IFNRγ, IFNRγ/IL-28Rα, IL-12R1/IL-12Rβ2, IL-12R1/IL-23R, and IL-10Rα/IL-2Rγ.

In some embodiments, the single-domain antibodies and antigen-binding fragments thereof can be conjugated to various other chemical entities, including antibody-drug conjugates (ADCs).

Examples of modifications to the C-terminal VTVSS motif (SEQ ID NO:474) of IL10R agonist compounds of the present disclosure are provided in Table 5, SEQ ID NOS:121-135, 138-141, 144-155, 158-175, 179, 182-195, and 199.

### Recombinant Production

Alternatively, the humanized IL-10 agonist compounds of the present disclosure are produced by recombinant DNA technology. In the typical practice of recombinant production of polypeptides, a nucleic acid sequence encoding the desired polypeptide is incorporated into an expression vector suitable for the host cell in which expression will be accomplished. The nucleic acid sequence is operably linked to one or more expression control sequences encoded by the vector and functional in the target host cell. The recombinant protein may be recovered through disruption of the host cell or from the cell medium if a secretion leader sequence (signal peptide) is incorporated into the polypeptide.

### Nucleic Acid Sequences Encoding IL-10 Agonist Compounds

In some embodiments, the disclosure provides nucleic acid sequences encoding an IL-10 agonist compound of any of SEQ ID NOS:1-24, 121-135, 138-141, 144-155, 158-175, 179, 182-195, 199, and 500-523.

In some embodiments, the humanized IL-10 agonist compound is produced by recombinant methods using a nucleic acid sequence encoding the IL-10 agonist compound (e.g., a single-domain antibody polypeptide of an IL-10 agonist compound) (or fusion protein comprising the IL-10 agonist compounds (e.g., single-domain antibody polypeptides of the IL-10 agonist compounds)). The nucleic acid sequence encoding the desired hzIL-10 agonist compound can be synthesized by chemical means using an oligonucleotide synthesizer.

The nucleic acid molecules are not limited to sequences that encode polypeptides; some or all of the non-coding sequences that lie upstream or downstream from a coding sequence (e.g., the coding sequence of IL-2) can also be included. Those of ordinary skill in the art of molecular biology are familiar with routine procedures for isolating nucleic acid molecules. They can, for example, be generated by the treatment of genomic DNA with restriction endonucleases, or by performance of the polymerase chain reaction (PCR). In the event the nucleic acid molecule is a ribonucleic acid (RNA), molecules can be produced, for example, by in vitro transcription.

The nucleic acid molecules encoding the IL-10 agonist compound (e.g., a single-domain antibody polypeptide of an IL-10 agonist compound) (and fusions thereof) may contain naturally occurring sequences or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand).

Nucleic acid sequences encoding the IL-10 agonist compound (e.g., a single-domain antibody polypeptide of an IL-10 agonist compound) may be obtained from various commercial sources that provide custom-made nucleic acid sequences. Amino acid sequence variants of the IL-10 agonist compounds (e.g., single-domain antibody polypeptides of the IL-10 agonist compounds) of the present disclosure are prepared by introducing appropriate nucleotide changes into the coding sequence based on the genetic code which is well-known in the art. Such variants represent insertions, substitutions, and/or specified deletions of, residues as noted. Any combination of insertion, substitution, and/or specified deletion is made to arrive at the final construct, provided that the final construct possesses the desired biological activity as defined herein.

Methods for constructing a DNA sequence encoding an IL-10 agonist compound and expressing those sequences in a suitably transformed host include but are not limited to using a PCR-assisted mutagenesis technique. Mutations that consist of deletions or additions of amino acid residues to an IL-10 agonist compound can also be made with standard recombinant techniques. In the event of a nucleotide deletion or addition, the nucleic acid molecule encoding an IL-10 agonist compound is optionally digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences.

An IL-10 agonist compound of the present disclosure may be produced recombinantly not only directly, but also as a fusion polypeptide with a heterologous polypeptide, for example, a signal sequence or other polypeptide having a specific cleavage site at the N-terminus or C-terminus of the mature IL-10 agonist compounds. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. The inclusion of a signal sequence depends on whether it is desired to secrete the IL-10 agonist compound (e.g., a single-domain antibody polypeptide of an IL-10 agonist compound) from the recombinant cells in which it is made. If the chosen cells are prokaryotic, it generally is preferred that the DNA sequence not encode a signal sequence. When the recombinant host cell is a yeast cell such as Saccharomyces cerevisiae, the alpha mating factor secretion signal sequence may be employed to achieve extracellular secretion of the IL-10 agonist compound (e.g., a single-domain antibody polypeptide of an IL-10 agonist compound) into the culture medium as described in Singh, U.S. Pat. No. 7,198,919 B1 issued Apr. 3, 2007.

In the event the IL-10 agonist compound (e.g., a single-domain antibody polypeptide of an IL-10 agonist compound) to be expressed is to be expressed as a chimera (e.g., a fusion protein comprising an IL-10 agonist compound and a heterologous polypeptide sequence), the chimeric protein can be encoded by a hybrid nucleic acid molecule comprising a first sequence that encodes all or part of the IL-10 agonist compound (e.g., a single-domain antibody polypeptide of an IL-10 agonist compound) and a second sequence that encodes all or part of the heterologous polypeptide. For example, subject IL-10 agonist compounds described herein may be fused to a hexa-/octa-histidine tag (H6, H7, H8, disclosed as SEQ ID NOS 451-453, respectively) to facilitate the purification of bacterially expressed protein, or to a hemagglutinin tag to facilitate purification of protein expressed in eukaryotic cells. By first and second, it should not be understood as limiting to the orientation of the elements of the fusion protein, and a heterologous polypeptide can be linked at either the N-terminus and/or C-terminus of the IL-10 agonist compounds (e.g., single-domain antibody polypeptides of the IL-10 agonist compounds). For example, the N-terminus may be linked to a targeting domain, and the C-terminus is linked to a hexa-histidine tag (SEQ ID NO:451) purification handle.

The complete amino acid sequence of the polypeptide (or fusion/chimera) to be expressed can be used to construct a back-translated gene. A DNA oligomer containing a nucleotide sequence coding an IL-10 agonist compound can be synthesized. For example, several small oligonucleotides coding for portions of the desired polypeptide can be synthesized and then ligated. The individual oligonucleotides typically contain 5′ or 3′ overhangs for complementary assembly.

### Codon Optimization

In some embodiments, the nucleic acid sequence encoding the IL-10 agonist compound (e.g., a single-domain antibody polypeptide of an IL-10 agonist compound) may be “codon-optimized” to facilitate expression in a particular host cell type. Techniques for codon optimization in a wide variety of expression systems, including mammalian, yeast, and bacterial host cells, are well known in the art and online tools are available to provide codon-optimized sequences for expression in a variety of host cell types. See e.g., Al-Hawash et al. 2017. Gene Reports. 9:46-53 and Mauro and Chappell. Recombinant Protein Expression in Mammalian Cells: Methods and Protocols., edited by David Hacker (Human Press New York). Additionally, there are a variety of web-based on-line software packages that are freely available to assist in the preparation of codon-optimized nucleic acid sequences.

### Expression Vectors

Once assembled (by synthesis, site-directed mutagenesis or another method), the nucleic acid sequence encoding an IL-10 agonist compound will be inserted into an expression vector. A variety of expression vectors for uses in various host cells are available and are typically selected based on the host cell for expression. An expression vector typically includes but is not limited to one or more of the following: an origin of replication, one or more marker genes, an enhancer element, a promoter, and a transcription termination sequence. Vectors include viral vectors, plasmid vectors, integrating vectors, and the like. Plasmids are examples of non-viral vectors.

To facilitate efficient expression of the recombinant polypeptide, the nucleic acid sequence encoding the polypeptide sequence to be expressed is operably linked to transcriptional and translational regulatory control sequences that are functional in the chosen expression host.

### Selectable Marker

Expression vectors usually contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, for example, ampicillin, neomycin, methotrexate, or tetracycline, (b) complement auxotrophic deficiencies, or (c) supply critical nutrients not available from complex media.

### Regulatory Control Sequences

Expression vectors for an IL-10 agonist compound of the present disclosure contain a regulatory sequence that is recognized by the host organism and is operably linked to a nucleic acid sequence encoding the IL-10 agonist compounds (e.g., single-domain antibody polypeptides of the IL-10 agonist compounds). The terms “regulatory control sequence,” “regulatory sequence,” or “expression control sequence” are used interchangeably herein to refer to promoters, enhancers, and other expression control elements (e.g., polyadenylation signals). See, for example, Goeddel. 1990. Gene Expression Technology: Methods in Enzymology 185 (Academic Press, San Diego, CA USA Regulatory sequences include those that direct constitute expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of the protein desired, and the like. In selecting an expression control sequence, a variety of factors understood by one of skill in the art are to be considered. These include, for example, the relative strength of the sequence, its controllability, and its compatibility with the actual DNA sequence encoding the IL-10 agonist compounds (e.g., single-domain antibody polypeptides of the IL-10 agonist compounds), particularly as regards potential secondary structures.

### Promoters

In some embodiments, the regulatory sequence is a promoter, which is selected based on, for example, the cell type in which expression is sought. Promoters are untranslated sequences located upstream (5′) to the start codon of a structural gene (generally within about 100 to 1000 bp) that control the transcription and translation of a particular nucleic acid sequence to which they are operably linked. Such promoters typically fall into two classes, inducible and constitutive. Inducible promoters are promoters that initiate increased levels of transcription from DNA under their control in response to some change in culture conditions, for example, the presence or absence of a nutrient or a change in temperature. A large number of promoters recognized by a variety of potential host cells are well known.

A T7 promoter can be used in bacteria, a polyhedrin promoter can be used in insect cells, and a cytomegalovirus or metallothionein promoter can be used in mammalian cells. Also, in the case of higher eukaryotes, tissue-specific, and cell type-specific promoters are widely available. These promoters are so named for their ability to direct the expression of a nucleic acid molecule in a given tissue or cell type within the body. Skilled artisans are well aware of numerous promoters and other regulatory elements which can be used to direct the expression of nucleic acids.

Transcription from vectors in mammalian host cells may be controlled, for example, by promoters obtained from the genomes of viruses such as polyomavirus, fowlpox virus, adenovirus (such as human adenovirus serotype 5), bovine papillomavirus, avian sarcoma virus, cytomegalovirus, a retrovirus (such as murine stem cell virus), hepatitis-B virus and most preferably Simian Virus 40 (SV40), from heterologous mammalian promoters, for example, the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication.

### Enhancers

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, alpha-fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence but is preferably located at a site 5′ from the promoter. Expression vectors used in eukaryotic host cells will also contain sequences necessary for the termination of transcription and for stabilizing the mRNA. Such sequences are commonly available from the 5′ and, occasionally, 3′, untranslated regions of eukaryotic or viral DNAs or cDNAs. Construction of suitable vectors containing one or more of the above-listed components employs standard techniques.

In addition to sequences that facilitate transcription of the inserted nucleic acid molecule, vectors can contain origins of replication, and other genes that encode a selectable marker. For example, the neomycin-resistance (neoR) gene imparts G418 resistance to cells in which it is expressed and thus permits phenotypic selection of the transfected cells. Additional examples of marker or reporter genes include beta-lactamase, chloramphenicol acetyltransferase (CAT), adenosine deaminase (ADA), dihydrofolate reductase (DHFR), hygromycin-B-phosphotransferase (HPH), thymidine kinase (TK), lacZ (encoding beta-galactosidase), and xanthine guanine phosphoribosyltransferase (XGPRT). Those of skill in the art can readily determine whether a given regulatory element or selectable marker is suitable for use in a particular experimental context.

Proper assembly of the expression vector can be confirmed by nucleotide sequencing, restriction mapping, and expression of a biologically active polypeptide in a suitable host.

### Host Cells

The present disclosure further provides prokaryotic or eukaryotic cells that contain and express a nucleic acid molecule that encodes an IL-10 agonist compound. A cell of the present disclosure is a transfected cell, i.e., a cell into which a nucleic acid molecule, for example, a nucleic acid molecule encoding a mutant IL-2 polypeptide, has been introduced by means of recombinant DNA techniques. The progeny of such a cell is also considered within the scope of the present disclosure.

Host cells are typically selected in accordance with their compatibility with the chosen expression vector, the toxicity of the product coded for by the DNA sequences of this invention, their secretion characteristics, their ability to fold the polypeptides correctly, their fermentation or culture requirements, and the ease of purification of the products coded for by the DNA sequences. Suitable host cells for cloning or expressing the DNA in the vectors herein are the prokaryote, yeast, or higher eukaryote cells.

In some embodiments, the recombinant IL-10 agonist compound can also be made in eukaryotes, such as yeast or human cells. Suitable eukaryotic host cells include insect cells (examples of Baculovirus vectors available for expression of proteins in cultured insect cells (e.g., Sf9 cells) include the pAc series (Smith et al. 1983. Mol Cell Biol. 3:2156-2165) and the pVL series (Lucklow and Summers. 1989. Virology. 170:31-39)); yeast cells (examples of vectors for expression in yeast S. cerevisiae include pYepSecl (Baldari et al. 1987. EMBO J. 6:229-234), pMFa (Kurjan and Herskowitz. 1982. Cell. 30:933-943), pJRY88 (Schultz et al. 1987. Gene. 54:113-123), pYES2 (Invitrogen Corporation, San Diego, Calif.), and pPicZ (Invitrogen Corporation, San Diego, Calif.); or mammalian cells (mammalian expression vectors include pCDM8 (Seed. 1987. Nature. 329:840) and pMT2PC (Kaufman et al. 0.1987. EMBO J. 6:187:195)).

Examples of useful mammalian host cell lines are mouse L cells (L-M[TK-], ATCC #CRL-2648), monkey kidney CV1 line transformed by SV40 (COS-7, ATCC CRL 1651); human embryonic kidney line (HEK293 or HEK293 cells subcloned for growth in suspension culture; baby hamster kidney cells (BHK, ATCC CCL 10); Chinese hamster ovary cells-DHFR (CHO); mouse Sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2). In mammalian cells, the expression vector's control functions are often provided by viral regulatory elements. For example, commonly used promoters are derived from polyoma, Adenovirus 2, cytomegalovirus, and Simian Virus 40.

The IL-10 agonist compound (e.g., a single-domain antibody polypeptide of an IL-10 agonist compound) may be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans of ordinary skill are able to make such a determination. Furthermore, if guidance is required in selecting an expression system, skilled artisans may consult Ausubel et al. 1993. Current Protocols in Molecular Biology. John Wiley and Sons, New York, N.Y.) and Pouwels et al. 1987. Cloning Vectors: A Laboratory Manual. Suppl.).

In some embodiments, an IL-10 agonist compound obtained will be glycosylated or unglycosylated depending on the host organism used to produce the mutein. If bacteria are chosen as the host then the IL-10 agonist compound (e.g., a single-domain antibody polypeptide of an IL-10 agonist compound) produced will be unglycosylated. Eukaryotic cells, on the other hand, will typically result in glycosylation of the IL-10 agonist compounds (e.g., single-domain antibody polypeptides of the IL-10 agonist compounds).

In some embodiments, it is possible that an amino acid sequence (particularly a CDR sequence) of a sdAb to be incorporated into an IL-10 agonist compound may contain a glycosylation motif, particularly an N-linked glycosylation motif of the sequence Asn-X-Ser (N-X-S) or Asn-X-Thr (N-X-T), wherein X is any amino acid except for proline. In such instances, it is desirable to eliminate such N-linked glycosylation motifs by modifying the sequence of the N-linked glycosylation motif to prevent glycosylation. In some embodiments, the N-linked glycosylation motif is disrupted by the incorporation of conservative amino acid substitution of the Asn (N) residue of the N-linked glycosylation motif.

For other additional expression systems for both prokaryotic and eukaryotic cells, see Chapters 16 and 17 of Sambrook et al. 1989. Molecular Cloning: A Laboratory Manual (2nd ed., Cold Spring Harbor Laboratory Press, Plainview, N.Y.) and Goeddel. 1990. Gene Expression Technology: Methods in Enzymology 185 (Academic Press, San Diego, Calif.).

### Transfection

The expression constructs of the disclosure can be introduced into host cells to thereby produce an IL-10 agonist compound disclosed herein. The expression vector comprising a nucleic acid sequence encoding the IL-10 agonist compound (e.g., a single-domain antibody polypeptide of an IL-10 agonist compound) is introduced into the prokaryotic or eukaryotic host cells via conventional transformation or transfection techniques. Suitable methods for transforming or transfecting host cells can be found in Sambrook et al. 1989. Molecular Cloning: A Laboratory Manual (2d ed., Cold Spring Harbor Laboratory Press, Plainview, N.Y.) and other standard molecular biology laboratory manuals. To facilitate transfection of the target cells, the target cell may be exposed directly to the non-viral vector may under conditions that facilitate uptake of the non-viral vector. Examples of conditions that facilitate the uptake of foreign nucleic acid by mammalian cells are well known in the art and include but are not limited to chemical means (such as Lipofectamine®, Thermo-Fisher Scientific), high salt, and magnetic fields (electroporation).

### Cell Culture

Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH, and the like, are those previously used with the host cell selected for expression and will be apparent to the ordinarily skilled artisan.

### Recovery of Recombinant Proteins

Recombinantly produced IL-10 agonist compound polypeptides can be recovered from the culture medium as a secreted polypeptide if a secretion leader sequence is employed. Alternatively, the IL-10 agonist compound (e.g., a single-domain antibody polypeptide of an IL-10 agonist compound) polypeptides can also be recovered from host cell lysates. A protease inhibitor, such as phenyl methyl sulfonyl fluoride (PMSF) may be employed during the recovery phase from cell lysates to inhibit proteolytic degradation during purification, and antibiotics may be included to prevent the growth of adventitious contaminants.

Various purification steps are known in the art and find use, e.g., affinity chromatography. Affinity chromatography makes use of the highly specific binding sites usually present in biological macromolecules, separating molecules on their ability to bind a particular ligand. Covalent bonds attach the ligand to an insoluble, porous support medium in a manner that overtly presents the ligand to the protein sample, thereby using the natural specific binding of one molecular species to separate and purify a second species from a mixture. Antibodies are commonly used in affinity chromatography. Size selection steps may also be used, e.g., gel filtration chromatography (also known as size-exclusion chromatography or molecular sieve chromatography) is used to separate proteins according to their size. In gel filtration, a protein solution is passed through a column that is packed with semipermeable porous resin. The semipermeable resin has a range of pore sizes that determines the size of proteins that can be separated with the column.

A recombinantly IL-10 agonist compound by the transformed host can be purified according to any suitable method. Recombinant IL-10 agonist compounds can be isolated from inclusion bodies generated in E. coli, or from conditioned medium from either mammalian or yeast cultures producing a given mutein using cation exchange, gel filtration, and or reverse phase liquid chromatography. The substantially purified forms of the recombinant IL-10 agonist compound can be purified from the expression system using routine biochemical procedures, and can be used, for example, as therapeutic agents, as described herein.

In some embodiments, where the IL-10 agonist compound (e.g., a single-domain antibody polypeptide of an IL-10 agonist compound) is expressed with a purification tag as discussed above, this purification handle may be used for isolation of the IL-10 agonist compound (e.g., a single-domain antibody polypeptide of an IL-10 agonist compound) from the cell lysate or cell medium. Where the purification tag is a chelating peptide, methods for the isolation of such molecules using immobilized metal affinity chromatography are well-known in the art. See, for example, Smith, et al. U.S. Pat. No. 4,569,794.

The biological activity of the IL-10 agonist compounds (e.g., single-domain antibody polypeptides of the IL-10 agonist compounds) recovered can be assayed for activating by any suitable method known in the art and may be evaluated as substantially purified forms or as part of the cell lysate or cell medium when secretion leader sequences are employed for expression.

### Pharmaceutical Formulations

In some embodiments, the subject IL-10 agonist compound (and/or nucleic acids encoding the IL-10 agonist compound (e.g., a single-domain antibody polypeptide of an IL-10 agonist compound) or recombinant cells incorporating a nucleic acid sequence and modified to express the IL-10 agonist compounds (e.g., single-domain antibody polypeptides of the IL-10 agonist compounds)) can be incorporated into compositions, including pharmaceutical compositions. Such compositions typically include the polypeptide or nucleic acid molecule and a pharmaceutically acceptable carrier. A pharmaceutical composition is formulated to be compatible with its intended route of administration and is compatible with the therapeutic use for which the IL-10 agonist compound (e.g., a single-domain antibody polypeptide of an IL-10 agonist compound) is to be administered to the subject in need of treatment or prophylaxis.

### Carriers

Carriers include a sterile diluent such as water for injection, saline solution, fixed oils, polyethylene glycols, glycerine, propylene glycol or other synthetic solvents. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, for example, sodium dodecyl sulfate. For intravenous administration, suitable carriers include physiological saline, bacteriostatic water, Cremophor ELTM (BASF, Parsippany, N.J.) or phosphate buffered saline (PBS).

### Buffers

The term buffers includes buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, for example, 7.5).

### Dispersions

Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

### Preservatives

The pharmaceutical formulations for parenteral administration to a subject should be sterile and should be fluid to facilitate easy syringability. It should be stable under the conditions of manufacture and storage and are preserved against the contamination. Prevention of the action of microorganisms can be achieved by various antibacterial and antifungal agents, for example, agents such as benzyl alcohol or methyl parabens; antioxidants such as ascorbic acid or sodium bisulfite; chelating agents such as ethylenediaminetetraacetic acid, parabens, chlorobutanol, phenol, ascorbic acid, thimerosal, and the like. Sterile solutions can be prepared by incorporating the active compound in the required amount in an appropriate solvent with one or a combination of ingredients enumerated above, as required, followed by filtered sterilization.

### Tonicity Agents

In many cases, it will be preferable to include isotonic agents, for example, sugars, polyalcohols such as mannitol, sorbitol, sodium chloride in the composition.

### Routes of Administration

In some embodiments of the therapeutic methods of the present disclosure involve the administration of a pharmaceutical formulation comprising a IL-10 agonist compound (and/or nucleic acids encoding the IL-10 agonist compound (e.g., a single-domain antibody polypeptide of an IL-10 agonist compound) or recombinantly modified host cells expressing the IL-10 agonist compounds (e.g., single-domain antibody polypeptides of the IL-10 agonist compounds)) to a subject in need of treatment. The pharmaceutical formulation comprising a IL-10 agonist compounds of the present disclosure may be administered to a subject in need of treatment or prophyaxis by a variety of routes of administration, including parenteral administration, oral, topical, or inhalation routes.

### Parenteral Administration

In some embodiments, the methods of the present disclosure involve the parenteral administration of a pharmaceutical formulation comprising a IL-10 agonist compound (and/or nucleic acids encoding the IL-10 agonist compound (e.g., a single-domain antibody polypeptide of an IL-10 agonist compound) or recombinantly modified host cells expressing the IL-10 agonist compounds (e.g., single-domain antibody polypeptides of the IL-10 agonist compounds)) to a subject in need of treatment. Examples of parenteral routes of administration include, for example, intravenous, intradermal, subcutaneous, transdermal (topical), transmucosal, and rectal administration. Parenteral formulations comprise solutions or suspensions used for parenteral application can include vehicles the carriers and buffers. Pharmaceutical formulations for parenteral administration include sterile aqueous solutions (where water soluble) or dispersions and sterile powders for the extemporaneous preparation of sterile injectable solutions or dispersion. The parenteral preparation can be enclosed in ampoules, disposable syringes or multiple dose vials made of glass or plastic. In one embodiment, the formulation is provided in a prefilled syringe for parenteral administration.

### Oral Administration

In some embodiments, the methods of the present disclosure involve the oral administration of a pharmaceutical formulation comprising an IL-10 agonist compound (and/or nucleic acids encoding the IL-10 agonist compound (e.g., a single-domain antibody polypeptide of an IL-10 agonist compound) or recombinantly modified host cells expressing the IL-10 agonist compounds (e.g., single-domain antibody polypeptides of the IL-10 agonist compounds)) to a subject in need of treatment. Oral compositions, if used, generally include an inert diluent or an edible carrier. For the purpose of oral therapeutic administration, the active compound can be incorporated with excipients and used in the form of tablets, troches, or capsules, for example, gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

### Inhalation Formulations

In some embodiments, the methods of the present disclosure involve the inhaled administration of a pharmaceutical formulation comprising an IL-10 agonist compound (and/or nucleic acids encoding the IL-10 agonist compound (e.g., a single-domain antibody polypeptide of an IL-10 agonist compound) or recombinantly modified host cells expressing the IL-10 agonist compounds (e.g., single-domain antibody polypeptides of the IL-10 agonist compounds)) to a subject in need of treatment. In the event of administration by inhalation, subject IL-10 agonist compounds, or the nucleic acids encoding them, are delivered in the form of an aerosol spray from pressured container or dispenser which contains a suitable propellant, for example, a gas such as carbon dioxide, or a nebulizer. Such methods include those described in U.S. Pat. No. 6,468,798.

### Mucosal and Transdermal Formulations

In some embodiments, the methods of the present disclosure involve the mucosal or transdermal administration of a pharmaceutical formulation comprising an IL-10 agonist compound (and/or nucleic acids encoding the IL-10 agonist compound (e.g., a single-domain antibody polypeptide of an IL-10 agonist compound) or recombinantly modified host cells expressing the IL-10 agonist compounds (e.g., single-domain antibody polypeptides of the IL-10 agonist compounds)) to a subject in need of treatment. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories (e.g., with conventional suppository bases such as cocoa butter and other glycerides) or retention enemas for rectal delivery. For transdermal administration, the active compounds are formulated into ointments, salves, gels, or creams as generally known in the art and may incorporate permeation enhancers such as ethanol or lanolin.

### Extended Release and Depot Formulations

In some embodiments of the method of the present disclosure, the IL-10 agonist compound (e.g., a single-domain antibody polypeptide of an IL-10 agonist compound) is administered to a subject in need of treatment in a formulation to provide extended release of the IL-10 agonist compound (e.g., a single-domain antibody polypeptide of an IL-10 agonist compound) agent. Examples of extended-release formulations of the injectable compositions can be brought about by including in the composition an agent that delays absorption, for example, aluminum monostearate and gelatin. In one embodiment, the subject IL-10 agonist compounds or nucleic acids are prepared with carriers that will protect the IL-10 agonist compounds (e.g., single-domain antibody polypeptides of the IL-10 agonist compounds) against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

### Administration of Nucleic Acids Encoding the IL-10 Agonist Compounds

In some embodiments of the method of the present disclosure, delivery of the IL-10 agonist compound (e.g., a single-domain antibody polypeptide of an IL-10 agonist compound) to a subject in need of treatment is achieved by the administration of a nucleic acid encoding the IL-10 agonist compounds (e.g., single-domain antibody polypeptides of the IL-10 agonist compounds). Methods for the administration of a nucleic acid encoding an IL-10 agonist compound to a subject are achieved by transfection or infection using methods known in the art, including but not limited to the methods described in McCaffrey et al. 2002. Nature. 418:6893), Xia et al. 2002. Nature Biotechnol. 20:1006-1010), or Putnam. 1996. Am J Health Syst Pharm. 53:151-160, 325. In some embodiments, the IL-10 agonist compound (e.g., a single-domain antibody polypeptide of an IL-10 agonist compound) is administered to a subject by the administration of a pharmaceutically acceptable formulation of a recombinant expression vector comprising a nucleic acid sequence encoding the IL-10 agonist compound (e.g., a single-domain antibody polypeptide of an IL-10 agonist compound) operably linked to one or more expression control sequences operable in a mammalian subject. In some embodiments, the expression control sequence may be selected that is operable in a limited range of cell types (or single cell type) to facilitate the selective expression of the IL-10 agonist compound (e.g., a single-domain antibody polypeptide of an IL-10 agonist compound) in a particular target cell type. In one embodiment, the recombinant expression vector is a viral vector. In some embodiments, the recombinant vector is a recombinant viral vector. In some embodiments, the recombinant viral vector is a recombinant adenoassociated virus (rAAV) or recombinant adenovirus (rAd), such as a replication-deficient adenovirus derived from human adenovirus serotypes 3 and/or 5. In some embodiments, the replication-deficient adenovirus has one or more modifications to the E1 region which interfere with the ability of the virus to initiate the cell cycle and/or apoptotic pathways in a human cell. The replication-deficient adenoviral vector may optionally comprise deletions in the E3 domain. In some embodiments, the adenovirus is a replication-competent adenovirus. In some embodiments, the adenovirus is a replication-competent recombinant virus engineered to selectively replicate in the target cell type.

In some embodiments, for the administration of IL-10 agonist compounds to the subject for the treatment of diseases of the intestinal tract or bacterial infections in a subject, the nucleic acid encoding the IL-10 agonist compound (e.g., a single-domain antibody polypeptide of an IL-10 agonist compound) may be delivered to the subject by the administration of a recombinantly modified bacteriophage vector encoding the IL-10 agonist compounds (e.g., single-domain antibody polypeptides of the IL-10 agonist compounds). As used herein, the terms “procaryotic virus,” “bacteriophage” and “phage” are used interchangeably to describe any of a variety of bacterial viruses that infect and replicate within a bacterium. Bacteriophage selectively infects procaryotic cells, restricting the expression of the IL-10 agonist compound (e.g., a single-domain antibody polypeptide of an IL-10 agonist compound) to procaryotic cells in the subject while avoiding expression in mammalian cells. A wide variety of bacteriophages capable of selecting a broad range of bacterial cells have been identified and characterized extensively in the scientific literature. In some embodiments, the phage is modified to remove adjacent motifs (PAM). Elimination of the Cas9 sequences from the phage genome reduces the ability of the Cas9 endonuclease of the target procaryotic cell to neutralize the invading phage encoding the IL-10 agonist compounds (e.g., single-domain antibody polypeptides of the IL-10 agonist compounds).

### Administration of Recombinantly Modified Cells Expressing IL-10 Agonist Proteins

In some embodiments of the method of the present disclosure, delivery of the IL-10 agonist compound (e.g., a single-domain antibody polypeptide of an IL-10 agonist compound) to a subject in need of treatment is achieved by the administration of recombinant host cells modified to express the IL-10 agonist compound (e.g., a single-domain antibody polypeptide of an IL-10 agonist compound) may be administered in the therapeutic and prophylactic applications described herein. In some embodiments, the recombinant host cells are mammalian cells, for example, human cells.

In some embodiments, the nucleic acid sequence encoding the IL-10 agonist compound (e.g., a single-domain antibody polypeptide of an IL-10 agonist compound) (or vectors comprising the same) may be maintained extrachromosomally in the recombinantly modified host cell for administration. In other embodiments, the nucleic acid sequence encoding the IL-10 agonist compound (e.g., a single-domain antibody polypeptide of an IL-10 agonist compound) may be incorporated into the genome of the host cell to be administered using at least one endonuclease to facilitate the incorporation or insertion of a nucleic acid sequence into the genomic sequence of the cell. As used herein, the term “endonuclease” is used to refer to a wild-type or variant enzyme capable of catalyzing the cleavage of bonds between nucleic acids within a DNA or RNA molecule, preferably a DNA molecule. Endonucleases are referred to as “rare-cutting” endonucleases when such endonucleases have a polynucleotide recognition site greater than about 12 base pairs (bp) in length, more preferably of 14-55 bp. Rare-cutting endonucleases can be used for inactivating genes at a locus or to integrate transgenes by homologous recombination (HR) i.e. by inducing DNA double-strand breaks (DSBs) at a locus and insertion of exogenous DNA at this locus by gene repair mechanism. Examples of rare-cutting endonucleases include homing endonucleases (Grizot et al. 2009. Nucleic Acids Research. 37(16):5405-5419), chimeric Zinc-Finger nucleases (ZFN) resulting from the fusion of engineered zinc-finger domains (Porteus M and Carroll D. 2005. Gene targeting using zinc finger nucleases. Nature Biotechnology 23(3):967-973, a TALEN-nuclease, a Cas9 endonuclease from CRISPR system as or a modified restriction endonuclease to extended sequence specificity (Eisenschmidt et al. 2005. Nucl Acids Res. 33(22): 7039-7047).

In some embodiments, particularly for the administration of IL-10 agonist compounds to the intestinal tract, the IL-10 agonist compound (e.g., a single-domain antibody polypeptide of an IL-10 agonist compound) may be delivered to the subject by a recombinantly modified procaryotic cell (e.g., Lactobacillus lacti). The use of engineered procaryotic cells for the delivery of recombinant proteins to the intestinal tract is known in the art. See, e.g., Lin et al. 2017. Microb Cell Fact. 16:148. In some embodiments, the engineered bacterial cell expressing the IL-10 agonist compound (e.g., a single-domain antibody polypeptide of an IL-10 agonist compound) may be administered orally, typically in aqueous suspension, or rectally (e.g., enema).

### Methods of Use

The present disclosure further provides methods of treating a subject suffering from a disease disorder or condition by the administration of a therapeutically effective amount of an IL-10 agonist compound (or nucleic acid encoding an IL-10 agonist compound including recombinant viruses encoding the IL-10 agonist compounds (e.g., single-domain antibody polypeptides of the IL-10 agonist compounds)) of the present disclosure.

### Inflammatory and Autoimmune Disorders

Disorders amenable to treatment with an IL-10 agonist compound (including pharmaceutically acceptable formulations comprising an IL-10 agonist compounds and/or the nucleic acid molecules that encode them including recombinant viruses encoding such an IL-10 agonist compounds) of the present disclosure include inflammatory or autoimmune diseases including but not limited to, organ rejection, graft versus host disease, autoimmune thyroid disease, multiple sclerosis, allergy, asthma, neurodegenerative diseases including Alzheimer's disease, systemic lupus erythramatosis (SLE), autoinflammatory diseases, inflammatory bowel disease (IBD), Crohn's disease, diabetes including Type 1 or type 2 diabetes, inflammation, autoimmune disease, atopic diseases, paraneoplastic autoimmune diseases, cartilage inflammation, arthritis, rheumatoid arthritis, juvenile arthritis, juvenile rheumatoid arthritis, juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reiter's Syndrome, SEA Syndrome (Seronegativity Enthesopathy Arthropathy Syndrome), juvenile dermatomyositis, juvenile psoriatic arthritis, juvenile scleroderma, juvenile systemic lupus erythematosus, juvenile vasculitis, pauciarticular rheumatoidarthritis, polyarticular rheumatoidarthritis, systemic onset rheumatoidarthritis, ankylosing spondylitis, enteropathic arthritis, reactive arthritis, Reiter's syndrome, SEA Syndrome (Seronegativity, Enthesopathy, Arthropathy Syndrome).

Other examples of proliferative and/or differentiative disorders amenable to treatment with IL-10 agonist compounds (including pharmaceutically acceptable formulations comprising IL-10 agonist compounds and/or the nucleic acid molecules that encode them including recombinant viruses encoding such IL-10 agonist compounds) of the present disclosure include, but are not limited to, skin disorders. The skin disorder may involve the aberrant activity of a cell or a group of cells or layers in the dermal, epidermal, or hypodermal layer, or an abnormality in the dermal-epidermal junction. For example, the skin disorder may involve aberrant activity of keratinocytes (e.g., hyperproliferative basal and immediately suprabasal keratinocytes), melanocytes, Langerhans cells, Merkel cells, immune cell, and other cells found in one or more of the epidermal layers, for example, the Stratum basale (Stratum germinativum), Stratum spinosum, Stratum granulosum, Stratum lucidum or Stratum corneum. In other embodiments, the disorder may involve aberrant activity of a dermal cell, for example, a dermal endothelial, fibroblast, immune cell (e.g., mast cell or macrophage) found in a dermal layer, for example, the papillary layer or the reticular layer.

Examples of inflammatory or autoimmune skin disorders include psoriasis, psoriatic arthritis, dermatitis (eczema), for example, exfoliative dermatitis or atopic dermatitis, Pityriasis rubra pilaris, pityriasis rosacea, parapsoriasis, Pityriasis lichenoiders, Lichen planus, Lichen nitidus, Ichthyosiform dermatosis, keratodermas, dermatosis, alopecia areata, pyoderma gangrenosum, vitiligo, pemphigoid (e.g., ocular cicatricial pemphigoid or bullous pemphigoid), urticaria, prokeratosis, rheumatoid arthritis that involves hyperproliferation and inflammation of epithelial-related cells lining the joint capsule; dermatitises such as seborrheic dermatitis and solar dermatitis; keratoses such as seborrheic keratosis, senile keratosis, actinic keratosis, photo-induced keratosis, and keratosis follicularis; acne vulgaris; keloids and prophylaxis against keloid formation; nevi; warts including verruca, condyloma or condyloma acuminatum, and human papilloma viral (HPV) infections such as venereal warts; leukoplakia; Lichen planus; and keratitis. The skin disorder can be dermatitis, for example, atopic dermatitis or allergic dermatitis, or psoriasis.

The compositions of the present disclosure (including pharmaceutically acceptable formulations comprising IL-10 agonist compounds and/or the nucleic acid molecules that encode them including recombinant viruses encoding such IL-10 agonist compounds) can also be administered to a patient who is suffering from (or may suffer from) psoriasis or psoriatic disorders. The term “psoriasis” is intended to have its medical meaning, namely, a disease which afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling. Hyperproliferation of keratinocytes is a key feature of psoriatic epidermal hyperplasia along with epidermal inflammation and reduced differentiation of keratinocytes. Multiple mechanisms have been invoked to explain the keratinocyte hyperproliferation that characterizes psoriasis. Disordered cellular immunity has also been implicated in the pathogenesis of psoriasis. Examples of psoriatic disorders include chronic stationary psoriasis, plaque psoriasis, moderate to severe plaque psoriasis, psoriasis vulgaris, eruptive psoriasis, psoriatic erythroderma, generalized pustular psoriasis, annular pustular psoriasis, or localized pustular psoriasis.

### Treatment of Neoplastic Disease

The present disclosure provides methods of use of IL-10 agonist compounds in the treatment of a subject suffering from a neoplastic disease disorder or condition by the administration of a therapeutically effective amount of an IL-10 agonist compound (or nucleic acid encoding an IL-10 agonist compound including recombinant vectors encoding IL-10 agonist compounds) as described herein.

The compositions and methods of the present disclosure are useful in the treatment of subject suffering from a neoplastic disease characterized by the presence neoplasms, including benign and malignant neoplasms, and neoplastic disease.

Examples of benign neoplasms amenable to treatment using the compositions and methods of the present disclosure include but are not limited to adenomas, fibromas, hemangiomas, and lipomas. Examples of pre-malignant neoplasms amenable to treatment using the compositions and methods of the present disclosure include but are not limited to hyperplasia, atypia, metaplasia, and dysplasia. Examples of malignant neoplasms amenable to treatment using the compositions and methods of the present disclosure include but are not limited to carcinomas (cancers arising from epithelial tissues such as the skin or tissues that line internal organs), leukemias, lymphomas, and sarcomas typically derived from bone fat, muscle, blood vessels or connective tissues). Also included in the term neoplasms are viral induced neoplasms such as warts and EBV induced disease (i.e., infectious mononucleosis), scar formation, hyperproliferative vascular disease including intimal smooth muscle cell hyperplasia, restenosis, and vascular occlusion and the like.

The term “neoplastic disease” includes cancers characterized by solid tumors and non-solid tumors including but not limited to breast cancers; sarcomas (including but not limited to osteosarcomas and angiosarcomas and fibrosarcomas), leukemias, lymphomas, genitourinary cancers (including but not limited to ovarian, urethral, bladder, and prostate cancers); gastrointestinal cancers (including but not limited to colon esophageal and stomach cancers); lung cancers; myelomas; pancreatic cancers; liver cancers; kidney cancers; endocrine cancers; skin cancers; and brain or central and peripheral nervous (CNS) system tumors, malignant or benign, including gliomas and neuroblastomas, astrocytomas, myelodysplastic disorders; cervical carcinoma-in-situ; intestinal polyposes; oral leukoplakias; histiocytoses, hyperprofroliferative scars including keloid scars, hemangiomas; hyperproliferative arterial stenosis, psoriasis, inflammatory arthritis; hyperkeratoses and papulosquamous eruptions including arthritis.

The term neoplastic disease includes carcinomas. The term “carcinoma” refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The term neoplastic disease includes adenocarcinomas. An “adenocarcinoma” refers to a carcinoma derived from glandular tissue or in which the tumor cells form recognizable glandular structures.

As used herein, the term “hematopoietic neoplastic disorders” refers to neoplastic diseases involving hyperplastic/neoplastic cells of hematopoietic origin, for example, arising from myeloid, lymphoid or erythroid lineages, or precursor cells thereof.

Myeloid neoplasms include, but are not limited to, myeloproliferative neoplasms, myeloid and lymphoid disorders with eosinophilia, myeloproliferative/myelodysplastic neoplasms, myelodysplastic syndromes, acute myeloid leukemia and related precursor neoplasms, and acute leukemia of ambiguous lineage. Exemplary myeloid disorders amenable to treatment in accordance with the present disclosure include, but are not limited to, acute promyeloid leukemia (APML), acute myelogenous leukemia (AML) and chronic myelogenous leukemia (CML).

Lymphoid neoplasms include, but are not limited to, precursor lymphoid neoplasms, mature B-cell neoplasms, mature T-cell neoplasms, Hodgkin's Lymphoma, and immunodeficiency-associated lymphoproliferative disorders. Exemplary lymphic disorders amenable to treatment in accordance with the present disclosure include, but are not limited to, acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM).

In some instances, the hematopoietic neoplastic disorder arises from poorly differentiated acute leukemias (e.g., erythroblastic leukemia and acute megakaryoblastic leukemia). As used herein, the term “hematopoietic neoplastic disorders” refers malignant lymphomas including, but are not limited to, non-Hodgkins lymphoma and variants thereof, peripheral T cell lymphomas, adult T-cell leukemia/lymphoma (ATL), cutaneous T cell lymphoma (CTCL), large granular lymphocytic leukemia (LGF), Hodgkin's disease and Reed-Steinberg disease.

The determination of whether a subject is “suffering from a neoplastic disease” refers to a determination made by a physician with respect to a subject based on the available information accepted in the field for the identification of a disease, disorder or condition including but not limited to X-ray, CT-scans, conventional laboratory diagnostic tests (e.g., blood count, etc.), genomic data, protein expression data, immunohistochemistry, that the subject requires or will benefit from treatment.

The adaptive immune system recognizes the display of certain cell surface proteins in response to tumor mutations facilitating the recognition and elimination of neoplastic cells. Tumors that possess a higher tumor mutation burden (TMB) are more likely to exhibit such “tumor antigens.” Indeed, clinical experience shows that tumors comprised of neoplastic cells exhibiting a high tumor mutation burden are more likely to respond to immune therapies, including immune checkpoint blockade (Rizvi et al. 2015. Science. 348(6230):124-128; Marabelle et al. 2020. Lancet Oncol. 21(10):1353-1365). The tumor mutation burden is useful as a biomarker to identify tumors with increased sensitivity to immune therapies such as those provided in the present disclosure.

In some embodiments, the compositions and methods of the present disclosure are useful in the treatment of neoplastic disease associated with the formation of solid tumors exhibiting an intermediate or high tumor mutational burden (TMB). In some embodiments, the compositions and compositions and methods of the present disclosure are useful in the treatment of immune-sensitive solid tumors exhibiting an intermediate or high tumor mutational burden (TMB). Examples of neoplastic diseases associated with the formation of solid tumors having an intermediate or high tumor mutational burden amenable to treatment with the compositions and methods of the present disclosure include but are not limited to non-small cell lung cancer and renal cell cancer. In one embodiment, the compositions and methods are useful in the treatment of non-small cell lung cancer (NSCLC) exhibiting an intermediate or high TMB. NSCLC cells typically harbor a significant number of mutations and are therefore more sensitive to immune therapies. The current standard of care for NSCLC is stratified by the cancer-initiating mechanisms and generally follows the recommendations of NCCN or ASCO. A large proportion of NSCLC has increased TMB and is therefore initially more sensitive to immune therapies. However, most tumors eventually relapse on immune checkpoint inhibition. Patients with relapsed tumors typically show reduced T cell infiltration in the tumor, systemic T cell exhaustion and a suppressed immune response compared to the lesions prior to immune checkpoint inhibition. Therefore, improved immune therapies are required, re-activating and expanding the exhausted, rare tumor-infiltrating T cells.

Combination of IL-10 Agonist Compounds with Supplemental Therapeutic Agents

The present disclosure provides for the use of the IL-10 agonist compounds (e.g., single-domain antibody polypeptides of the IL-10 agonist compounds) of the present disclosure in combination with one or more additional active agents (“supplemental agents”). Such further combinations are referred to interchangeably as “supplemental combinations” or “supplemental combination therapy” and those therapeutic agents that are used in combination with IL-10 agonist compounds of the present disclosure are referred to as “supplemental agents.” As used herein, the term “supplemental agents” includes agents that can be administered or introduced separately, for example, formulated separately for separate administration (e.g., as may be provided in a kit) and/or therapies that can be administered or introduced in combination with the hIL-10 agonist compounds.

As used herein, the term “in combination with” when used in reference to the administration of multiple agents to a subject refers to the administration of a first agent at least one additional (i.e. second, third, fourth, fifth, etc.) agent to a subject. For purposes of the present invention, one agent (e.g., hIL-10 agonist compound) is considered to be administered in combination with a second agent (e.g., a modulator of an immune checkpoint pathway) if the biological effect resulting from the administration of the first agent persists in the subject at the time of administration of the second agent such that the therapeutic effects of the first agent and second agent overlap. For example, the PD1 immune checkpoint inhibitors (e.g., nivolumab or pembrolizumab) are typically administered by IV infusion every two weeks or every three weeks while the hIL-10 agonist compounds of the present disclosure are typically administered more frequently, e.g., daily, BID, or weekly. However, the administration of the first agent (e.g., pembrolizumab) provides a therapeutic effect over an extended time and the administration of the second agent (e.g., an hIL-10 agonist compound) provides its therapeutic effect while the therapeutic effect of the first agent remains ongoing such that the second agent is considered to be administered in combination with the first agent, even though the first agent may have been administered at a point in time significantly distant (e.g., days or weeks) from the time of administration of the second agent. In one embodiment, one agent is considered to be administered in combination with a second agent if the first and second agents are administered simultaneously (within 30 minutes of each other), contemporaneously, or sequentially. In some embodiments, a first agent is deemed to be administered “contemporaneously” with a second agent if the first and second agents are administered within about 24 hours of each other, preferably within about 12 hours of each other, preferably within about 6 hours of each other, preferably within about 2 hours of each other, or preferably within about 30 minutes of each other. The term “in combination with” shall also be understood to apply to the situation where a first agent and a second agent are co-formulated in a single pharmaceutically acceptable formulation and the co-formulation is administered to a subject. In certain embodiments, the hIL-10 agonist compound and the supplemental agent(s) are administered or applied sequentially, for example, where one agent is administered prior to one or more other agents. In other embodiments, the hIL-10 agonist compound and the supplemental agent(s) are administered simultaneously, for example, where two or more agents are administered at or about the same time; the two or more agents may be present in two or more separate formulations or combined into a single formulation (i.e., a co-formulation). Regardless of whether the agents are administered sequentially or simultaneously, they are considered to be administered in combination for purposes of the present disclosure.

### Establishing Optimum Combinatorial Therapies

Further embodiments comprise a method or model for determining the optimum amount of an agent(s) in a combination. An optimum amount can be, for example, an amount that achieves an optimal effect in a subject or subject population, or an amount that achieves a therapeutic effect while minimizing or eliminating the adverse effects associated with one or more of the agents. In some embodiments, the methods involving the combination of an hIL-10 agonist compound and a supplemental agent which is known to be, or has been determined to be, effective in treating or preventing a disease, disorder, or condition described herein (e.g., a cancerous condition) in a subject (e.g., a human) or a subject population, and an amount of one agent is titrated while the amount of the other agent(s) is held constant. By manipulating the amounts of the agent(s) in this manner, a clinician is able to determine the ratio of agents most effective for, for example, treating a particular disease, disorder or condition, or eliminating the adverse effects or reducing the adverse effects such that are acceptable under the circumstances.

### Supplemental Agents Useful in the Treatment of Inflammatory or Autoimmune Disorders

In some embodiments, the method further comprises administering the IL-10 agonist compound (e.g., a single-domain antibody polypeptide of an IL-10 agonist compound) of the present disclosure in combination with one or more supplemental agents selected from the group consisting of a corticosteroid, a Janus kinase inhibitor, a calcineurin inhibitor, a mTor inhibitor, an IMDH inhibitor, a biologic, a vaccine, and a therapeutic antibody. In certain embodiments, the therapeutic antibody is an antibody that binds a protein selected from the group consisting of BLyS, CD11a, CD20, CD25, CD3, CD52, IgEIL-12/IL-23, IL-17α, IL-1β, IL-4Rα, IL-5, IL-6R, integrin-α4β7, RANKL, TNFα, VEGF-A, and VLA-4.

In some embodiments, the supplemental agent is one or more agents selected from the group consisting of corticosteroids (including but not limited to prednisone, budesonide, prednilisone), Janus kinase inhibitors (including but not limited to tofacitinib (Xeljanz®), calcineurin inhibitors (including but not limited to cyclosporine and tacrolimus), mTor inhibitors (including but not limited to sirolimus and everolimus), IMDH inhibitors (including but not limited to azathioprine, leflunomide and mycophenolate), biologics such as abatcept (Orencia®) or etanercept (Enbrel®), and therapeutic antibodies.

Examples of therapeutic antibodies that may be administered as supplemental agents in combination with the IL-10 agonist compounds (e.g., single-domain antibody polypeptides of the IL-10 agonist compounds) of the present disclosure in the treatment of autoimmune disease include but are not limited to anti-CD25 antibodies (e.g., daclizumab and basiliximab), anti-VLA-4 antibodies (e.g., natalizumab), anti-CD52 antibodies (e.g., alemtuzumab), anti-CD20 antibodies (e.g., rituximab, ocrelizumab), anti-TNF antibodies (e.g., infliximab, and adalimumab), anti-IL6R antibodies (e.g., tocilizumab), anti-TNFα antibodies (e.g., adalimumab (Humira®), golimumab, and infliximab), anti-integrin-α4β7 antibodies (e.g., vedolizumab), anti-IL17α antibodies (e.g., brodalumab or secukinumab), anti-IL-4Rα antibodies (e.g., dupilumab), anti-RANKL antibodies, IL-6R antibodies, anti-IL-1B antibodies (e.g., canakinumab), anti-CD11a antibodies (e.g., efalizumab), anti-CD3 antibodies (e.g., muramonab), anti-IL-5 antibodies (e.g., mepolizumab, reslizumab), anti-BlyS antibodies (e.g., belimumab); and anti-IL-12/IL-23 antibodies (e.g ustekinumab). Many therapeutic antibodies have been approved for clinical use against autoimmune disease. Examples of antibodies approved by the United States Food and Drug Administration (FDA) for use in the treatment of autoimmune diseases in a subject suffering therefrom that may be administered as supplemental agents in combination with the IL-10 agonist compounds (e.g., single-domain antibody polypeptides of the IL-10 agonist compounds) of the present disclosure (and optionally additional supplemental agents) for the treatment of the indicated autoimmune disease include but are not limited toatezolizumab, olaratumab, ixekizumab, trastuzumab, infliximab, rituximab, edrecolomab, daratumumab, elotuzumab, necitumumab, dinutuximab, nivolumab, blinatumomab, pembrolizumab, pertuzumab, brentuximab vedotin, ipilimumab, ofatumumab, certolizumab pegol, catumaxomab, panitumumab, bevacizumab, ramucirumab, siltuximab, enfortumab vedotin, polatuzumab vedotin, [fam]-trastuzumab deruxtecan, cemiplimab, moxetumomab pasudotox, mogamuizumab, tildrakizumab, ibalizumab, durvalumab, inotuzumab, ozogamicin, avelumab, simertinib, ado-trastuzumab emtansine, cetuximab, tositumomab-I131, ibritumomab tiuxetan, gemtuzumab, and ozogamicin.

The foregoing antibodies useful as supplemental agents in the practice of the methods of the present disclosure may be administered alone or in the form of any antibody drug conjugate (ADC) comprising the antibody, linker, and one or more drugs (e.g., 1, 2, 3, 4, 5, 6, 7, or 8 drugs) or in modified form (e.g., PEGylated).

### Supplemental Agents Useful in the Treatment of Neoplastic Disease

In some embodiments, the supplemental agent is a chemotherapeutic agent. In some embodiments, the supplemental agent is a “cocktail” of multiple chemotherapeutic agents. In some embodiments the chemotherapeutic agent or cocktail is administered in combination with one or more physical methods (e.g., radiation therapy). The term “chemotherapeutic agents” includes but is not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins such as bleomycin A₂, cactinomycin, calicheamicin, carabicin, caminomycin, carzinophilin, chromomycins, dactinomycin, daunorubicin and derivaties such as demethoxy-daunomycin, 11-deoxydaunorubicin, 13-deoxydaunorubicin, detorubicin, 6-diazo-5-oxo-L-norleucine, doxorubicin, epirubicin, esorubicin, idarubicin, marcellomycin, mitomycins such as mitomycin C, N-methyl mitomycin C; mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate, dideazatetrahydrofolic acid, and folinic acid; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, for example, paclitaxel, nab-paclitaxel and doxetaxel; chlorambucil; gemcitabine; 6-thioguanine; mercaptopurine; methotrexate; platinum and platinum coordination complexes such as cisplatin, oxaplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine (DMFO); retinoic acid; esperamicins; capecitabine; taxanes such as paclitaxel, docetaxel, cabazitaxel; carminomycin, adriamycins such as 4′-epiadriamycin, 4-adriamycin-14-benzoate, adriamycin-14-octanoate, adriamycin-14-naphthaleneacetate; cholchicine and pharmaceutically acceptable salts, acids or derivatives of any of the above.

The term “chemotherapeutic agents” also includes anti-hormonal agents that act to regulate or inhibit hormone action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, leuprolide, and goserelin; and pharmaceutically acceptable salts, acids or derivatives of any of the above.

In some embodiments, a supplemental agent isone or more chemical or biological agents identified in the art as useful in the treatment of neoplastic disease, including, but not limited to, a cytokines or cytokine antagonists such as IL-12, INFα, or anti-epidermal growth factor receptor, irinotecan; tetrahydrofolate antimetabolites such as pemetrexed; antibodies against tumor antigens, a complex of a monoclonal antibody and toxin, a T-cell adjuvant, bone marrow transplant, or antigen presenting cells (e.g., dendritic cell therapy), anti-tumor vaccines, replication competent viruses, signal transduction inhibitors (e.g., Gleevec® or Herceptin®) or an immunomodulator to achieve additive or synergistic suppression of tumor growth, non-steroidal anti-inflammatory drugs (NSAIDs), cyclooxygenase-2 (COX-2) inhibitors, steroids, TNF antagonists (e.g., Remicade® and Enbrel®), interferon-β1a (Avonex®), and interferon-β1b (Betaseron®) as well as combinations of one or more of the foregoing as practiced in known chemotherapeutic treatment regimens including but not limited to TAC, FOLFOX, TPC, FEC, ADE, FOLFOX-6, EPOCH, CHOP, CMF, CVP, BEP, OFF, FLOX, CVD, TC, FOLFIRI, PCV, FOLFOXIRI, ICE-V, XELOX, and others that are readily appreciated by the skilled clinician in the art.

In some embodiments, the hIL-10 agonist compound is administered in combination with BRAF/MEK inhibitors, kinase inhibitors such as sunitinib, PARP inhibitors such as olaparib, EGFR inhibitors such as osimertinib (Ahn et al. 2016. J Thorac Oncol. 11:S115), IDO inhibitors such as epacadostat, and oncolytic viruses such as talimogene laherparepvec (T-VEC).

In some embodiments, a “supplemental agent” is a therapeutic antibody (including bi-specific and tri-specific antibodies which bind to one or more tumor-associated antigens including but not limited to bispecific T cell engagers (BITEs), dual affinity retargeting (DART) constructs, and trispecific killer engager (TriKE) constructs).

In some embodiments, the therapeutic antibody is an antibody that binds to at least one tumor antigen selected from the group consisting of HER2 (e.g., trastuzumab, pertuzumab, ado-trastuzumab emtansine), nectin-4 (e.g., enfortumab), CD79 (e.g., polatuzumab vedotin), CTLA4 (e.g., ipilumumab), CD22 (e.g., moxetumomab pasudotox), CCR4 (e.g., magamuizumab), IL23p19 (e.g., tildrakizumab), PDL1 (e.g., durvalumab, avelumab, atezolizumab), IL-17a (e.g., ixekizumab), CD38 (e.g., daratumumab), SLAMF7 (e.g., elotuzumab), CD20 (e.g., rituximab, tositumomab, ibritumomab and ofatumumab), CD30 (e.g., brentuximab vedotin), CD33 (e.g., gemtuzumab ozogamicin), CD52 (e.g., alemtuzumab), EpCam, CEA, fpA33, TAG-72, CAIX, PSMA, PSA, folate binding protein, GD2 (e.g., dinuntuximab), GD3, IL-6 (e.g., silutxumab) GM2, Le^{y}, VEGF (e.g., bevacizumab), VEGFR, VEGFR2 (e.g., ramucirumab), PDGFRα (e.g., olartumumab), EGFR (e.g., cetuximab, panitumumab and necitumumab), ERBB2 (e.g., trastuzumab), ERBB3, MET, IGF1R, EPHA3, TRAIL R1, TRAIL R2, RANKL RAP, tenascin, integrin αVβ3, and integrin α4β1.

Examples of antibody therapeutics which are FDA approved and may be used as supplemental agents for use in the treatment of neoplastic disease include those provided in the table below.

| Name | Tradename(s) | Target; format | Indication |
|---|---|---|---|
| [fam]-trastuzumab | Enhertu | HER2; Humanized IgG1 ADC | HER2+ breast cancer |
| deruxtecan | | | |
| Enfortumab vedotin | Padcev | Nectin-4; Human IgG1 ADC | Urothelial cancer |
| Polatuzumab vedotin | Polivy | CD79β; Humanized IgG1 ADC | Diffuse large B-cell lymphoma |
| Cemiplimab | Libtayo | PD-1; Human mAb | Cutaneous squamous cell |
| | | | carcinoma |
| Moxetumomab | Lumoxiti | CD22; Murine IgG1 dsFv | Hairy cell leukemia |
| pasudotox | | immunotoxin | |
| Mogamuizumab | Poteligeo | CCR4; Humanized IgG1 | Cutaneous T cell lymphoma |
| Tildrakizumab | Ilumya | IL-23p19; Humanized IgG1 | Plaque psoriasis |
| Ibalizumab | Trogarzo | CD4; Humanized IgG4 | HIV infection |
| Durvalumab | IMFINZI | PD-L1; Human IgG1 | Bladder cancer |
| Inotuzumab | BESPONSA | CD22; Humanized IgG4, ADC | Hematological malignancy |
| ozogamicin | | | |
| Avelumab | Bavencio | PD-L1; Human IgG1 | Merkel cell carcinoma |
| Atezolizumab | Tecentriq | PD-L1; Humanized IgG1 | Bladder cancer |
| Olaratumab | Lartruvo | PDGRFα; Human IgG1 | Soft tissue sarcoma |
| Ixekizumab | Taltz | IL-17α; Humanized IgG4 | Psoriasis |
| Daratumumab | Darzalex | CD38; Human IgG1 | Multiple myeloma |
| Elotuzumab | Empliciti | SLAMF7; Humanized IgG1 | Multiple myeloma |
| Necitumumab | Portrazza | EGFR; Human IgG1 | Non-small cell lung cancer |
| Dinutuximab | Unituxin | GD2; Chimeric IgG1 | Neuroblastoma |
| Nivolumab | Opdivo | PD1; Human IgG4 | Melanoma, non-small cell lung |
| | | | cancer |
| Blinatumomab | Blincyto | CD19, CD3; Murine bispecific | Acute lymphoblastic leukemia |
| | | tandem scFv | |
| Pembrolizumab | Keytruda | PD1; Humanized IgG4 | Melanoma |
| Ramucirumab | Cyramza | VEGFR2; Human IgG1 | Gastric cancer |
| Siltuximab | Sylvant | IL-6; Chimeric IgG1 | Castleman disease |
| Obinutuzumab | Gazyva | CD20; Humanized IgG1; | Chronic lymphocytic leukemia |
| | | Glycoengineered | |
| Ado-trastuzumab | Kadcyla | HER2; Humanized IgG1, ADC | Breast cancer |
| emtansine | | | |
| Pertuzumab | Perjeta | HER2; Humanized IgG1 | Breast Cancer |
| Brentuximab vedotin | Adcetris | CD30; Chimeric IgG1, ADC | Hodgkin lymphoma, systemic |
| | | | anaplastic large cell lymphoma |
| Ipilimumab | Yervoy | CTLA-4; Human IgG1 | Metastatic melanoma |
| Ofatumumab | Arzerra | CD20; Human IgG1 | Chronic lymphocytic leukemia |
| Certolizumab pegol | Cimzia | TNF; Humanized Fab, pegylated | Crohn disease |
| Catumaxomab | Removab | EPCAM/CD3; Rat/mouse | Malignant ascites |
| | | bispecific mAb | |
| Panitumumab | Vectibix | EGFR; Human IgG2 | Colorectal cancer |
| Bevacizumab | Avastin | VEGF; Humanized IgG1 | Colorectal cancer |
| Cetuximab | Erbitux | EGFR; Chimeric IgG1 | Colorectal cancer |
| Tositumomab-1131 | Bexxar | CD20; Murine IgG2a | Non-Hodgkin lymphoma |
| Ibritumomab | Zevalin | CD20; Murine IgG1 | Non-Hodgkin lymphoma |
| tiuxetan | | | |
| Gemtuzumab | Mylotarg | CD33; Humanized IgG4, ADC | Acute myeloid leukemia |
| ozogamicin | | | |
| Trastuzumab | Herceptin | HER2; Humanized IgG1 | Breast cancer |
| Infliximab | Remicade | TNF; Chimeric IgG1 | Crohn disease |
| Rituximab | MabThera, | CD20; Chimeric IgG1 | Non-Hodgkin lymphoma |
| | Rituxan | | |
| Edrecolomab | Panorex | EpCAM; Murine IgG2a | Colorectal cancer |

In some embodiments, where the antibody is a bispecific antibody targeting a first and second tumor antigen such as HER2 and HER3 (abbreviated HER2×HER3), FAP×DR-5 bispecific antibodies, CEA×CD3 bispecific antibodies, CD20×CD3 bispecific antibodies, EGFR-EDV-miR16 trispecific antibodies, gp100×CD3 bispecific antibodies, Ny-eso×CD3 bispecific antibodies, EGFR×cMet bispecific antibodies, BCMA×CD3 bispecific antibodies, EGFR-EDV bispecific antibodies, CLEC12A×CD3 bispecific antibodies, HER2×HER3 bispecific antibodies, Lgr5×EGFR bispecific antibodies, PD1×CTLA-4 bispecific antibodies, CD123×CD3 bispecific antibodies, gpA33×CD3 bispecific antibodies, B37-H-3×CD3 bispecific antibodies, LAG-3×PD1 bispecific antibodies, DLL4×VEGF bispecific antibodies, Cadherin-P×CD3 bispecific antibodies, BCMA×CD3 bispecific antibodies, DLL4×VEGF bispecific antibodies, CD20×CD3 bispecific antibodies, Ang-2×VEGF-A bispecific antibodies, CD20×CD3 bispecific antibodies, CD123×CD3 bispecific antibodies, SSTR2×CD3 bispecific antibodies, PD1×CTLA-4 bispecific antibodies, HER2×HER2 bispecific antibodies, GPC3×CD3 bispecific antibodies, PSMA×CD3 bispecific antibodies, LAG-3×PD-L1 bispecific antibodies, CD38×CD3 bispecific antibodies, HER2×CD3 bispecific antibodies, GD2×CD3 bispecific antibodies, and CD33×CD3 bispecific antibodies. Such therapeutic antibodies may be further conjugated to one or more chemotherapeutic agents (e.g antibody drug conjugates or ADCs) directly or through a linker, especially acid, base or enzymatically labile linkers.

In some embodiments, a supplemental agent is one or more non-pharmacological modalities (e.g., localized radiation therapy or total body radiation therapy or surgery). By way of example, the present disclosure contemplates treatment regimens wherein a radiation phase is preceded or followed by treatment with a treatment regimen comprising an IL-10 agonist compound and one or more supplemental agents. In some embodiments, the present disclosure further contemplates the use of an IL-10 agonist compound in combination with surgery (e.g., tumor resection). In some embodiments, the present disclosure further contemplates the use of an IL-10 agonist compound in combination with bone marrow transplantation, peripheral blood stem cell transplantation or other types of transplantation therapy.

In some embodiments, a “supplemental agent” is an immune checkpoint modulator for the treatment and/or prevention neoplastic disease in a subject as well as diseases, disorders or conditions associated with neoplastic disease. The term “immune checkpoint pathway” refers to biological response that is triggered by the binding of a first molecule (e.g., a protein such as PD1) that is expressed on an antigen presenting cell (APC) to a second molecule (e.g., a protein such as PDL1) that is expressed on an immune cell (e.g., a T-cell) which modulates the immune response, either through stimulation (e.g., upregulation of T-cell activity) or inhibition (e.g., downregulation of T-cell activity) of the immune response. The molecules that are involved in the formation of the binding pair that modulate the immune response are commonly referred to as “immune checkpoints.” The biological responses modulated by such immune checkpoint pathways are mediated by intracellular signaling pathways that lead to downstream immune effector pathways, such as cell activation, cytokine production, cell migration, cytotoxic factor secretion, and antibody production. Immune checkpoint pathways are commonly triggered by the binding of a first cell surface expressed molecule to a second cell surface molecule associated with the immune checkpoint pathway (e.g., binding of PD1 to PDL1, CTLA4 to CD28, etc.). The activation of immune checkpoint pathways can lead to stimulation or inhibition of the immune response.

In one embodiment, the immune checkpoint pathway modulator is an antagonist of a negative immune checkpoint pathway that inhibits the binding of PD1 to PDL1 and/or PDL2 (“PD1 pathway inhibitor”). PD1 pathway inhibitors result in the stimulation of a range of favorable immune response such as reversal of T-cell exhaustion, restoration cytokine production, and expansion of antigen-dependent T-cells. PD1 pathway inhibitors have been recognized as effective variety of cancers receiving approval from the USFDA for the treatment of variety of cancers including melanoma, lung cancer, kidney cancer, Hodgkins lymphoma, head and neck cancer, bladder cancer and urothelial cancer.

The term PD1 pathway inhibitors includes monoclonal antibodies that interfere with the binding of PD1 to PDL1 and/or PDL2. Antibody PD1 pathway inhibitors are well known in the art. Examples of commercially available PD1 pathway inhibitors that monoclonal antibodies that interfere with the binding of PD1 to PDL1 and/or PDL2 include nivolumab (Opdivo®, BMS-936558, MDX1106, commercially available from BristolMyers Squibb, Princeton NJ), pembrolizumab (Keytruda® MK-3475, lambrolizumab, commercially available from Merck and Company, Kenilworth NJ), and atezolizumab (Tecentriq®, Genentech/Roche, South San Francisco CA). Additional PD1 pathway inhibitors antibodies are in clinical development including but not limited to durvalumab (MEDI4736, Medimmune/AstraZeneca), pidilizumab (CT-011, CureTech), PDR001 (Novartis), BMS-936559 (MDX1105, BristolMyers Squibb), and avelumab (MSB0010718C, Merck Serono/Pfizer) and SHR-1210 (Incyte). Additional antibody PD1 pathway inhibitors are described in U.S. Pat. No. 8,217,149 (Genentech, Inc); U.S. Pat. No. 8,168,757 (Merck Sharp and Dohme Corp.), U.S. Pat. No. 8,008,449 (Medarex, Inc.), U.S. Pat. No. 7,943,743 (Medarex, Inc).

In some embodiments, the methods of the disclosure may include the combination of the administration of an IL-10 agonist compounds with supplemental agents in the form of cell therapies for the treatment of neoplastic, autoimmune or inflammatory diseases. Examples of cell therapies that are amenable to use in combination with the methods of the present disclosure include but are not limited to engineered T cell products comprising one or more activated CAR-T cells, engineered TCR cells, tumor infiltrating lymphocytes (TILs), engineered Treg cells. As engineered T-cell products are commonly activated ex vivo prior to their administration to the subject and therefore provide upregulated levels of CD25, cell products comprising such activated engineered T cells types are amenable to further support via the administration of an CD25 biased IL-10 agonist compound as described herein. Examples of commercially available CAR-T cell products that may be modified to incorporate an orthogonal receptor of the present invention include axicabtagene ciloleucel (marketed as Yescarta® commercially available from Gilead Pharmaceuticals) and tisagenlecleucel (marketed as Kymriah® commercially available from Novartis).

### Prophylactic Applications

In some embodiments where the IL-10 agonist compound (e.g., a single-domain antibody polypeptide of an IL-10 agonist compound) is used in prophylaxis of disease, the supplementary agent may be a vaccine. The IL-10 agonist compound (e.g., a single-domain antibody polypeptide of an IL-10 agonist compound) of the present invention may be administered to a subject in combination with vaccines as an adjuvant to enhance the immune response to the vaccine in accordance with the teaching of Doyle, et al U.S. Pat. No. 5,800,819. Examples of vaccines that may be combined with the IL-10 agonist compound (e.g., a single-domain antibody polypeptide of an IL-10 agonist compound) of the present invention include are HSV vaccines, Bordetella pertussis, Escherichia coli vaccines, pneumococcal vaccines including multivalent pneumococcal vaccines such as Prevnar® 13, diptheria, tetanus and pertussis vaccines (including combination vaccines such as Pediatrix®) and Pentacel®), varicella vaccines, Haemophilus influenzae type B vaccines, human papilloma virus vaccines such as Garasil®, polio vaccines, Leptospirosis vaccines, combination respiratory vaccine, Moraxella vaccines, and attenuated live or killed virus vaccine products such as bovine respiratory disease vaccine (RSV), multivalent human influenza vaccines such as Fluzone® and Quadravlent Fluzone®), feline leukemia vaccine, transmissible gastroenteritis vaccine, COVID-19 vaccine, and rabies vaccine.

### EXAMPLES

### Example 1—Synthesis of DNA Encoding IL-10 Agonist Compounds

DNA was synthesized consisting of a mouse IGHV3 signal peptide and nucleic acid sequences of Table 6 (SEQ ID NOS:190-211), encoding the IL-10R single-domain antibodies of Table 1 (SEQ ID NOS:1-24), which included nucleic acid sequence encoding an anti-IL-10Rα VHH antibody fragment and one nucleic acid sequence encoding an anti-IL-10Rβ VHH antibody fragment separated by a linker sequence by GGGS (SEQ ID NO: 439). An Ala-Ser (“AS”) linker was constructed and used to join Histidine tag molecules to the C-terminus, encoding DR2463 (SEQ ID NO:1). The codon-optimized DNA sequences encoding these constructs are provided as SEQ ID NOS:200-223.

### Example 2—Recombinant Production and Purification

Codon optimized DNA inserts (SEQ ID NOS:200-223) were generated and cloned into modified pcDNA3.4 (Genscript) for small scale expression in HEK293 cells in 24 well plates. Supernatants were collected and IL-10 agonist proteins were purified in substantial accordance with the following procedure. Using a Hamilton Star automated system, 96×4 ml of supernatants in 4×24-well blocks were re-arrayed into 4×96-well, 1 mL blocks. PhyNexus micropipette tips (Biotage, San Jose CA) holding 80 μL of Ni-Excel IMAC resin (Cytiva) are equilibrated wash buffer: PBS pH 7.4, 30 mM imidazole. PhyNexus tips were dipped and cycled through 14 cycles of 1 mL pipetting across all 4×96-well blocks. PhyNexus tips were washed in 2×1 mL blocks holding wash buffer. PhyNexus tips were eluted in 3×0.36 mL blocks holding elution buffer: PBS pH 7.4, 400 mM Imidazole. PhyNexus tips were regenerated in 3×1 mL blocks of 0.5 M sodium hydroxide.

The purified protein eluates were quantified using a Biacore® T200 as in substantial accordance with the following procedure. 10 μL of the first 96×0.36 mL eluates were transferred to a Biacore® 96-well microplate and diluted to 60 uL in HBS-EP+ buffer (10 mM Hepes pH 7.4, 150 mM NaCl, 1 mM EDTA, 0.05% Tween 20). Each of the samples was injected on a CM5 series S chip previously functionalized with anti-histidine capture antibody (Cytiva): injection is performed for 18 seconds at 5 uL/min. Capture levels were recorded 60 seconds after buffer wash. A standard curve of known VHH concentrations (270, 90, 30, 10, 3.3, 1.1 pg/mL) was acquired in each of the 4 Biacore chip flow cells to eliminate cell-to-cell surface variability. The captures were interpolated against the standard curve using a non-linear model including specific and unspecific, one-site binding. Concentrations in the first elution block varied from 12 to 452 μg/mL corresponding to a 4-149 pg. SDS-PAGE analysis of 5 randomly picked samples was performed to ensure molecular weight of eluates corresponded to expected values (~30 KDa).

The concentration of the proteins was normalized using the Hamilton Star automated system in substantial accordance with the following procedure. Concentration values are imported in an Excel spreadsheet where pipetting volumes were calculated to perform dilution to 50 μg/mL in 0.22 mL. The spreadsheet was imported in a Hamilton Star method dedicated to performing dilution pipetting using the first elution block and elution buffer as diluent. The final, normalized plate was sterile filtered using 0.22 μm filter plates (Corning) and the material used for the following in vitro assays.

### Example 3—Evaluation of Binding Affinity of Humanized IL-10 Agonist Compounds Via Surface Plasmon Resonance

The IL-10 agonist compounds (e.g., single-domain antibody polypeptides of the IL-10 agonist compounds) of Table 1 were evaluated for binding via SPR as follows. Experiments were performed on a Biacore T200 instrument (Cytiva). Fe-fused IL-10Rα and IL-10Rβ receptors (Sino Biologicals, RnD) were immobilized as ligands (1-5 μg/mL) on Anti-human capture (AHC) or Protein G chip (Cytiva) at 5 μL/min for 18-60 seconds achieving the load level (RU) listed in the Tables 17 and 18. Dual VHHs were flowed (analytes) at 30 min in high performance mode as a two-fold dilution series from 800 to 12.5 nM. Sensograms were double-referenced by subtracting one or more cycles of buffer injection (no analyte) and one of a ligand-free flow cell.

Data was analyzed in Biacore Eval software and globally fit with a 1:1 binding model. Tables 17 and 18 show that all constructs (except DR2499, DR2504 in Table 17) demonstrated binding activity.

TABLE A
Humanized IL-10 agonist Protein Binding Affinity (hzIL-10Rα-Fc)

| | | | | Affinity | Rmax | Load | Surface |
|---|---|---|---|---|---|---|---|
| Analyte | Ligand | k_{ON} (1/Ms) | k_{OFF} (1/s) | (nM) | (RU) | (RU) | Activity |
| DR2499 | hIL-10Rα-Fc | | | No | | 110.9 | N/A |
| | (RnD) | | | binding | | | |
| DR2500 | | 5.8E+04 | 9.0E−04 | 15.5 | 36 | 110.8 | 41% |
| DR2501 | | 8.3E+04 | 1.4E−02 | 172.0 | 30.6 | 110.8 | 35% |
| DR2502 | | 1.2E+05 | 1.3E−03 | 11.2 | 31 | 110.7 | 35% |
| DR2503 | | 1.7E+05 | 3.3E−04 | 1.9 | 37.2 | 110.6 | 42% |
| DR2504 | | | | No | | 110.7 | N/A |
| | | | | binding | | | |
| DR2505 | | 7.9E+04 | 2.4E−03 | 30.2 | 28.7 | 110.6 | 32% |
| DR2506 | | 5.0E+04 | 2.6E−03 | 52.8 | 33.1 | 110.7 | 37% |
| DR2463 | | 1.6E+05 | 4.2E−04 | 2.7 | 36.6 | 110.8 | 41% |
| DR841 | | 2.5E+05 | 5.3E−04 | 2.1 | 35.6 | 110.6 | 40% |
| DR2520 | hIL-10Rα-Fc | 1.3E+05 | 5.2E−04 | 4.0 | 40 | 137 | 37% |
| DR2519 | (RnD) | 1.3E+05 | 5.4E−04 | 4.2 | 40 | 128 | 39% |
| DR2485 | | 7.3E+04 | 6.4E−04 | 8.8 | 39 | 132 | 37% |
| DR2457 | hIL-10Rα-Fc | 2.6E+05 | 3.9E−04 | 1.5 | 42 | 115 | |
| DR841 | (RnD) | 2.8E+05 | 6.1E−04 | 2.2 | 39 | 114 | |
| DR2463 | | 2.3E+05 | 6.0E−04 | 2.6 | 39 | 114 | |
| DR2459 | | 2.2E+05 | 6.0E−04 | 2.8 | 36 | 114 | |
| DR2458 | | 1.5E+05 | 4.6E−04 | 3.1 | 42 | 115 | |
| DR2462 | | 1.7E+05 | 5.6E−04 | 3.4 | 39 | 114 | |
| DR2460 | | 1.5E+05 | 5.2E−04 | 3.5 | 40 | 114 | |
| DR2464 | | 1.1E+05 | 5.9E−04 | 5.4 | 37 | 114 | |
| DR2461 | | 9.9E+04 | 5.7E−04 | 5.8 | 40 | 114 | |
| DR1525 | | 6.0E+04 | 9.3E−04 | 15.4 | 38 | 114 | |
| DR2466 | | 4.0E+04 | 1.1E−03 | 26.2 | 39 | 114 | |
| DR2465 | | 3.3E+04 | 1.1E−03 | 34.1 | 44 | 114 | |

TABLE B
Humanized IL-10 agonist Protein Binding Affinity (hzIL-10Rβ-Fc)

| | | k_{ON} | k_{OFF} | Affinity | Rmax | Load | Surface |
|---|---|---|---|---|---|---|---|
| Analyte | Ligand | (1/Ms) | (1/s) | (nM) | (RU) | (RU) | Activity |
| DR2499 | hIL-10Rβ-Fc | 9.9E+04 | 1.2E−03 | 12.3 | 65.9 | 191 | 49% |
| DR2500 | (SinoBiol) | 6.1E+04 | 1.4E−03 | 22.4 | 66.6 | 191 | 50% |
| DR2501 | | 5.2E+04 | 1.4E−03 | 27.2 | 68.2 | 191 | 51% |
| DR2502 | | 1.0E+05 | 1.2E−03 | 11.1 | 65.9 | 191 | 49% |
| DR2503 | | 6.7E+04 | 1.4E−03 | 20.4 | 67.5 | 191 | 51% |
| DR2504 | | 7.0E+04 | 1.3E−03 | 18.0 | 67.4 | 191 | 51% |
| DR2505 | | 7.1E+04 | 1.1E−03 | 15.9 | 63.9 | 191 | 48% |
| DR2506 | | 4.7E+04 | 1.3E−03 | 27.2 | 67.2 | 191 | 50% |
| DR2463 | | 7.2E+04 | 1.4E−03 | 19.7 | 68.5 | 191 | 51% |
| DR841 | | 1.0E+05 | 6.0E−04 | 6.0 | 68.4 | 190 | 51% |
| DR2520 | hIL-10Rβ-Fc | 8.0E+04 | 1.4E−03 | 18.0 | 46 | 150 | 36% |
| DR2519 | (SinoBiol) | 5.4E+04 | 1.6E−03 | 29.0 | 45 | 139 | 38% |
| DR2485 | | 4.4E+04 | 1.6E−03 | 35.0 | 44 | 144 | 36% |
| DR841 | hIL-10Rβ-Fc | 9.7E+04 | 6.3E−04 | 6.5 | 86 | 216 | |
| DR2457 | (SinoBiol) | 1.1E+05 | 1.2E−03 | 11.0 | 89 | 219 | |
| DR2463 | | 1.1E+05 | 1.5E−03 | 13.9 | 86 | 217 | |
| DR2459 | | 9.3E+04 | 1.5E−03 | 16.0 | 86 | 217 | |
| DR2462 | | 8.5E+04 | 1.5E−03 | 17.7 | 86 | 217 | |
| DR1525 | | 6.8E+04 | 1.2E−03 | 18.3 | 85 | 216 | |
| DR2465 | | 6.9E+04 | 1.4E−03 | 20.8 | 86 | 216 | |
| DR2466 | | 6.4E+04 | 1.4E−03 | 22.3 | 84 | 216 | |
| DR2458 | | 6.5E+04 | 1.5E−03 | 23.3 | 86 | 217 | |
| DR2460 | | 6.2E+04 | 1.6E−03 | 24.9 | 84 | 217 | |
| DR2464 | | 5.6E+04 | 1.5E−03 | 27.1 | 83 | 216 | |
| DR2461 | | 5.4E+04 | 1.5E−03 | 27.2 | 83 | 217 | |

### Example 4—IL-10 Agonist Protein Activity Assay in Monocytes

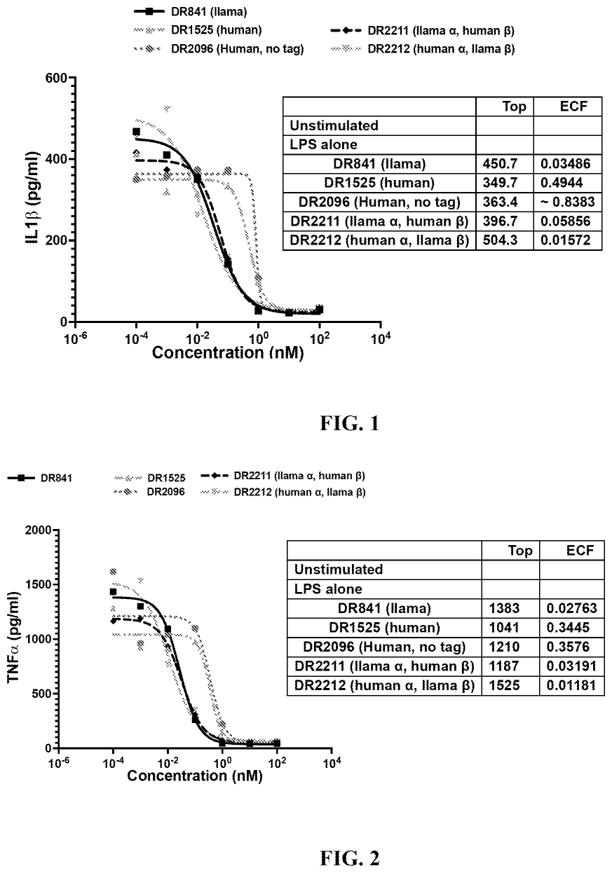

Human PBMCs were isolated from LRS chambers obtained from non-smoking human donors by Ficoll gradient purification. Human Monocytes were purified from the PBMCs by positive selection using Human CD14 Microbeads (Miltenyi Biotech-130-050-201) following manufacturer's protocol. Monocytes were treated with 1 ng/ml of LPS (Millipore Sigma: L2630) in the presence of varying concentrations of the IL10Rα/IL10Rβ VHH dimers for 48 hours at 37° C. Post-treatment, cells were spun down at 400 g for 5 min and supernatants were collected. Supernatants were assessed for pro-inflammatory cytokines using the MSD kit (Meso Scale Discovery: K151A9H-4) following manufacturer's protocol. The data was graphed using Prism software. FIG. 1 and FIG. 2 show that the humanized SCAs DR1525 (humanized) and DR2096 (DR241 humanization construct DR1525 with L11S, untagged) were less potent than the parental llama molecule DR841 in an LPS-induced monocyte secretion assay.

### Example 5—IL-10 Agonist Protein Agonist Protein in Monocyte LPS Assay

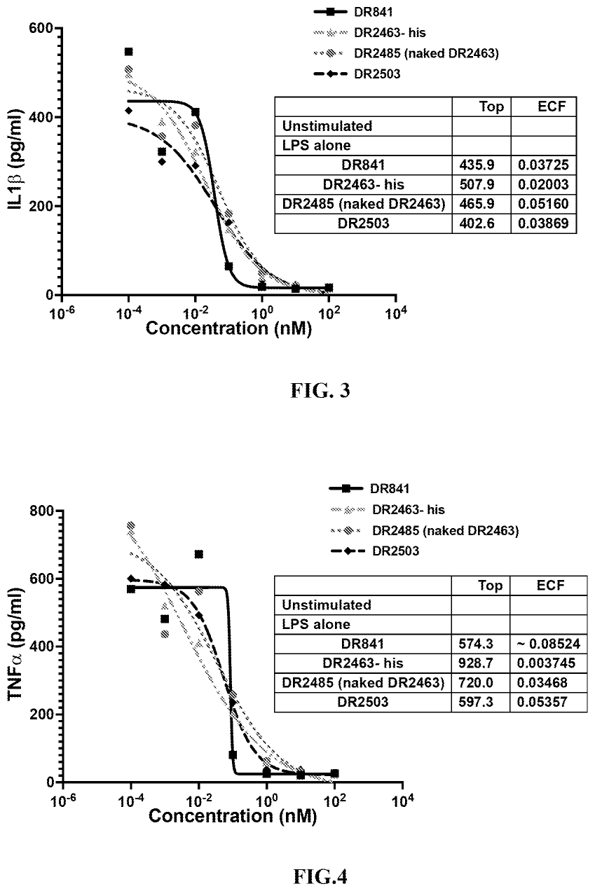

Human PBMCs were isolated from LRS chambers obtained from non-smoking human donors by Ficoll gradient purification. Human Monocytes were purified from the PBMCs by positive selection using Human CD14 Microbeads (Miltenyi Biotech-130-050-201) following manufacturer's protocol. Monocytes were treated with 1 ng/ml of LPS (Millipore Sigma: L2630) in the presence of varying concentrations of the IL10Rα/IL10Rβ VHH dimers for 48 hours at 37° C. Post-treatment, cells were spun down at 400 g for 5 min and supernatants were collected. Supernatants were assessed for pro-inflammatory cytokines using the MSD kit (Meso Scale Discovery: K151A9H-4) following manufacturer's protocol. The data was graphed using Prism software. FIG. 3 and FIG. 4 show that the humanized IL10Rα-IL10Rβ VHH dimer DR2503 (Table 1, SEQ ID NO:19) has a comparable potency to the parental llama molecule DR841 (Table 14, SEQ ID NO:465) in an LPS-induced monocyte secretion assay. DR2503 has the amino acid sequence of DR2463 (Table 1, SEQ ID NO:1) with resurfacing modifications R58Y in CDR2. The DR841 control is a non-humanized IL-10Rα/IL-10Rβ VHH dimer polypeptide variant having an Q1E and S31A amino acid substitution (to eliminate the Asn29-Cys30-Ser30 N-linked glycosylation motif) and has the amino acid sequence of SEQ ID NO:465 (Table 14).

### Example 6—IL-10 Agonist Protein Activity in Monocyte LPS Assay

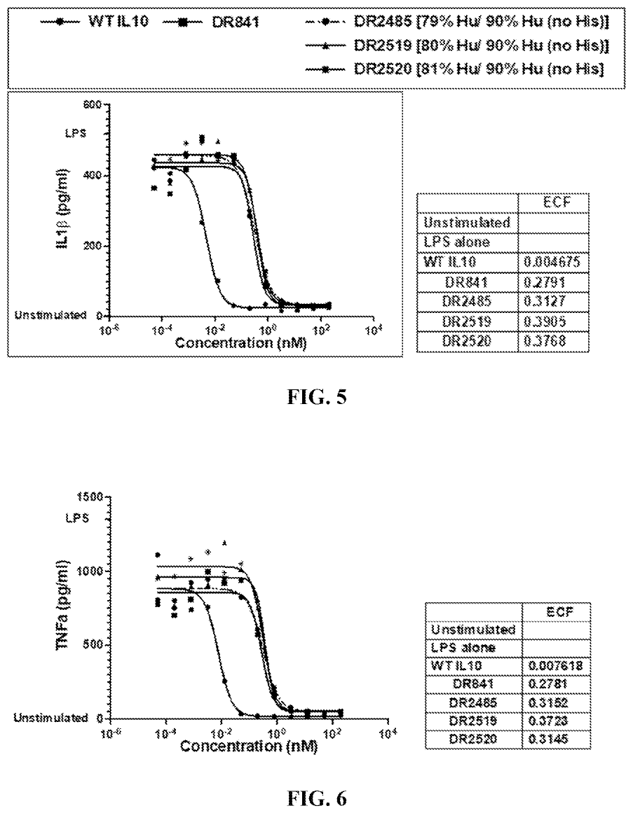

Human PBMCs were isolated from LRS chambers obtained from non-smoking human donors by Ficoll gradient purification. Human Monocytes were purified from the PBMCs by positive selection using Human CD14 Microbeads (Miltenyi Biotech-130-050-201) following manufacturer's protocol. Monocytes were treated with 1 ng/ml of LPS (Millipore Sigma: L2630) in the presence of varying concentrations of the IL10Rα/IL10Rβ VHH dimers for 48 hours at 37° C. Post-treatment, cells were spun down at 400 g for 5 min and supernatants were collected. Supernatants were assessed for pro-inflammatory cytokines IL-1p and TNFα using the MSD kit (Meso Scale Discovery: K151A9H-4) substantially following manufacturer's protocol. The data was graphed using Prism software, as shown in FIGS. 5 and 6, which show that humanized IL10Rα/IL10Rβ VHH dimers DR2485 (SEQ ID NO:2), DR2519 (SEQ ID NO:3), and DR2520 (SEQ ID NO:4) in Table 1 retain the ability to suppress the secretion of proinflammatory cytokines IL-1β and TNFα similar to the non-humanized parental VHH dimer DR841 (Table 14, SEQ ID NO:465).

### Example 7—IL-10 Agonist Protein Activity Assay in T-Cells

Human PBMCs were isolated from LRS chambers obtained from non-smoking human donors by Ficoll gradient purification. Human CD8 T cells were purified from the PBMCs by negative selection using a kit (Miltenyi Biotech—130-096-495) following manufacturer's protocol. Isolated CD8 T cells were blasted using the Human T cell activation and expansion kit (Miltenyi Biotech-130-091-441) in the presence of 100 pM Human IL-2 for 72 hours at 37° C. The blasted CD8 T cells were washed and the beads were removed magnetically following which the cells were treated with varying concentrations of the IL-10Rα/IL-10Rβ VHH dimers (0.05 pM-200 nM) for 72 hours at 37° C. Post-treatment, cells were spun down at 400 g for 5 min and supernatants were collected. Supernatants were assessed for cytokines (IFNγ and Granzyme B) using the MSD kit (Meso Scale Discovery: K151AEL-4) following manufacturer's protocol. The data was graphed using Prism software.

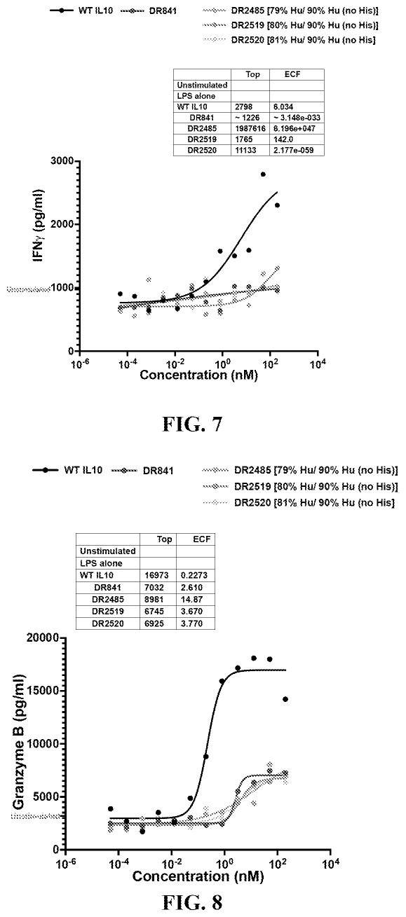

FIGS. 7 and 8 show that the humanized IL10Rα/IL10Rβ VHH dimers IL-10 agonist compounds DR2485 (SEQ ID NO:2), DR2519 (SEQ ID NO:3), and DR2520 (SEQ ID NO:4) have reduced activity in the induction of IFNγ and granzyme B. Accordingly, the humanized IL10Rα/IL10Rβ VHH dimer IL-10 agonist compounds are biased in showing preferential activity on monocytes compared to CD8+ T-cells, by avoiding CD8 activation.

TABLE 1
Humanized IL-10Rα/IL-10Rβ VHH Dimer Amino Acid Sequences

| Amino Acid | | |
|---|---|---|
| Number | | |
| corresponding | | |
| to DNA | Amino Acid Sequence of hzIL-10Rα/IL-10Rβ VHH Dimer | SEQ ID |
| number | (CDRs Underlined) | NO: |
| DR2463aa | EVQLLESGGGLVQAGGSLRLSCAASGYSNCAYDMTWYRQAPGKEREFVSAIHSDGSTRYA | 1 |
| | DSVKGRFFISRDNSKNTVYLQMNSLRPEDTAVYYCKTDPLHCRAHGGSWYSVRANYWGQG | |
| | TLVTVSSGGGSEVQLVESGGGLVQPGGSLRLSCAASGYTHSSYCMGWVRQAPGKGLEGVS | |
| | AIDVDGSTTYADSVKGRFTISRDNSKNTLYLQMNSLRAEDTAVYYCAAEFADCSSNYFLP | |
| | PGAVRYWGQGTLVTVSSASHHHHHH | |
| DR2485aa | EVQLLESGGGLVQAGGSLRLSCAASGYSNCAYDMTWYRQAPGKEREFVSAIHSDGSTRYA | 2 |
| | DSVKGRFFISRDNSKNTVYLQMNSLRPEDTAVYYCKTDPLHCRAHGGSWYSVRANYWGQG | |
| | TLVTVSSGGGSEVQLVESGGGLVQPGGSLRLSCAASGYTHSSYCMGWVRQAPGKGLEGVS | |
| | AIDVDGSTTYADSVKGRFTISRDNSKNTLYLQMNSLRAEDTAVYYCAAEFADCSSNYFLP | |
| | PGAVRYWGQGTTVTVSS | |
| DR2519aa | EVQLLESGGGLVQAGGSLRLSCAASGYSNCAYDMTWYRQAPGKEREFVSAIHSDGSTYYA | 3 |
| | DSVKGRFFISRDNSKNTVYLQMNSLRPEDTAVYYCKTDPLHCRAHGGSWYSVRANYWGQG | |
| | TLVTVSSGGGSEVQLVESGGGLVQPGGSLRLSCAASGYTHSSYCMGWVRQAPGKGLEGVS | |
| | AIDVDGSTTYADSVKGRFTISRDNSKNTLYLQMNSLRAEDTAVYYCAAEFADCSSNYFLP | |
| | PGAVRYWGQGTTVTVSS | |
| DR2520aa | EVQLLESGGGLVQAGGSLRLSCAASGYSNCAYDMTWYRQAPGKEREFVSAIHSSGSTYYA | 4 |
| | DSVKGRFFISRDNSKNTVYLQMNSLRPEDTAVYYCKTDPLHCRAHGGSWYSVRANYWGQG | |
| | TLVTVSSGGGSEVQLVESGGGLVQPGGSLRLSCAASGYTHSSYCMGWVRQAPGKGLEGVS | |
| | AIDVDGSTTYADSVKGRFTISRDNSKNTLYLQMNSLRAEDTAVYYCAAEFADCSSNYFLP | |
| | PGAVRYWGQGTTVTVSS | |
| DR2457aa | EVQLQESGGGSVQPGGSLRLSCAASGYSNCAYDMTWYRQAPGKEREFVSAIHSDGSTRYA | 5 |
| | DSVKGRFFISQDNAKNTVYLQMNSLKPEDTAMYYCKTDPLHCRAHGGSWYSVRANYWGQG | |
| | TQVTVSSGGGSEVQLVESGGGLVQPGGSLRLSCAASGYTHSSYCMGWVRQAPGKGLEGVS | |
| | AIDVDGSTTYADSVKGRFTISRDNSKNTLYLQMNSLRAEDTAVYYCAAEFADCSSNYFLP | |
| | PGAVRYWGQGTLVTVSSASHHHHHH | |
| DR2458aa | EVQLQESGGGLVQAGGSLRLSCAASGYSNCAYDMTWYRQAPGKEREFVSAIHSDGSTRYA | 6 |
| | DSVKGRFFISQDNSKNTVYLQMNSLKPEDTAVYYCKTDPLHCRAHGGSWYSVRANYWGQG | |
| | TLVTVSSGGGSEVQLVESGGGLVQPGGSLRLSCAASGYTHSSYCMGWVRQAPGKGLEGVS | |
| | AIDVDGSTTYADSVKGRFTISRDNSKNTLYLQMNSLRAEDTAVYYCAAEFADCSSNYFLP | |
| | PGAVRYWGQGTLVTVSSASHHHHHH | |
| DR2459aa | EVQLQESGGGLVQAGGSLRLSCAASGYSNCAYDMTWYRQAPGKEREFVSAIHSDGSTRYA | 7 |
| | DSVKGRFFISRDNSKNTVYLQMNSLKPEDTAVYYCKTDPLHCRAHGGSWYSVRANYWGQG | |
| | TLVTVSSGGGSEVQLVESGGGLVQPGGSLRLSCAASGYTHSSYCMGWVRQAPGKGLEGVS | |
| | AIDVDGSTTYADSVKGRFTISRDNSKNTLYLQMNSLRAEDTAVYYCAAEFADCSSNYFLP | |
| | PGAVRYWGQGTLVTVSSASHHHHHH | |
| DR2460aa | EVQLLESGGGLVQAGGSLRLSCAASGYSNCAYDMTWYRQAPGKEREFVSAIHSDGSTRYA | 8 |
| | DSVKGRFFISQDNSKNTVYLQMNSLKPEDTAVYYCKTDPLHCRAHGGSWYSVRANYWGQG | |
| | TLVTVSSGGGSEVQLVESGGGLVQPGGSLRLSCAASGYTHSSYCMGWVRQAPGKGLEGVS | |
| | AIDVDGSTTYADSVKGRFTISRDNSKNTLYLQMNSLRAEDTAVYYCAAEFADCSSNYFLP | |
| | PGAVRYWGQGTLVTVSSASHHHHHH | |
| DR2461aa | EVQLLESGGGLVQAGGSLRLSCAASGYSNCAYDMTWYRQAPGKEREFVSAIHSDGSTRYA | 9 |
| | DSVKGRFFISRDNSKNTVYLQMNSLKPEDTAVYYCKTDPLHCRAHGGSWYSVRANYWGQG | |
| | TLVTVSSGGGSEVQLVESGGGLVQPGGSLRLSCAASGYTHSSYCMGWVRQAPGKGLEGVS | |
| | AIDVDGSTTYADSVKGRFTISRDNSKNTLYLQMNSLRAEDTAVYYCAAEFADCSSNYFLP | |
| | PGAVRYWGQGTLVTVSSASHHHHHH | |
| DR2462aa | EVQLQESGGGLVQAGGSLRLSCAASGYSNCAYDMTWYRQAPGKEREFVSAIHSDGSTRYA | 10 |
| | DSVKGRFFISRDNSKNTVYLQMNSLRPEDTAVYYCKTDPLHCRAHGGSWYSVRANYWGQG | |
| | TLVTVSSGGGSEVQLVESGGGLVQPGGSLRLSCAASGYTHSSYCMGWVRQAPGKGLEGVS | |
| | AIDVDGSTTYADSVKGRFTISRDNSKNTLYLQMNSLRAEDTAVYYCAAEFADCSSNYFLP | |
| | PGAVRYWGQGTLVTVSSASHHHHHH | |
| DR2464aa | EVQLLESGGGLVQAGGSLRLSCAASGYSNCAYDMTWYRQAPGKEREFVSAIHSDGSTRYA | 11 |
| | DSVKGRFFISRDNSKNTVYLQMNSLRAEDTAVYYCKTDPLHCRAHGGSWYSVRANYWGQG | |
| | TLVTVSSGGGSEVQLVESGGGLVQPGGSLRLSCAASGYTHSSYCMGWVRQAPGKGLEGVS | |
| | AIDVDGSTTYADSVKGRFTISRDNSKNTLYLQMNSLRAEDTAVYYCAAEFADCSSNYFLP | |
| | PGAVRYWGQGTLVTVSSASHHHHHH | |
| DR2465aa | EVQLLESGGGLVQAGGSLRLSCAASGYSNCAYDMTWYRQAPGKEREFVSAIHSDGSTRYA | 12 |
| | DSVKGRFFISRDNSKNTLYLQMNSLRPEDTAVYYCKTDPLHCRAHGGSWYSVRANYWGQG | |
| | TLVTVSSGGGSEVQLVESGGGLVQPGGSLRLSCAASGYTHSSYCMGWVRQAPGKGLEGVS | |
| | AIDVDGSTTYADSVKGRFTISRDNSKNTLYLQMNSLRAEDTAVYYCAAEFADCSSNYFLP | |
| | PGAVRYWGQGTLVTVSSASHHHHHH | |
| DR2466aa | EVQLLESGGGLVQAGGSLRLSCAASGYSNCAYDMTWYRQAPGKEREFVSAIHSDGSTRYA | 13 |
| | DSVKGRFFISRDNSKNTLYLQMNSLRAEDTAVYYCKTDPLHCRAHGGSWYSVRANYWGQG | |
| | TLVTVSSGGGSEVQLVESGGGLVQPGGSLRLSCAASGYTHSSYCMGWVRQAPGKGLEGVS | |
| | AIDVDGSTTYADSVKGRFTISRDNSKNTLYLQMNSLRAEDTAVYYCAAEFADCSSNYFLP | |
| | PGAVRYWGQGTLVTVSSASHHHHHH | |
| DR2499aa | EVQLLESGGGLVQAGGSLRLSCAASGYSNCAYAMTWYRQAPGKEREFVSAIHSDGSTRYA | 14 |
| (DR2463aa | DSVKGRFFISRDNSKNTVYLQMNSLRPEDTAVYYCKTDPLHCRAHGGSWYSVRANYWGQG | |
| D33A | TLVTVSSGGGSEVQLVESGGGLVQPGGSLRLSCAASGYTHSSYCMGWVRQAPGKGLEGVS | |
| resurfacing) | AIDVDGSTTYADSVKGRFTISRDNSKNTLYLQMNSLRAEDTAVYYCAAEFADCSSNYFLP | |
| | PGAVRYWGQGTLVTVSSASHHHHHH | |
| DR2500aa | EVQLLESGGGLVQAGGSLRLSCAASGYSNCAYDMSWYRQAPGKEREFVSAIHSDGSTRYA | 15 |
| (DR2463aa | DSVKGRFFISRDNSKNTVYLQMNSLRPEDTAVYYCKTDPLHCRAHGGSWYSVRANYWGQG | |
| T35S | TLVTVSSGGGSEVQLVESGGGLVQPGGSLRLSCAASGYTHSSYCMGWVRQAPGKGLEGVS | |
| resurfacing) | AIDVDGSTTYADSVKGRFTISRDNSKNTLYLQMNSLRAEDTAVYYCAAEFADCSSNYFLP | |
| | PGAVRYWGQGTLVTVSSASHHHHHH | |
| DR2501aa | EVQLLESGGGLVQAGGSLRLSCAASGYSNCAYDMTWYRQAPGKEREFVSAISSDGSTRYA | 16 |
| (DR2463aa | DSVKGRFFISRDNSKNTVYLQMNSLRPEDTAVYYCKTDPLHCRAHGGSWYSVRANYWGQG | |
| H52S, | TLVTVSSGGGSEVQLVESGGGLVQPGGSLRLSCAASGYTHSSYCMGWVRQAPGKGLEGVS | |
| CDR2 | AIDVDGSTTYADSVKGRFTISRDNSKNTLYLQMNSLRAEDTAVYYCAAEFADCSSNYFLP | |
| resurfacing) | PGAVRYWGQGTLVTVSSASHHHHHH | |
| DR0000aa | EVQLLESGGGLVQAGGSLRLSCAASGYSNCAYDMTWYRQAPGKEREFVSAIHGDGSTRYA | 17 |
| (DR2463aa | DSVKGRFFISRDNSKNTVYLQMNSLRPEDTAVYYCKTDPLHCRAHGGSWYSVRANYWGQG | |
| S53G, | TLVTVSSGGGSEVQLVESGGGLVQPGGSLRLSCAASGYTHSSYCMGWVRQAPGKGLEGVS | |
| CDR2 | AIDVDGSTTYADSVKGRFTISRDNSKNTLYLQMNSLRAEDTAVYYCAAEFADCSSNYFLP | |
| resurfacing) | PGAVRYWGQGTLVTVSSASHHHHHH | |
| DR2502aa | EVQLLESGGGLVQAGGSLRLSCAASGYSNCAYDMTWYRQAPGKEREFVSAIHSSGSTRYA | 18 |
| (DR2463 | DSVKGRFFISRDNSKNTVYLQMNSLRPEDTAVYYCKTDPLHCRAHGGSWYSVRANYWGQG | |
| D54S, | TLVTVSSGGGSEVQLVESGGGLVQPGGSLRLSCAASGYTHSSYCMGWVRQAPGKGLEGVS | |
| CDR2 | AIDVDGSTTYADSVKGRFTISRDNSKNTLYLQMNSLRAEDTAVYYCAAEFADCSSNYFLP | |
| resurfacing) | PGAVRYWGQGTLVTVSSASHHHHHH | |
| DR2503aa | EVQLLESGGGLVQAGGSLRLSCAASGYSNCAYDMTWYRQAPGKEREFVSAIHSDGSTYYA | 19 |
| (DR2463aa | DSVKGRFFISRDNSKNTVYLQMNSLRPEDTAVYYCKTDPLHCRAHGGSWYSVRANYWGQG | |
| R58Y, | TLVTVSSGGGSEVQLVESGGGLVQPGGSLRLSCAASGYTHSSYCMGWVRQAPGKGLEGVS | |
| CDR2 | AIDVDGSTTYADSVKGRFTISRDNSKNTLYLQMNSLRAEDTAVYYCAAEFADCSSNYFLP | |
| resurfacing) | PGAVRYWGQGTLVTVSSASHHHHHH | |
| DR2504aa | EVQLLESGGGLVQAGGSLRLSCAASGYSNCAYAMSWYRQAPGKEREFVSAIHSDGSTRYA | 20 |
| (DR2463aa | DSVKGRFFISRDNSKNTVYLQMNSLRPEDTAVYYCKTDPLHCRAHGGSWYSVRANYWGQG | |
| D33A, | TLVTVSSGGGSEVQLVESGGGLVQPGGSLRLSCAASGYTHSSYCMGWVRQAPGKGLEGVS | |
| T55S | AIDVDGSTTYADSVKGRFTISRDNSKNTLYLQMNSLRAEDTAVYYCAAEFADCSSNYFLP | |
| CDR1 | PGAVRYWGQGTLVTVSSASHHHHHH | |
| Resurfacing) | | |
| DR2505aa | EVQLLESGGGLVQAGGSLRLSCAASGYSNCAYDMTWYRQAPGKEREFVSAISGSGSTYYA | 21 |
| (DR2463aa | DSVKGRFFISRDNSKNTVYLQMNSLRPEDTAVYYCKTDPLHCRAHGGSWYSVRANYWGQG | |
| H72S, | TLVTVSSGGGSEVQLVESGGGLVQPGGSLRLSCAASGYTHSSYCMGWVRQAPGKGLEGVS | |
| S73G, | AIDVDGSTTYADSVKGRFTISRDNSKNTLYLQMNSLRAEDTAVYYCAAEFADCSSNYFLP | |
| D74S, | PGAVRYWGQGTLVTVSSASHHHHHH | |
| R78Y, | | |
| CDR2 | | |
| Resurfacing) | | |
| DR2506aa | EVQLLESGGGLVQAGGSLRLSCAASGYSNCAYAMSWYRQAPGKEREFVSAISGSGSTYYA | 22 |
| (DR2463aa | DSVKGRFFISRDNSKNTVYLQMNSLRPEDTAVYYCKTDPLHCRAHGGSWYSVRANYWGQG | |
| D33A, | TLVTVSSGGGSEVQLVESGGGLVQPGGSLRLSCAASGYTHSSYCMGWVRQAPGKGLEGVS | |
| H72S, | AIDVDGSTTYADSVKGRFTISRDNSKNTLYLQMNSLRAEDTAVYYCAAEFADCSSNYFLP | |
| S73G, | PGAVRYWGQGTLVTVSSASHHHHHH | |
| D74S, | | |
| R78Y, | | |
| CDR1 + CDR2 | | |
| resurfacing) | | |
| DR1525aa | EVQLLESGGGLVQPGGSLRLSCAASGYSNCAYDMTWYRQAPGKEREFVSAIHSDGSTRYA | 23 |
| | DSVKGRFFISRDNSKNTLYLQMNSLRAEDTAVYYCKTDPLHCRAHGGSWYSVRANYWGQG | |
| | TLVTVSSGGGSEVQLVESGGGLVQPGGSLRLSCAASGYTHSSYCMGWVRQAPGKGLEGVS | |
| | AIDVDGSTTYADSVKGRFTISRDNSKNTLYLQMNSLRAEDTAVYYCAAEFADCSSNYFLP | |
| | PGAVRYWGQGTLVTVSSASHHHHHH | |
| DR2096aa | EVQLLESGGGSVQPGGSLRLSCAASGYSNCAYDMTWYRQAPGKEREFVSAIHSDGSTRYA | 24 |
| | DSVKGRFFISRDNSKNTLYLQMNSLRAEDTAVYYCKTDPLHCRAHGGSWYSVRANYWGQG | |
| | TLVTVSSGGGSEVQLVESGGGLVQPGGSLRLSCAASGYTHSSYCMGWVRQAPGKGLEGVS | |
| | AIDVDGSTTYADSVKGRFTISRDNSKNTLYLQMNSLRAEDTAVYYCAAEFADCSSNYFLP | |
| | PGAVRYWGQGTLVTVSAAS | |

TABLE 2
Humanized IL-10Rα VHHs Amino Acid Sequences

| VHH Amino | | |
|---|---|---|
| Acid | | |
| Number | | |
| (corresponding | | |
| to DNA | VHH Amino Acid Sequence | SEQ ID |
| number) | (CDRs Underlined) | NO: |
| DR2463aa | EVQLLESGGGLVQAGGSLRLSCAASGYSNCAYDMTWYRQAPGKEREFVSAIHSDGSTR | 25 |
| hzIL-10Rα | YADSVKGRFFISRDNSKNTVYLQMNSLRPEDTAVYYCKTDPLHCRAHGGSWYSVRANY | |
| | WGQGTLVTVSS | |
| DR2485aa | EVQLLESGGGLVQAGGSLRLSCAASGYSNCAYDMTWYRQAPGKEREFVSAIHSDGSTR | 26 |
| hzIL-10Rα | YADSVKGRFFISRDNSKNTVYLQMNSLRPEDTAVYYCKTDPLHCRAHGGSWYSVRANY | |
| | WGQGTLVTVSS | |
| DR2519aa | EVQLLESGGGLVQAGGSLRLSCAASGYSNCAYDMTWYRQAPGKEREFVSAIHSDGSTY | 27 |
| hzIL-10Rα | YADSVKGRFFISRDNSKNTVYLQMNSLRPEDTAVYYCKTDPLHCRAHGGSWYSVRANY | |
| | WGQGTLVTVSS | |
| DR2520aa | EVQLLESGGGLVQAGGSLRLSCAASGYSNCAYDMTWYRQAPGKEREFVSAIHSSGSTY | 28 |
| hzIL-10Rα | YADSVKGRFFISRDNSKNTVYLQMNSLRPEDTAVYYCKTDPLHCRAHGGSWYSVRANY | |
| | WGQGTLVTVSS | |
| DR2457aa | EVQLQESGGGSVQPGGSLRLSCAASGYSNCAYDMTWYRQAPGKEREFVSAIHSDGSTR | 29 |
| hzIL-10Rα | YADSVKGRFFISQDNAKNTVYLQMNSLKPEDTAMYYCKTDPLHCRAHGGSWYSVRANY | |
| | WGQGTQVTVSS | |
| DR2458aa | EVQLQESGGGLVQAGGSLRLSCAASGYSNCAYDMTWYRQAPGKEREFVSAIHSDGSTR | 30 |
| hzIL-10Rα | YADSVKGRFFISQDNSKNTVYLQMNSLKPEDTAVYYCKTDPLHCRAHGGSWYSVRANY | |
| | WGQGTLVTVSS | |
| DR2459aa | EVQLQESGGGLVQAGGSLRLSCAASGYSNCAYDMTWYRQAPGKEREFVSAIHSDGSTR | 31 |
| hzIL-10Rα | YADSVKGRFFISRDNSKNTVYLQMNSLKPEDTAVYYCKTDPLHCRAHGGSWYSVRANY | |
| | WGQGTLVTVSS | |
| DR2460aa | EVQLLESGGGLVQAGGSLRLSCAASGYSNCAYDMTWYRQAPGKEREFVSAIHSDGSTR | 32 |
| hzIL-10Rα | YADSVKGRFFISQDNSKNTVYLQMNSLKPEDTAVYYCKTDPLHCRAHGGSWYSVRANY | |
| | WGQGTLVTVSS | |
| DR2461aa | EVQLLESGGGLVQAGGSLRLSCAASGYSNCAYDMTWYRQAPGKEREFVSAIHSDGSTR | 33 |
| hzIL-10Rα | YADSVKGRFFISRDNSKNTVYLQMNSLKPEDTAVYYCKTDPLHCRAHGGSWYSVRANY | |
| | WGQGTLVTVSS | |
| DR2462aa | EVQLQESGGGLVQAGGSLRLSCAASGYSNCAYDMTWYRQAPGKEREFVSAIHSDGSTR | 34 |
| hzIL-10Rα | YADSVKGRFFISRDNSKNTVYLQMNSLRPEDTAVYYCKTDPLHCRAHGGSWYSVRANY | |
| | WGQGTLVTVSS | |
| DR2464aa | EVQLLESGGGLVQAGGSLRLSCAASGYSNCAYDMTWYRQAPGKEREFVSAIHSDGSTR | 35 |
| hzIL-10Rα | YADSVKGRFFISRDNSKNTVYLQMNSLRAEDTAVYYCKTDPLHCRAHGGSWYSVRANY | |
| | WGQGTLVTVSS | |
| DR2465aa | VQLLESGGGLVQAGGSLRLSCAASGYSNCAYDMTWYRQAPGKEREFVSAIHSDGSTRY | 36 |
| hzIL-10Rα | ADSVKGRFFISRDNSKNTLYLQMNSLRPEDTAVYYCKTDPLHCRAHGGSWYSVRANYW | |
| | GQGTLVTVSS | |
| DR2466aa | EVQLLESGGGLVQAGGSLRLSCAASGYSNCAYDMTWYRQAPGKEREFVSAIHSDGSTR | 37 |
| hzIL-10Rα | YADSVKGRFFISRDNSKNTLYLQMNSLRAEDTAVYYCKTDPLHCRAHGGSWYSVRANY | |
| | WGQGTLVTVSS | |
| DR2499aa | EVQLLESGGGLVQAGGSLRLSCAASGYSNCAYAMTWYRQAPGKEREFVSAIHSDGSTR | 38 |
| hzIL-10Rα | YADSVKGRFFISRDNSKNTVYLQMNSLRPEDTAVYYCKTDPLHCRAHGGSWYSVRANY | |
| | WGQGTLVTVSS | |
| DR2500aa | EVQLLESGGGLVQAGGSLRLSCAASGYSNCAYDMSWYRQAPGKEREFVSAIHSDGSTR | 39 |
| hzIL-10Rα | YADSVKGRFFISRDNSKNTVYLQMNSLRPEDTAVYYCKTDPLHCRAHGGSWYSVRANY | |
| | WGQGTLVTVSS | |
| DR2501aa | EVQLLESGGGLVQAGGSLRLSCAASGYSNCAYDMTWYRQAPGKEREFVSAISSDGSTR | 40 |
| hzIL-10Rα | YADSVKGRFFISRDNSKNTVYLQMNSLRPEDTAVYYCKTDPLHCRAHGGSWYSVRANY | |
| | WGQGTLVTVSS | |
| DR0000aa | EVQLLESGGGLVQAGGSLRLSCAASGYSNCAYDMTWYRQAPGKEREFVSAIHGDGSTR | 41 |
| hzIL-10Rα | YADSVKGRFFISRDNSKNTVYLQMNSLRPEDTAVYYCKTDPLHCRAHGGSWYSVRANY | |
| | WGQGTLVTVSS | |
| DR2502aa | EVQLLESGGGLVQAGGSLRLSCAASGYSNCAYDMTWYRQAPGKEREFVSAIHSSGSTR | 42 |
| hzIL-10Rα | YADSVKGRFFISRDNSKNTVYLQMNSLRPEDTAVYYCKTDPLHCRAHGGSWYSVRANY | |
| | WGQGTLVTVSS | |
| DR2503aa | EVQLLESGGGLVQAGGSLRLSCAASGYSNCAYDMTWYRQAPGKEREFVSAIHSDGSTY | 43 |
| hzIL-10Rα | YADSVKGRFFISRDNSKNTVYLQMNSLRPEDTAVYYCKTDPLHCRAHGGSWYSVRANY | |
| | WGQGTLVTVSS | |
| DR2504aa | EVQLLESGGGLVQAGGSLRLSCAASGYSNCAYAMSWYRQAPGKEREFVSAIHSDGSTR | 44 |
| hzIL-10Rα | YADSVKGRFFISRDNSKNTVYLQMNSLRPEDTAVYYCKTDPLHCRAHGGSWYSVRANY | |
| | WGQGTLVTVSS | |
| DR2505 | EVQLLESGGGLVQAGGSLRLSCAASGYSNCAYDMTWYRQAPGKEREFVSAISGSGSTY | 45 |
| hzIL-10Rα | YADSVKGRFFISRDNSKNTVYLQMNSLRPEDTAVYYCKTDPLHCRAHGGSWYSVRANY | |
| | WGQGTLVTVSS | |
| DR2506aa | EVQLLESGGGLVQAGGSLRLSCAASGYSNCAYAMSWYRQAPGKEREFVSAISGSGSTY | 46 |
| hzIL-10Rα | YADSVKGRFFISRDNSKNTVYLQMNSLRPEDTAVYYCKTDPLHCRAHGGSWYSVRANY | |
| | WGQGTLVTVSS | |
| DR1525aa | EVQLLESGGGLVQPGGSLRLSCAASGYSNCAYDMTWYRQAPGKEREFVSAIHSDGSTR | 47 |
| hzIL-10Rα | YADSVKGRFFISRDNSKNTLYLQMNSLRAEDTAVYYCKTDPLHCRAHGGSWYSVRANY | |
| | WGQGTLVTVSS | |
| DR2096aa | EVQLLESGGGSVQPGGSLRLSCAASGYSNCAYDMTWYRQAPGKEREFVSAIHSDGSTR | 48 |
| hzIL-10Rα | YADSVKGRFFISRDNSKNTLYLQMNSLRAEDTAVYYCKTDPLHCRAHGGSWYSVRANY | |
| | WGQGTLVTVSS | |

TABLE 3
Humanized IL-10Rβ VHH Amino Acid Sequences

| VHH Amino | | |
|---|---|---|
| Acid | | |
| Number | | |
| (corresponding | | |
| to DNA | hzIL-10Rβ VHH Amino Acid Sequence | SEQ ID |
| number) | (VHRs Underlined) | NO: |
| DR2463aa | EVQLVESGGGLVQPGGSLRLSCAASGYTHSSYCMGWVRQAPGKGLEGVSAIDVDGSTT | 49 |
| hzIL-10Rβ | YADSVKGRFTISRDNSKNTLYLQMNSLRAEDTAVYYCAAEFADCSSNYFLPPGAVRYW | |
| | GQGTLVTVSS | |
| DR2485aa | EVQLVESGGGLVQPGGSLRLSCAASGYTHSSYCMGWVRQAPGKGLEGVSAIDVDGSTT | 50 |
| hzIL-10Rβ | YADSVKGRFTISRDNSKNTLYLQMNSLRAEDTAVYYCAAEFADCSSNYFLPPGAVRYW | |
| | GQGTTVTVSS | |
| DR2519aa | EVQLVESGGGLVQPGGSLRLSCAASGYTHSSYCMGWVRQAPGKGLEGVSAIDVDGSTT | 50 |
| hzIL-10Rβ | YADSVKGRFTISRDNSKNTLYLQMNSLRAEDTAVYYCAAEFADCSSNYFLPPGAVRYW | |
| | GQGTTVTVSS | |
| DR2520aa | EVQLVESGGGLVQPGGSLRLSCAASGYTHSSYCMGWVRQAPGKGLEGVSAIDVDGSTT | 50 |
| hzIL-10Rβ | YADSVKGRFTISRDNSKNTLYLQMNSLRAEDTAVYYCAAEFADCSSNYFLPPGAVRYW | |
| | GQGTTVTVSS | |
| DR2457aa | EVQLVESGGGLVQPGGSLRLSCAASGYTHSSYCMGWVRQAPGKGLEGVSAIDVDGSTT | 49 |
| hzIL-10Rβ | YADSVKGRFTISRDNSKNTLYLQMNSLRAEDTAVYYCAAEFADCSSNYFLPPGAVRYW | |
| | GQGTLVTVSS | |
| DR2458aa | EVQLVESGGGLVQPGGSLRLSCAASGYTHSSYCMGWVRQAPGKGLEGVSAIDVDGSTT | 49 |
| hzIL-10Rβ | YADSVKGRFTISRDNSKNTLYLQMNSLRAEDTAVYYCAAEFADCSSNYFLPPGAVRYW | |
| | GQGTLVTVSS | |
| DR2459aa | EVQLVESGGGLVQPGGSLRLSCAASGYTHSSYCMGWVRQAPGKGLEGVSAIDVDGSTT | 49 |
| hzIL-10Rβ | YADSVKGRFTISRDNSKNTLYLQMNSLRAEDTAVYYCAAEFADCSSNYFLPPGAVRYW | |
| | GQGTLVTVSS | |
| DR2460aa | EVQLVESGGGLVQPGGSLRLSCAASGYTHSSYCMGWVRQAPGKGLEGVSAIDVDGSTT | 49 |
| hzIL-10Rβ | YADSVKGRFTISRDNSKNTLYLQMNSLRAEDTAVYYCAAEFADCSSNYFLPPGAVRYW | |
| | GQGTLVTVSS | |
| DR2461aa | EVQLVESGGGLVQPGGSLRLSCAASGYTHSSYCMGWVRQAPGKGLEGVSAIDVDGSTT | 49 |
| hzIL-10Rβ | YADSVKGRFTISRDNSKNTLYLQMNSLRAEDTAVYYCAAEFADCSSNYFLPPGAVRYW | |
| | GQGTLVTVSS | |
| DR2462aa | EVQLVESGGGLVQPGGSLRLSCAASGYTHSSYCMGWVRQAPGKGLEGVSAIDVDGSTT | 49 |
| hzIL-10Rβ | YADSVKGRFTISRDNSKNTLYLQMNSLRAEDTAVYYCAAEFADCSSNYFLPPGAVRYW | |
| | GQGTLVTVSS | |
| DR2464aa | EVQLVESGGGLVQPGGSLRLSCAASGYTHSSYCMGWVRQAPGKGLEGVSAIDVDGSTT | 49 |
| hzIL-10Rβ | YADSVKGRFTISRDNSKNTLYLQMNSLRAEDTAVYYCAAEFADCSSNYFLPPGAVRYW | |
| | GQGTLVTVSS | |
| DR2465aa | EVQLVESGGGLVQPGGSLRLSCAASGYTHSSYCMGWVRQAPGKGLEGVSAIDVDGSTT | 49 |
| hzIL-10Rβ | YADSVKGRFTISRDNSKNTLYLQMNSLRAEDTAVYYCAAEFADCSSNYFLPPGAVRYW | |
| | GQGTLVTVSS | |
| DR2466aa | EVQLVESGGGLVQPGGSLRLSCAASGYTHSSYCMGWVRQAPGKGLEGVSAIDVDGSTT | 49 |
| hzIL-10Rβ | YADSVKGRFTISRDNSKNTLYLQMNSLRAEDTAVYYCAAEFADCSSNYFLPPGAVRYW | |
| | GQGTLVTVSS | |
| DR2499aa | EVQLVESGGGLVQPGGSLRLSCAASGYTHSSYCMGWVRQAPGKGLEGVSAIDVDGSTT | 49 |
| hzIL-10Rβ | YADSVKGRFTISRDNSKNTLYLQMNSLRAEDTAVYYCAAEFADCSSNYFLPPGAVRYW | |
| | GQGTLVTVSS | |
| DR2501aa | EVQLVESGGGLVQPGGSLRLSCAASGYTHSSYCMGWVRQAPGKGLEGVSAIDVDGSTT | 49 |
| hzIL-10Rβ | YADSVKGRFTISRDNSKNTLYLQMNSLRAEDTAVYYCAAEFADCSSNYFLPPGAVRYW | |
| | GQGTLVTVSS | |
| DR0000aa | EVQLVESGGGLVQPGGSLRLSCAASGYTHSSYCMGWVRQAPGKGLEGVSAIDVDGSTT | 49 |
| hzIL-10Rβ | YADSVKGRFTISRDNSKNTLYLQMNSLRAEDTAVYYCAAEFADCSSNYFLPPGAVRYW | |
| | GQGTLVTVSS | |
| DR2501aa-b | EVQLVESGGGLVQPGGSLRLSCAASGYTHSSYCMGWVRQAPGKGLEGVSAIDVDGSTT | 49 |
| hzIL-10Rβ | YADSVKGRFTISRDNSKNTLYLQMNSLRAEDTAVYYCAAEFADCSSNYFLPPGAVRYW | |
| | GQGTLVTVSS | |
| DR2502aa | EVQLVESGGGLVQPGGSLRLSCAASGYTHSSYCMGWVRQAPGKGLEGVSAIDVDGSTT | 49 |
| hzIL-10Rβ | YADSVKGRFTISRDNSKNTLYLQMNSLRAEDTAVYYCAAEFADCSSNYFLPPGAVRYW | |
| | GQGTLVTVSS | |
| DR2503aa | EVQLVESGGGLVQPGGSLRLSCAASGYTHSSYCMGWVRQAPGKGLEGVSAIDVDGSTT | 49 |
| hzIL-10Rβ | YADSVKGRFTISRDNSKNTLYLQMNSLRAEDTAVYYCAAEFADCSSNYFLPPGAVRYW | |
| | GQGTLVTVSS | |
| DR2504aa | EVQLVESGGGLVQPGGSLRLSCAASGYTHSSYCMGWVRQAPGKGLEGVSAIDVDGSTT | 49 |
| hzIL-10Rβ | YADSVKGRFTISRDNSKNTLYLQMNSLRAEDTAVYYCAAEFADCSSNYFLPPGAVRYW | |
| | GQGTLVTVSS | |
| DR2505aa | EVQLVESGGGLVQPGGSLRLSCAASGYTHSSYCMGWVRQAPGKGLEGVSAIDVDGSTT | 49 |
| hzIL-10Rβ | YADSVKGRFTISRDNSKNTLYLQMNSLRAEDTAVYYCAAEFADCSSNYFLPPGAVRYW | |
| | GQGTLVTVSS | |
| DR2506aa | EVQLVESGGGLVQPGGSLRLSCAASGYTHSSYCMGWVRQAPGKGLEGVSAIDVDGSTT | 49 |
| hzIL-10Rβ | YADSVKGRFTISRDNSKNTLYLQMNSLRAEDTAVYYCAAEFADCSSNYFLPPGAVRYW | |
| | GQGTLVTVSS | |
| DR1525aa | EVQLVESGGGLVQPGGSLRLSCAASGYTHSSYCMGWVRQAPGKGLEGVSAIDVDGSTT | 49 |
| | YADSVKGRFTISRDNSKNTLYLQMNSLRAEDTAVYYCAAEFADCSSNYFLPPGAVRYW | |
| | GQGTLVTVSS | |
| SEQ ID | EVQLVESGGGLVQPGGSLRLSCAASGYTHSSYCMGWVRQAPGKGLEGVSAIDVDGSTT | 51 |
| NO: 50 | YADSVKGRFTISRDNSKNTLYLQMNSLRAEDTAVYYCAAEFADCSSNYFLPPGAVRYW | |
| VTVSA (SEQ | GQGTLVTVSA | |
| ID NO: 479) | | |
| MODIFIED | | |

TABLE 4
Description of hzIL10Rα-IL10Rβ VHH Dimer

| Dimer | N-terminal | | C-terminal | |
|---|---|---|---|---|
| VHH | VHH | | VHH | C-terminal |
| (SEQ ID | (SEQ ID | | (SEQ ID | additions or |
| NO: ) | NO: ) | Linker | NO: ) | modifications |
| 1 | 25 | G3S (SEQ ID NO: 439) | 49 | ASH6 (SEQ ID |
| | | | | NO: 449) |
| 2 | 26 | G3S (SEQ ID NO: 439) | 50 | |
| 3 | 27 | G3S (SEQ ID NO: 439) | 50 | |
| 4 | 28 | G3S (SEQ ID NO: 439) | 50 | |
| 5 | 29 | G3S (SEQ ID NO: 439) | 49 | |
| 6 | 30 | G3S (SEQ ID NO: 439) | 49 | |
| 7 | 31 | G3S (SEQ ID NO: 439) | 49 | |
| 8 | 32 | G3S (SEQ ID NO: 439) | 49 | |
| 9 | 33 | G3S (SEQ ID NO: 439) | 49 | |
| 10 | 34 | G3S (SEQ ID NO: 439) | 49 | |
| 11 | 35 | G3S (SEQ ID NO: 439) | 49 | |
| 12 | 36 | G3S (SEQ ID NO: 439) | 49 | |
| 13 | 37 | G3S (SEQ ID NO: 439) | 49 | |
| 14 | 38 | G3S (SEQ ID NO: 439) | 49 | |
| 15 | 39 | G3S (SEQ ID NO: 439) | 49 | |
| 16 | 40 | G3S (SEQ ID NO: 439) | 49 | |
| 17 | 41 | G3S (SEQ ID NO: 439) | 49 | |
| 18 | 42 | G3S (SEQ ID NO: 439) | 49 | |
| 19 | 43 | G3S (SEQ ID NO: 439) | 49 | |
| 20 | 44 | G3S (SEQ ID NO: 439) | 49 | |
| 21 | 45 | G3S (SEQ ID NO: 439) | 49 | |
| 22 | 46 | G3S (SEQ ID NO: 439) | 49 | |
| 23 | 47 | G3S (SEQ ID NO: 439) | 49 | ASH6 (SEQ ID |
| | | | | NO: 449) |
| 24 | 48 | G3S (SEQ ID NO: 439) | 51 | VTVSAAS (SEQ |
| | | | | ID NO: 498) |

TABLE 5
hzIL10Ra-IL10RB VHH Dimers for DR2463, DR2485, DR2519, DR2520,
with Modified C-Terminus

| hzIL10Rα- | | |
|---|---|---|
| IL10Rβ VHH | | |
| Dimer | Amino Acid Sequence of hzIL10Rα-IL10Rβ VHH Dimer with | SEQ ID |
| Modified | C-terminal Modifications (in bold) | NO: |
| DR2463aa-1 | EVQLLESGGGLVQAGGSLRLSCAASGYSNCAYDMTWYRQAPGKEREFVSAIHSDG | 121 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLGTVSS | |
| DR2463aa-2 | EVQLLESGGGLVQAGGSLRLSCAASGYSNCAYDMTWYRQAPGKEREFVSAIHSDG | 122 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLLTVSS | |
| DR2463aa-3 | EVQLLESGGGLVQAGGSLRLSCAASGYSNCAYDMTWYRQAPGKEREFVSAIHSDG | 123 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLLTVSSAA | |
| DR2463aa-4 | EVQLLESGGGLVQAGGSLRLSCAASGYSNCAYDMTWYRQAPGKEREFVSAIHSDG | 124 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVSA | |
| DR2463aa-5 | EVQLLESGGGLVQAGGSLRLSCAASGYSNCAYDMTWYRQAPGKEREFVSAIHSDG | 125 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVSAAA | |
| DR2463aa-6 | EVQLLESGGGLVQAGGSLRLSCAASGYSNCAYDMTWYRQAPGKEREFVSAIHSDG | 126 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVCS | |
| DR2463aa-7 | EVQLLESGGGLVQAGGSLRLSCAASGYSNCAYDMTWYRQAPGKEREFVSAIHSDG | 127 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVCSA | |
| DR2463aa-8 | EVQLLESGGGLVQAGGSLRLSCAASGYSNCAYDMTWYRQAPGKEREFVSAIHSDG | 128 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVCSAA | |
| DR2463aa-9 | EVQLLESGGGLVQAGGSLRLSCAASGYSNCAYDMTWYRQAPGKEREFVSAIHSDG | 129 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVSC | |
| DR2463aa-10 | EVQLLESGGGLVQAGGSLRLSCAASGYSNCAYDMTWYRQAPGKEREFVSAIHSDG | 130 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVSCAA | |
| DR2463aa- | EVQLLESGGGSVQAGGSLRLSCAASGYSNCAYDMTWYRQAPGKEREFVSAIHSDG | 131 |
| 11 (L11S) | STRYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLLTVSS | |
| DR2463aa- | EVQLLESGGGSVQAGGSLRLSCAASGYSNCAYDMTWYRQAPGKEREFVSAIHSDG | 132 |
| 12 (L11S) | STRYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLLTVSSA | |
| DR2463aa- | EVQLLESGGGSVQAGGSLRLSCAASGYSNCAYDMTWYRQAPGKEREFVSAIHSDG | 133 |
| 13 (L11S) | STRYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLLTVSSAA | |
| DR2463aa- | EVQLLESGGGSVQAGGSLRLSCAASGYSNCAYDMTWYRQAPGKEREFVSAIHSDG | 134 |
| 14 (L11S) | STRYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVTT | |
| DR2463aa- | EVQLLESGGGSVQAGGSLRLSCAASGYSNCAYDMTWYRQAPGKEREFVSAIHSDG | 135 |
| 15 (L11S) | STRYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVTTAA | |
| DR2463aa- | EVQLLESGGGSVQAGGSLRLSCAASGYSNCAYDMTWYRQAPGKEREFVSAIHSDG | 134 |
| 16 (L11S) | STRYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVTT | |
| DR2463aa- | EVQLLESGGGSVQAGGSLRLSCAASGYSNCAYDMTWYRQAPGKEREFVSAIHSDG | 135 |
| 17 (L11S) | STRYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVTTAA | |
| DR2463aa-18 | EVQLLESGGGLVQAGGSLRLSCAASGYSNCAYDMTWYRQAPGKEREFVSAIHSDG | 138 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTLTVSS | |
| DR2463aa-19 | EVQLLESGGGLVQAGGSLRLSCAASGYSNCAYDMTWYRQAPGKEREFVSAIHSDG | 139 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTLTVSSA | |
| DR2463aa-20 | EVQLLESGGGLVQAGGSLRLSCAASGYSNCAYDMTWYRQAPGKEREFVSAIHSDG | 140 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTLTVSSAA | |
| DR2485aa-1 | EVQLLESGGGLVQAGGSLRLSCAASGYSNCAYDMTWYRQAPGKEREFVSAIHSDH | 141 |
| | SDGSTRYADSVKGRFFISRDNSKNTVYLQMNSLRPEDTAVYYCKTDPLHCRAHGG | |
| | SWYSVRANYWGQGTLVTVSSGGGSEVQLVESGGGLVQPGGSLRLSCAASGYTHSS | |
| | YCMGWVRQAPGKGLEGVSAIDVDGSTTYADSVKGRFTISRDNSKNTLYLQMNSLR | |
| | AEDTAVYYCAAEFADCSSNYFLPPGAVRYWGQGTTGTVSS | |
| DR2485aa-2 | EVQLLESGGGLVQAGGSLRLSCAASGYSNCAYDMTWYRQAPGKEREFVSAIHSDG | 138 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTLTVSS | |
| DR2485aa-3 | EVQLLESGGGLVQAGGSLRLSCAASGYSNCAYDMTWYRQAPGKEREFVSAIHSDG | 140 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTLTVSSAA | |
| DR2485aa-4 | EVQLLESGGGLVQAGGSLRLSCAASGYSNCAYDMTWYRQAPGKEREFVSAIHSDG | 144 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTVTVSA | |
| DR2485aa-5 | EVQLLESGGGLVQAGGSLRLSCAASGYSNCAYDMTWYRQAPGKEREFVSAIHSDG | 145 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTVTVSAAA | |
| DR2485aa-6 | EVQLLESGGGLVQAGGSLRLSCAASGYSNCAYDMTWYRQAPGKEREFVSAIHSDG | 146 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTVTVCS | |
| DR2485aa-7 | EVQLLESGGGLVQAGGSLRLSCAASGYSNCAYDMTWYRQAPGKEREFVSAIHSDG | 147 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTVTVCSA | |
| DR2485aa-8 | EVQLLESGGGLVQAGGSLRLSCAASGYSNCAYDMTWYRQAPGKEREFVSAIHSDG | 148 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTVTVCSAA | |
| DR2485aa-9 | EVQLLESGGGLVQAGGSLRLSCAASGYSNCAYDMTWYRQAPGKEREFVSAIHSDG | 149 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTVTVSC | |
| DR2485aa-10 | EVQLLESGGGLVQAGGSLRLSCAASGYSNCAYDMTWYRQAPGKEREFVSAIHSDG | 150 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTVTVSCAA | |
| DR2485aa- | EVQLLESGGGSVQAGGSLRLSCAASGYSNCAYDMTWYRQAPGKEREFVSAIHSDG | 151 |
| 11 (L11S) | STRYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLLTVSS | |
| DR2485aa- | EVQLLESGGGSVQAGGSLRLSCAASGYSNCAYDMTWYRQAPGKEREFVSAIHSDG | 152 |
| 12 (L11S) | STRYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLLTVSSA | |
| DR2485aa- | EVQLLESGGGSVQAGGSLRLSCAASGYSNCAYDMTWYRQAPGKEREFVSAIHSDG | 153 |
| 13 (L11S) | STRYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLLTVSSAA | |
| DR2485aa- | EVQLLESGGGSVQAGGSLRLSCAASGYSNCAYDMTWYRQAPGKEREFVSAIHSDG | 154 |
| 14 (L11S) | STRYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVTT | |
| DR2485aa- | EVQLLESGGGSVQAGGSLRLSCAASGYSNCAYDMTWYRQAPGKEREFVSAIHSDG | 155 |
| 15 (L11S) | STRYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVTTAA | |
| DR2485aa- | EVQLLESGGGSVQAGGSLRLSCAASGYSNCAYDMTWYRQAPGKEREFVSAIHSDG | 154 |
| 16 (L11S) | STRYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVTT | |
| DR2485aa- | EVQLLESGGGSVQAGGSLRLSCAASGYSNCAYDMTWYRQAPGKEREFVSAIHSDG | 155 |
| 17 (L11S) | STRYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVTTAA | |
| DR2485aa-18 | EVQLLESGGGLVQAGGSLRLSCAASGYSNCAYDMTWYRQAPGKEREFVSAIHSDG | 158 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTLTVSS | |
| DR2485aa-19 | EVQLLESGGGLVQAGGSLRLSCAASGYSNCAYDMTWYRQAPGKEREFVSAIHSDG | 159 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTLTVSSA | |
| DR2485aa-20 | EVQLLESGGGLVQAGGSLRLSCAASGYSNCAYDMTWYRQAPGKEREFVSAIHSDG | 160 |
| | STRYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTLTVSSAA | |
| DR2519aa-1 | EVQLLESGGGLVQAGGSLRLSCAASGYSNCAYDMTWYRQAPGKEREFVSAIHSDG | 161 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTGTVSS | |
| DR2519aa-2 | EVQLLESGGGLVQAGGSLRLSCAASGYSNCAYDMTWYRQAPGKEREFVSAIHSDG | 162 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTLTVSS | |
| DR2519aa-3 | EVQLLESGGGLVQAGGSLRLSCAASGYSNCAYDMTWYRQAPGKEREFVSAIHSDG | 163 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTLTVSSAA | |
| DR2519aa-4 | EVQLLESGGGLVQAGGSLRLSCAASGYSNCAYDMTWYRQAPGKEREFVSAIHSDG | 164 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTVTVSA | |
| DR2519aa-5 | EVQLLESGGGLVQAGGSLRLSCAASGYSNCAYDMTWYRQAPGKEREFVSAIHSDG | 165 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTVTVSAAA | |
| DR2519aa-6 | EVQLLESGGGLVQAGGSLRLSCAASGYSNCAYDMTWYRQAPGKEREFVSAIHSDG | 166 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTVTVCS | |
| DR2519aa-7 | EVQLLESGGGLVQAGGSLRLSCAASGYSNCAYDMTWYRQAPGKEREFVSAIHSDG | 167 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTVTVCSA | |
| DR2519aa-8 | EVQLLESGGGLVQAGGSLRLSCAASGYSNCAYDMTWYRQAPGKEREFVSAIHSDG | 168 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTVTVCSAA | |
| DR2519aa-9 | EVQLLESGGGLVQAGGSLRLSCAASGYSNCAYDMTWYRQAPGKEREFVSAIHSDG | 169 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTVTVSC | |
| DR2519aa-10 | EVQLLESGGGLVQAGGSLRLSCAASGYSNCAYDMTWYRQAPGKEREFVSAIHSDG | 170 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTVTVSCAA | |
| DR2519aa- | EVQLLESGGGSVQAGGSLRLSCAASGYSNCAYDMTWYRQAPGKEREFVSAIHSDG | 171 |
| 11 (L11S) | STYYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLLTVSS | |
| DR2519aa- | EVQLLESGGGSVQAGGSLRLSCAASGYSNCAYDMTWYRQAPGKEREFVSAIHSDG | 172 |
| 12 (L11S) | STYYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLLTVSSA | |
| DR2519aa- | EVQLLESGGGSVQAGGSLRLSCAASGYSNCAYDMTWYRQAPGKEREFVSAIHSDG | 173 |
| 13 (L11S) | STYYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLLTVSSAA | |
| DR2519aa- | EVQLLESGGGSVQAGGSLRLSCAASGYSNCAYDMTWYRQAPGKEREFVSAIHSDG | 174 |
| 14 (L11S) | STYYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVTT | |
| DR2519aa- | EVQLLESGGGSVQAGGSLRLSCAASGYSNCAYDMTWYRQAPGKEREFVSAIHSDG | 175 |
| 15 (L11S) | STYYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVTTAA | |
| DR2519aa- | EVQLLESGGGSVQAGGSLRLSCAASGYSNCAYDMTWYRQAPGKEREFVSAIHSDG | 174 |
| 16 (L11S) | STYYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVTT | |
| DR2519aa- | EVQLLESGGGSVQAGGSLRLSCAASGYSNCAYDMTWYRQAPGKEREFVSAIHSDG | 175 |
| 17 (L11S) | STYYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVTTAA | |
| DR2519aa-18 | EVQLLESGGGLVQAGGSLRLSCAASGYSNCAYDMTWYRQAPGKEREFVSAIHSDG | 162 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTLTVSS | |
| DR2519aa-19 | EVQLLESGGGLVQAGGSLRLSCAASGYSNCAYDMTWYRQAPGKEREFVSAIHSDG | 179 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTLTVSSA | |
| DR2519aa-20 | EVQLLESGGGLVQAGGSLRLSCAASGYSNCAYDMTWYRQAPGKEREFVSAIHSDG | 163 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTLTVSSAA | |
| DR2520aa-1 | EVQLLESGGGLVQAGGSLRLSCAASGYSNCAYDMTWYRQAPGKEREFVSAIHSSG | 181 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTGTVSS | |
| DR2520aa-2 | EVQLLESGGGLVQAGGSLRLSCAASGYSNCAYDMTWYRQAPGKEREFVSAIHSSG | 182 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTLTVSS | |
| DR2520aa-3 | EVQLLESGGGLVQAGGSLRLSCAASGYSNCAYDMTWYRQAPGKEREFVSAIHSSG | 183 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTLTVSSAA | |
| DR2520aa-4 | EVQLLESGGGLVQAGGSLRLSCAASGYSNCAYDMTWYRQAPGKEREFVSAIHSSG | 184 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTVTVSA | |
| DR2520aa-5 | EVQLLESGGGLVQAGGSLRLSCAASGYSNCAYDMTWYRQAPGKEREFVSAIHSSG | 185 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTVTVSAAA | |
| DR2520aa-6 | EVQLLESGGGLVQAGGSLRLSCAASGYSNCAYDMTWYRQAPGKEREFVSAIHSSG | 186 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTVTVCS | |
| DR2520aa-7 | EVQLLESGGGLVQAGGSLRLSCAASGYSNCAYDMTWYRQAPGKEREFVSAIHSSG | 187 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTVTVCSA | |
| DR2520aa-8 | EVQLLESGGGLVQAGGSLRLSCAASGYSNCAYDMTWYRQAPGKEREFVSAIHSSG | 188 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTVTVCSAA | |
| DR2520aa-9 | EVQLLESGGGLVQAGGSLRLSCAASGYSNCAYDMTWYRQAPGKEREFVSAIHSSG | 189 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTVTVSC | |
| DR2520aa-10 | EVQLLESGGGLVQAGGSLRLSCAASGYSNCAYDMTWYRQAPGKEREFVSAIHSSG | 190 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTVTVSCAA | |
| DR2520aa- | EVQLLESGGGSVQAGGSLRLSCAASGYSNCAYDMTWYRQAPGKEREFVSAIHSSG | 191 |
| 11 (L11S) | STYYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLLTVSS | |
| DR2520aa- | EVQLLESGGGSVQAGGSLRLSCAASGYSNCAYDMTWYRQAPGKEREFVSAIHSSG | 192 |
| 12 (L11S) | STYYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLLTVSSA | |
| DR2520aa- | EVQLLESGGGSVQAGGSLRLSCAASGYSNCAYDMTWYRQAPGKEREFVSAIHSSG | 193 |
| 13 (L11S) | STYYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLLTVSSAA | |
| DR2520aa- | EVQLLESGGGSVQAGGSLRLSCAASGYSNCAYDMTWYRQAPGKEREFVSAIHSSG | 194 |
| 14 (L11S) | STYYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVTT | |
| DR2520aa- | EVQLLESGGGSVQAGGSLRLSCAASGYSNCAYDMTWYRQAPGKEREFVSAIHSSG | 195 |
| 15 (L11S) | STYYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVTTAA | |
| DR2520aa- | EVQLLESGGGSVQAGGSLRLSCAASGYSNCAYDMTWYRQAPGKEREFVSAIHSSG | 194 |
| 16 (L11S) | STYYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVTT | |
| DR2520aa- | EVQLLESGGGSVQAGGSLRLSCAASGYSNCAYDMTWYRQAPGKEREFVSAIHSSG | 195 |
| 17 (L11S) | STYYADSVKGRFFISRDNSKNTVYLQMNSLRPEDTAVYYCKTDPLHCRAHGGSWY | |
| | SVRANYWGQGTLVTVSSGGGSEVQLVESGGGSVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVTTAA | |
| DR2520aa-18 | EVQLLESGGGLVQAGGSLRLSCAASGYSNCAYDMTWYRQAPGKEREFVSAIHSSG | 182 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTLTVSS | |
| DR2520aa-19 | EVQLLESGGGLVQAGGSLRLSCAASGYSNCAYDMTWYRQAPGKEREFVSAIHSSG | 199 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTLTVSSA | |
| DR2520aa-20 | EVQLLESGGGLVQAGGSLRLSCAASGYSNCAYDMTWYRQAPGKEREFVSAIHSSG | 183 |
| | STYYADSVKGRFFISRDNSKNTVYLQMNSLRPEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTTLTVSSAA | |
| DR2096aa-1 | EVQLLESGGGSVQPGGSLRLSCAASGYSNCAYDMTWYRQAPGKEREFVSAIHSDG | 24 |
| | STRYADSVKGRFFISRDNSKNTLYLQMNSLRAEDTAVYYCKTDPLHCRAHGGSWY | |
| | SVRANYWGQGTLVTVSSGGGSEVQLVESGGGLVQPGGSLRLSCAASGYTHSSYCM | |
| | GWVRQAPGKGLEGVSAIDVDGSTTYADSVKGRFTISRDNSKNTLYLQMNSLRAED | |
| | TAVYYCAAEFADCSSNYFLPPGAVRYWGQGTLVTVSAAS | |

TABLE 6
Humanized IL-10Rα-IL-10Rβ VHH Dimers
Nucleic Acid Sequences

| IL- | | |
|---|---|---|
| 10Rα/IL- | | |
| 10Rβ VHH | | |
| Dimer DNA | | SEQ ID |
| Number | DNA Sequence | NO: |
| DR2463 | GAGGTGCAGCTCCTGGAATCTGGCGGGGGCCTGGTCCAGGCTGGCGGTAGCCTCCG | 200 |
| | CCTTAGTTGTGCTGCCAGCGGTTACAGCAACTGCGCTTACGACATGACGTGGTATC | |
| | GCCAGGCCCCAGGGAAGGAGAGGGAGTTCGTCAGCGCTATCCATTCCGATGGGAGC | |
| | ACACGCTACGCTGACTCCGTGAAAGGGAGGTTCTTTATCTCTCGTGACAACAGCAA | |
| | AAACACAGTGTACCTCCAGATGAACTCATTGAGACCAGAGGATACAGCTGTGTATT | |
| | ACTGTAAGACAGACCCTCTGCACTGTCGCGCCCACGGGGGCTCCTGGTATTCTGTG | |
| | CGTGCCAACTATTGGGGCCAGGGAACACTGGTTACCGTCTCTAGCGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGTCTGCT | |
| DR2485 | GAGGTGCAGCTCCTGGAATCTGGCGGGGGCCTGGTCCAGGCTGGCGGTAGCCTCCG | 201 |
| | CCTTAGTTGTGCTGCCAGCGGTTACAGCAACTGCGCTTACGACATGACGTGGTATC | |
| | GCCAGGCCCCAGGGAAGGAGAGGGAGTTCGTCAGCGCTATCCATTCCGATGGGAGC | |
| | ACACGCTACGCTGACTCCGTGAAAGGGAGGTTCTTTATCTCTCGTGACAACAGCAA | |
| | AAACACAGTGTACCTCCAGATGAACTCATTGAGACCAGAGGATACAGCTGTGTATT | |
| | ACTGTAAGACAGACCCTCTGCACTGTCGCGCCCACGGGGGCTCCTGGTATTCTGTG | |
| | CGTGCCAACTATTGGGGCCAGGGAACACTGGTTACCGTCTCTAGCGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCACGGTAACTGTCTCGTCT | |
| DR2519 | GAGGTGCAGCTCCTGGAATCTGGCGGGGGCCTGGTCCAGGCTGGCGGTAGCCTCCG | 202 |
| | CCTTAGTTGTGCTGCCAGCGGTTACAGCAACTGCGCTTACGACATGACGTGGTATC | |
| | GCCAGGCCCCAGGGAAGGAGAGGGAGTTCGTCAGCGCTATCCATTCCGATGGGAGC | |
| | ACATATTACGCTGACTCCGTGAAAGGGAGGTTCTTTATCTCTCGTGACAACAGCAA | |
| | AAACACAGTGTACCTCCAGATGAACTCATTGAGACCAGAGGATACAGCTGTGTATT | |
| | ACTGTAAGACAGACCCTCTGCACTGTCGCGCCCACGGGGGCTCCTGGTATTCTGTG | |
| | CGTGCCAACTATTGGGGCCAGGGAACACTGGTTACCGTCTCTAGCGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCACGGTAACTGTCTCGTCT | |
| DR2520 | GAGGTGCAGCTCCTGGAATCTGGCGGGGGCCTGGTCCAGGCTGGCGGTAGCCTCCG | 203 |
| | CCTTAGTTGTGCTGCCAGCGGTTACAGCAACTGCGCTTACGACATGACGTGGTATC | |
| | GCCAGGCCCCAGGGAAGGAGAGGGAGTTCGTCAGCGCTATCCATTCCTCTGGGAGC | |
| | ACATATTACGCTGACTCCGTGAAAGGGAGGTTCTTTATCTCTCGTGACAACAGCAA | |
| | AAACACAGTGTACCTCCAGATGAACTCATTGAGACCAGAGGATACAGCTGTGTATT | |
| | ACTGTAAGACAGACCCTCTGCACTGTCGCGCCCACGGGGGCTCCTGGTATTCTGTG | |
| | CGTGCCAACTATTGGGGCCAGGGAACACTGGTTACCGTCTCTAGCGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCACGGTAACTGTCTCGTCT | |
| DR2457 | GAAGTCCAGCTCCAGGAATCAGGAGGTGGCTCTGTGCAACCCGGTGGCTCCCTGCG | 204 |
| | CCTCAGCTGTGCCGCTTCTGGATATTCTAACTGCGCTTACGACATGACTTGGTATA | |
| | GGCAGGCACCGGGCAAGGAGCGCGAGTTCGTTTCTGCCATTCACTCTGATGGATCT | |
| | ACCCGCTATGCCGATAGCGTGAAGGGTCGCTTTTTCATCTCCCAGGATAACGCTAA | |
| | AAATACTGTGTACCTTCAGATGAACAGCCTGAAACCTGAAGACACCGCCATGTACT | |
| | ATTGTAAGACAGACCCGTTGCATTGCAGAGCCCACGGAGGCTCATGGTATTCCGTC | |
| | CGGGCGAACTACTGGGGCCAGGGCACTCAGGTGACCGTATCCTCTGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGTCTGCT | |
| DR2458 | GAGGTTCAACTCCAGGAGAGCGGCGGGGGCCTGGTGCAAGCTGGGGGCAGTCTGCG | 205 |
| | CTTGAGCTGCGCTGCATCCGGCTATTCCAACTGTGCCTATGACATGACGTGGTATA | |
| | GGCAGGCCCCTGGGAAGGAGCGTGAGTTCGTGAGTGCTATCCACAGCGACGGCTCC | |
| | ACTAGATACGCGGACTCCGTTAAAGGAAGATTTTTCATCAGTCAGGACAACAGCAA | |
| | GAATACCGTGTATTTGCAGATGAATAGTCTGAAGCCCGAGGACACAGCAGTCTACT | |
| | ATTGTAAGACTGACCCACTGCACTGTCGTGCGCATGGAGGCTCTTGGTACTCCGTG | |
| | AGAGCCAATTATTGGGGGCAGGGCACCCTTGTTACCGTATCCTCCGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGTCTGCT | |
| DR2459 | GAGGTGCAGCTCCAGGAGAGCGGCGGTGGCCTCGTCCAAGCCGGGGGCTCCCTGCG | 206 |
| | TTTGTCTTGCGCGGCCAGCGGATATTCCAACTGTGCGTATGACATGACATGGTATC | |
| | GCCAGGCTCCAGGCAAGGAGCGTGAGTTTGTGTCTGCAATCCACTCTGATGGCTCC | |
| | ACACGCTACGCCGACAGCGTGAAGGGCCGCTTCTTTATCTCCAGAGATAACAGCAA | |
| | GAACACTGTCTATCTCCAGATGAACAGCCTGAAGCCCGAAGACACCGCCGTTTATT | |
| | ACTGTAAGACCGACCCTCTGCATTGCAGAGCCCACGGCGGTTCCTGGTATTCCGTC | |
| | CGCGCTAACTATTGGGGACAGGGGACCCTTGTGACAGTCAGCTCCGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGTCTGCT | |
| DR2460 | GAGGTGCAGTTGCTGGAGTCTGGTGGGGGCCTCGTGCAGGCCGGAGGCTCCCTGCG | 207 |
| | CCTGTCCTGCGCTGCCTCTGGATACTCTAATTGCGCATACGACATGACATGGTATC | |
| | GGCAGGCACCCGGAAAGGAGCGCGAGTTCGTATCAGCAATCCATAGTGACGGTTCT | |
| | ACTCGCTATGCAGACTCTGTGAAAGGACGCTTCTTTATTAGCCAGGACAACTCTAA | |
| | AAACACTGTCTATCTCCAAATGAACTCCCTCAAGCCAGAAGATACTGCCGTGTATT | |
| | ACTGCAAGACCGACCCACTGCATTGTCGTGCGCATGGAGGGTCTTGGTATTCAGTA | |
| | CGTGCAAACTACTGGGGACAGGGCACCCTGGTCACTGTGTCCTCTGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGTCTGCT | |
| DR2461 | GAGGTACAGCTCTTGGAGTCCGGCGGTGGCTTGGTGCAGGCTGGAGGTAGCTTGCG | 208 |
| | CCTGTCCTGCGCCGCTTCCGGCTACAGCAACTGCGCCTACGACATGACGTGGTATC | |
| | GGCAGGCTCCAGGTAAGGAGCGCGAGTTCGTCAGTGCCATTCACTCCGACGGGAGC | |
| | ACTAGATATGCCGACTCTGTCAAGGGACGTTTTTTCATTAGTCGCGACAACTCTAA | |
| | GAACACCGTGTACCTTCAGATGAACAGCCTGAAGCCCGAGGACACTGCCGTGTACT | |
| | ATTGCAAGACCGACCCCCTCCATTGCAGGGCTCATGGGGGCTCTTGGTATTCAGTG | |
| | AGAGCCAACTATTGGGGACAGGGTACGCTGGTGACCGTGTCATCTGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGTCTGCT | |
| DR2462 | GAGGTACAGCTCTTGGAGTCCGGCGGTGGCTTGGTGCAGGCTGGAGGTAGCTTGCG | 209 |
| | CCTGTCCTGCGCCGCTTCCGGCTACAGCAACTGCGCCTACGACATGACGTGGTATC | |
| | GGCAGGCTCCAGGTAAGGAGCGCGAGTTCGTCAGTGCCATTCACTCCGACGGGAGC | |
| | ACTAGATATGCCGACTCTGTCAAGGGACGTTTTTTCATTAGTCGCGACAACTCTAA | |
| | GAACACCGTGTACCTTCAGATGAACAGCCTGAAGCCCGAGGACACTGCCGTGTACT | |
| | ATTGCAAGACCGACCCCCTCCATTGCAGGGCTCATGGGGGCTCTTGGTATTCAGTG | |
| | AGAGCCAACTATTGGGGACAGGGTACGCTGGTGACCGTGTCATCTGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGTCTGCT | |
| DR2464 | GAGGTTCAGTTGCTGGAATCTGGGGGCGGTCTGGTCCAGGCTGGGGGCTCTCTGAG | 210 |
| | GCTGAGCTGCGCCGCATCTGGCTACTCCAACTGCGCCTATGATATGACTTGGTACA | |
| | GGCAGGCTCCCGGCAAGGAGCGCGAGTTCGTGAGCGCCATTCACTCTGATGGTTCC | |
| | ACAAGGTATGCTGATAGTGTTAAAGGACGCTTTTTCATTAGCCGGGACAATAGCAA | |
| | AAACACAGTCTATCTCCAGATGAACTCCCTCCGGGCGGAGGATACCGCTGTCTATT | |
| | ACTGCAAGACTGACCCTTTGCACTGTAGAGCACACGGAGGCAGCTGGTATTCTGTG | |
| | AGAGCCAACTATTGGGGACAAGGAACCCTCGTCACGGTGTCTAGCGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGTCTGCT | |
| DR2465 | GAGGTGCAGCTCCTGGAGTCTGGCGGTGGCCTGGTGCAAGCTGGCGGTTCCCTTCG | 211 |
| | TCTGTCCTGCGCAGCCAGTGGCTATTCTAACTGTGCTTACGACATGACATGGTATC | |
| | GTCAGGCACCGGGGAAGGAGCGCGAGTTCGTATCAGCTATCCACTCTGACGGCAGT | |
| | ACCAGATATGCCGACAGCGTCAAAGGGCGTTTTTTCATCTCCCGCGACAACTCCAA | |
| | AAATACCCTGTATCTTCAGATGAACTCCCTCAGGCCTGAGGACACTGCCGTCTATT | |
| | ACTGCAAGACTGACCCCCTCCACTGTCGCGCTCATGGGGGTAGTTGGTACTCTGTG | |
| | CGTGCTAATTACTGGGGGCAGGGCACCCTGGTGACCGTTAGCTCAGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGTCTGCT | |
| DR2466 | GAGGTGCAGTTGCTGGAGAGCGGGGGCGGGCTCGTGCAGGCGGGAGGCAGCTTGCG | 212 |
| | CTTGTCATGTGCTGCCAGCGGTTATAGCAACTGCGCCTACGACATGACATGGTATC | |
| | GCCAGGCTCCCGGTAAAGAGAGGGAGTTTGTATCCGCGATCCACAGTGACGGTAGC | |
| | ACCCGCTACGCGGATTCCGTGAAAGGACGTTTCTTTATTTCTAGGGATAATAGCAA | |
| | GAATACACTGTACTTGCAAATGAACTCCCTCCGCGCCGAGGACACTGCTGTGTATT | |
| | ACTGCAAAACTGATCCACTGCACTGTCGCGCTCACGGGGGTTCTTGGTATTCAGTA | |
| | CGCGCCAACTACTGGGGCCAGGGTACTCTCGTGACGGTGAGCAGCGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGTCTGCT | |
| DR2499 | GAGGTGCAGCTCCTGGAATCTGGCGGGGGCCTGGTCCAGGCTGGCGGTAGCCTCCG | 213 |
| | CCTTAGTTGTGCTGCCAGCGGTTACAGCAACTGCGCTTACGCCATGACGTGGTATC | |
| | GCCAGGCCCCAGGGAAGGAGAGGGAGTTCGTCAGCGCTATCCATTCCGATGGGAGC | |
| | ACACGCTACGCTGACTCCGTGAAAGGGAGGTTCTTTATCTCTCGTGACAACAGCAA | |
| | AAACACAGTGTACCTCCAGATGAACTCATTGAGACCAGAGGATACAGCTGTGTATT | |
| | ACTGTAAGACAGACCCTCTGCACTGTCGCGCCCACGGGGGCTCCTGGTATTCTGTG | |
| | CGTGCCAACTATTGGGGCCAGGGAACACTGGTTACCGTCTCTAGCGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGTCTGCT | |
| DR2500 | GAGGTGCAGCTCCTGGAATCTGGCGGGGGCCTGGTCCAGGCTGGCGGTAGCCTCCG | 214 |
| | CCTTAGTTGTGCTGCCAGCGGTTACAGCAACTGCGCTTACGCCATGACGTGGTATC | |
| | GCCAGGCCCCAGGGAAGGAGAGGGAGTTCGTCAGCGCTATCCATTCCGATGGGAGC | |
| | ACACGCTACGCTGACTCCGTGAAAGGGAGGTTCTTTATCTCTCGTGACAACAGCAA | |
| | AAACACAGTGTACCTCCAGATGAACTCATTGAGACCAGAGGATACAGCTGTGTATT | |
| | ACTGTAAGACAGACCCTCTGCACTGTCGCGCCCACGGGGGCTCCTGGTATTCTGTG | |
| | CGTGCCAACTATTGGGGCCAGGGAACACTGGTTACCGTCTCTAGCGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGTCTGCT | |
| DR2501 | GAGGTGCAGCTCCTGGAATCTGGCGGGGGCCTGGTCCAGGCTGGCGGTAGCCTCCG | 215 |
| | CCTTAGTTGTGCTGCCAGCGGTTACAGCAACTGCGCTTACGACATGACGTGGTATC | |
| | GCCAGGCCCCAGGGAAGGAGAGGGAGTTCGTCAGCGCTATCTCTTCCGATGGGAGC | |
| | ACACGCTACGCTGACTCCGTGAAAGGGAGGTTCTTTATCTCTCGTGACAACAGCAA | |
| | AAACACAGTGTACCTCCAGATGAACTCATTGAGACCAGAGGATACAGCTGTGTATT | |
| | ACTGTAAGACAGACCCTCTGCACTGTCGCGCCCACGGGGGCTCCTGGTATTCTGTG | |
| | CGTGCCAACTATTGGGGCCAGGGAACACTGGTTACCGTCTCTAGCGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGTCTGCT | |
| DR0000 | GAGGTGCAGCTCCTGGAATCTGGCGGGGGCCTGGTCCAGGCTGGCGGTAGCCTCCG | 216 |
| | CCTTAGTTGTGCTGCCAGCGGTTACAGCAACTGCGCTTACGACATGACGTGGTATC | |
| | GCCAGGCCCCAGGGAAGGAGAGGGAGTTCGTCAGCGCTATCTCTTCCGATGGGAGC | |
| | ACACGCTACGCTGACTCCGTGAAAGGGAGGTTCTTTATCTCTCGTGACAACAGCAA | |
| | AAACACAGTGTACCTCCAGATGAACTCATTGAGACCAGAGGATACAGCTGTGTATT | |
| | ACTGTAAGACAGACCCTCTGCACTGTCGCGCCCACGGGGGCTCCTGGTATTCTGTG | |
| | CGTGCCAACTATTGGGGCCAGGGAACACTGGTTACCGTCTCTAGCGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGTCTG | |
| DR2502 | GAGGTGCAGCTCCTGGAATCTGGCGGGGGCCTGGTCCAGGCTGGCGGTAGCCTCCG | 217 |
| | CCTTAGTTGTGCTGCCAGCGGTTACAGCAACTGCGCTTACGACATGACGTGGTATC | |
| | GCCAGGCCCCAGGGAAGGAGAGGGAGTTCGTCAGCGCTATCCATTCCAGTGGGAGC | |
| | ACACGCTACGCTGACTCCGTGAAAGGGAGGTTCTTTATCTCTCGTGACAACAGCAA | |
| | AAACACAGTGTACCTCCAGATGAACTCATTGAGACCAGAGGATACAGCTGTGTATT | |
| | ACTGTAAGACAGACCCTCTGCACTGTCGCGCCCACGGGGGCTCCTGGTATTCTGTG | |
| | CGTGCCAACTATTGGGGCCAGGGAACACTGGTTACCGTCTCTAGCGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGTCTG | |
| DR2503 | GAGGTGCAGCTCCTGGAATCTGGCGGGGGCCTGGTCCAGGCTGGCGGTAGCCTCCG | 218 |
| | CCTTAGTTGTGCTGCCAGCGGTTACAGCAACTGCGCTTACGACATGACGTGGTATC | |
| | GCCAGGCCCCAGGGAAGGAGAGGGAGTTCGTCAGCGCTATCCATTCCGATGGGAGC | |
| | ACATACTACGCTGACTCCGTGAAAGGGAGGTTCTTTATCTCTCGTGACAACAGCAA | |
| | AAACACAGTGTACCTCCAGATGAACTCATTGAGACCAGAGGATACAGCTGTGTATT | |
| | ACTGTAAGACAGACCCTCTGCACTGTCGCGCCCACGGGGGCTCCTGGTATTCTGTG | |
| | CGTGCCAACTATTGGGGCCAGGGAACACTGGTTACCGTCTCTAGCGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGTCTGCT | |
| DR2504 | GAGGTGCAGCTCCTGGAATCTGGCGGGGGCCTGGTCCAGGCTGGCGGTAGCCTCCG | 219 |
| | CCTTAGTTGTGCTGCCAGCGGTTACAGCAACTGCGCTTACGCCATGTCGTGGTATC | |
| | GCCAGGCCCCAGGGAAGGAGAGGGAGTTCGTCAGCGCTATCCATTCCGATGGGAGC | |
| | ACACGCTACGCTGACTCCGTGAAAGGGAGGTTCTTTATCTCTCGTGACAACAGCAA | |
| | AAACACAGTGTACCTCCAGATGAACTCATTGAGACCAGAGGATACAGCTGTGTATT | |
| | ACTGTAAGACAGACCCTCTGCACTGTCGCGCCCACGGGGGCTCCTGGTATTCTGTG | |
| | CGTGCCAACTATTGGGGCCAGGGAACACTGGTTACCGTCTCTAGCGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGTCTGCT | |
| DR2505 | GAGGTGCAGCTCCTGGAATCTGGCGGGGGCCTGGTCCAGGCTGGCGGTAGCCTCCG | 220 |
| | CCTTAGTTGTGCTGCCAGCGGTTACAGCAACTGCGCTTACGACATGACGTGGTATC | |
| | GCCAGGCCCCAGGGAAGGAGAGGGAGTTCGTCAGCGCTATCTCTGGCAGTGGGAGC | |
| | ACATACTACGCTGACTCCGTGAAAGGGAGGTTCTTTATCTCTCGTGACAACAGCAA | |
| | AAACACAGTGTACCTCCAGATGAACTCATTGAGACCAGAGGATACAGCTGTGTATT | |
| | ACTGTAAGACAGACCCTCTGCACTGTCGCGCCCACGGGGGCTCCTGGTATTCTGTG | |
| | CGTGCCAACTATTGGGGCCAGGGAACACTGGTTACCGTCTCTAGCGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGTCTGCT | |
| DR2506 | GAGGTGCAGCTCCTGGAATCTGGCGGGGGCCTGGTCCAGGCTGGCGGTAGCCTCCG | 221 |
| | CCTTAGTTGTGCTGCCAGCGGTTACAGCAACTGCGCTTACGCCATGTCGTGGTATC | |
| | GCCAGGCCCCAGGGAAGGAGAGGGAGTTCGTCAGCGCTATCTCTGGCAGTGGGAGC | |
| | ACATACTACGCTGACTCCGTGAAAGGGAGGTTCTTTATCTCTCGTGACAACAGCAA | |
| | AAACACAGTGTACCTCCAGATGAACTCATTGAGACCAGAGGATACAGCTGTGTATT | |
| | ACTGTAAGACAGACCCTCTGCACTGTCGCGCCCACGGGGGCTCCTGGTATTCTGTG | |
| | CGTGCCAACTATTGGGGCCAGGGAACACTGGTTACCGTCTCTAGCGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGTCTGCT | |
| DR1525 | GAGGTCCAGTTGCAGGAGTCCGGTGGCGGGTTGGTCCAAGCAGGCGGAAGCCTCCG | 222 |
| | CCTTAGTTGTGCGGCCTCTGGCTACTCTAACTGCGCCTATGATATGACGTGGTATC | |
| | GCCAAGCTCCAGGCAAAGAAAGAGAGTTCGTCAGCGCCATTCACTCTGATGGCTCC | |
| | ACTAGGTACGCAGACAGCGTCAAGGGTCGTTTTTTCATCTCCCGCGATAACTCCAA | |
| | GAACACCGTGTACCTCCAAATGAACTCCCTGAGACCGGAGGACACTGCTGTGTACT | |
| | ATTGCAAGACCGATCCTTTGCACTGTAGGGCGCATGGAGGCAGTTGGTACTCCGTT | |
| | AGAGCCAACTACTGGGGGCAAGGAACCCTGGTCACTGTCAGCTCCGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGTCTGCT | |
| DR2096 | GAAGTGCAGCTCCTGGAGAGCGGCGGTGGCTCGGTGCAGCCAGGCGGTTCTCTGCG | 223 |
| | CTTGTCCTGTGCTGCCTCCGGCTACTCAAACTGTGCTTACGATATGACTTGGTATC | |
| | GCCAGGCCCCTGGTAAGGAGCGCGAGTTCGTCTCTGCCATTCATTCTGACGGCTCA | |
| | ACCCGTTATGCAGACTCCGTTAAGGGGCGTTTTTTCATTTCCCGCGACAACTCCAA | |
| | GAACACCCTGTACCTTCAGATGAACTCCCTCCGCGCCGAGGATACCGCCGTATACT | |
| | ATTGCAAGACCGACCCCCTGCATTGCAGGGCTCACGGCGGGTCCTGGTACTCCGTG | |
| | CGTGCCAACTATTGGGGACAGGGGACTCTGGTCACAGTCTCGAGTGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGGCAGCT | |

TABLE 7
IL-10Rα VHH CDRs

| Ab DNA | IL-10Rα | SEQ | | SEQ | | SEQ |
|---|---|---|---|---|---|---|
| Number | CDR1 | ID# | IL-10Rα CDR2 | ID# | IL-10Rα CDR3 | ID# |
| DR2463 | YSNCAYDMT | 224 | AIHSDGSTRYADSVKG | 229 | KTDPLHCRAHGGSWYSVRANY | 236 |
| DR2457 | | | | | | |
| DR2458 | | | | | | |
| DR2459 | | | | | | |
| DR2460 | | | | | | |
| DR2461 | | | | | | |
| DR2462 | | | | | | |
| DR2464 | | | | | | |
| DR2465 | | | | | | |
| DR2466 | | | | | | |
| DR2096 | | | | | | |
| DR2519 | | | | | | |
| DR2485 | YSNCAYDMT | 224 | AIHSDGSTYYADSVKG | 230 | KTDPLHCRAHGGSWYSVRANY | 236 |
| DR2503 | | | | | | |
| DR2520 | YSNCAYDMT | 224 | AIHSSGSTYYADSVKG | 231 | KTDPLHCRAHGGSWYSVRANY | 236 |
| DR2499 | YSNCAYAMT | 225 | AIHSDGSTRYADSVKG | 229 | KTDPLHCRAHGGSWYSVRANY | 236 |
| DR2500 | YSNCAYDMS | 226 | AIHSDGSTRYADSVKG | 229 | KTDPLHCRAHGGSWYSVRANY | 236 |
| DR2501 | YSNCAYDMT | 224 | AISSDGSTRYADSVKG | 232 | KTDPLHCRAHGGSWYSVRANY | 236 |
| DR0000 | YSNCAYDMT | 224 | AIHGDGSTRYADSVKG | 233 | KTDPLHCRAHGGSWYSVRANY | 236 |
| DR2502 | YSNCAYDMT | 224 | AIHSSGSTRYADSVKG | 234 | KTDPLHCRAHGGSWYSVRANY | 236 |
| DR2504 | YSNCAYAMS | 227 | AIHSDGSTRYADSVKG | 229 | KTDPLHCRAHGGSWYSVRANY | 236 |
| DR2505 | YSNCAYDMT | 224 | AISGSGSTYYADSVKG | 235 | KTDPLHCRAHGGSWYSVRANY | 236 |
| DR2506 | YSNCAYAMS | 227 | AISGSGSTYYADSVKG | 235 | KTDPLHCRAHGGSWYSVRANY | 236 |
| DR1525 | YSNCAYDM | 228 | AIHSDGSTRYADSVKG | 229 | KTDPLHCRAHGGSWYSVRANY | 236 |

TABLE 8
IL-10Rβ VHH CDRs

| | | SEQ | | SEQ | | SEQ |
|---|---|---|---|---|---|---|
| Ab DNA | IL-10Rβ | ID | | ID | | ID |
| Number | CDR1 | NO | IL-10Rβ CDR2 | NO | IL-10Rβ CDR3 | NO |
| DR2463 | YTHSSYCMG | 296 | AIDVDGSTTYADSVKG | 297 | AAEFADCSSNYFLPPGAVRY | 298 |
| DR2485 | | | | | | |
| DR2519 | | | | | | |
| DR2520 | | | | | | |
| DR2457 | | | | | | |
| DR2458 | | | | | | |
| DR2459 | | | | | | |
| DR2460 | | | | | | |
| DR2461 | | | | | | |
| DR2462 | | | | | | |
| DR2464 | | | | | | |
| DR2465 | | | | | | |
| DR2466 | | | | | | |
| DR2499 | | | | | | |
| DR2500 | | | | | | |
| DR2501 | | | | | | |
| DR0000 | | | | | | |
| DR2502 | | | | | | |
| DR2503 | | | | | | |
| DR2504 | | | | | | |
| DR2505 | | | | | | |
| DR2506 | | | | | | |
| DR1525 | | | | | | |
| DR2096 | | | | | | |

TABLE 9
Humanized hIL-10Rα VHH Nucleic Acid Sequences

| VHH DNA | | SEQ ID |
|---|---|---|
| Number | DNA Sequence | NO: |
| DR2463 | GAGGTGCAGCTCCTGGAATCTGGCGGGGGCCTGGTCCAGGCTGGCGGTAGCCTCCGCC | 368 |
| | TTAGTTGTGCTGCCAGCGGTTACAGCAACTGCGCTTACGACATGACGTGGTATCGCCA | |
| | GGCCCCAGGGAAGGAGAGGGAGTTCGTCAGCGCTATCCATTCCGATGGGAGCACACGC | |
| | TACGCTGACTCCGTGAAAGGGAGGTTCTTTATCTCTCGTGACAACAGCAAAAACACAG | |
| | TGTACCTCCAGATGAACTCATTGAGACCAGAGGATACAGCTGTGTATTACTGTAAGAC | |
| | AGACCCTCTGCACTGTCGCGCCCACGGGGGCTCCTGGTATTCTGTGCGTGCCAACTAT | |
| | TGGGGCCAGGGAACACTGGTTACCGTCTCTAGC | |
| DR2485 | GAGGTGCAGCTCCTGGAATCTGGCGGGGGCCTGGTCCAGGCTGGCGGTAGCCTCCGCC | 369 |
| | TTAGTTGTGCTGCCAGCGGTTACAGCAACTGCGCTTACGACATGACGTGGTATCGCCA | |
| | GGCCCCAGGGAAGGAGAGGGAGTTCGTCAGCGCTATCCATTCCGATGGGAGCACACGC | |
| | TACGCTGACTCCGTGAAAGGGAGGTTCTTTATCTCTCGTGACAACAGCAAAAACACAG | |
| | TGTACCTCCAGATGAACTCATTGAGACCAGAGGATACAGCTGTGTATTACTGTAAGAC | |
| | AGACCCTCTGCACTGTCGCGCCCACGGGGGCTCCTGGTATTCTGTGCGTGCCAACTAT | |
| | TGGGGCCAGGGAACACTGGTTACCGTCTCTAGCG | |
| DR2519 | GAGGTGCAGCTCCTGGAATCTGGCGGGGGCCTGGTCCAGGCTGGCGGTAGCCTCCGCC | 370 |
| | TTAGTTGTGCTGCCAGCGGTTACAGCAACTGCGCTTACGACATGACGTGGTATCGCCA | |
| | GGCCCCAGGGAAGGAGAGGGAGTTCGTCAGCGCTATCCATTCCGATGGGAGCACATAC | |
| | TACGCTGACTCCGTGAAAGGGAGGTTCTTTATCTCTCGTGACAACAGCAAAAACACAG | |
| | TGTACCTCCAGATGAACTCATTGAGACCAGAGGATACAGCTGTGTATTACTGTAAGAC | |
| | AGACCCTCTGCACTGTCGCGCCCACGGGGGCTCCTGGTATTCTGTGCGTGCCAACTAT | |
| | TGGGGCCAGGGAACACTGGTTACCGTCTCTAGC | |
| DR2520 | GAGGTGCAGCTCCTGGAATCTGGCGGGGGCCTGGTCCAGGCTGGCGGTAGCCTCCGCC | 371 |
| | TTAGTTGTGCTGCCAGCGGTTACAGCAACTGCGCTTACGACATGACGTGGTATCGCCA | |
| | GGCCCCAGGGAAGGAGAGGGAGTTCGTCAGCGCTATCCATTCCTCTGGGAGCACATAT | |
| | TACGCTGACTCCGTGAAAGGGAGGTTCTTTATCTCTCGTGACAACAGCAAAAACACAG | |
| | TGTACCTCCAGATGAACTCATTGAGACCAGAGGATACAGCTGTGTATTACTGTAAGAC | |
| | AGACCCTCTGCACTGTCGCGCCCACGGGGGCTCCTGGTATTCTGTGCGTGCCAACTAT | |
| | TGGGGCCAGGGAACACTGGTTACCGTCTCTAGC | |
| DR2457 | GAAGTCCAGCTCCAGGAATCAGGAGGTGGCTCTGTGCAACCCGGTGGCTCCCTGCGCC | 372 |
| | TCAGCTGTGCCGCTTCTGGATATTCTAACTGCGCTTACGACATGACTTGGTATAGGCA | |
| | GGCACCGGGCAAGGAGCGCGAGTTCGTTTCTGCCATTCACTCTGATGGATCTACCCGC | |
| | TATGCCGATAGCGTGAAGGGTCGCTTTTTCATCTCCCAGGATAACGCTAAAAATACTG | |
| | TGTACCTTCAGATGAACAGCCTGAAACCTGAAGACACCGCCATGTACTATTGTAAGAC | |
| | AGACCCGTTGCATTGCAGAGCCCACGGAGGCTCATGGTATTCCGTCCGGGCGAACTAC | |
| | TGGGGCCAGGGCACTCAGGTGACCGTATCCTCT | |
| DR2458 | GAGGTTCAACTCCAGGAGAGCGGCGGGGGCCTGGTGCAAGCTGGGGGCAGTCTGCGCT | 373 |
| | TGAGCTGCGCTGCATCCGGCTATTCCAACTGTGCCTATGACATGACGTGGTATAGGCA | |
| | GGCCCCTGGGAAGGAGCGTGAGTTCGTGAGTGCTATCCACAGCGACGGCTCCACTAGA | |
| | TACGCGGACTCCGTTAAAGGAAGATTTTTCATCAGTCAGGACAACAGCAAGAATACCG | |
| | TGTATTTGCAGATGAATAGTCTGAAGCCCGAGGACACAGCAGTCTACTATTGTAAGAC | |
| | TGACCCACTGCACTGTCGTGCGCATGGAGGCTCTTGGTACTCCGTGAGAGCCAATTAT | |
| | TGGGGGCAGGGCACCCTTGTTACCGTATCCTCC | |
| DR2459 | GAGGTGCAGCTCCAGGAGAGCGGCGGTGGCCTCGTCCAAGCCGGGGGCTCCCTGCGTT | 374 |
| | TGTCTTGCGCGGCCAGCGGATATTCCAACTGTGCGTATGACATGACATGGTATCGCCA | |
| | GGCTCCAGGCAAGGAGCGTGAGTTTGTGTCTGCAATCCACTCTGATGGCTCCACACGC | |
| | TACGCCGACAGCGTGAAGGGCCGCTTCTTTATCTCCAGAGATAACAGCAAGAACACTG | |
| | TCTATCTCCAGATGAACAGCCTGAAGCCCGAAGACACCGCCGTTTATTACTGTAAGAC | |
| | CGACCCTCTGCATTGCAGAGCCCACGGCGGTTCCTGGTATTCCGTCCGCGCTAACTAT | |
| | TGGGGACAGGGGACCCTTGTGACAGTCAGCTCC | |
| DR2460 | GAGGTGCAGTTGCTGGAGTCTGGTGGGGGCCTCGTGCAGGCCGGAGGCTCCCTGCGCC | 375 |
| | TGTCCTGCGCTGCCTCTGGATACTCTAATTGCGCATACGACATGACATGGTATCGGCA | |
| | GGCACCCGGAAAGGAGCGCGAGTTCGTATCAGCAATCCATAGTGACGGTTCTACTCGC | |
| | TATGCAGACTCTGTGAAAGGACGCTTCTTTATTAGCCAGGACAACTCTAAAAACACTG | |
| | TCTATCTCCAAATGAACTCCCTCAAGCCAGAAGATACTGCCGTGTATTACTGCAAGAC | |
| | CGACCCACTGCATTGTCGTGCGCATGGAGGGTCTTGGTATTCAGTACGTGCAAACTAC | |
| | TGGGGACAGGGCACCCTGGTCACTGTGTCCTCT | |
| DR2461 | GAGGTACAGCTCTTGGAGTCCGGCGGTGGCTTGGTGCAGGCTGGAGGTAGCTTGCGCC | 376 |
| | TGTCCTGCGCCGCTTCCGGCTACAGCAACTGCGCCTACGACATGACGTGGTATCGGCA | |
| | GGCTCCAGGTAAGGAGCGCGAGTTCGTCAGTGCCATTCACTCCGACGGGAGCACTAGA | |
| | TATGCCGACTCTGTCAAGGGACGTTTTTTCATTAGTCGCGACAACTCTAAGAACACCG | |
| | TGTACCTTCAGATGAACAGCCTGAAGCCCGAGGACACTGCCGTGTACTATTGCAAGAC | |
| | CGACCCCCTCCATTGCAGGGCTCATGGGGGCTCTTGGTATTCAGTGAGAGCCAACTAT | |
| | TGGGGACAGGGTACGCTGGTGACCGTGTCATCT | |
| DR2462 | GAGGTCCAGTTGCAGGAGTCCGGTGGCGGGTTGGTCCAAGCAGGCGGAAGCCTCCGCC | 377 |
| | TTAGTTGTGCGGCCTCTGGCTACTCTAACTGCGCCTATGATATGACGTGGTATCGCCA | |
| | AGCTCCAGGCAAAGAAAGAGAGTTCGTCAGCGCCATTCACTCTGATGGCTCCACTAGG | |
| | TACGCAGACAGCGTCAAGGGTCGTTTTTTCATCTCCCGCGATAACTCCAAGAACACCG | |
| | TGTACCTCCAAATGAACTCCCTGAGACCGGAGGACACTGCTGTGTACTATTGCAAGAC | |
| | CGATCCTTTGCACTGTAGGGCGCATGGAGGCAGTTGGTACTCCGTTAGAGCCAACTAC | |
| | TGGGGGCAAGGAACCCTGGTCACTGTCAGCTCC | |
| DR2464 | GAGGTTCAGTTGCTGGAATCTGGGGGCGGTCTGGTCCAGGCTGGGGGCTCTCTGAGGC | 378 |
| | TGAGCTGCGCCGCATCTGGCTACTCCAACTGCGCCTATGATATGACTTGGTACAGGCA | |
| | GGCTCCCGGCAAGGAGCGCGAGTTCGTGAGCGCCATTCACTCTGATGGTTCCACAAGG | |
| | TATGCTGATAGTGTTAAAGGACGCTTTTTCATTAGCCGGGACAATAGCAAAAACACAG | |
| | TCTATCTCCAGATGAACTCCCTCCGGGCGGAGGATACCGCTGTCTATTACTGCAAGAC | |
| | TGACCCTTTGCACTGTAGAGCACACGGAGGCAGCTGGTATTCTGTGAGAGCCAACTAT | |
| | TGGGGACAAGGAACCCTCGTCACGGTGTCTAGC | |
| DR2465 | GAGGTGCAGCTCCTGGAGTCTGGCGGTGGCCTGGTGCAAGCTGGCGGTTCCCTTCGTC | 379 |
| | TGTCCTGCGCAGCCAGTGGCTATTCTAACTGTGCTTACGACATGACATGGTATCGTCA | |
| | GGCACCGGGGAAGGAGCGCGAGTTCGTATCAGCTATCCACTCTGACGGCAGTACCAGA | |
| | TATGCCGACAGCGTCAAAGGGCGTTTTTTCATCTCCCGCGACAACTCCAAAAATACCC | |
| | TGTATCTTCAGATGAACTCCCTCAGGCCTGAGGACACTGCCGTCTATTACTGCAAGAC | |
| | TGACCCCCTCCACTGTCGCGCTCATGGGGGTAGTTGGTACTCTGTGCGTGCTAATTAC | |
| | TGGGGGCAGGGCACCCTGGTGACCGTTAGCTCA | |
| DR2466 | GAGGTGCAGTTGCTGGAGAGCGGGGGCGGGCTCGTGCAGGCGGGAGGCAGCTTGCGCT | 380 |
| | TGTCATGTGCTGCCAGCGGTTATAGCAACTGCGCCTACGACATGACATGGTATCGCCA | |
| | GGCTCCCGGTAAAGAGAGGGAGTTTGTATCCGCGATCCACAGTGACGGTAGCACCCGC | |
| | TACGCGGATTCCGTGAAAGGACGTTTCTTTATTTCTAGGGATAATAGCAAGAATACAC | |
| | TGTACTTGCAAATGAACTCCCTCCGCGCCGAGGACACTGCTGTGTATTACTGCAAAAC | |
| | TGATCCACTGCACTGTCGCGCTCACGGGGGTTCTTGGTATTCAGTACGCGCCAACTAC | |
| | TGGGGCCAGGGTACTCTCGTGACGGTGAGCAGC | |
| DR2499 | GAGGTGCAGCTCCTGGAATCTGGCGGGGGCCTGGTCCAGGCTGGCGGTAGCCTCCGCC | 381 |
| | TTAGTTGTGCTGCCAGCGGTTACAGCAACTGCGCTTACGCCATGACGTGGTATCGCCA | |
| | GGCCCCAGGGAAGGAGAGGGAGTTCGTCAGCGCTATCCATTCCGATGGGAGCACACGC | |
| | TACGCTGACTCCGTGAAAGGGAGGTTCTTTATCTCTCGTGACAACAGCAAAAACACAG | |
| | TGTACCTCCAGATGAACTCATTGAGACCAGAGGATACAGCTGTGTATTACTGTAAGAC | |
| | AGACCCTCTGCACTGTCGCGCCCACGGGGGCTCCTGGTATTCTGTGCGTGCCAACTAT | |
| | TGGGGCCAGGGAACACTGGTTACCGTCTCTAGC | |
| DR2500 | GAGGTGCAGCTCCTGGAATCTGGCGGGGGCCTGGTCCAGGCTGGCGGTAGCCTCCGCC | 381 |
| | TTAGTTGTGCTGCCAGCGGTTACAGCAACTGCGCTTACGCCATGACGTGGTATCGCCA | |
| | GGCCCCAGGGAAGGAGAGGGAGTTCGTCAGCGCTATCCATTCCGATGGGAGCACACGC | |
| | TACGCTGACTCCGTGAAAGGGAGGTTCTTTATCTCTCGTGACAACAGCAAAAACACAG | |
| | TGTACCTCCAGATGAACTCATTGAGACCAGAGGATACAGCTGTGTATTACTGTAAGAC | |
| | AGACCCTCTGCACTGTCGCGCCCACGGGGGCTCCTGGTATTCTGTGCGTGCCAACTAT | |
| | TGGGGCCAGGGAACACTGGTTACCGTCTCTAGC | |
| DR2501 | GAGGTGCAGCTCCTGGAATCTGGCGGGGGCCTGGTCCAGGCTGGCGGTAGCCTCCGCC | 383 |
| | TTAGTTGTGCTGCCAGCGGTTACAGCAACTGCGCTTACGACATGTCGTGGTATCGCCA | |
| | GGCCCCAGGGAAGGAGAGGGAGTTCGTCAGCGCTATCCATTCCGATGGGAGCACACGC | |
| | TACGCTGACTCCGTGAAAGGGAGGTTCTTTATCTCTCGTGACAACAGCAAAAACACAG | |
| | TGTACCTCCAGATGAACTCATTGAGACCAGAGGATACAGCTGTGTATTACTGTAAGAC | |
| | AGACCCTCTGCACTGTCGCGCCCACGGGGGCTCCTGGTATTCTGTGCGTGCCAACTAT | |
| | TGGGGCCAGGGAACACTGGTTACCGTCTCTAGC | |
| DR0000 | GAGGTGCAGCTCCTGGAATCTGGCGGGGGCCTGGTCCAGGCTGGCGGTAGCCTCCGCC | 384 |
| | TTAGTTGTGCTGCCAGCGGTTACAGCAACTGCGCTTACGACATGACGTGGTATCGCCA | |
| | GGCCCCAGGGAAGGAGAGGGAGTTCGTCAGCGCTATCTCTTCCGATGGGAGCACACGC | |
| | TACGCTGACTCCGTGAAAGGGAGGTTCTTTATCTCTCGTGACAACAGCAAAAACACAG | |
| | TGTACCTCCAGATGAACTCATTGAGACCAGAGGATACAGCTGTGTATTACTGTAAGAC | |
| | AGACCCTCTGCACTGTCGCGCCCACGGGGGCTCCTGGTATTCTGTGCGTGCCAACTAT | |
| | TGGGGCCAGGGAACACTGGTTACCGTCTCTAGC | |
| DR2502 | GAGGTGCAGCTCCTGGAATCTGGCGGGGGCCTGGTCCAGGCTGGCGGTAGCCTCCGCC | 385 |
| | TTAGTTGTGCTGCCAGCGGTTACAGCAACTGCGCTTACGACATGACGTGGTATCGCCA | |
| | GGCCCCAGGGAAGGAGAGGGAGTTCGTCAGCGCTATCCATGGCGATGGGAGCACACGC | |
| | TACGCTGACTCCGTGAAAGGGAGGTTCTTTATCTCTCGTGACAACAGCAAAAACACAG | |
| | TGTACCTCCAGATGAACTCATTGAGACCAGAGGATACAGCTGTGTATTACTGTAAGAC | |
| | AGACCCTCTGCACTGTCGCGCCCACGGGGGCTCCTGGTATTCTGTGCGTGCCAACTAT | |
| | TGGGGCCAGGGAACACTGGTTACCGTCTCTAGC | |
| DR2503 | GAGGTGCAGCTCCTGGAATCTGGCGGGGGCCTGGTCCAGGCTGGCGGTAGCCTCCGCC | 386 |
| | TTAGTTGTGCTGCCAGCGGTTACAGCAACTGCGCTTACGACATGACGTGGTATCGCCA | |
| | GGCCCCAGGGAAGGAGAGGGAGTTCGTCAGCGCTATCCATTCCAGTGGGAGCACACGC | |
| | TACGCTGACTCCGTGAAAGGGAGGTTCTTTATCTCTCGTGACAACAGCAAAAACACAG | |
| | TGTACCTCCAGATGAACTCATTGAGACCAGAGGATACAGCTGTGTATTACTGTAAGAC | |
| | AGACCCTCTGCACTGTCGCGCCCACGGGGGCTCCTGGTATTCTGTGCGTGCCAACTAT | |
| | TGGGGCCAGGGAACACTGGTTACCGTCTCTAGC | |
| DR2504 | GAGGTGCAGCTCCTGGAATCTGGCGGGGGCCTGGTCCAGGCTGGCGGTAGCCTCCGCC | 370 |
| | TTAGTTGTGCTGCCAGCGGTTACAGCAACTGCGCTTACGACATGACGTGGTATCGCCA | |
| | GGCCCCAGGGAAGGAGAGGGAGTTCGTCAGCGCTATCCATTCCGATGGGAGCACATAC | |
| | TACGCTGACTCCGTGAAAGGGAGGTTCTTTATCTCTCGTGACAACAGCAAAAACACAG | |
| | TGTACCTCCAGATGAACTCATTGAGACCAGAGGATACAGCTGTGTATTACTGTAAGAC | |
| | AGACCCTCTGCACTGTCGCGCCCACGGGGGCTCCTGGTATTCTGTGCGTGCCAACTAT | |
| | TGGGGCCAGGGAACACTGGTTACCGTCTCTAGC | |
| DR2505 | GAGGTGCAGCTCCTGGAATCTGGCGGGGGCCTGGTCCAGGCTGGCGGTAGCCTCCGCC | 388 |
| | TTAGTTGTGCTGCCAGCGGTTACAGCAACTGCGCTTACGCCATGTCGTGGTATCGCCA | |
| | GGCCCCAGGGAAGGAGAGGGAGTTCGTCAGCGCTATCCATTCCGATGGGAGCACACGC | |
| | TACGCTGACTCCGTGAAAGGGAGGTTCTTTATCTCTCGTGACAACAGCAAAAACACAG | |
| | TGTACCTCCAGATGAACTCATTGAGACCAGAGGATACAGCTGTGTATTACTGTAAGAC | |
| | AGACCCTCTGCACTGTCGCGCCCACGGGGGCTCCTGGTATTCTGTGCGTGCCAACTAT | |
| | TGGGGCCAGGGAACACTGGTTACCGTCTCTAGC | |
| DR2506 | GAGGTGCAGCTCCTGGAATCTGGCGGGGGCCTGGTCCAGGCTGGCGGTAGCCTCCGCC | 389 |
| | TTAGTTGTGCTGCCAGCGGTTACAGCAACTGCGCTTACGACATGACGTGGTATCGCCA | |
| | GGCCCCAGGGAAGGAGAGGGAGTTCGTCAGCGCTATCTCTGGCAGTGGGAGCACATAC | |
| | TACGCTGACTCCGTGAAAGGGAGGTTCTTTATCTCTCGTGACAACAGCAAAAACACAG | |
| | TGTACCTCCAGATGAACTCATTGAGACCAGAGGATACAGCTGTGTATTACTGTAAGAC | |
| | AGACCCTCTGCACTGTCGCGCCCACGGGGGCTCCTGGTATTCTGTGCGTGCCAACTAT | |
| | TGGGGCCAGGGAACACTGGTTACCGTCTCTAGC | |
| DR1525 | GAGGTCCAGTTGCAGGAGTCCGGTGGCGGGTTGGTCCAAGCAGGCGGAAGCCTCCGCC | 377 |
| | TTAGTTGTGCGGCCTCTGGCTACTCTAACTGCGCCTATGATATGACGTGGTATCGCCA | |
| | AGCTCCAGGCAAAGAAAGAGAGTTCGTCAGCGCCATTCACTCTGATGGCTCCACTAGG | |
| | TACGCAGACAGCGTCAAGGGTCGTTTTTTCATCTCCCGCGATAACTCCAAGAACACCG | |
| | TGTACCTCCAAATGAACTCCCTGAGACCGGAGGACACTGCTGTGTACTATTGCAAGAC | |
| | CGATCCTTTGCACTGTAGGGCGCATGGAGGCAGTTGGTACTCCGTTAGAGCCAACTAC | |
| | TGGGGGCAAGGAACCCTGGTCACTGTCAGCTCC | |
| DR2096 | GAAGTGCAGCTCCTGGAGAGCGGCGGTGGCTCGGTGCAGCCAGGCGGTTCTCTGCGCT | 391 |
| | TGTCCTGTGCTGCCTCCGGCTACTCAAACTGTGCTTACGATATGACTTGGTATCGCCA | |
| | GGCCCCTGGTAAGGAGCGCGAGTTCGTCTCTGCCATTCATTCTGACGGCTCAACCCGT | |
| | TATGCAGACTCCGTTAAGGGGCGTTTTTTCATTTCCCGCGACAACTCCAAGAACACCC | |
| | TGTACCTTCAGATGAACTCCCTCCGCGCCGAGGATACCGCCGTATACTATTGCAAGAC | |
| | CGACCCCCTGCATTGCAGGGCTCACGGCGGGTCCTGGTACTCCGTGCGTGCCAACTAT | |
| | TGGGGACAGGGGACTCTGGTCACAGTCTCGAGT | |

TABLE 10
Humanized IL-10Rβ VHH Nucleic Acid Sequences

| IL-10Rβ | | |
|---|---|---|
| VHH DNA | | SEQ ID |
| Number | DNA Sequence | NO: |
| DR2463- | GAGGTGCAGCTGGTGGAGAGCGGAGGAGGGTTAGTGCAGCCGGGCGGTTCTCTCAGAC | 392 |
| hzIL-10Rβ | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCCTGGTAACTGTCTCGTCTGCTAGCCACCATCACCATCACCACTGAT | |
| | AAGCGGCCGC | |
| DR2485- | GAGGTGCAGCTGGTGGAGAGCGGAGGAGGGTTAGTGCAGCCGGGCGGTTCTCTCAGAC | 393 |
| hzIL-10Rβ | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCACGGTAACTGTCTCGTCTTAGTGAGCGGCCGC | |
| DR2519- | GAGGTGCAGCTGGTGGAGAGCGGAGGAGGGTTAGTGCAGCCGGGCGGTTCTCTCAGAC | 393 |
| hzIL-10Rβ | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCACGGTAACTGTCTCGTCTTAGTGAGCGGCCGC | |
| DR2520- | GAGGTGCAGCTGGTGGAGAGCGGAGGAGGGTTAGTGCAGCCGGGCGGTTCTCTCAGAC | 393 |
| hzIL-10Rβ | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCACGGTAACTGTCTCGTCTTAGTGAGCGGCCGC | |
| DR2457 | GAGGTGCAGCTGGTGGAGAGCGGAGGAGGGTTAGTGCAGCCGGGCGGTTCTCTCAGAC | 576 |
| | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCCTGGTAACTGTCTCGTCT | |
| DR2458 | GAGGTGCAGCTGGTGGAGAGCGGAGGAGGGTTAGTGCAGCCGGGCGGTTCTCTCAGAC | 576 |
| | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCCTGGTAACTGTCTCGTCT | |
| DR2459 | GAGGTGCAGCTGGTGGAGAGCGGAGGAGGGTTAGTGCAGCCGGGCGGTTCTCTCAGAC | 576 |
| | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCCTGGTAACTGTCTCGTCT | |
| DR2460 | GAGGTGCAGCTGGTGGAGAGCGGAGGAGGGTTAGTGCAGCCGGGCGGTTCTCTCAGAC | 576 |
| | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCCTGGTAACTGTCTCGTCT | |
| DR2461 | GAGGTGCAGCTGGTGGAGAGCGGAGGAGGGTTAGTGCAGCCGGGCGGTTCTCTCAGAC | 576 |
| | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCCTGGTAACTGTCTCGTCT | |
| DR2462 | GAGGTGCAGCTGGTGGAGAGCGGAGGAGGGTTAGTGCAGCCGGGCGGTTCTCTCAGAC | 576 |
| | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCCTGGTAACTGTCTCGTCT | |
| DR2464 | GAGGTGCAGCTGGTGGAGAGCGGAGGAGGGTTAGTGCAGCCGGGCGGTTCTCTCAGAC | 576 |
| | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCCTGGTAACTGTCTCGTCT | |
| DR2465 | GAGGTGCAGCTGGTGGAGAGCGGAGGAGGGTTAGTGCAGCCGGGCGGTTCTCTCAGAC | 576 |
| | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCCTGGTAACTGTCTCGTCT | |
| DR2466 | GAGGTGCAGCTGGTGGAGAGCGGAGGAGGGTTAGTGCAGCCGGGCGGTTCTCTCAGAC | 576 |
| | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCCTGGTAACTGTCTCGTCT | |
| DR2499 | GAGGTGCAGCTGGTGGAGAGCGGAGGAGGGTTAGTGCAGCCGGGCGGTTCTCTCAGAC | 576 |
| | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCCTGGTAACTGTCTCGTCT | |
| DR2500 | GAGGTGCAGCTGGTGGAGAGCGGAGGAGGGTTAGTGCAGCCGGGCGGTTCTCTCAGAC | 576 |
| | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCCTGGTAACTGTCTCGTCT | |
| DR2501 | GAGGTGCAGCTGGTGGAGAGCGGAGGAGGGTTAGTGCAGCCGGGCGGTTCTCTCAGAC | 576 |
| | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCCTGGTAACTGTCTCGTCT | |
| DR0000 | GAGGTGCAGCTGGTGGAGAGCGGAGGAGGGTTAGTGCAGCCGGGCGGTTCTCTCAGAC | 576 |
| | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCCTGGTAACTGTCTCGTCT | |
| DR2502 | GAGGTGCAGCTGGTGGAGAGCGGAGGAGGGTTAGTGCAGCCGGGCGGTTCTCTCAGAC | 576 |
| | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCCTGGTAACTGTCTCGTCT | |
| DR2503 | GAGGTGCAGCTGGTGGAGAGCGGAGGAGGGTTAGTGCAGCCGGGCGGTTCTCTCAGAC | 576 |
| | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCCTGGTAACTGTCTCGTCT | |
| DR2504 | GAGGTGCAGCTGGTGGAGAGCGGAGGAGGGTTAGTGCAGCCGGGCGGTTCTCTCAGAC | 576 |
| | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCCTGGTAACTGTCTCGTCT | |
| DR2505 | GAGGTGCAGCTGGTGGAGAGCGGAGGAGGGTTAGTGCAGCCGGGCGGTTCTCTCAGAC | 576 |
| | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCCTGGTAACTGTCTCGTCT | |
| DR2506 | GAGGTGCAGCTGGTGGAGAGCGGAGGAGGGTTAGTGCAGCCGGGCGGTTCTCTCAGAC | 576 |
| | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCCTGGTAACTGTCTCGTCT | |
| DR1525 | GAGGTGCAGCTGGTGGAGAGCGGAGGAGGGTTAGTGCAGCCGGGCGGTTCTCTCAGAC | 576 |
| | TTAGCTGCGCCGCCTCCGGGTACACGCACTCTTCCTATTGCATGGGCTGGGTCAGGCA | |
| | GGCGCCTGGGAAGGGCCTGGAGGGAGTTAGTGCCATCGATGTCGATGGCTCAACAACC | |
| | TATGCAGACAGCGTTAAGGGTCGGTTCACCATTTCACGCGACAATAGCAAAAACACTC | |
| | TGTATTTGCAGATGAACAGTCTCCGTGCCGAAGACACAGCTGTCTACTACTGTGCAGC | |
| | TGAATTTGCTGATTGTTCCTCTAATTATTTCCTACCCCCAGGGGCAGTGCGATACTGG | |
| | GGACAAGGCACCCTGGTAACTGTCTCGTCT | |
| DR2096 | GAGGTGCAGCTGGTGGAGAGCGGAGGAGGGTTAGTGCAGCCGGGCGGTTCTCTCA | 415 |
| | GACTTAGCTGCGCCGCCTCCGGGTACACGCACTCTTCCTATTGCATGGGCTGGGT | |
| | CAGGCAGGCGCCTGGGAAGGGCCTGGAGGGAGTTAGTGCCATCGATGTCGATGGC | |
| | TCAACAACCTATGCAGACAGCGTTAAGGGTCGGTTCACCATTTCACGCGACAATA | |
| | GCAAAAACACTCTGTATTTGCAGATGAACAGTCTCCGTGCCGAAGACACAGCTGT | |
| | CTACTACTGTGCAGCTGAATTTGCTGATTGTTCCTCTAATTATTTCCTACCCCCA | |
| | GGGGCAGTGCGATACTGGGGACAAGGCACCCTGGTAACTGTCTCGGCAGCTAGC | |

TABLE 11
VHH Dimer Exemplary Linkers

| | | SEQ ID |
|---|---|---|
| Abbreviation | Amino Acid Sequence | NO: |
| G4S | GGGGS | 416 |
| G5S | GGGGGS | 417 |
| GGSG | GGSG | 418 |
| SG3 | SGGG | 419 |
| GS² | GSGS | 420 |
| GS³ | GSGSGS | 421 |
| GS⁴ | GSGSGSGS | 422 |
| GS⁵ | GSGSGSGSGS | 423 |
| 6X, GGS² | GGSGGS | 424 |
| GGS³ | GGSGGSGGS | 425 |
| 10x | GGSGGSGGSG | 426 |
| GGSG² | GGSGGGSG | 427 |
| GGSG³ | GGSGGGSGGGSG | 428 |
| GGSG⁴ | GGSGGGSGGGSGGGSG | 429 |
| GGSG⁵ | GGSGGGSGGGSGGGSGGGSG | 430 |
| G4S² | GGGGSGGGGS | 431 |
| 8X, GGGS | GGGSGGGS | 432 |
| GGSGG | GGSGG | 433 |
| (GSGGSₘ)ₙ | GSGGS | 434 |
| GSGSG | GSGSG | 435 |
| GSGG | GSGG | 436 |
| GGGSG | GGGSG | 437 |
| GSSSG | GSSSG | 438 |
| 4X, G3S | GGGS | 439 |
| huIgG4 Fc | ASRVESKYGPPCPPCPAPEFLGGPSVFLFPPKPKDTLMISRTPEVTCVVVD | 440 |
| | VSQEDPEVQFNWYVDGVEVHNAKTKPREEQFGSTYRVVSVLTVLHQDWLNG | |
| | KEYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRWQ | |
| | EGNVFSCSVMHEALHNHYTQKSLSLSLG | |

TABLE 12
Chelating Peptides

| Name | Amino Acid Sequence | SEQ ID NO: |
|---|---|---|
| HAAH | (His)ₐ-(AA)_{b}-(His)_{c} | 448 |
| ASH6 | Ala-Ser-His-His-His-His-His-His | 449 |
| H5 | His-His-His-His-His | 450 |
| H6 | His-His-His-His-His-His | 451 |
| H7 | His-His-His-His-His-His-His | 452 |
| H8 | His-His-His-His-His-His-His-His | 453 |
| H9 | His-His-His-His-His-His-His-His-His | 454 |
| HWHMY | His-Trp-His-Met-Tyr | 455 |
| H3MY | His-His-His-Met-Tyr | 456 |
| H4Y | His-His-His-His-Tyr | 457 |
| HWHWH | His-Trp-His-Trp-His | 458 |
| HWH3 | His-Trp-His-His-His | 459 |
| H4YMH3 | His-His-His-His-Tyr-Met-His-His-His- | 460 |
| H4 | His-His-His-His | 461 |
| HWHHH | His-Trp-His-His-His- | 462 |
| HGHG3HGH | His-Gly-His-Gly-Gly-Gly-His-Gly-His | 463 |
| HGHG6HGH | His-Gly-His-Gly-Gly-Gly-Gly-Gly-Gly-His-Gly- | 464 |
| | His | |

TABLE 13
Amino Acid Abbreviations

| | | |
|---|---|---|
| G | Glycine | Gly |
| P | Proline | Pro |
| A | Alanine | Ala |
| V | Valine | Val |
| L | Leucine | Leu |
| I | Isoleucine | Ile |
| M | Methionine | Met |
| C | Cysteine | Cys |
| F | Phenylalanine | Phe |
| Y | Tyrosine | Tyr |
| W | Tryptophan | Trp |
| H | Histidine | His |
| K | Lysine | Lys |
| R | Arginine | Arg |
| Q | Glutamine | Gln |
| N | Asparagine | Asn |
| E | Glutamic Acid | Glu |
| D | Aspartic Acid | Asp |
| S | Serine | Ser |
| T | Threonine | Thr |

TABLE 14
DR841 AMINO ACID AND NUCLEOTIDE SEQUENCES

| | | SEQ ID |
|---|---|---|
| NAME | AMINO ACID SEQUENCE | NO: |
| DR841aa | DEVQLQESGGGSVQAGGSLRLSCAASGYSNCAYDMTWYRQAPGKEREF | 465 |
| IL-10Rα/ | VSAIHSDGSTRYADSVKGRFFISQDNAKNTVYLQMNSLKPEDTAMYYC | |
| IL-10Rβ | KTDPLHCRAHGGSWYSVRANYWGQGTQVTVSSGGGSQVQLQESGGGSV | |
| VHH Dimer | EAGGSLRLSCAASGYTHSSYCMGWFRQAPGKEREGVAAIDVDGSTTYA | |
| | DSVKGRFTISKDNAKNTLYLQMNSLKPEDTGMYYCAAEFADCSSNYFL | |
| | PPGAVRYWGQGTQVTVSSASHHHHHH | |
| DR841aa | DEVQLQESGGGSVQAGGSLRLSCAASGYSNCAYDMTWYRQAPGKEREF | 466 |
| IL-10Rα | VSAIHSDGSTRYADSVKGRFFISQDNAKNTVYLOMNSLKPEDTAMYYC | |
| VHH | KTDPLHCRAHGGSWYSVRANYWGQGTQVTVSS | |
| CDR1 | YSNCAYDMT | 467 |
| CDR2 | AIHSDGSTRYADSVKG | 229 |
| CDR3 | KTDPLHCRAHGGSWYSVRANY | 236 |
| DR841aa | QVQLQESGGGSVEAGGSLRLSCAASGYTHSSYCMGWFRQAPGKEREGV | 470 |
| IL10Rβ VHH | AAIDVDGSTTYADSVKGRFTISKDNAKNTLYLQMNSLKPEDTGMYYCA | |
| | AEFADCSSNYFLPPGAVRYWGQGTQVTVSS | |
| CDR1 | YTHSSYCMG | 471 |
| CDR2 | AIDVDGSTTYADSVKG | 472 |
| CDR3 | AAEFADCSSNYFLPPGAVRY | 473 |
| DR841nt | ACCGGTGTCCACTCTGAGGTGCAGCTGCAAGAGAGCGGAGGAGGAAGC | 580 |
| | GTCCAAGCCGGAGGCTCTCTGAGACTGAGCTGTGCCGCCAGCGGCTAC | |
| | TCCAACTGCGCCTACGACATGACTTGGTATAGGCAAGCCCCCGGCAAG | |
| | GAGAGGGAGTTCGTGTCCGCCATCCACAGCGACGGCAGCACTAGATAC | |
| | GCCGACAGCGTGAAGGGAAGGTTCTTCATCAGCCAAGATAACGCCAAG | |
| | AACACAGTGTATCTGCAGATGAACTCCCTCAAGCCAGAGGACACTGCC | |
| | ATGTACTACTGCAAGACAGACCCACTGCACTGCAGAGCCCATGGCGGC | |
| | AGCTGGTATAGCGTGAGGGCCAACTACTGGGGCCAAGGCACACAAGTG | |
| | ACAGTCTCGAGCGGCGGAGGATCCCAAGTGCAGCTGCAAGAGAGCGGA | |
| | GGAGGCAGCGTCGAAGCTGGAGGATCTCTGAGGCTGAGCTGTGCTGCC | |
| | AGCGGCTACACTCACAGCAGCTACTGTATGGGCTGGTTCAGACAAGCC | |
| | CCCGGCAAGGAGAGGGAAGGCGTGGCTGCCATCGACGTGGATGGCAGC | |
| | ACTACTTACGCCGACAGCGTGAAGGGAAGGTTCACTATCAGCAAGGAC | |
| | AACGCCAAGAACACACTCTATCTGCAGATGAACAGCCTCAAGCCAGAG | |
| | GACACTGGCATGTACTACTGCGCCGCCGAGTTCGCCGATTGCAGCAGC | |
| | AACTACTTTCTGCCTCCCGGCGCCGTCAGATATTGGGGCCAAGGCACT | |
| | CAAGTGACAGTCTCGTCTGCTAGCCACCATCACCATCACCACTGATAA | |
| | GCGGCCGC | |

TABLE 15
C-Terminal Modifications to Reduce Immunogenicity

| | AMINO ACID SEQUENCE | |
|---|---|---|
| | (C-terminal VTVSS | |
| | (SEQ ID NO: 474) | |
| | mutations in bold, | SEQ ID |
| DNA # | where applicable) | NO: |
| DR2485 (wild-type) | VTVSS | 474 |
| DR2486 | VTVSSAA | 475 |
| DR2487 | GTVSS | 476 |
| DR2491 | LTVSS | 477 |
| DR2492 | LTVSSAA | 478 |
| DR2493 | VTVSA | 479 |
| DR2494 | VTVSAAA | 480 |
| DR2495 | VTVCS | 481 |
| DR2496 | VTVCSA | 482 |
| DR2497 | VTVSC | 483 |
| DR2498 | VTVSCAA | 484 |
| DR2651 | LLTVSS | 485 |
| DR2652 | LLTVSSA | 486 |
| DR2653 | LLTVSSAA | 487 |
| DR2657 | LLTVSS (L11S) | 488 |
| DR2658 | LLTVSSA (L11S) | 489 |
| DR2659 | LLTVSSAA (L11S) | 490 |
| DR2673 | LVTVTT | 491 |
| DR2674 | LVTVTTAA | 492 |
| DR2675 | LVTVTT (L11S) | 493 |
| DR2676 | LVTVTTAA (L11S) | 494 |
| DR2654 | TLTVSS | 495 |
| DR2655 | TLTVSSA | 496 |
| DR2656 | TLTVSSAA | 497 |
| DR2096 | VTVSAAS | 498 |
| Wild-type | LVTVSS | 499 |

TABLE 16
Means-Plus-Function Linking Table
Examples of Functions Linked to Structures/Materials/Acts

| | | Exemplified |
|---|---|---|
| | | structures/materials/acts |
| Claim No. | Recited Function | disclosed in Specification |
| 21 | “means for inducing | DR2463aa (SEQ ID NO: 1); |
| | intracellular signaling in | DR2485aa (SEQ ID NO: 2); |
| | the cell expressing IL-10Rα | DR2519aa(SEQ ID NO: 3); |
| | and IL-10Rβ, wherein the | DR2520aa(SEQ ID NO: 4); |
| | means comprises (i) a single- | DR2457aa(SEQ ID NO: 5); |
| | domain antibody that binds to | DR2458aa(SEQ ID NO: 6); |
| | IL-10Rα (IL-10Rα sdAb) and is | DR2459aa(SEQ ID NO: 7); |
| | at least 79% humanized | DR2460aa(SEQ ID NO: 8); |
| | relative to SEQ ID NO: 465, | DR2461aa(SEQ ID NO: 9); |
| | linked to (ii) a single- | DR2462aa(SEQ ID NO: 10); |
| | domain antibody that binds to | DR2464aa(SEQ ID NO: 11); |
| | IL-10Rβ (IL-10Rβ sdAb) and | DR2465aa(SEQ ID NO: 12); |
| | that is greater than [90]% | DR2466aa(SEQ ID NO: 13); |
| | humanized relative to SEQ ID | DR2499aa(DR2463aa D33A |
| | NO: 470” | resurfacing) (SEQ ID NO: 14); |
| | | DR2500aa(DR2463aa T35S |
| | | resurfacing) (SEQ ID NO: 15); |
| | | DR2501aa(DR2463aa H52S, CDR2 |
| | | resurfacing) (SEQ ID NO: 16); |
| | | DR0000aa(DR2463aa S53G, CDR2 |
| | | resurfacing) (SEQ ID NO: 17); |
| | | DR2502aa(DR2463 D54S, CDR2 |
| | | resurfacing) (SEQ ID NO: 18); |
| | | DR2503aa(DR2463aa R58Y, CDR2 |
| | | resurfacing) (SEQ ID NO: 19); |
| | | DR2504aa (DR2463aa D33A, T55S, |
| | | CDR1 resurfacing) (SEQ ID |
| | | NO: 20); |
| | | DR2505aa (DR2463aa H72S, S73G, |
| | | D74S, R78Y, CDR2 resurfacing) |
| | | (SEQ ID NO: 21); |
| | | DR2506aa (DR2463aa D33A, H72S, |
| | | S73G, D74S, R78Y, CDR1 + CDR2 |
| | | resurfacing) (SEQ ID NO: 22) |
| 25 | “wherein dimerizing the | Example 4 |
| | extracellular domains of an | Example 5 |
| | IL-10Rα subunit and an IL- | Example 6 |
| | 10Rβ subunit of the IL-10 | Example 7 |
| | receptor (IL-10R) on a cell | |
| | induces pSTAT3 signaling” | |
| 26 | “wherein the intracellular | Example 4 |
| | pSTAT-3 signaling in the | Example 5 |
| | monocyte cell expressing | Example 6 |
| | IL10Rα and IL10R is greater | Example 7 |
| | than the pSTAT-3 signaling in | |
| | a T-cell expressing IL10Rα | |
| | and IL10Rβ.” | |

TABLE 17
Humanized IL-10Rα/IL-10Rβ VHH Dimer Amino Acid Sequences (Untagged)

| Amino Acid | | |
|---|---|---|
| Number | | SEQ |
| corresponding | Amino Acid Sequence of hzIL-10Rα/IL-10Rβ VHH Dimer | ID |
| to DNA number | Untagged (CDRs Underlined) | NO: |
| DR2463- | EVQLLESGGGLVQAGGSLRLSCAASGYSNCAYDMTWYRQAPGKEREFVSAIHSD | 500 |
| untagged | GSTRYADSVKGRFFISRDNSKNTVYLQMNSLRPEDTAVYYCKTDPLHCRAHGGS | |
| aa | WYSVRANYWGQGTLVTVSSGGGSEVQLVESGGGLVQPGGSLRLSCAASGYTHSS | |
| | YCMGWVRQAPGKGLEGVSAIDVDGSTTYADSVKGRFTISRDNSKNTLYLQMNSL | |
| | RAEDTAVYYCAAEFADCSSNYFLPPGAVRYWGQGTLVTVSS | |
| DR2485 aa | EVQLLESGGGLVQAGGSLRLSCAASGYSNCAYDMTWYRQAPGKEREFVSAIHSD | 501 |
| | GSTRYADSVKGRFFISRDNSKNTVYLQMNSLRPEDTAVYYCKTDPLHCRAHGGS | |
| | WYSVRANYWGQGTLVTVSSGGGSEVQLVESGGGLVQPGGSLRLSCAASGYTHSS | |
| | YCMGWVRQAPGKGLEGVSAIDVDGSTTYADSVKGRFTISRDNSKNTLYLQMNSL | |
| | RAEDTAVYYCAAEFADCSSNYFLPPGAVRYWGQGTTVTVSS | |
| DR2519 aa | EVQLLESGGGLVQAGGSLRLSCAASGYSNCAYDMTWYRQAPGKEREFVSAIHSD | 502 |
| | GSTYYADSVKGRFFISRDNSKNTVYLQMNSLRPEDTAVYYCKTDPLHCRAHGGS | |
| | WYSVRANYWGQGTLVTVSSGGGSEVQLVESGGGLVQPGGSLRLSCAASGYTHSS | |
| | YCMGWVRQAPGKGLEGVSAIDVDGSTTYADSVKGRFTISRDNSKNTLYLQMNSL | |
| | RAEDTAVYYCAAEFADCSSNYFLPPGAVRYWGQGTTVTVSS | |
| DR2520 aa | EVQLLESGGGLVQAGGSLRLSCAASGYSNCAYDMTWYRQAPGKEREFVSAIHSS | 503 |
| | GSTYYADSVKGRFFISRDNSKNTVYLQMNSLRPEDTAVYYCKTDPLHCRAHGGS | |
| | WYSVRANYWGQGTLVTVSSGGGSEVQLVESGGGLVQPGGSLRLSCAASGYTHSS | |
| | YCMGWVRQAPGKGLEGVSAIDVDGSTTYADSVKGRFTISRDNSKNTLYLQMNSL | |
| | RAEDTAVYYCAAEFADCSSNYFLPPGAVRYWGQGTTVTVSS | |
| DR2457- | EVQLQESGGGSVQPGGSLRLSCAASGYSNCAYDMTWYRQAPGKEREFVSAIHSD | 504 |
| untagged | GSTRYADSVKGRFFISQDNAKNTVYLQMNSLKPEDTAMYYCKTDPLHCRAHGGS | |
| aa | WYSVRANYWGQGTQVTVSSGGGSEVQLVESGGGLVQPGGSLRLSCAASGYTHSS | |
| | YCMGWVRQAPGKGLEGVSAIDVDGSTTYADSVKGRFTISRDNSKNTLYLQMNSL | |
| | RAEDTAVYYCAAEFADCSSNYFLPPGAVRYWGQGTLVTVSS | |
| DR2458- | EVQLQESGGGLVQAGGSLRLSCAASGYSNCAYDMTWYRQAPGKEREFVSAIHSD | 505 |
| untagged | GSTRYADSVKGRFFISQDNSKNTVYLQMNSLKPEDTAVYYCKTDPLHCRAHGGS | |
| aa | WYSVRANYWGQGTLVTVSSGGGSEVQLVESGGGLVQPGGSLRLSCAASGYTHSS | |
| | YCMGWVRQAPGKGLEGVSAIDVDGSTTYADSVKGRFTISRDNSKNTLYLQMNSL | |
| | RAEDTAVYYCAAEFADCSSNYFLPPGAVRYWGQGTLVTVSS | |
| DR2459- | EVQLQESGGGLVQAGGSLRLSCAASGYSNCAYDMTWYRQAPGKEREFVSAIHSD | 506 |
| untagged | GSTRYADSVKGRFFISRDNSKNTVYLQMNSLKPEDTAVYYCKTDPLHCRAHGGS | |
| aa | WYSVRANYWGQGTLVTVSSGGGSEVQLVESGGGLVQPGGSLRLSCAASGYTHSS | |
| | YCMGWVRQAPGKGLEGVSAIDVDGSTTYADSVKGRFTISRDNSKNTLYLQMNSL | |
| | RAEDTAVYYCAAEFADCSSNYFLPPGAVRYWGQGTLVTVSS | |
| DR2460- | EVQLLESGGGLVQAGGSLRLSCAASGYSNCAYDMTWYRQAPGKEREFVSAIHSD | 507 |
| untagged | GSTRYADSVKGRFFISQDNSKNTVYLQMNSLKPEDTAVYYCKTDPLHCRAHGGS | |
| aa | WYSVRANYWGQGTLVTVSSGGGSEVQLVESGGGLVQPGGSLRLSCAASGYTHSS | |
| | YCMGWVRQAPGKGLEGVSAIDVDGSTTYADSVKGRFTISRDNSKNTLYLQMNSL | |
| | RAEDTAVYYCAAEFADCSSNYFLPPGAVRYWGQGTLVTVSS | |
| DR2461- | EVQLLESGGGLVQAGGSLRLSCAASGYSNCAYDMTWYRQAPGKEREFVSAIHSD | 508 |
| untagged | GSTRYADSVKGRFFISRDNSKNTVYLQMNSLKPEDTAVYYCKTDPLHCRAHGGS | |
| aa | WYSVRANYWGQGTLVTVSSGGGSEVQLVESGGGLVQPGGSLLSCAASGYTHSSY | |
| | CMGWVRQAPGKGLEGVSAIDVDGSTTYADSVKGRFTISRDNSKNTLYLQMNSLR | |
| | AEDTAVYYCAAEFADCSSNYFLPPGAVRYWGQGTLVTVSS | |
| DR2462- | EVQLQESGGGLVQAGGSLRLSCAASGYSNCAYDMTWYRQAPGKEREFVSAIHSD | 509 |
| untagged | GSTRYADSVKGRFFISRDNSKNTVYLQMNSLRPEDTAVYYCKTDPLHCRAHGGS | |
| aa | WYSVRANYWGQGTLVTVSSGGGSEVQLVESGGGLVQPGGSLRLSCAASGYTHSS | |
| | YCMGWVRQAPGKGLEGVSAIDVDGSTTYADSVKGRFTISRDNSKNTLYLQMNSL | |
| | RAEDTAVYYCAAEFADCSSNYFLPPGAVRYWGQGTLVTVSS | |
| DR2464- | EVQLLESGGGLVQAGGSLRLSCAASGYSNCAYDMTWYRQAPGKEREFVSAIHSD | 510 |
| untagged | GSTRYADSVKGRFFISRDNSKNTVYLQMNSLRAEDTAVYYCKTDPLHCRAHGGS | |
| aa | WYSVRANYWGQGTLVTVSSGGGSEVQLVESGGGLVQPGGSLRLSCAASGYTHSS | |
| | YCMGWVRQAPGKGLEGVSAIDVDGSTTYADSVKGRFTISRDNSKNTLYLQMNSL | |
| | RAEDTAVYYCAAEFADCSSNYFLPPGAVRYWGQGTLVTVSS | |
| DR2465- | EVQLLESGGGLVQAGGSLRLSCAASGYSNCAYDMTWYRQAPGKEREFVSAIHSD | 511 |
| untagged | GSTRYADSVKGRFFISRDNSKNTLYLQMNSLRPEDTAVYYCKTDPLHCRAHGGS | |
| aa | WYSVRANYWGQGTLVTVSSGGGSEVQLVESGGGLVQPGGSLRLSCAASGYTHSS | |
| | YCMGWVRQAPGKGLEGVSAIDVDGSTTYADSVKGRFTISRDNSKNTLYLQMNSL | |
| | RAEDTAVYYCAAEFADCSSNYFLPPGAVRYWGQGTLVTVSS | |
| DR2466- | EVQLLESGGGLVQAGGSLRLSCAASGYSNCAYDMTWYRQAPGKEREFVSAIHSD | 512 |
| untagged | GSTRYADSVKGRFFISRDNSKNTLYLQMNSLRAEDTAVYYCKTDPLHCRAHGGS | |
| aa | WYSVRANYWGQGTLVTVSSGGGSEVQLVESGGGLVQPGGSLRLSCAASGYTHSS | |
| | YCMGWVRQAPGKGLEGVSAIDVDGSTTYADSVKGRFTISRDNSKNTLYLQMNSL | |
| | RAEDTAVYYCAAEFADCSSNYFLPPGAVRYWGQGTLVTVSS | |
| DR2499- | EVQLLESGGGLVQAGGSLRLSCAASGYSNCAYAMTWYRQAPGKEREFVSAIHSD | 513 |
| untagged | GSTRYADSVKGRFFISRDNSKNTVYLQMNSLRPEDTAVYYCKTDPLHCRAHGGS | |
| aa | WYSVRANYWGQGTLVTVSSGGGSEVQLVESGGGLVQPGGSLRLSCAASGYTHSS | |
| (DR2463aa | YCMGWVRQAPGKGLEGVSAIDVDGSTTYADSVKGRFTISRDNSKNTLYLQMNSL | |
| D33A | RAEDTAVYYCAAEFADCSSNYFLPPGAVRYWGQGTLVTVSS | |
| resurfacing) | | |
| DR2500- | EVQLLESGGGLVQAGGSLRLSCAASGYSNCAYDMSWYRQAPGKEREFVSAIHSD | 514 |
| untagged | GSTRYADSVKGRFFISRDNSKNTVYLQMNSLRPEDTAVYYCKTDPLHCRAHGGS | |
| aa | WYSVRANYWGQGTLVTVSSGGGSEVQLVESGGGLVQPGGSLRLSCAASGYTHSS | |
| (DR2463aa | YCMGWVRQAPGKGLEGVSAIDVDGSTTYADSVKGRFTISRDNSKNTLYLQMNSL | |
| T35S | RAEDTAVYYCAAEFADCSSNYFLPPGAVRYWGQGTLVTVSS | |
| resurfacing) | | |
| DR2501- | EVQLLESGGGLVQAGGSLRLSCAASGYSNCAYDMTWYRQAPGKEREFVSAISSD | 515 |
| untagged | GSTRYADSVKGRFFISRDNSKNTVYLQMNSLRPEDTAVYYCKTDPLHCRAHGGS | |
| aa | WYSVRANYWGQGTLVTVSSGGGSEVQLVESGGGLVQPGGSLRLSCAASGYTHSS | |
| (DR2463aa | YCMGWVRQAPGKGLEGVSAIDVDGSTTYADSVKGRFTISRDNSKNTLYLQMNSL | |
| H52S, CDR2 | RAEDTAVYYCAAEFADCSSNYFLPPGAVRYWGQGTLVTVSS | |
| resurfacing) | | |
| DR0000- | EVQLLESGGGLVQAGGSLRLSCAASGYSNCAYDMTWYRQAPGKEREFVSAIHGD | 516 |
| untagged | GSTRYADSVKGRFFISRDNSKNTVYLQMNSLRPEDTAVYYCKTDPLHCRAHGGS | |
| aa | WYSVRANYWGQGTLVTVSSGGGSEVQLVESGGGLVQPGGSLRLSCAASGYTHSS | |
| (DR2463aa | YCMGWVRQAPGKGLEGVSAIDVDGSTTYADSVKGRFTISRDNSKNTLYLQMNSL | |
| S53G, | RAEDTAVYYCAAEFADCSSNYFLPPGAVRYWGQGTLVTVSS | |
| CDR2 | | |
| resurfacing) | | |
| DR2502- | EVQLLESGGGLVQAGGSLRLSCAASGYSNCAYDMTWYRQAPGKEREFVSAIHSS | 517 |
| untagged | GSTRYADSVKGRFFISRDNSKNTVYLQMNSLRPEDTAVYYCKTDPLHCRAHGGS | |
| aa | WYSVRANYWGQGTLVTVSSGGGSEVQLVESGGGLVQPGGSLRLSCAASGYTHSS | |
| (DR2463 | YCMGWVRQAPGKGLEGVSAIDVDGSTTYADSVKGRFTISRDNSKNTLYLQMNSL | |
| D54S, CDR2 | RAEDTAVYYCAAEFADCSSNYFLPPGAVRYWGQGTLVTVSS | |
| resurfacing) | | |
| DR2503- | EVQLLESGGGLVQAGGSLRLSCAASGYSNCAYDMTWYRQAPGKEREFVSAIHSD | 518 |
| untagged | GSTYYADSVKGRFFISRDNSKNTVYLQMNSLRPEDTAVYYCKTDPLHCRAHGGS | |
| aa | WYSVRANYWGQGTLVTVSSGGGSEVQLVESGGGLVQPGGSLRLSCAASGYTHSS | |
| (DR2463aa | YCMGWVRQAPGKGLEGVSAIDVDGSTTYADSVKGRFTISRDNSKNTLYLQMNSL | |
| R58Y, CDR2 | RAEDTAVYYCAAEFADCSSNYFLPPGAVRYWGQGTLVTVSS | |
| resurfacing) | | |
| DR2504- | EVQLLESGGGLVQAGGSLRLSCAASGYSNCAYAMSWYRQAPGKEREFVSAIHSD | 519 |
| untagged | GSTRYADSVKGRFFISRDNSKNTVYLQMNSLRPEDTAVYYCKTDPLHCRAHGGS | |
| aa | WYSVRANYWGQGTLVTVSSGGGSEVQLVESGGGLVQPGGSLRLSCAASGYTHSS | |
| (DR2463aa | YCMGWVRQAPGKGLEGVSAIDVDGSTTYADSVKGRFTISRDNSKNTLYLQMNSL | |
| D33A, | RAEDTAVYYCAAEFADCSSNYFLPPGAVRYWGQGTLVTVSS | |
| T55S, | | |
| CDR1 | | |
| resurfacing) | | |
| DR2505- | EVQLLESGGGLVQAGGSLRLSCAASGYSNCAYDMTWYRQAPGKEREFVSAISGS | 520 |
| untagged | GSTYYADSVKGRFFISRDNSKNTVYLQMNSLRPEDTAVYYCKTDPLHCRAHGGS | |
| aa | WYSVRANYWGQGTLVTVSSGGGSEVQLVESGGGLVQPGGSLRLSCAASGYTHSS | |
| (DR2463aa | YCMGWVRQAPGKGLEGVSAIDVDGSTTYADSVKGRFTISRDNSKNTLYLQMNSL | |
| H72S | RAEDTAVYYCAAEFADCSSNYFLPPGAVRYWGQGTLVTVSS | |
| S73G, | | |
| D74S, | | |
| R78Y, | | |
| CDR2 | | |
| Resurfacing) | | |
| DR2506- | EVQLLESGGGLVQAGGSLRLSCAASGYSNCAYAMSWYRQAPGKEREFVSAISGS | 521 |
| untagged | GSTYYADSVKGRFFISRDNSKNTVYLQMNSLRPEDTAVYYCKTDPLHCRAHGGS | |
| aa | WYSVRANYWGQGTLVTVSSGGGSEVQLVESGGGLVQPGGSLRLSCAASGYTHSS | |
| (DR2463aa | YCMGWVRQAPGKGLEGVSAIDVDGSTTYADSVKGRFTISRDNSKNTLYLQMNSL | |
| D33A, | RAEDTAVYYCAAEFADCSSNYFLPPGAVRYWGQGTLVTVSS | |
| H72S, | | |
| S73G, | | |
| D74S, | | |
| R78Y, | | |
| CDR1 + CDR2 | | |
| resurfacing) | | |
| DR1525- | EVQLLESGGGLVQPGGSLRLSCAASGYSNCAYDMTWYRQAPGKEREFVSAIHSD | 522 |
| untagged | GSTRYADSVKGRFFISRDNSKNTLYLQMNSLRAEDTAVYYCKTDPLHCRAHGGS | |
| aa | WYSVRANYWGQGTLVTVSSGGGSEVQLVESGGGLVQPGGSLRLSCAASGYTHSS | |
| | YCMGWVRQAPGKGLEGVSAIDVDGSTTYADSVKGRFTISRDNSKNTLYLQMNSL | |
| | RAEDTAVYYCAAEFADCSSNYFLPPGAVRYWGQGTLVTVSS | |
| DR2096- | EVQLLESGGGSVQPGGSLRLSCAASGYSNCAYDMTWYRQAPGKEREFVSAIHSD | 523 |
| untagged | GSTRYADSVKGRFFISRDNSKNTLYLQMNSLRAEDTAVYYCKTDPLHCRAHGGS | |
| aa | WYSVRANYWGQGTLVTVSSGGGSEVQLVESGGGLVQPGGSLRLSCAASGYTHSS | |
| | YCMGWVRQAPGKGLEGVSAIDVDGSTTYADSVKGRFTISRDNSKNTLYLQMNSL | |
| | RAEDTAVYYCAAEFADCSSNYFLPPGAVRYWGQGTLVTVSS | |

TABLE 18
Nucleic Acid Sequences Encoding
Humanized IL-10Rα-IL-10Rβ VHH Dimers With PolyHis Tags

| IL-10Rα/ | | |
|---|---|---|
| IL-10Rβ | | |
| VHH Dimer | | SEQ ID |
| DNA Number | DNA Sequence | NO: |
| DR2463 | GAGGTGCAGCTCCTGGAATCTGGCGGGGGCCTGGTCCAGGCTGGCGGTAGCCTCCGCCTTA | 524 |
| | GTTGTGCTGCCAGCGGTTACAGCAACTGCGCTTACGACATGACGTGGTATCGCCAGGCCCC | |
| | AGGGAAGGAGAGGGAGTTCGTCAGCGCTATCCATTCCGATGGGAGCACACGCTACGCTGAC | |
| | TCCGTGAAAGGGAGGTTCTTTATCTCTCGTGACAACAGCAAAAACACAGTGTACCTCCAGA | |
| | TGAACTCATTGAGACCAGAGGATACAGCTGTGTATTACTGTAAGACAGACCCTCTGCACTG | |
| | TCGCGCCCACGGGGGCTCCTGGTATTCTGTGCGTGCCAACTATTGGGGCCAGGGAACACTG | |
| | GTTACCGTCTCTAGCGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCCTGGTAACTGTCTCGTCTGCTAGCCACCATCACCATCACC | |
| | AC | |
| DR2485+ | GAGGTGCAGCTCCTGGAATCTGGCGGGGGCCTGGTCCAGGCTGGCGGTAGCCTCCGCCTTA | 525 |
| His | GTTGTGCTGCCAGCGGTTACAGCAACTGCGCTTACGACATGACGTGGTATCGCCAGGCCCC | |
| | AGGGAAGGAGAGGGAGTTCGTCAGCGCTATCCATTCCGATGGGAGCACACGCTACGCTGAC | |
| | TCCGTGAAAGGGAGGTTCTTTATCTCTCGTGACAACAGCAAAAACACAGTGTACCTCCAGA | |
| | TGAACTCATTGAGACCAGAGGATACAGCTGTGTATTACTGTAAGACAGACCCTCTGCACTG | |
| | TCGCGCCCACGGGGGCTCCTGGTATTCTGTGCGTGCCAACTATTGGGGCCAGGGAACACTG | |
| | GTTACCGTCTCTAGCGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCACGGTAACTGTCTCGTCTGCTAGCCACCATCACCATCACC | |
| | AC | |
| DR2519+ | GAGGTGCAGCTCCTGGAATCTGGCGGGGGCCTGGTCCAGGCTGGCGGTAGCCTCCGCCTTA | 526 |
| His | GTTGTGCTGCCAGCGGTTACAGCAACTGCGCTTACGACATGACGTGGTATCGCCAGGCCCC | |
| | AGGGAAGGAGAGGGAGTTCGTCAGCGCTATCCATTCCGATGGGAGCACATATTACGCTGAC | |
| | TCCGTGAAAGGGAGGTTCTTTATCTCTCGTGACAACAGCAAAAACACAGTGTACCTCCAGA | |
| | TGAACTCATTGAGACCAGAGGATACAGCTGTGTATTACTGTAAGACAGACCCTCTGCACTG | |
| | TCGCGCCCACGGGGGCTCCTGGTATTCTGTGCGTGCCAACTATTGGGGCCAGGGAACACTG | |
| | GTTACCGTCTCTAGCGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCACGGTAACTGTCTCGTCTGCTAGCCACCATCACCATCACC | |
| | AC | |
| DR2520+ | GAGGTGCAGCTCCTGGAATCTGGCGGGGGCCTGGTCCAGGCTGGCGGTAGCCTCCGCCTTA | 527 |
| His | GTTGTGCTGCCAGCGGTTACAGCAACTGCGCTTACGACATGACGTGGTATCGCCAGGCCCC | |
| | AGGGAAGGAGAGGGAGTTCGTCAGCGCTATCCATTCCTCTGGGAGCACATATTACGCTGAC | |
| | TCCGTGAAAGGGAGGTTCTTTATCTCTCGTGACAACAGCAAAAACACAGTGTACCTCCAGA | |
| | TGAACTCATTGAGACCAGAGGATACAGCTGTGTATTACTGTAAGACAGACCCTCTGCACTG | |
| | TCGCGCCCACGGGGGCTCCTGGTATTCTGTGCGTGCCAACTATTGGGGCCAGGGAACACTG | |
| | \|GTTACCGTCTCTAGCGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCACGGTAACTGTCTCGTCTGCTAGCCACCATCACCATCACC | |
| | AC | |
| DR2457 | GAAGTCCAGCTCCAGGAATCAGGAGGTGGCTCTGTGCAACCCGGTGGCTCCCTGCGCCTCA | 528 |
| | GCTGTGCCGCTTCTGGATATTCTAACTGCGCTTACGACATGACTTGGTATAGGCAGGCACC | |
| | GGGCAAGGAGCGCGAGTTCGTTTCTGCCATTCACTCTGATGGATCTACCCGCTATGCCGAT | |
| | AGCGTGAAGGGTCGCTTTTTCATCTCCCAGGATAACGCTAAAAATACTGTGTACCTTCAGA | |
| | TGAACAGCCTGAAACCTGAAGACACCGCCATGTACTATTGTAAGACAGACCCGTTGCATTG | |
| | CAGAGCCCACGGAGGCTCATGGTATTCCGTCCGGGCGAACTACTGGGGCCAGGGCACTCAG | |
| | GTGACCGTATCCTCTGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCCTGGTAACTGTCTCGTCTGCTAGCCACCATCACCATCACC | |
| | AC | |
| DR2458 | GAGGTTCAACTCCAGGAGAGCGGCGGGGGCCTGGTGCAAGCTGGGGGCAGTCTGCGCTTGA | 529 |
| | GCTGCGCTGCATCCGGCTATTCCAACTGTGCCTATGACATGACGTGGTATAGGCAGGCCCC | |
| | TGGGAAGGAGCGTGAGTTCGTGAGTGCTATCCACAGCGACGGCTCCACTAGATACGCGGAC | |
| | TCCGTTAAAGGAAGATTTTTCATCAGTCAGGACAACAGCAAGAATACCGTGTATTTGCAGA | |
| | TGAATAGTCTGAAGCCCGAGGACACAGCAGTCTACTATTGTAAGACTGACCCACTGCACTG | |
| | TCGTGCGCATGGAGGCTCTTGGTACTCCGTGAGAGCCAATTATTGGGGGCAGGGCACCCTT | |
| | GTTACCGTATCCTCCGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCCTGGTAACTGTCTCGTCTGCTAGCCACCATCACCATCACC | |
| | AC | |
| DR2459 | GAGGTGCAGCTCCAGGAGAGCGGCGGTGGCCTCGTCCAAGCCGGGGGCTCCCTGCGTTTGT | 530 |
| | CTTGCGCGGCCAGCGGATATTCCAACTGTGCGTATGACATGACATGGTATCGCCAGGCTCC | |
| | AGGCAAGGAGCGTGAGTTTGTGTCTGCAATCCACTCTGATGGCTCCACACGCTACGCCGAC | |
| | AGCGTGAAGGGCCGCTTCTTTATCTCCAGAGATAACAGCAAGAACACTGTCTATCTCCAGA | |
| | TGAACAGCCTGAAGCCCGAAGACACCGCCGTTTATTACTGTAAGACCGACCCTCTGCATTG | |
| | CAGAGCCCACGGCGGTTCCTGGTATTCCGTCCGCGCTAACTATTGGGGACAGGGGACCCTT | |
| | GTGACAGTCAGCTCCGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCCTGGTAACTGTCTCGTCTGCTAGCCACCATCACCATCACC | |
| | AC | |
| DR2460 | GAGGTGCAGTTGCTGGAGTCTGGTGGGGGCCTCGTGCAGGCCGGAGGCTCCCTGCGCCTGT | 531 |
| | CCTGCGCTGCCTCTGGATACTCTAATTGCGCATACGACATGACATGGTATCGGCAGGCACC | |
| | CGGAAAGGAGCGCGAGTTCGTATCAGCAATCCATAGTGACGGTTCTACTCGCTATGCAGAC | |
| | TCTGTGAAAGGACGCTTCTTTATTAGCCAGGACAACTCTAAAAACACTGTCTATCTCCAAA | |
| | TGAACTCCCTCAAGCCAGAAGATACTGCCGTGTATTACTGCAAGACCGACCCACTGCATTG | |
| | TCGTGCGCATGGAGGGTCTTGGTATTCAGTACGTGCAAACTACTGGGGACAGGGCACCCTG | |
| | GTCACTGTGTCCTCTGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCCTGGTAACTGTCTCGTCTGCTAGCCACCATCACCATCACC | |
| | AC | |
| DR2461 | GAGGTACAGCTCTTGGAGTCCGGCGGTGGCTTGGTGCAGGCTGGAGGTAGCTTGCGCCTGT | 532 |
| | CCTGCGCCGCTTCCGGCTACAGCAACTGCGCCTACGACATGACGTGGTATCGGCAGGCTCC | |
| | AGGTAAGGAGCGCGAGTTCGTCAGTGCCATTCACTCCGACGGGAGCACTAGATATGCCGAC | |
| | TCTGTCAAGGGACGTTTTTTCATTAGTCGCGACAACTCTAAGAACACCGTGTACCTTCAGA | |
| | TGAACAGCCTGAAGCCCGAGGACACTGCCGTGTACTATTGCAAGACCGACCCCCTCCATTG | |
| | CAGGGCTCATGGGGGCTCTTGGTATTCAGTGAGAGCCAACTATTGGGGACAGGGTACGCTG | |
| | GTGACCGTGTCATCTGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCCTGGTAACTGTCTCGTCTGCTAGCCACCATCACCATCACC | |
| | AC | |
| DR2462 | GAGGTCCAGTTGCAGGAGTCCGGTGGCGGGTTGGTCCAAGCAGGCGGAAGCCTCCGCCTTA | 533 |
| | GTTGTGCGGCCTCTGGCTACTCTAACTGCGCCTATGATATGACGTGGTATCGCCAAGCTCC | |
| | AGGCAAAGAAAGAGAGTTCGTCAGCGCCATTCACTCTGATGGCTCCACTAGGTACGCAGAC | |
| | AGCGTCAAGGGTCGTTTTTTCATCTCCCGCGATAACTCCAAGAACACCGTGTACCTCCAAA | |
| | TGAACTCCCTGAGACCGGAGGACACTGCTGTGTACTATTGCAAGACCGATCCTTTGCACTG | |
| | TAGGGCGCATGGAGGCAGTTGGTACTCCGTTAGAGCCAACTACTGGGGGCAAGGAACCCTG | |
| | GTCACTGTCAGCTCCGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCCTGGTAACTGTCTCGTCTGCTAGCCACCATCACCATCACC | |
| | AC | |
| DR2464 | GAGGTTCAGTTGCTGGAATCTGGGGGCGGTCTGGTCCAGGCTGGGGGCTCTCTGAGGCTGA | 534 |
| | GCTGCGCCGCATCTGGCTACTCCAACTGCGCCTATGATATGACTTGGTACAGGCAGGCTCC | |
| | CGGCAAGGAGCGCGAGTTCGTGAGCGCCATTCACTCTGATGGTTCCACAAGGTATGCTGAT | |
| | AGTGTTAAAGGACGCTTTTTCATTAGCCGGGACAATAGCAAAAACACAGTCTATCTCCAGA | |
| | TGAACTCCCTCCGGGCGGAGGATACCGCTGTCTATTACTGCAAGACTGACCCTTTGCACTG | |
| | TAGAGCACACGGAGGCAGCTGGTATTCTGTGAGAGCCAACTATTGGGGACAAGGAACCCTC | |
| | GTCACGGTGTCTAGCGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCCTGGTAACTGTCTCGTCTGCTAGCCACCATCACCATCACC | |
| | AC | |
| DR2465 | GAGGTGCAGCTCCTGGAGTCTGGCGGTGGCCTGGTGCAAGCTGGCGGTTCCCTTCGTCTGT | 535 |
| | CCTGCGCAGCCAGTGGCTATTCTAACTGTGCTTACGACATGACATGGTATCGTCAGGCACC | |
| | GGGGAAGGAGCGCGAGTTCGTATCAGCTATCCACTCTGACGGCAGTACCAGATATGCCGAC | |
| | AGCGTCAAAGGGCGTTTTTTCATCTCCCGCGACAACTCCAAAAATACCCTGTATCTTCAGA | |
| | TGAACTCCCTCAGGCCTGAGGACACTGCCGTCTATTACTGCAAGACTGACCCCCTCCACTG | |
| | TCGCGCTCATGGGGGTAGTTGGTACTCTGTGCGTGCTAATTACTGGGGGCAGGGCACCCTG | |
| | GTGACCGTTAGCTCAGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCCTGGTAACTGTCTCGTCTGCTAGCCACCATCACCATCACC | |
| | AC | |
| DR2466 | GAGGTGCAGTTGCTGGAGAGCGGGGGCGGGCTCGTGCAGGCGGGAGGCAGCTTGCGCTTGT | 536 |
| | CATGTGCTGCCAGCGGTTATAGCAACTGCGCCTACGACATGACATGGTATCGCCAGGCTCC | |
| | CGGTAAAGAGAGGGAGTTTGTATCCGCGATCCACAGTGACGGTAGCACCCGCTACGCGGAT | |
| | TCCGTGAAAGGACGTTTCTTTATTTCTAGGGATAATAGCAAGAATACACTGTACTTGCAAA | |
| | TGAACTCCCTCCGCGCCGAGGACACTGCTGTGTATTACTGCAAAACTGATCCACTGCACTG | |
| | TCGCGCTCACGGGGGTTCTTGGTATTCAGTACGCGCCAACTACTGGGGCCAGGGTACTCTC | |
| | GTGACGGTGAGCAGCGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCCTGGTAACTGTCTCGTCTGCTAGCCACCATCACCATCACC | |
| | AC | |
| DR2499 | GAGGTGCAGCTCCTGGAATCTGGCGGGGGCCTGGTCCAGGCTGGCGGTAGCCTCCGCCTTA | 537 |
| | GTTGTGCTGCCAGCGGTTACAGCAACTGCGCTTACGCCATGACGTGGTATCGCCAGGCCCC | |
| | AGGGAAGGAGAGGGAGTTCGTCAGCGCTATCCATTCCGATGGGAGCACACGCTACGCTGAC | |
| | TCCGTGAAAGGGAGGTTCTTTATCTCTCGTGACAACAGCAAAAACACAGTGTACCTCCAGA | |
| | TGAACTCATTGAGACCAGAGGATACAGCTGTGTATTACTGTAAGACAGACCCTCTGCACTG | |
| | TCGCGCCCACGGGGGCTCCTGGTATTCTGTGCGTGCCAACTATTGGGGCCAGGGAACACTG | |
| | GTTACCGTCTCTAGCGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCCTGGTAACTGTCTCGTCTGCTAGCCACCATCACCATCACC | |
| | AC | |
| DR2500 | GAGGTGCAGCTCCTGGAATCTGGCGGGGGCCTGGTCCAGGCTGGCGGTAGCCTCCGCC | 538 |
| | TTAGTTGTGCTGCCAGCGGTTACAGCAACTGCGCTTACGACATGTCGTGGTATCGCCA | |
| | GGCCCCAGGGAAGGAGAGGGAGTTCGTCAGCGCTATCCATTCCGATGGGAGCACACGC | |
| | TACGCTGACTCCGTGAAAGGGAGGTTCTTTATCTCTCGTGACAACAGCAAAAACACAG | |
| | TGTACCTCCAGATGAACTCATTGAGACCAGAGGATACAGCTGTGTATTACTGTAAGAC | |
| | AGACCCTCTGCACTGTCGCGCCCACGGGGGCTCCTGGTATTCTGTGCGTGCCAACTAT | |
| | TGGGGCCAGGGAACACTGGTTACCGTCTCTAGCGGAGGAGGATCCGAGGTGCAGCTGG | |
| | TGGAGAGCGGAGGAGGGTTAGTGCAGCCGGGCGGTTCTCTCAGACTTAGCTGCGCCGC | |
| | CTCCGGGTACACGCACTCTTCCTATTGCATGGGCTGGGTCAGGCAGGCGCCTGGGAAG | |
| | GGCCTGGAGGGAGTTAGTGCCATCGATGTCGATGGCTCAACAACCTATGCAGACAGCG | |
| | TTAAGGGTCGGTTCACCATTTCACGCGACAATAGCAAAAACACTCTGTATTTGCAGAT | |
| | GAACAGTCTCCGTGCCGAAGACACAGCTGTCTACTACTGTGCAGCTGAATTTGCTGAT | |
| | TGTTCCTCTAATTATTTCCTACCCCCAGGGGCAGTGCGATACTGGGGACAAGGCACCC | |
| | TGGTAACTGTCTCGTCTGCTAGCCACCATCACCATCACCAC | |
| DR2501 | GAGGTGCAGCTCCTGGAATCTGGCGGGGGCCTGGTCCAGGCTGGCGGTAGCCTCCGCCTTA | 539 |
| | GTTGTGCTGCCAGCGGTTACAGCAACTGCGCTTACGACATGACGTGGTATCGCCAGGCCCC | |
| | AGGGAAGGAGAGGGAGTTCGTCAGCGCTATCTCTTCCGATGGGAGCACACGCTACGCTGAC | |
| | TCCGTGAAAGGGAGGTTCTTTATCTCTCGTGACAACAGCAAAAACACAGTGTACCTCCAGA | |
| | TGAACTCATTGAGACCAGAGGATACAGCTGTGTATTACTGTAAGACAGACCCTCTGCACTG | |
| | TCGCGCCCACGGGGGCTCCTGGTATTCTGTGCGTGCCAACTATTGGGGCCAGGGAACACTG | |
| | GTTACCGTCTCTAGCGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCCTGGTAACTGTCTCGTCTGCTAGCCACCATCACCATCACC | |
| | AC | |
| DR0000 | GAGGTGCAGCTCCTGGAATCTGGCGGGGGCCTGGTCCAGGCTGGCGGTAGCCTCCGCCTTA | 540 |
| | GTTGTGCTGCCAGCGGTTACAGCAACTGCGCTTACGACATGACGTGGTATCGCCAGGCCCC | |
| | AGGGAAGGAGAGGGAGTTCGTCAGCGCTATCCATGGCGATGGGAGCACACGCTACGCTGAC | |
| | TCCGTGAAAGGGAGGTTCTTTATCTCTCGTGACAACAGCAAAAACACAGTGTACCTCCAGA | |
| | TGAACTCATTGAGACCAGAGGATACAGCTGTGTATTACTGTAAGACAGACCCTCTGCACTG | |
| | TCGCGCCCACGGGGGCTCCTGGTATTCTGTGCGTGCCAACTATTGGGGCCAGGGAACACTG | |
| | GTTACCGTCTCTAGCGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCCTGGTAACTGTCTCGTCTGCTAGCCACCATCACCATCACC | |
| | AC | |
| DR2502 | GAGGTGCAGCTCCTGGAATCTGGCGGGGGCCTGGTCCAGGCTGGCGGTAGCCTCCGCCTTA | 541 |
| | GTTGTGCTGCCAGCGGTTACAGCAACTGCGCTTACGACATGACGTGGTATCGCCAGGCCCC | |
| | AGGGAAGGAGAGGGAGTTCGTCAGCGCTATCCATTCCAGTGGGAGCACACGCTACGCTGAC | |
| | TCCGTGAAAGGGAGGTTCTTTATCTCTCGTGACAACAGCAAAAACACAGTGTACCTCCAGA | |
| | TGAACTCATTGAGACCAGAGGATACAGCTGTGTATTACTGTAAGACAGACCCTCTGCACTG | |
| | TCGCGCCCACGGGGGCTCCTGGTATTCTGTGCGTGCCAACTATTGGGGCCAGGGAACACTG | |
| | GTTACCGTCTCTAGCGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCCTGGTAACTGTCTCGTCTGCTAGCCACCATCACCATCACC | |
| | AC | |
| DR2503 | GAGGTGCAGCTCCTGGAATCTGGCGGGGGCCTGGTCCAGGCTGGCGGTAGCCTCCGCCTTA | 542 |
| | GTTGTGCTGCCAGCGGTTACAGCAACTGCGCTTACGACATGACGTGGTATCGCCAGGCCCC | |
| | AGGGAAGGAGAGGGAGTTCGTCAGCGCTATCCATTCCGATGGGAGCACATACTACGCTGAC | |
| | TCCGTGAAAGGGAGGTTCTTTATCTCTCGTGACAACAGCAAAAACACAGTGTACCTCCAGA | |
| | TGAACTCATTGAGACCAGAGGATACAGCTGTGTATTACTGTAAGACAGACCCTCTGCACTG | |
| | TCGCGCCCACGGGGGCTCCTGGTATTCTGTGCGTGCCAACTATTGGGGCCAGGGAACACTG | |
| | GTTACCGTCTCTAGCGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCCTGGTAACTGTCTCGTCTGCTAGCCACCATCACCATCACC | |
| | AC | |
| DR2504 | GAGGTGCAGCTCCTGGAATCTGGCGGGGGCCTGGTCCAGGCTGGCGGTAGCCTCCGCCTTA | 543 |
| | GTTGTGCTGCCAGCGGTTACAGCAACTGCGCTTACGCCATGTCGTGGTATCGCCAGGCCCC | |
| | AGGGAAGGAGAGGGAGTTCGTCAGCGCTATCCATTCCGATGGGAGCACACGCTACGCTGAC | |
| | TCCGTGAAAGGGAGGTTCTTTATCTCTCGTGACAACAGCAAAAACACAGTGTACCTCCAGA | |
| | TGAACTCATTGAGACCAGAGGATACAGCTGTGTATTACTGTAAGACAGACCCTCTGCACTG | |
| | TCGCGCCCACGGGGGCTCCTGGTATTCTGTGCGTGCCAACTATTGGGGCCAGGGAACACTG | |
| | GTTACCGTCTCTAGCGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCCTGGTAACTGTCTCGTCTGCTAGCCACCATCACCATCACC | |
| | AC | |
| DR2505 | GAGGTGCAGCTCCTGGAATCTGGCGGGGGCCTGGTCCAGGCTGGCGGTAGCCTCCGCCTTA | 544 |
| | GTTGTGCTGCCAGCGGTTACAGCAACTGCGCTTACGACATGACGTGGTATCGCCAGGCCCC | |
| | AGGGAAGGAGAGGGAGTTCGTCAGCGCTATCTCTGGCAGTGGGAGCACATACTACGCTGAC | |
| | TCCGTGAAAGGGAGGTTCTTTATCTCTCGTGACAACAGCAAAAACACAGTGTACCTCCAGA | |
| | TGAACTCATTGAGACCAGAGGATACAGCTGTGTATTACTGTAAGACAGACCCTCTGCACTG | |
| | TCGCGCCCACGGGGGCTCCTGGTATTCTGTGCGTGCCAACTATTGGGGCCAGGGAACACTG | |
| | GTTACCGTCTCTAGCGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCCTGGTAACTGTCTCGTCTGCTAGCCACCATCACCATCACC | |
| | AC | |
| DR2506 | GAGGTGCAGCTCCTGGAATCTGGCGGGGGCCTGGTCCAGGCTGGCGGTAGCCTCCGCCTTA | 545 |
| | GTTGTGCTGCCAGCGGTTACAGCAACTGCGCTTACGCCATGTCGTGGTATCGCCAGGCCCC | |
| | AGGGAAGGAGAGGGAGTTCGTCAGCGCTATCTCTGGCAGTGGGAGCACATACTACGCTGAC | |
| | TCCGTGAAAGGGAGGTTCTTTATCTCTCGTGACAACAGCAAAAACACAGTGTACCTCCAGA | |
| | TGAACTCATTGAGACCAGAGGATACAGCTGTGTATTACTGTAAGACAGACCCTCTGCACTG | |
| | TCGCGCCCACGGGGGCTCCTGGTATTCTGTGCGTGCCAACTATTGGGGCCAGGGAACACTG | |
| | GTTACCGTCTCTAGCGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCCTGGTAACTGTCTCGTCTGCTAGCCACCATCACCATCACC | |
| | AC | |
| DR1525 | GAGGTCCAGTTGCAGGAGTCCGGTGGCGGGTTGGTCCAAGCAGGCGGAAGCCTCCGCCTTA | 546 |
| | GTTGTGCGGCCTCTGGCTACTCTAACTGCGCCTATGATATGACGTGGTATCGCCAAGCTCC | |
| | AGGCAAAGAAAGAGAGTTCGTCAGCGCCATTCACTCTGATGGCTCCACTAGGTACGCAGAC | |
| | AGCGTCAAGGGTCGTTTTTTCATCTCCCGCGATAACTCCAAGAACACCGTGTACCTCCAAA | |
| | TGAACTCCCTGAGACCGGAGGACACTGCTGTGTACTATTGCAAGACCGATCCTTTGCACTG | |
| | TAGGGCGCATGGAGGCAGTTGGTACTCCGTTAGAGCCAACTACTGGGGGCAAGGAACCCTG | |
| | GTCACTGTCAGCTCCGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCCTGGTAACTGTCTCGTCTGCTAGCCACCATCACCATCACC | |
| | AC | |
| DR2096+ | GAAGTGCAGCTCCTGGAGAGCGGCGGTGGCTCGGTGCAGCCAGGCGGTTCTCTGCGCTTGT | 547 |
| His | CCTGTGCTGCCTCCGGCTACTCAAACTGTGCTTACGATATGACTTGGTATCGCCAGGCCCC | |
| | TGGTAAGGAGCGCGAGTTCGTCTCTGCCATTCATTCTGACGGCTCAACCCGTTATGCAGAC | |
| | TCCGTTAAGGGGCGTTTTTTCATTTCCCGCGACAACTCCAAGAACACCCTGTACCTTCAGA | |
| | TGAACTCCCTCCGCGCCGAGGATACCGCCGTATACTATTGCAAGACCGACCCCCTGCATTG | |
| | CAGGGCTCACGGCGGGTCCTGGTACTCCGTGCGTGCCAACTATTGGGGACAGGGGACTCTG | |
| | GTCACAGTCTCGAGTGGAGGAGGATCCGAGGTGCAGCTGGTGGAGAGCGGAGGAGGGTTAG | |
| | TGCAGCCGGGCGGTTCTCTCAGACTTAGCTGCGCCGCCTCCGGGTACACGCACTCTTCCTA | |
| | TTGCATGGGCTGGGTCAGGCAGGCGCCTGGGAAGGGCCTGGAGGGAGTTAGTGCCATCGAT | |
| | GTCGATGGCTCAACAACCTATGCAGACAGCGTTAAGGGTCGGTTCACCATTTCACGCGACA | |
| | ATAGCAAAAACACTCTGTATTTGCAGATGAACAGTCTCCGTGCCGAAGACACAGCTGTCTA | |
| | CTACTGTGCAGCTGAATTTGCTGATTGTTCCTCTAATTATTTCCTACCCCCAGGGGCAGTG | |
| | CGATACTGGGGACAAGGCACCCTGGTAACTGTCTCGGCAGCTAGCCACCATCACCATCACC | |
| | AC | |
| DR2463 | GAGGTGCAGCTCCTGGAATCTGGCGGGGGCCTGGTCCAGGCTGGCGGTAGCCTCCGCCT | 548 |
| (−His) | TAGTTGTGCTGCCAGCGGTTACAGCAACTGCGCTTACGACATGACGTGGTATCGCCAGG | |
| | CCCCAGGGAAGGAGAGGGAGTTCGTCAGCGCTATCCATTCCGATGGGAGCACACGCTAC | |
| | GCTGACTCCGTGAAAGGGAGGTTCTTTATCTCTCGTGACAACAGCAAAAACACAGTGTA | |
| | CCTCCAGATGAACTCATTGAGACCAGAGGATACAGCTGTGTATTACTGTAAGACAGACC | |
| | CTCTGCACTGTCGCGCCCACGGGGGCTCCTGGTATTCTGTGCGTGCCAACTATTGGGGC | |
| | CAGGGAACACTGGTTACCGTCTCTAGCGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCCTGGTAACTGTCTC | |
| | GTCT | |
| DR2485 | GAGGTGCAGCTCCTGGAATCTGGCGGGGGCCTGGTCCAGGCTGGCGGTAGCCTCCGCCT | 549 |
| | TAGTTGTGCTGCCAGCGGTTACAGCAACTGCGCTTACGACATGACGTGGTATCGCCAGG | |
| | CCCCAGGGAAGGAGAGGGAGTTCGTCAGCGCTATCCATTCCGATGGGAGCACACGCTAC | |
| | GCTGACTCCGTGAAAGGGAGGTTCTTTATCTCTCGTGACAACAGCAAAAACACAGTGTA | |
| | CCTCCAGATGAACTCATTGAGACCAGAGGATACAGCTGTGTATTACTGTAAGACAGACC | |
| | CTCTGCACTGTCGCGCCCACGGGGGCTCCTGGTATTCTGTGCGTGCCAACTATTGGGGC | |
| | CAGGGAACACTGGTTACCGTCTCTAGCGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCACGGTAACTGTCTC | |
| | GTCT | |
| DR2519 | GAGGTGCAGCTCCTGGAATCTGGCGGGGGCCTGGTCCAGGCTGGCGGTAGCCTCCGCCT | 550 |
| | TAGTTGTGCTGCCAGCGGTTACAGCAACTGCGCTTACGACATGACGTGGTATCGCCAGG | |
| | CCCCAGGGAAGGAGAGGGAGTTCGTCAGCGCTATCCATTCCGATGGGAGCACATATTAC | |
| | GCTGACTCCGTGAAAGGGAGGTTCTTTATCTCTCGTGACAACAGCAAAAACACAGTGTA | |
| | CCTCCAGATGAACTCATTGAGACCAGAGGATACAGCTGTGTATTACTGTAAGACAGACC | |
| | CTCTGCACTGTCGCGCCCACGGGGGCTCCTGGTATTCTGTGCGTGCCAACTATTGGGGC | |
| | CAGGGAACACTGGTTACCGTCTCTAGCGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCACGGTAACTGTCTC | |
| | GTCT | |
| DR2520 | GAGGTGCAGCTCCTGGAATCTGGCGGGGGCCTGGTCCAGGCTGGCGGTAGCCTCCGCCT | 551 |
| | TAGTTGTGCTGCCAGCGGTTACAGCAACTGCGCTTACGACATGACGTGGTATCGCCAGG | |
| | CCCCAGGGAAGGAGAGGGAGTTCGTCAGCGCTATCCATTCCTCTGGGAGCACATATTAC | |
| | GCTGACTCCGTGAAAGGGAGGTTCTTTATCTCTCGTGACAACAGCAAAAACACAGTGTA | |
| | CCTCCAGATGAACTCATTGAGACCAGAGGATACAGCTGTGTATTACTGTAAGACAGACC | |
| | CTCTGCACTGTCGCGCCCACGGGGGCTCCTGGTATTCTGTGCGTGCCAACTATTGGGGC | |
| | CAGGGAACACTGGTTACCGTCTCTAGCGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCACGGTAACTGTCTC | |
| | GTCT | |
| DR2457 | GAAGTCCAGCTCCAGGAATCAGGAGGTGGCTCTGTGCAACCCGGTGGCTCCCTGCGCCT | 552 |
| (−His) | CAGCTGTGCCGCTTCTGGATATTCTAACTGCGCTTACGACATGACTTGGTATAGGCAGG | |
| | CACCGGGCAAGGAGCGCGAGTTCGTTTCTGCCATTCACTCTGATGGATCTACCCGCTAT | |
| | GCCGATAGCGTGAAGGGTCGCTTTTTCATCTCCCAGGATAACGCTAAAAATACTGTGTA | |
| | CCTTCAGATGAACAGCCTGAAACCTGAAGACACCGCCATGTACTATTGTAAGACAGACC | |
| | CGTTGCATTGCAGAGCCCACGGAGGCTCATGGTATTCCGTCCGGGCGAACTACTGGGGC | |
| | CAGGGCACTCAGGTGACCGTATCCTCTGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCCTGGTAACTGTCTC | |
| | GTCT | |
| DR2458 | GAGGTTCAACTCCAGGAGAGCGGCGGGGGCCTGGTGCAAGCTGGGGGCAGTCTGCGCTT | 553 |
| (−His) | GAGCTGCGCTGCATCCGGCTATTCCAACTGTGCCTATGACATGACGTGGTATAGGCAGG | |
| | CCCCTGGGAAGGAGCGTGAGTTCGTGAGTGCTATCCACAGCGACGGCTCCACTAGATAC | |
| | GCGGACTCCGTTAAAGGAAGATTTTTCATCAGTCAGGACAACAGCAAGAATACCGTGTA | |
| | TTTGCAGATGAATAGTCTGAAGCCCGAGGACACAGCAGTCTACTATTGTAAGACTGACC | |
| | CACTGCACTGTCGTGCGCATGGAGGCTCTTGGTACTCCGTGAGAGCCAATTATTGGGGG | |
| | CAGGGCACCCTTGTTACCGTATCCTCCGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCCTGGTAACTGTCTC | |
| | GTCT | |
| DR2459 | GAGGTGCAGCTCCAGGAGAGCGGCGGTGGCCTCGTCCAAGCCGGGGGCTCCCTGCGTTT | 554 |
| (−His) | GTCTTGCGCGGCCAGCGGATATTCCAACTGTGCGTATGACATGACATGGTATCGCCAGG | |
| | CTCCAGGCAAGGAGCGTGAGTTTGTGTCTGCAATCCACTCTGATGGCTCCACACGCTAC | |
| | GCCGACAGCGTGAAGGGCCGCTTCTTTATCTCCAGAGATAACAGCAAGAACACTGTCTA | |
| | TCTCCAGATGAACAGCCTGAAGCCCGAAGACACCGCCGTTTATTACTGTAAGACCGACC | |
| | CTCTGCATTGCAGAGCCCACGGCGGTTCCTGGTATTCCGTCCGCGCTAACTATTGGGGA | |
| | CAGGGGACCCTTGTGACAGTCAGCTCCGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCCTGGTAACTGTCTC | |
| | GTCT | |
| DR2460 | GAGGTGCAGTTGCTGGAGTCTGGTGGGGGCCTCGTGCAGGCCGGAGGCTCCCTGCGCCT | 555 |
| (−His) | GTCCTGCGCTGCCTCTGGATACTCTAATTGCGCATACGACATGACATGGTATCGGCAGG | |
| | CACCCGGAAAGGAGCGCGAGTTCGTATCAGCAATCCATAGTGACGGTTCTACTCGCTAT | |
| | GCAGACTCTGTGAAAGGACGCTTCTTTATTAGCCAGGACAACTCTAAAAACACTGTCTA | |
| | TCTCCAAATGAACTCCCTCAAGCCAGAAGATACTGCCGTGTATTACTGCAAGACCGACC | |
| | CACTGCATTGTCGTGCGCATGGAGGGTCTTGGTATTCAGTACGTGCAAACTACTGGGGA | |
| | CAGGGCACCCTGGTCACTGTGTCCTCTGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCCTGGTAACTGTCTC | |
| | GTCT | |
| DR2461 | GAGGTACAGCTCTTGGAGTCCGGCGGTGGCTTGGTGCAGGCTGGAGGTAGCTTGCGCCT | 556 |
| (−His) | GTCCTGCGCCGCTTCCGGCTACAGCAACTGCGCCTACGACATGACGTGGTATCGGCAGG | |
| | CTCCAGGTAAGGAGCGCGAGTTCGTCAGTGCCATTCACTCCGACGGGAGCACTAGATAT | |
| | GCCGACTCTGTCAAGGGACGTTTTTTCATTAGTCGCGACAACTCTAAGAACACCGTGTA | |
| | CCTTCAGATGAACAGCCTGAAGCCCGAGGACACTGCCGTGTACTATTGCAAGACCGACC | |
| | CCCTCCATTGCAGGGCTCATGGGGGCTCTTGGTATTCAGTGAGAGCCAACTATTGGGGA | |
| | CAGGGTACGCTGGTGACCGTGTCATCTGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCCTGGTAACTGTCTC | |
| | GTCT | |
| DR2462 | GAGGTCCAGTTGCAGGAGTCCGGTGGCGGGTTGGTCCAAGCAGGCGGAAGCCTCCGCCT | 557 |
| | TAGTTGTGCGGCCTCTGGCTACTCTAACTGCGCCTATGATATGACGTGGTATCGCCAAG | |
| | CTCCAGGCAAAGAAAGAGAGTTCGTCAGCGCCATTCACTCTGATGGCTCCACTAGGTAC | |
| | GCAGACAGCGTCAAGGGTCGTTTTTTCATCTCCCGCGATAACTCCAAGAACACCGTGTA | |
| | CCTCCAAATGAACTCCCTGAGACCGGAGGACACTGCTGTGTACTATTGCAAGACCGATC | |
| | CTTTGCACTGTAGGGCGCATGGAGGCAGTTGGTACTCCGTTAGAGCCAACTACTGGGGG | |
| | CAAGGAACCCTGGTCACTGTCAGCTCCGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCCTGGTAACTGTCTC | |
| | GTCT | |
| DR2464 | GAGGTTCAGTTGCTGGAATCTGGGGGCGGTCTGGTCCAGGCTGGGGGCTCTCTGAGGCT | 558 |
| | GAGCTGCGCCGCATCTGGCTACTCCAACTGCGCCTATGATATGACTTGGTACAGGCAGG | |
| | CTCCCGGCAAGGAGCGCGAGTTCGTGAGCGCCATTCACTCTGATGGTTCCACAAGGTAT | |
| | GCTGATAGTGTTAAAGGACGCTTTTTCATTAGCCGGGACAATAGCAAAAACACAGTCTA | |
| | TCTCCAGATGAACTCCCTCCGGGCGGAGGATACCGCTGTCTATTACTGCAAGACTGACC | |
| | CTTTGCACTGTAGAGCACACGGAGGCAGCTGGTATTCTGTGAGAGCCAACTATTGGGGA | |
| | CAAGGAACCCTCGTCACGGTGTCTAGCGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCCTGGTAACTGTCTC | |
| | GTCT | |
| DR2465 | GAGGTGCAGCTCCTGGAGTCTGGCGGTGGCCTGGTGCAAGCTGGCGGTTCCCTTCGTCT | 559 |
| | GTCCTGCGCAGCCAGTGGCTATTCTAACTGTGCTTACGACATGACATGGTATCGTCAGG | |
| | CACCGGGGAAGGAGCGCGAGTTCGTATCAGCTATCCACTCTGACGGCAGTACCAGATAT | |
| | GCCGACAGCGTCAAAGGGCGTTTTTTCATCTCCCGCGACAACTCCAAAAATACCCTGTA | |
| | TCTTCAGATGAACTCCCTCAGGCCTGAGGACACTGCCGTCTATTACTGCAAGACTGACC | |
| | CCCTCCACTGTCGCGCTCATGGGGGTAGTTGGTACTCTGTGCGTGCTAATTACTGGGGG | |
| | CAGGGCACCCTGGTGACCGTTAGCTCAGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCCTGGTAACTGTCTC | |
| | GTCT | |
| DR2466 | GAGGTGCAGTTGCTGGAGAGCGGGGGCGGGCTCGTGCAGGCGGGAGGCAGCTTGCGCTT | 560 |
| | GTCATGTGCTGCCAGCGGTTATAGCAACTGCGCCTACGACATGACATGGTATCGCCAGG | |
| | CTCCCGGTAAAGAGAGGGAGTTTGTATCCGCGATCCACAGTGACGGTAGCACCCGCTAC | |
| | GCGGATTCCGTGAAAGGACGTTTCTTTATTTCTAGGGATAATAGCAAGAATACACTGTA | |
| | CTTGCAAATGAACTCCCTCCGCGCCGAGGACACTGCTGTGTATTACTGCAAAACTGATC | |
| | CACTGCACTGTCGCGCTCACGGGGGTTCTTGGTATTCAGTACGCGCCAACTACTGGGGC | |
| | CAGGGTACTCTCGTGACGGTGAGCAGCGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCCTGGTAACTGTCTC | |
| | GTCT | |
| DR2499 | GAGGTGCAGCTCCTGGAATCTGGCGGGGGCCTGGTCCAGGCTGGCGGTAGCCTCCGCCT | 561 |
| | TAGTTGTGCTGCCAGCGGTTACAGCAACTGCGCTTACGCCATGACGTGGTATCGCCAGG | |
| | CCCCAGGGAAGGAGAGGGAGTTCGTCAGCGCTATCCATTCCGATGGGAGCACACGCTAC | |
| | GCTGACTCCGTGAAAGGGAGGTTCTTTATCTCTCGTGACAACAGCAAAAACACAGTGTA | |
| | CCTCCAGATGAACTCATTGAGACCAGAGGATACAGCTGTGTATTACTGTAAGACAGACC | |
| | CTCTGCACTGTCGCGCCCACGGGGGCTCCTGGTATTCTGTGCGTGCCAACTATTGGGGC | |
| | CAGGGAACACTGGTTACCGTCTCTAGCGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCCTGGTAACTGTCTC | |
| | GTCT | |
| DR2500 | GAGGTGCAGCTCCTGGAATCTGGCGGGGGCCTGGTCCAGGCTGGCGGTAGCCTCCG | 562 |
| | CCTTAGTTGTGCTGCCAGCGGTTACAGCAACTGCGCTTACGACATGTCGTGGTATC | |
| | GCCAGGCCCCAGGGAAGGAGAGGGAGTTCGTCAGCGCTATCCATTCCGATGGGAGC | |
| | ACACGCTACGCTGACTCCGTGAAAGGGAGGTTCTTTATCTCTCGTGACAACAGCAA | |
| | AAACACAGTGTACCTCCAGATGAACTCATTGAGACCAGAGGATACAGCTGTGTATT | |
| | ACTGTAAGACAGACCCTCTGCACTGTCGCGCCCACGGGGGCTCCTGGTATTCTGTG | |
| | CGTGCCAACTATTGGGGCCAGGGAACACTGGTTACCGTCTCTAGCGGAGGAGGATC | |
| | CGAGGTGCAGCTGGTGGAGAGCGGAGGAGGGTTAGTGCAGCCGGGCGGTTCTCTCA | |
| | GACTTAGCTGCGCCGCCTCCGGGTACACGCACTCTTCCTATTGCATGGGCTGGGTC | |
| | AGGCAGGCGCCTGGGAAGGGCCTGGAGGGAGTTAGTGCCATCGATGTCGATGGCTC | |
| | AACAACCTATGCAGACAGCGTTAAGGGTCGGTTCACCATTTCACGCGACAATAGCA | |
| | AAAACACTCTGTATTTGCAGATGAACAGTCTCCGTGCCGAAGACACAGCTGTCTAC | |
| | TACTGTGCAGCTGAATTTGCTGATTGTTCCTCTAATTATTTCCTACCCCCAGGGGC | |
| | AGTGCGATACTGGGGACAAGGCACCCTGGTAACTGTCTCGTCT | |
| DR2501 | GAGGTGCAGCTCCTGGAATCTGGCGGGGGCCTGGTCCAGGCTGGCGGTAGCCTCCGCCT | 563 |
| | TAGTTGTGCTGCCAGCGGTTACAGCAACTGCGCTTACGACATGACGTGGTATCGCCAGG | |
| | CCCCAGGGAAGGAGAGGGAGTTCGTCAGCGCTATCTCTTCCGATGGGAGCACACGCTAC | |
| | GCTGACTCCGTGAAAGGGAGGTTCTTTATCTCTCGTGACAACAGCAAAAACACAGTGTA | |
| | CCTCCAGATGAACTCATTGAGACCAGAGGATACAGCTGTGTATTACTGTAAGACAGACC | |
| | CTCTGCACTGTCGCGCCCACGGGGGCTCCTGGTATTCTGTGCGTGCCAACTATTGGGGC | |
| | CAGGGAACACTGGTTACCGTCTCTAGCGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCCTGGTAACTGTCTC | |
| | GTCT | |
| DR0000 | GAGGTGCAGCTCCTGGAATCTGGCGGGGGCCTGGTCCAGGCTGGCGGTAGCCTCCGCCT | 564 |
| | TAGTTGTGCTGCCAGCGGTTACAGCAACTGCGCTTACGACATGACGTGGTATCGCCAGG | |
| | CCCCAGGGAAGGAGAGGGAGTTCGTCAGCGCTATCCATGGCGATGGGAGCACACGCTAC | |
| | GCTGACTCCGTGAAAGGGAGGTTCTTTATCTCTCGTGACAACAGCAAAAACACAGTGTA | |
| | CCTCCAGATGAACTCATTGAGACCAGAGGATACAGCTGTGTATTACTGTAAGACAGACC | |
| | CTCTGCACTGTCGCGCCCACGGGGGCTCCTGGTATTCTGTGCGTGCCAACTATTGGGGC | |
| | CAGGGAACACTGGTTACCGTCTCTAGCGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCCTGGTAACTGTCTC | |
| | GTCT | |
| DR2502 | GAGGTGCAGCTCCTGGAATCTGGCGGGGGCCTGGTCCAGGCTGGCGGTAGCCTCCGCCT | 565 |
| | TAGTTGTGCTGCCAGCGGTTACAGCAACTGCGCTTACGACATGACGTGGTATCGCCAGG | |
| | CCCCAGGGAAGGAGAGGGAGTTCGTCAGCGCTATCCATTCCAGTGGGAGCACACGCTAC | |
| | GCTGACTCCGTGAAAGGGAGGTTCTTTATCTCTCGTGACAACAGCAAAAACACAGTGTA | |
| | CCTCCAGATGAACTCATTGAGACCAGAGGATACAGCTGTGTATTACTGTAAGACAGACC | |
| | CTCTGCACTGTCGCGCCCACGGGGGCTCCTGGTATTCTGTGCGTGCCAACTATTGGGGC | |
| | CAGGGAACACTGGTTACCGTCTCTAGCGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCCTGGTAACTGTCTC | |
| | GTCT | |
| DR2503 | GAGGTGCAGCTCCTGGAATCTGGCGGGGGCCTGGTCCAGGCTGGCGGTAGCCTCCGCCT | 566 |
| | TAGTTGTGCTGCCAGCGGTTACAGCAACTGCGCTTACGACATGACGTGGTATCGCCAGG | |
| | CCCCAGGGAAGGAGAGGGAGTTCGTCAGCGCTATCCATTCCGATGGGAGCACATACTAC | |
| | GCTGACTCCGTGAAAGGGAGGTTCTTTATCTCTCGTGACAACAGCAAAAACACAGTGTA | |
| | CCTCCAGATGAACTCATTGAGACCAGAGGATACAGCTGTGTATTACTGTAAGACAGACC | |
| | CTCTGCACTGTCGCGCCCACGGGGGCTCCTGGTATTCTGTGCGTGCCAACTATTGGGGC | |
| | CAGGGAACACTGGTTACCGTCTCTAGCGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCCTGGTAACTGTCTC | |
| | GTCT | |
| DR2504 | GAGGTGCAGCTCCTGGAATCTGGCGGGGGCCTGGTCCAGGCTGGCGGTAGCCTCCGCCT | 567 |
| | TAGTTGTGCTGCCAGCGGTTACAGCAACTGCGCTTACGCCATGTCGTGGTATCGCCAGG | |
| | CCCCAGGGAAGGAGAGGGAGTTCGTCAGCGCTATCCATTCCGATGGGAGCACACGCTAC | |
| | GCTGACTCCGTGAAAGGGAGGTTCTTTATCTCTCGTGACAACAGCAAAAACACAGTGTA | |
| | CCTCCAGATGAACTCATTGAGACCAGAGGATACAGCTGTGTATTACTGTAAGACAGACC | |
| | CTCTGCACTGTCGCGCCCACGGGGGCTCCTGGTATTCTGTGCGTGCCAACTATTGGGGC | |
| | CAGGGAACACTGGTTACCGTCTCTAGCGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCCTGGTAACTGTCTC | |
| | GTCT | |
| DR2505 | GAGGTGCAGCTCCTGGAATCTGGCGGGGGCCTGGTCCAGGCTGGCGGTAGCCTCCGCCT | 568 |
| | TAGTTGTGCTGCCAGCGGTTACAGCAACTGCGCTTACGACATGACGTGGTATCGCCAGG | |
| | CCCCAGGGAAGGAGAGGGAGTTCGTCAGCGCTATCTCTGGCAGTGGGAGCACATACTAC | |
| | GCTGACTCCGTGAAAGGGAGGTTCTTTATCTCTCGTGACAACAGCAAAAACACAGTGTA | |
| | CCTCCAGATGAACTCATTGAGACCAGAGGATACAGCTGTGTATTACTGTAAGACAGACC | |
| | CTCTGCACTGTCGCGCCCACGGGGGCTCCTGGTATTCTGTGCGTGCCAACTATTGGGGC | |
| | CAGGGAACACTGGTTACCGTCTCTAGCGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCCTGGTAACTGTCTC | |
| | GTCT | |
| DR2506 | GAGGTGCAGCTCCTGGAATCTGGCGGGGGCCTGGTCCAGGCTGGCGGTAGCCTCCGCCT | 569 |
| | TAGTTGTGCTGCCAGCGGTTACAGCAACTGCGCTTACGCCATGTCGTGGTATCGCCAGG | |
| | CCCCAGGGAAGGAGAGGGAGTTCGTCAGCGCTATCTCTGGCAGTGGGAGCACATACTAC | |
| | GCTGACTCCGTGAAAGGGAGGTTCTTTATCTCTCGTGACAACAGCAAAAACACAGTGTA | |
| | CCTCCAGATGAACTCATTGAGACCAGAGGATACAGCTGTGTATTACTGTAAGACAGACC | |
| | CTCTGCACTGTCGCGCCCACGGGGGCTCCTGGTATTCTGTGCGTGCCAACTATTGGGGC | |
| | CAGGGAACACTGGTTACCGTCTCTAGCGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCCTGGTAACTGTCTC | |
| | GTCT | |
| DR1525 | GAGGTCCAGTTGCAGGAGTCCGGTGGCGGGTTGGTCCAAGCAGGCGGAAGCCTCCGCCT | 570 |
| | TAGTTGTGCGGCCTCTGGCTACTCTAACTGCGCCTATGATATGACGTGGTATCGCCAAG | |
| | CTCCAGGCAAAGAAAGAGAGTTCGTCAGCGCCATTCACTCTGATGGCTCCACTAGGTAC | |
| | GCAGACAGCGTCAAGGGTCGTTTTTTCATCTCCCGCGATAACTCCAAGAACACCGTGTA | |
| | CCTCCAAATGAACTCCCTGAGACCGGAGGACACTGCTGTGTACTATTGCAAGACCGATC | |
| | CTTTGCACTGTAGGGCGCATGGAGGCAGTTGGTACTCCGTTAGAGCCAACTACTGGGGG | |
| | CAAGGAACCCTGGTCACTGTCAGCTCCGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCCTGGTAACTGTCTC | |
| | GTCT | |
| DR2096 | GAAGTGCAGCTCCTGGAGAGCGGCGGTGGCTCGGTGCAGCCAGGCGGTTCTCTGCGCTT | 571 |
| | GTCCTGTGCTGCCTCCGGCTACTCAAACTGTGCTTACGATATGACTTGGTATCGCCAGG | |
| | CCCCTGGTAAGGAGCGCGAGTTCGTCTCTGCCATTCATTCTGACGGCTCAACCCGTTAT | |
| | GCAGACTCCGTTAAGGGGCGTTTTTTCATTTCCCGCGACAACTCCAAGAACACCCTGTA | |
| | CCTTCAGATGAACTCCCTCCGCGCCGAGGATACCGCCGTATACTATTGCAAGACCGACC | |
| | CCCTGCATTGCAGGGCTCACGGCGGGTCCTGGTACTCCGTGCGTGCCAACTATTGGGGA | |
| | CAGGGGACTCTGGTCACAGTCTCGAGTGGAGGAGGATCCGAGGTGCAGCTGGTGGAGAG | |
| | CGGAGGAGGGTTAGTGCAGCCGGGCGGTTCTCTCAGACTTAGCTGCGCCGCCTCCGGGT | |
| | ACACGCACTCTTCCTATTGCATGGGCTGGGTCAGGCAGGCGCCTGGGAAGGGCCTGGAG | |
| | GGAGTTAGTGCCATCGATGTCGATGGCTCAACAACCTATGCAGACAGCGTTAAGGGTCG | |
| | GTTCACCATTTCACGCGACAATAGCAAAAACACTCTGTATTTGCAGATGAACAGTCTCC | |
| | GTGCCGAAGACACAGCTGTCTACTACTGTGCAGCTGAATTTGCTGATTGTTCCTCTAAT | |
| | TATTTCCTACCCCCAGGGGCAGTGCGATACTGGGGACAAGGCACCCTGGTAACTGTCTC | |
| | GGCA | |

1. An IL-10 agonist compound comprising a first single-domain antibody polypeptide joined to a second single-domain antibody polypeptide, wherein
the first single-domain antibody polypeptide specifically binds to the α subunit of the IL10 receptor (IL10Rα) and comprises:
a CDR1 comprising an amino acid sequence selected from the group consisting of SEQ ID NOS:224-228;
a CDR2 comprising an amino acid sequence selected from the group consisting of SEQ ID NO:229-235; and
a CDR3 comprising an amino acid sequence selected from the group consisting of SEQ ID NO:236; and
wherein the second single-domain antibody polypeptide specifically binds to the R subunit of the IL10 receptor (IL10Rβ) and comprises:
a CDR1 comprising an amino acid sequence selected from the group consisting of SEQ ID NO:296;
a CDR2 comprising an amino acid sequence selected from the group consisting of SEQ ID NO:297; and
a CDR3 comprising an amino acid sequence selected from the group consisting of SEQ ID NO:298.

2. The IL-10 agonist compound of claim 1,
wherein the first single-domain antibody polypeptide comprises a combination of CDR1, CDR2, and CDR3 amino acid sequences selected from one of the rows of the following table:
IL-10Rα VHH CDRs

| CDR1 SEQ ID NO: | CDR2 SEQ ID NO: | CDR3 SEQ ID NO: |
|---|---|---|
| 224 | 229 | 236 |
| 224 | 230 | 236 |
| 224 | 231 | 236 |
| 225 | 229 | 236 |
| 226 | 229 | 236 |
| 224 | 232 | 236 |
| 224 | 233 | 236 |
| 224 | 234 | 236 |
| 227 | 229 | 236 |
| 224 | 235 | 236 |
| 227 | 235 | 236 |
| 228 | 229 | 236 |

and wherein the second single-domain antibody polypeptide comprises a combination of CDR1, CDR2, and CDR3 amino acid sequences selected from one of the rows of the following table:
IL-10Rβ VHH CDRs

| CDR1 SEQ ID NO: | CDR2 SEQ ID NO: | CDR3 SEQ ID NO: |
|---|---|---|
| 296 | 297 | 298 |

3. The IL-10 agonist compound of claim 2, wherein the first single-domain antibody polypeptide comprises
a CDR1 comprising the amino acid sequence of SEQ ID NO:224;
a CDR2 comprising the amino acid sequence of SEQ ID NO:229; and
a CDR3 comprising the amino acid sequence of SEQ ID NO:236; and
wherein the second single-domain antibody polypeptide comprises
a CDR1 comprising the amino acid sequence of SEQ ID NO:296;
a CDR2 comprising the amino acid sequence of SEQ ID NO:297; and
a CDR3 comprising the amino acid sequence of SEQ ID NO:298.

4. The IL-10 agonist compound of claim 2, wherein the first single-domain antibody polypeptide comprises
a CDR1 comprising the amino acid sequence of SEQ ID NO:224;
a CDR2 comprising the amino acid sequence of SEQ ID NO:230; and
a CDR3 comprising the amino acid sequence of SEQ ID NO:236; and
wherein the second single-domain antibody polypeptide comprises
a CDR1 comprising the amino acid sequence of SEQ ID NO:296;
a CDR2 comprising the amino acid sequence of SEQ ID NO:297; and
a CDR3 comprising the amino acid sequence of SEQ ID NO:298.

5. The IL-10 agonist compound of claim 2, wherein the first single-domain antibody polypeptide comprises
a CDR1 comprising the amino acid sequence of SEQ ID NO:224;
a CDR2 comprising the amino acid sequence of SEQ ID NO:231; and
a CDR3 comprising the amino acid sequence of SEQ ID NO:236; and
wherein the second single-domain antibody polypeptide comprises
a CDR1 comprising the amino acid sequence of SEQ ID NO:296;
a CDR2 comprising the amino acid sequence of SEQ ID NO:297; and
a CDR3 comprising the amino acid sequence of SEQ ID NO:298.

6. The IL-10 agonist compound of any one of claims 1 to 5, comprising:
a first single-domain antibody polypeptide comprising an amino acid sequence selected from the group consisting of SEQ ID NOS:25-48; and
a second single-domain antibody polypeptide comprising an amino acid sequence selected from the group consisting of SEQ ID NO:49 and 50.

7. The IL-10 agonist compound of any one of claims 1 to 6, wherein the first single-domain antibody polypeptide and second single-domain antibody polypeptide are joined by a linker.

8. The IL-10 agonist compound of any one of claims 1 to 7, wherein the C-terminus of the first single-domain antibody polypeptide is joined to the N-terminus of the second single-domain antibody polypeptide, optionally wherein the C-terminus of the first single-domain antibody polypeptide is joined to the N-terminus of the second single-domain antibody polypeptide via a linker.

9. The IL-10 agonist compound of any one of claims 1 to 7, wherein the N-terminus of the first single-domain antibody polypeptide is joined to the C-terminus of the second single-domain antibody polypeptide, optionally wherein the N-terminus of the first single-domain antibody polypeptide is joined to the C-terminus of the second single-domain antibody polypeptide via a linker.

10. The IL-10 agonist compound of any one of claims 1 to 9, comprising a polypeptide comprising at least 95% amino acid sequence identity to an amino acid sequence selected from the group consisting of SEQ ID NOS:1-24 or 500-523.

11. The IL-10 agonist compound of claim 10, comprising a polypeptide comprising an amino acid sequence selected from the group consisting of SEQ ID NOS:1-24 or 500-523.

12. The IL-10 agonist compound of claim 11, comprising a polypeptide comprising the amino acid sequence of SEQ ID NO:500.

13. The IL-10 agonist compound of claim 11, comprising a polypeptide comprising the amino acid sequence of SEQ ID NO:501.

14. The IL-10 agonist compound of claim 11, comprising a polypeptide comprising the amino acid sequence of SEQ ID NO:502.

15. The IL-10 agonist compound of claim 11, comprising a polypeptide comprising the amino acid sequence of SEQ ID NO:503.

16. The IL-10 agonist compound of claim 11, comprising a polypeptide comprising the amino acid sequence of SEQ ID NO:504.

17. The IL-10 agonist compound of any one of claims 1 to 16, wherein the IL-10 agonist compound comprises a C-terminal amino acid modification that reduces immunogenicity resulting from pre-existing antibodies.

18. The IL-10 agonist compound of any one of claims 1 to 17, wherein the IL-10 agonist compound comprises a C-terminal amino acid modification and a C-terminal amino acid sequence selected from the group consisting of SEQ ID NOS:474-499.

19. The compound of any one of claims 1 to 18, wherein the compound is selected from the group consisting of SEQ ID NOS: 121-135, 138-141, 144-155, 158-175, 179, 182-195, 199.

20. An IL-10 agonist compound comprising an IL-10R binding molecule polypeptide of the following formula #1: 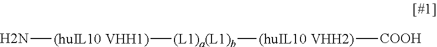
wherein:
“-” represents a covalent bond;
L1 is a linker;
a and b are independently selected from the integers 0 or 1;
“H2N” denotes the amino terminus; and
“COOH” denotes the carboxy terminus of the polypeptide.

21. A pharmaceutically acceptable formulation of an IL-10 agonist compound of any of claims 1-20.

22. A nucleic acid sequence encoding an IL-10 agonist compound of any of claims 1-20.

23. A recombinant vector comprising a nucleic acid of claim 22.

24. A method of treating a mammalian subject suffering from an autoimmune disease, infectious disease, or inflammatory disease by the administration of a therapeutically effective amount of an IL-10 agonist compound of any of claims 1-20.

25. A method of treating a mammalian subject suffering from a neoplastic disease by the administration of a therapeutically effective amount of an IL-10 agonist compound of any of claims 1-20.

26. A composition comprising:
means for inducing intracellular signaling in a cell expressing IL-10Rα and IL-10Rβ, wherein the means comprises (i) a single-domain antibody that binds to IL-10Rα (IL-10Rα sdAb) and is humanized relative to UniProt V3-23 (UniProt No. P01764), joined to (ii) a single-domain antibody that binds to IL-10Rβ (IL-10R sdAb) and that is humanized relative to Uniprot VH3-66 (UniProt A0A0C4DH42); and
a pharmaceutically acceptable carrier.

27. The composition of claim 26, wherein the cell expressing IL-10Rα and IL-10Rβ is a monocyte.

28. The composition of claim 26, wherein dimerizing the extracellular domains of an IL-10Rα subunit and an IL-10Rβ subunit of the IL-10 receptor (IL-10R) on a cell induces pSTAT3 signaling.

29. The composition of claim 26, wherein the intracellular pSTAT-3 signaling in the monocyte cell expressing IL10Rα and IL10R is greater than the pSTAT-3 signaling in a T-cell expressing IL10Rα and IL10Rβ.
